SEQUENCE LISTING (148 sequences)

SEQ ID NO: 1 - DNA, 145 bp, Adeno-associated virus 2
  source 1..145: mol_type = genomic DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 2 - DNA, 113 bp, synthetic construct
  misc_feature 1..113: note = Synthetic polynucleotide
  source 1..113: mol_type = other DNA
tccctctctgcgcgctcgctcgctcactgaggccgcccgggcaaagcccgggcgtcgggcgacctttggtcgcccggcctcagtgagcgagcgagcgcgcagagagggagtgg

SEQ ID NO: 3 - DNA, 138 bp, synthetic construct
  misc_feature 1..138: note = Synthetic polynucleotide
  source 1..138: mol_type = other DNA
tccctctctgcgcgctcgctcgctcactgaggccgcccgggcaaagcccgggcgtcgggcgacctttggtcgcccggcctcagtgagcgagcgagcgcgcagagagggagtggactagctccatcactaggggttcct

SEQ ID NO: 4 - DNA, 20 bp, synthetic construct
  misc_feature 1..20: note = Synthetic oligonucleotide
  source 1..20: mol_type = other DNA
ctccatcactaggggttcct

SEQ ID NO: 5 - DNA, 145 bp, Adeno-associated virus 2
  source 1..145: mol_type = genomic DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 6 - protein, 736 aa, Adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 7 - protein, 735 aa, Adeno-associated virus 2 [sequence omitted]

SEQ ID NO: 8 - protein, 736 aa, Adeno-associated virus 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 9 - protein, 736 aa, Adeno-associated virus 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 10 - protein, 734 aa, Adeno-associated virus 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 11 - protein, 724 aa, Adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 12 - protein, 736 aa, Adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 13 - protein, 737 aa, Adeno-associated virus 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 14 - protein, 738 aa, Adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 15 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - protein, 738 aa, Adeno-associated virus 10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 17 - protein, 733 aa, Adeno-associated virus 11
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 18 - protein, 742 aa, Adeno-associated virus 12
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 19 - protein, 733 aa, Adeno-associated virus 13
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQERLQEDTSFGGNLGRAVFQAKKRILEPLGLVEEAAKTAPGKKRPVEQSPAEPDSSSGIGKSGQQPARKRLNFGQTGDTESVPDPQPLGQPPAAPSGVGSTTMASGGGAPMADNNEGADGVGNSSGNWHCDSQWLGDRVITTSTRTWALPTYNNHLYKQISSQSGATNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYHLNGRDSLVNPGPAMASHKDDKEKFFPMHGTLIFGKEGTNANNADLENVMITDEEEIRTTNPVATEQYGTVSNNLQNSNAGPTTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 20 - protein, 737 aa, synthetic construct
  REGION 1..737: note = Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 21 - protein, 743 aa, synthetic construct
  REGION 1..743: note = Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 22 - protein, 736 aa, synthetic construct
  REGION 1..736: note = Synthetic polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGSNTMAAGGGAPMADNNEGADGVGNASGNWHCDSTWLGDRVITTSTRTWALPTYNNHLYKQISSQSGGSTNDNTYFGYSTPWGYFDFNRFHCHFSPRDWQRLINNNWGFRPKKLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTQTTSGTAGNRTLQFSQAGPSSMANQAKNWLPGPCYRQQRVSKTTNQNNNSNFAWTGATKYHLNGRDSLVNPGPAMATHKDDEDKFFPMSGVLIFGKQGAGNSNVDLDNVMITNEEEIKTTNPVATEEYGTVATNLQSANTAPATGTVNSQGALPGMVWQDRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 23 - protein, 736 aa, synthetic construct; identical to SEQ ID NO: 22
  REGION 1..736: note = Synthetic polypeptide

SEQ ID NO: 24 - protein, 745 aa, synthetic construct
  REGION 1..745: note = Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVTTTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 25 - DNA, 143 bp, Adeno-associated virus 1
  source 1..143: mol_type = genomic DNA
ttgcccactccctctctgcgcgctcgctcgctcggtggggcctgcggaccaaaggtccgcagacggcagagctctgctctgccggccccaccgagcgagcgagcgcgcagagagggagtgggcaactccatcactaggggtaa

SEQ ID NO: 26 - DNA, 143 bp, Adeno-associated virus 1
  source 1..143: mol_type = genomic DNA
ttacccctagtgatggagttgcccactccctctctgcgcgctcgctcgctcggtggggcctgcggaccaaaggtccgcagacggcagagctctgctctgccggccccaccgagcgagcgagcgcgcagagagggagtgggcaa

SEQ ID NO: 27 - DNA, 145 bp, Adeno-associated virus 2
  source 1..145: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 28 - DNA, 145 bp, Adeno-associated virus 2
  source 1..145: mol_type = genomic DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 29 - DNA, 143 bp, Adeno-associated virus 3
  source 1..143: mol_type = genomic DNA
ttggccactccctctatgcgcactcgctcgctcggtggggcctggcgaccaaaggtcgccagacggacgtgctttgcacgtccggccccaccgagcgagcgagtgcgcatagagggagtggccaactccatcactagaggtat

SEQ ID NO: 30 - DNA, 143 bp, Adeno-associated virus 3
  source 1..143: mol_type = genomic DNA
atacctctagtgatggagttggccactccctctatgcgcactcgctcgctcggtggggcctggcgaccaaaggtcgccagacggacgtgctttgcacgtccggccccaccgagcgagcgagtgcgcatagagggagtggccaa

SEQ ID NO: 31 - DNA, 146 bp, Adeno-associated virus 4
  source 1..146: mol_type = genomic DNA
ttggccactccctctatgcgcgctcgctcactcactcggccctggagaccaaaggtctccagactgccggcctctggccggcagggccgagtgagtgagcgagcgcgcatagagggagtggccaactccatcatctaggtttgccc

SEQ ID NO: 32 - DNA, 144 bp, Adeno-associated virus 4
  source 1..144: mol_type = genomic DNA
ggcaaaccagatgatggagttggccacattagctatgcgcgctcgctcactcactcggccctggagaccaaaggtctccagactgccggcctctggccggcagggccgagtgagtgagcgagcgcgcatagagggagtggccaa

SEQ ID NO: 33 - DNA, 167 bp, Adeno-associated virus 5
  source 1..167: mol_type = genomic DNA
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgta

SEQ ID NO: 34 - DNA, 167 bp, Adeno-associated virus 5
  source 1..167: mol_type = genomic DNA
tacaaaacctccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacaggggggagag

SEQ ID NO: 35 - DNA, 145 bp, Adeno-associated virus 6; identical to SEQ ID NO: 27
  source 1..145: mol_type = genomic DNA

SEQ ID NO: 36 - DNA, 143 bp, Adeno-associated virus 6
  source 1..143: mol_type = genomic DNA
atacccctagtgatggagttgcccactccctctatgcgcgctcgctcgctcggtggggccggcagagcagagctctgccgtctgcggacctttggtccgcaggccccaccgagcgagcgagcgcgcatagagggagtgggcaa

SEQ ID NO: 37 - DNA, 145 bp, Adeno-associated virus 7
  source 1..145: mol_type = genomic DNA
ttggccactccctctatgcgcgctcgctcgctcggtggggcctgcggaccaaaggtccgcagacggcagagctctgctctgccggccccaccgagcgagcgagcgcgcatagagggagtggccaactccatcactaggggtaccg

SEQ ID NO: 38 - DNA, 145 bp, Adeno-associated virus 7
  source 1..145: mol_type = genomic DNA
cggtacccctagtgatggagttggccactccctctatgcgcgctcgctcgctcggtggggcctgcggaccaaaggtccgcagacggcagagctctgctctgccggccccaccgagcgagcgagcgcgcatagagggagtggccaa

SEQ ID NO: 39 - DNA, 80 bp, synthetic construct
  misc_feature 1..80: note = Synthetic oligonucleotide
  source 1..80: mol_type = other DNA
gccacggcggaactcatcgccgcctgccttgcccgctgctggacaggggctcggctgttgggcactgacaattccgtggt

SEQ ID NO: 40 - protein, 289 aa, synthetic construct
  REGION 1..289: note = Synthetic polypeptide
MPLIFKIGYNVIPLQDVILPTPSSKVLKYLIQSGKLIPSLKDLITSRDKYKPIFISHLGFNQRRIFQTNGNLKTITKGSRLSSIIAFSTQANVLSEVADEGIFETVYGKFHIMIESIEIVEVEKLKEEVEKHMNDNIRVRFVSPTLLSSKVLLPPSLSERYKKIHAGYSTLPSVGLIVAYAYNVYCNLIGKKEVEVRAFKFGILSNALSRIIGYDLHPVTVAIGEDSKGNLRKARGVMGWIEFDIPDERLKRRALNYLLTSSYLGIGRSRGIGFGEIRLEFRKIEEKEG

SEQ ID NO: 41 - protein, 218 aa, synthetic construct
  REGION 1..218: note = Synthetic polypeptide
MDLEYMHISYPNILLNMRDGSKLRGYFAKKYIDEEIVHNHRDNAFVYKYPQIQFKIIDRSPLIIGIGSLGINFLESKRIFFEKELIISNDTNDITEVNVHKDMDHFGTTDKILKYQFKTPWMALNAKNSEIYKNSDEIDREEFLKRVLIGNILSMSKSLGYTIEEKLKVKINLKEVPVKFKNQNMVGFRGEFYINFDIPQYLGIGRNVSRGFGTVVKV

SEQ ID NO: 42 - protein, 242 aa, synthetic construct
  REGION 1..242: note = Synthetic polypeptide
MYRSRDFYVRVSGQRALFTNPATKGGSERSSYSVPTRQALNGIVDAIYYKPTFTNIVTEVKVINQIQTELQGVRALLHDYSADLSYVSYLSDVVYLIKFHFVWNEDRKDLNSDRLPAKHEAIMERSIRKGGRRDVFLGTRECLGLLDDISQEEYETTVSYYNGVNIDLGIMFHSFAYPKDKKTPLKSYFTKTVMKNGVITFKAQSECDIVNTLSSYAFKAPEEIKSVNDECMEYDAMEKGEN

SEQ ID NO: 43 - protein, 236 aa, synthetic construct
  REGION 1..236: note = Synthetic polypeptide
MRNEVQFELFGDYALFTDPLTKIGGEKLSYSVPTYQALKGIAESIYWKPTIVFVIDELRVMKPIQMESKGVRPIEYGGGNTLAHYTYLKDVHYQVKAHFEFNLHRPDLAFDRNEGKHYSILQRSLKAGGRRDIFLGARECQGYVAPCEFGSGDGFYDGQGKYHLGTMVHGFNYPDETGQHQLDVRLWSAVMENGYIQFPRPEDCPIVRPVKEMEPKIFNPDNVQSAEQLLHDLGGE

SEQ ID NO: 44 - protein, 260 aa, synthetic construct
  REGION 1..260: note = Synthetic polypeptide
MPNDPYSLYSIVIELGAAEKGFPTGILGRSLHSQVLQWFKQDNPFLATELHQSQISPFSISPLMGKRHAKLTKAGDRLFFRICLLRGDLLQPLLNGIEQTVNQSVCLDKFRFRLCQTHILPGSHPLAGASHYSLISQTPVSSKITLDFKSSTSFKVDRKIIQVFPLGEHVFNSLLRRWNNFAPEDLHFSQVDWSIPIAAFDVKTIPIHLKKVEIGAQGWVTYIFPNTEQAKIASVLSEFAFFSGVGRKTTMGMGQVQVRS

SEQ ID NO: 45 - protein, 199 aa, synthetic construct
  REGION 1..199: note = Synthetic polypeptide
MYLSKVIIARAWSRDLYQLHQGLWHLFPNRPDAARDFLFHVEKRNTPEGCHVLLQSAQMPVSTAVATVIKTKQVEFQLQVGVPLYFRLRANPIKTILDNQKRLDSKGNIKRCRVPLIKEAEQIAWLQRKLGNAARVEDVHPISERPQYFSGDGKSGKIQTVCFEGVLTINDAPALIDLVQQGIGPAKSMGCGLLSLAPL

SEQ ID NO: 46 - protein, 187 aa, synthetic construct
  REGION 1..187: note = Synthetic polypeptide
MDHYLDIRLRPDPEFPPAQLMSVLFGKLHQALVAQGGDRIGVSFPDLDESRSRLGERLRIHASADDLRALLARPWLEGLRDHLQFGEPAVVPHPTPYRQVSRVQAKSNPERLRRRLMRRHDLSEEEARKRIPDTVARALDLPFVTLRSQSTGQHFRLFIRHGPLQVTAEEGGFTCYGLSKGGFVPWF

SEQ ID NO: 47 - protein, 314 aa, synthetic construct
  REGION 1..314: note = Synthetic polypeptide
MAARRGGIRRTDLLRRSGQPRGRHRASAAESGLTWISPTLILVGFSHRGDRRMTEHLSRLTLTLEVDAPLERARVATLGPHLHGVLMESIPADYVQTLHTVPVNPYSQYALARSTTSLEWKISTLTNEARQQIVGPINDAAFAGFRLRASGIATQVTSRSLEQNPLSQFARIFYARPETRKFRVEFLTPTAFKQSGEYVFWPDPRLVFQSLAQKYGAIVDGEEPDPGLIAEFGQSVRLSAFRVASAPFAVGAARVPGFTGSATFTVRGVDTFASYIAALLWFGEFSGCGIKASMGMGAIRVQPLAPREKCVPKP

SEQ ID NO: 48 - protein, 264 aa, synthetic construct
  REGION 1..264: note = Synthetic polypeptide
MRFLIRLVPEDKDRAFKVPYNHQYYLQGLIYNAIKSSNPKLATYLHEVKGPKLFTYSLFMAEKREHPKGLPYFLGYKKGFFYFSTCVPEIAEALVNGLLMNPEVRLWDERFYLHEIKVLREPKKFNGSTFVTLSPIAVTVVRKGKSYDVPPMEKEFYSIIKDDLQDKYVMAYGDKPPSEFEMEVLIAKPKRFRIKPGIYQTAWHLVFRAYGNDDLLKVGYEVGFGEKNSLGFGMVKVEGNKTTKEAEEQEKITFNSREELKTGV

SEQ ID NO: 49 - protein, 236 aa, synthetic construct
  REGION 1..236: note = Synthetic polypeptide
MFVTQVIFNIGERTYPDRARAMVAELMDGVQPGLVATLMNYIPGTSTSRTEFPTVQFGGASDGFCLLGFGDGGGAIVRDAVPLIHAALARRMPDRIIQVEHKEHSLSAEARPYVLSYTVPRMVVQKKQRHAERLLHEAEGKAHLEGLFLRSLQRQAAAVGLPLPENLEVEFKGAVGDFAAKHNPNSKVAYRGLRGAVFDVNARLGGIWTAGFMLSKGYGQFNATHQLSGAVNALSE

SEQ ID NO: 50 - protein, 259 aa, synthetic construct
  REGION 1..259: note = Synthetic polypeptide
MHQTLIRINWPKGFKCPPAEFREKLAKSEMFPPEFFHYGTELAVWDKQTAEVEGKIKTVSKEKIIKTFDKPIPLNGRAPVRVIGGQAWAGVIADPEMEGMLIPHLGSILKVASSAAGCAVKIELEQRKFGISYTEYPVKYNLRELVLKRRCEDARSTDIESLIADRIWGGVSGESYYGIDGTCAKFGFEPPSREQLELRIFPMKNIGLHMKSSDGLSKEYMSLIDAEVWMNAKLEGVWQVGNLISRGYGRFIKSIGAQS

SEQ ID NO: 51 - DNA, 24 bp, synthetic construct
  misc_feature 1..24: note = Synthetic oligonucleotide
  source 1..24: mol_type = other DNA
gataatctcttatagaattgaaag

SEQ ID NO: 52 - DNA, 37 bp, synthetic construct
  misc_feature 1..37: note = Synthetic oligonucleotide
  source 1..37: mol_type = other DNA
ctaaaagaataacttgcaaaataacaagcattgaaac

SEQ ID NO: 53 - DNA, 32 bp, synthetic construct
  misc_feature 1..32: note = Synthetic oligonucleotide
  source 1..32: mol_type = other DNA
gtctcacccttcatgggtgagtggattgaaat

SEQ ID NO: 54 - DNA, 32 bp, synthetic construct
  misc_feature 1..32: note = Synthetic oligonucleotide
  source 1..32: mol_type = other DNA
gtcgcactcttcatgggtgcgtggattgaaat

SEQ ID NO: 55 - DNA, 37 bp, synthetic construct
  misc_feature 1..37: note = Synthetic oligonucleotide
  source 1..37: mol_type = other DNA
gtttcagtcccgtagtcgggatttagtggttggaaag

SEQ ID NO: 56 - DNA, 29 bp, synthetic construct
  misc_feature 1..29: note = Synthetic oligonucleotide
  source 1..29: mol_type = other DNA
gagttccccgcgccagcggggataaaccg

SEQ ID NO: 57 - DNA, 28 bp, synthetic construct
  misc_feature 1..28: note = Synthetic oligonucleotide
  source 1..28: mol_type = other DNA
gttcactgccgtataggcagctaagaaa

SEQ ID NO: 58 - DNA, 36 bp, synthetic construct
  misc_feature 1..36: note = Synthetic oligonucleotide
  source 1..36: mol_type = other DNA
gtcgtcagacccaaaaccccgagaggggacggaaac

SEQ ID NO: 59 - DNA, 30 bp, synthetic construct
  misc_feature 1..30: note = Synthetic oligonucleotide
  source 1..30: mol_type = other DNA
gttacaataagactaaaatagaattgaaag

SEQ ID NO: 60 - DNA, 29 bp, synthetic construct
  misc_feature 1..29: note = Synthetic oligonucleotide
  source 1..29: mol_type = other DNA
gtatttcccgcgtgcgcgggggtgagcgg

SEQ ID NO: 61 - DNA, 37 bp, synthetic construct
  misc_feature 1..37: note = Synthetic oligonucleotide
  source 1..37: mol_type = other DNA
tattggatacacccactcattggtgggtggttagaac

SEQ ID NO: 62 - RNA, 24 nt, synthetic construct
  misc_feature 1..24: note = Synthetic oligonucleotide
  source 1..24: mol_type = other RNA
gataatctcttatagaattgaaag

SEQ ID NO: 63 - RNA, 37 nt, synthetic construct
  misc_feature 1..37: note = Synthetic oligonucleotide
  source 1..37: mol_type = other RNA
ctaaaagaataacttgcaaaataacaagcattgaaac

SEQ ID NO: 64 - RNA, 32 nt, synthetic construct
  misc_feature 1..32: note = Synthetic oligonucleotide
  source 1..32: mol_type = other RNA
gtctcacccttcatgggtgagtggattgaaat

SEQ ID NO: 65 - RNA, 32 nt, synthetic construct
  misc_feature 1..32: note = Synthetic oligonucleotide
  source 1..32: mol_type = other RNA
gtcgcactcttcatgggtgcgtggattgaaat

SEQ ID NO: 66 - RNA, 37 nt, synthetic construct
  misc_feature 1..37: note = Synthetic oligonucleotide
  source 1..37: mol_type = other RNA
gtttcagtcccgtagtcgggatttagtggttggaaag

SEQ ID NO: 67 - RNA, 29 nt, synthetic construct
  misc_feature 1..29: note = Synthetic oligonucleotide
  source 1..29: mol_type = other RNA
gagttccccgcgccagcggggataaaccg

SEQ ID NO: 68 - RNA, 28 nt, synthetic construct
  misc_feature 1..28: note = Synthetic oligonucleotide
  source 1..28: mol_type = other RNA
gttcactgccgtataggcagctaagaaa

SEQ ID NO: 69 - RNA, 36 nt, synthetic construct
  misc_feature 1..36: note = Synthetic oligonucleotide
  source 1..36: mol_type = other RNA
gtcgtcagacccaaaaccccgagaggggacggaaac

SEQ ID NO: 70 - RNA, 30 nt, synthetic construct
  misc_feature 1..30: note = Synthetic oligonucleotide
  source 1..30: mol_type = other RNA
gttacaataagactaaaatagaattgaaag

SEQ ID NO: 71 - RNA, 29 nt, synthetic construct
  misc_feature 1..29: note = Synthetic oligonucleotide
  source 1..29: mol_type = other RNA
gtatttcccgcgtgcgcgggggtgagcgg

SEQ ID NO: 72 - RNA, 37 nt, synthetic construct
  misc_feature 1..37: note = Synthetic oligonucleotide
  source 1..37: mol_type = other RNA
tattggatacacccactcattggtgggtggttagaac

SEQ ID NO: 73 - DNA, 2133 bp, synthetic construct
  misc_feature 1..2133: note = Synthetic polynucleotide
  source 1..2133: mol_type = other DNA
atgcccaagaagaagcggaaggtgatgcctctgatcttcaagatcggctataacgtgatccccctgcaggacgtgatcctgcccaccccttccagcaaggtgctgaagtacctgatccagagcggcaagctgatccccagcctgaaggacctgatcaccagccgggacaagtacaagccaatcttcatctcccacctgggcttcaaccagcggaggattttccagaccaacggcaatctgaaaaccatcaccaagggcagtagactgagctccatcatcgccttcagcacccaggccaacgtgctgtccgaggtggccgatgaagggatcttcgaaaccgtgtacggaaagttccacatcatgatcgaaagcatcgagatcgtggaggtggaaaagctgaaggaggaggtggagaagcacatgaacgacaacatcagagtgagattcgtgtctcccacactgctgagctccaaggtgctgctgccccccagcctgtccgaaagatacaagaagatccacgccgggtacagcaccctgcccagcgtgggcctgatcgtggcctacgcctacaacgtgtactgcaatctgatcggcaagaaggaagtggaagtgcgggccttcaagtttggaatcctgagcaacgccctgtccagaatcatcggctacgacctgcaccctgtgaccgtggccatcggcgaggacagcaaggggaatctgagaaaggctcggggcgtgatgggctggatcgagttcgacatccccgacgaaagactgaagcggcgggccctgaactatctgctgaccagcagctacctgggcatcgggagatctcggggcatcggcttcggcgagatccggctggagttccggaagattgaagagaaggagggacccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 74 - DNA, 1920 bp, synthetic construct
  misc_feature 1..1920: note = Synthetic polynucleotide
  source 1..1920: mol_type = other DNA
atgcccaagaagaagcggaaggtgatggacctggagtacatgcacatctcctaccctaacatcctgctgaacatgcgggacggcagcaagctgcggggctacttcgccaagaagtacatcgacgaagagattgtgcacaaccacagagacaacgcctttgtgtacaagtacccccagatccagtttaagatcatcgatagaagccccctgatcatcggcattggctctctgggcatcaatttcctggagagcaagcggatcttcttcgagaaggaactgattatcagcaacgacaccaacgacatcaccgaggtgaacgtgcacaaggacatggatcacttcggcacgaccgacaagatcctgaagtaccagttcaagaccccttggatggcactgaacgccaagaatagcgagatctacaagaactctgacgagatcgaccgggaggagttcctgaagagagtgctgattgggaatatcctgagcatgtctaagagcctgggctataccatcgaagaaaagctgaaggtgaagattaacctgaaggaagtgcccgtgaagttcaagaaccagaacatggtgggctttcggggcgagttctacatcaacttcgacatccctcagtatctgggcatcggccggaatgtgtcccggggattcggcacagtggtgaaggtgcccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 75 - DNA, 1974 bp, synthetic construct
  misc_feature 1..1974: note = Synthetic polynucleotide
  source 1..1974: mol_type = other DNA
atgcccaagaagaagcggaaggtgatgagaaatgaagtgcagttcgagctgttcggcgactacgccctgttcaccgaccccctgaccaagatcggcggcgaaaagctgagctacagcgtgcctacctaccaggccctgaagggcatcgccgagagcatctactggaagcccaccatcgtgttcgtgatcgacgaactgcgggtcatgaagcccattcagatggagtctaagggcgtgaggcccatcgagtacggcggcggcaacaccctggcccactacacctacctgaaggatgtgcactaccaggtgaaggcccacttcgagttcaacctgcaccggcccgacctggccttcgatagaaacgagggcaagcactactccatcctgcagagaagcctgaaggccggcggcagaagagatattttcctgggcgcccgggagtgccagggctacgtggccccctgcgagttcggcagcggcgacggcttctacgacggccagggcaagtaccacctgggaaccatggtgcacggtttcaactaccccgacgaaaccggacagcaccagctggatgtgagactgtggtctgccgtcatggaaaacggctacatccagttcccccgccctgaggactgccccatcgtgcggcctgtgaaggagatggaacccaagatcttcaaccccgacaacgtgcagtccgccgaacagctgctgcacgacctgggcggcgaacccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 76 - DNA, 1992 bp, synthetic construct
  misc_feature 1..1992: note = Synthetic polynucleotide
  source 1..1992: mol_type = other DNA
atgcccaagaagaagcggaaggtgatgtacagaagccgggacttctacgtgagagtgtccggccagcgggccctgttcaccaaccccgccaccaagggcggctccgaacggagctcctactccgtgcctacccggcaggccctgaacgggattgtggacgccatctactacaagcccacgttcaccaacatcgtgaccgaggtgaaggtgattaaccagatccagaccgaactgcagggcgtgcgggccctgctgcatgactacagcgccgacctgagctacgtgtcctacctgagcgacgtggtgtacctgattaagtttcatttcgtgtggaacgaggatagaaaggacctgaatagcgaccggctgccagccaagcatgaggccatcatggagcggtctatccggaagggcggcagacgggacgtgttcctgggcaccagagaatgcctgggcctgctggacgacatcagccaggaagaatacgaaaccacagtgagctattacaatggggtgaacatcgacctgggcatcatgttccacagcttcgcttaccccaaggacaagaaaacccccctgaagtcctacttcacaaagaccgtgatgaagaacggcgtgatcaccttcaaggcccagtccgaatgcgatattgtgaacaccctgagctcctacgccttcaaggcccccgaggagatcaagagcgtgaacgacgagtgcatggagtacgacgccatggagaagggcgaaaaccccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 77 - DNA, 2046 bp, synthetic construct
  misc_feature 1..2046: note = Synthetic polynucleotide
  source 1..2046: mol_type = other DNA
atgcccaagaagaagcggaaggtgatgcccaacgatccctacagcctgtactccatcgtgatcgaactgggcgccgccgaaaagggattccccacaggcatcctgggcagaagcctgcatagccaggtgctgcagtggttcaagcaggataaccccttcctggccaccgagctgcaccagagccagatctcccccttctccatctctccactgatgggcaagcggcacgccaagctgaccaaggccggcgaccggctgttctttcggatctgcctgctgagaggagatctgctgcagcccctgctgaacggcattgagcagaccgtgaaccagagcgtgtgcctggacaagttccggttccggctgtgccagacccacatcctgcccggcagccaccctctggctggcgcctcccactatagcctgatcagccagaccccagtgagctccaagattaccctggacttcaagagttctacctccttcaaggtggaccggaagatcatccaagtgttccctctgggcgaacacgtgttcaacagcctgctcagacgctggaataacttcgcccccgaggacctgcacttctctcaggtggactggagcatccccatcgccgcattcgacgtgaaaaccatccccatccacctgaagaaggtcgagatcggcgcacagggctgggtgacctacatcttccccaacacagaacaggccaagatcgcctccgtgctgagcgaattcgccttcttcagcggagtgggacggaaaaccaccatgggcatgggccaggtgcaggtgcggtcccccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 78 - DNA, 1860 bp, synthetic construct
  misc_feature 1..1860: note = Synthetic polynucleotide
  source 1..1860: mol_type = other DNA
atgcctaagaagaagcggaaggtgtacctgagcaaggtgatcatcgccagagcctggagcagagacctgtaccagctgcaccagggcctgtggcacctgttccccaaccggcccgacgccgcccgggatttcctgttccacgtggagaagagaaacaccccggaaggctgccacgtgctgctgcagagcgcacagatgcctgtgagcaccgccgtggccaccgtgatcaagaccaagcaggtggagttccagctgcaggtgggcgtgcccctgtatttcaggctgcgggcgaatcccatcaagaccatcctggacaaccagaagcggctggacagcaagggcaacatcaagaggtgcagagtgcctctgatcaaggaggccgaacagatcgcctggctgcagcggaagctgggcaatgccgccagagtggaggacgtgcaccccatcagcgagcggccccagtacttctccggcgacggaaagagcggaaagatccagaccgtgtgcttcgagggcgtgctgaccatcaacgacgcacccgccctgatcgacctcgtgcagcaggggatcggccctgccaagtccatgggctgcggactgctgtccctggcccccctgcccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 79 - DNA, 1824 bp, synthetic construct
  misc_feature 1..1824: note = Synthetic polynucleotide
  source 1..1824: mol_type = other DNA
atgcctaagaagaagagaaaggtggaccactacctggacattagactgcgccctgacccagagttccctcctgcccagctgatgtctgtgctgtttggcaagctgcaccaggccctggtggcccagggcggtgacagaatcggagtgtctttccctgatctggacgaatctagatctagactgggagagagactgagaatccacgcgtctgccgacgacctgagagctctgctggccagaccatggctggaaggactgcgcgaccacctgcagttcggtgaacctgccgtggtgcctcacccaactccatacagacaggtgagtagagtgcaggcaaagtctaatccagagagactgagacgcagactgatgagaaggcatgacctgtccgaagaagaagccagaaagagaatcccagacacagtggccagagccctggatctgccttttgtgaccctgagaagccagtctaccggccagcacttcagactgtttattcgccacggaccactgcaggtgaccgccgaagagggaggttttacctgctacggactgagcaagggaggtttcgtgccttggttccccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 80 - DNA, 2208 bp, synthetic construct
  misc_feature 1..2208: note = Synthetic polynucleotide
  source 1..2208: mol_type = other DNA
atgcccaagaagaagcggaaggtgatggccgccagaagaggcggaatccggagaaccgacctgctgcggaggtctggccagcctcggggcagacaccgggcctccgccgccgagagcggcctgacatggatctcccctaccctgatcctggtgggcttcagccacaggggcgataggagaatgaccgagcacctgtccagactgaccctgaccctggaagtggatgcccccctggagagagcccgggtggccaccctgggcccccacctgcatggcgtgctgatggagtctatccccgccgactacgtgcagacactgcacacagtgccggtgaacccttacagccagtacgctctggcccggagcaccaccagcctggagtggaagatctccaccctgacaaatgaggcccggcagcagatcgtcggccccatcaacgacgccgccttcgccggcttccggctgcgggccagcggcatcgccacccaggtgacaagcagaagcctggagcagaaccccctgtcccagtttgccagaatcttctacgccaggcccgaaacccgcaagttcagagtggagttcctgacccccaccgccttcaagcagagcggcgagtacgtgttttggcccgatcccagactggtgttccagtccctggcccagaagtacggcgccatcgtggacggagaagagcccgaccccggcctgatcgccgagtttggccagtccgtgagactgagcgccttcagagtggccagcgccccttttgccgtgggcgccgccagggtgcccggattcaccggcagcgccaccttcaccgtgcggggagtggacaccttcgccagctacatcgccgctctgctgtggttcggcgagttcagcggatgcggcatcaaggcctccatgggaatgggcgccatccgggtgcagcctctggccccccgggagaagtgcgtgcccaagccccccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 81 - DNA, 2058 bp, synthetic construct
  misc_feature 1..2058: note = Synthetic polynucleotide
  source 1..2058: mol_type = other DNA
atgcccaagaagaagcggaaggtgatgagattcctgatcagactggtgcccgaggacaaggacagagccttcaaggtgccttacaaccaccagtactatctgcagggcctgatctacaacgccatcaagtcctccaaccccaagctggccacctacctgcacgaggtgaagggccccaagctgttcacctacagcctgttcatggccgaaaagcgggagcaccctaagggcctgccctactttctgggctacaagaagggcttcttctacttcagcacctgcgtgcccgagatcgccgaggccctggtgaacggcctgctgatgaatcccgaggtgcggctgtgggacgagagattctacctgcacgaaatcaaggtcctgcgggagcccaagaagttcaacggcagcaccttcgtgaccctgagccccatcgccgtgaccgtggtgagaaagggcaagtcctacgacgtgccccccatggaaaaggagttctacagcattatcaaggatgacctgcaggacaagtacgtgatggcctacggcgacaagccccccagtgagttcgagatggaagtgctgatcgccaagcccaagcggttccggatcaagcccggcatctatcagaccgcctggcacctggtgtttcgggcctacggcaatgacgacctgctgaaggtgggctacgaagtgggattcggggagaagaactccctgggattcggaatggtcaaggtggagggcaacaagaccaccaaggaagccgaagaacaggagaagatcaccttcaactcccgggaagagctgaaaacaggcgtgcccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 82 - DNA, 1971 bp, synthetic construct
  misc_feature 1..1971: note = Synthetic polynucleotide
  source 1..1971: mol_type = other DNA
atgcctaagaagaagcggaaggtgttcgtgacccaggtgatcttcaacatcggcgaacggacgtaccccgacagggctcgggctatggtggccgagctgatggatggcgtccagcctggcctggtggccaccctgatgaactacatccccggcaccagcacgagccggacagagttccccaccgtgcagttcggcggcgccagcgacggcttttgcctgctgggcttcggcgacggcggcggcgccatcgtgagagatgccgtgcccctgatccacgccgccctggcaaggcggatgcctgatcggatcatccaggtggaacacaaggagcacagcctgtccgccgaggcccggccctacgtgctgagctacaccgtgcctcggatggtggtgcagaagaagcagcggcacgccgagagactgctgcacgaagccgagggaaaggctcacctggagggcctgttcctgcggagcctgcagaggcaggccgccgccgtgggcctgcccctgcccgagaacctggaggtggagttcaagggagccgtgggcgacttcgccgcaaagcacaatccaaatagcaaggtggcctaccggggactgagaggcgccgtgttcgatgtgaacgccagactgggcggcatctggaccgccggattcatgctgagcaagggctacggccagtttaacgccacccaccagctgagcggcgccgtgaacgctctgtccgaacccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 83 - DNA, 2040 bp, synthetic construct
  misc_feature 1..2040: note = Synthetic polynucleotide
  source 1..2040: mol_type = other DNA
atgcccaagaagaagagaaaggtgcaccagaccctgatccggatcaactggcccaagggattcaagtgcccccctgccgagttccgggaaaagctggccaagagcgagatgttcccccccgagttcttccactacggcacggaactggccgtgtgggacaagcagaccgccgaggtggagggcaagatcaagaccgtgtccaaggagaagatcatcaagacctttgacaagcccatccccctgaatggccgggccccggtcagagtgatcggcggccaggcctgggccggcgtgatcgccgaccccgagatggagggcatgctgatcccacacctggggagcatcctgaaggtggccagcagcgcggccggatgcgcagtgaagatcgaactggaacagagaaagttcggcatcagctacaccgagtaccccgtgaagtacaacctgcgggagctggtgctgaagagaagatgcgaggacgcccggtctaccgatatcgagagcctgattgccgatagaatctggggcggcgtgtccggcgagagctactatggcatcgacggcacatgcgccaagtttggcttcgaaccccccagcagagagcagctggagctgcggatcttccccatgaagaacatcggactgcacatgaagtccagcgacggactgtccaaggagtacatgagcctgattgacgccgaggtgtggatgaacgctaagctggaaggagtgtggcaggtgggcaacctgatcagcaggggctacggccggttcatcaagtctatcggcgcccagtcccccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 84 - DNA, 867 bp, synthetic construct
  misc_feature 1..867: note = Synthetic polynucleotide
  source 1..867: mol_type = other DNA
atgcctctgatcttcaagatcggctataacgtgatccccctgcaggacgtgatcctgcccaccccttccagcaaggtgctgaagtacctgatccagagcggcaagctgatccccagcctgaaggacctgatcaccagccgggacaagtacaagccaatcttcatctcccacctgggcttcaaccagcggaggattttccagaccaacggcaatctgaaaaccatcaccaagggcagtagactgagctccatcatcgccttcagcacccaggccaacgtgctgtccgaggtggccgatgaagggatcttcgaaaccgtgtacggaaagttccacatcatgatcgaaagcatcgagatcgtggaggtggaaaagctgaaggaggaggtggagaagcacatgaacgacaacatcagagtgagattcgtgtctcccacactgctgagctccaaggtgctgctgccccccagcctgtccgaaagatacaagaagatccacgccgggtacagcaccctgcccagcgtgggcctgatcgtggcctacgcctacaacgtgtactgcaatctgatcggcaagaaggaagtggaagtgcgggccttcaagtttggaatcctgagcaacgccctgtccagaatcatcggctacgacctgcaccctgtgaccgtggccatcggcgaggacagcaaggggaatctgagaaaggctcggggcgtgatgggctggatcgagttcgacatccccgacgaaagactgaagcggcgggccctgaactatctgctgaccagcagctacctgggcatcgggagatctcggggcatcggcttcggcgagatccggctggagttccggaagattgaagagaaggaggga

SEQ ID NO: 85 - DNA, 654 bp, synthetic construct
  misc_feature 1..654: note = Synthetic polynucleotide
  source 1..654: mol_type = other DNA
atggacctggagtacatgcacatctcctaccctaacatcctgctgaacatgcgggacggcagcaagctgcggggctacttcgccaagaagtacatcgacgaagagattgtgcacaaccacagagacaacgcctttgtgtacaagtacccccagatccagtttaagatcatcgatagaagccccctgatcatcggcattggctctctgggcatcaatttcctggagagcaagcggatcttcttcgagaaggaactgattatcagcaacgacaccaacgacatcaccgaggtgaacgtgcacaaggacatggatcacttcggcacgaccgacaagatcctgaagtaccagttcaagaccccttggatggcactgaacgccaagaatagcgagatctacaagaactctgacgagatcgaccgggaggagttcctgaagagagtgctgattgggaatatcctgagcatgtctaagagcctgggctataccatcgaagaaaagctgaaggtgaagattaacctgaaggaagtgcccgtgaagttcaagaaccagaacatggtgggctttcggggcgagttctacatcaacttcgacatccctcagtatctgggcatcggccggaatgtgtcccggggattcggcacagtggtgaaggtg

SEQ ID NO: 86 - DNA, 708 bp, synthetic construct
  misc_feature 1..708: note = Synthetic polynucleotide
  source 1..708: mol_type = other DNA
atgagaaatgaagtgcagttcgagctgttcggcgactacgccctgttcaccgaccccctgaccaagatcggcggcgaaaagctgagctacagcgtgcctacctaccaggccctgaagggcatcgccgagagcatctactggaagcccaccatcgtgttcgtgatcgacgaactgcgggtcatgaagcccattcagatggagtctaagggcgtgaggcccatcgagtacggcggcggcaacaccctggcccactacacctacctgaaggatgtgcactaccaggtgaaggcccacttcgagttcaacctgcaccggcccgacctggccttcgatagaaacgagggcaagcactactccatcctgcagagaagcctgaaggccggcggcagaagagatattttcctgggcgcccgggagtgccagggctacgtggccccctgcgagttcggcagcggcgacggcttctacgacggccagggcaagtaccacctgggaaccatggtgcacggtttcaactaccccgacgaaaccggacagcaccagctggatgtgagactgtggtctgccgtcatggaaaacggctacatccagttcccccgccctgaggactgccccatcgtgcggcctgtgaaggagatggaacccaagatcttcaaccccgacaacgtgcagtccgccgaacagctgctgcacgacctgggcggcgaa

SEQ ID NO: 87 - DNA, 726 bp, synthetic construct
  misc_feature 1..726: note = Synthetic polynucleotide
  source 1..726: mol_type = other DNA
atgtacagaagccgggacttctacgtgagagtgtccggccagcgggccctgttcaccaaccccgccaccaagggcggctccgaacggagctcctactccgtgcctacccggcaggccctgaacgggattgtggacgccatctactacaagcccacgttcaccaacatcgtgaccgaggtgaaggtgattaaccagatccagaccgaactgcagggcgtgcgggccctgctgcatgactacagcgccgacctgagctacgtgtcctacctgagcgacgtggtgtacctgattaagtttcatttcgtgtggaacgaggatagaaaggacctgaatagcgaccggctgccagccaagcatgaggccatcatggagcggtctatccggaagggcggcagacgggacgtgttcctgggcaccagagaatgcctgggcctgctggacgacatcagccaggaagaatacgaaaccacagtgagctattacaatggggtgaacatcgacctgggcatcatgttccacagcttcgcttaccccaaggacaagaaaacccccctgaagtcctacttcacaaagaccgtgatgaagaacggcgtgatcaccttcaaggcccagtccgaatgcgatattgtgaacaccctgagctcctacgccttcaaggcccccgaggagatcaagagcgtgaacgacgagtgcatggagtacgacgccatggagaagggcgaaaac

SEQ ID NO: 88 - DNA, 780 bp, synthetic construct
  misc_feature 1..780: note = Synthetic polynucleotide
  source 1..780: mol_type = other DNA
atgcccaacgatccctacagcctgtactccatcgtgatcgaactgggcgccgccgaaaagggattccccacaggcatcctgggcagaagcctgcatagccaggtgctgcagtggttcaagcaggataaccccttcctggccaccgagctgcaccagagccagatctcccccttctccatctctccactgatgggcaagcggcacgccaagctgaccaaggccggcgaccggctgttctttcggatctgcctgctgagaggagatctgctgcagcccctgctgaacggcattgagcagaccgtgaaccagagcgtgtgcctggacaagttccggttccggctgtgccagacccacatcctgcccggcagccaccctctggctggcgcctcccactatagcctgatcagccagaccccagtgagctccaagattaccctggacttcaagagttctacctccttcaaggtggaccggaagatcatccaagtgttccctctgggcgaacacgtgttcaacagcctgctcagacgctggaataacttcgcccccgaggacctgcacttctctcaggtggactggagcatccccatcgccgcattcgacgtgaaaaccatccccatccacctgaagaaggtcgagatcggcgcacagggctgggtgacctacatcttccccaacacagaacaggccaagatcgcctccgtgctgagcgaattcgccttcttcagcggagtgggacggaaaaccaccatgggcatgggccaggtgcaggtgcggtcc

SEQ ID NO: 89 - DNA, 597 bp, synthetic construct
  misc_feature 1..597: note = Synthetic polynucleotide
  source 1..597: mol_type = other DNA
atgtacctgagcaaggtgatcatcgccagagcctggagcagagacctgtaccagctgcaccagggcctgtggcacctgttccccaaccggcccgacgccgcccgggatttcctgttccacgtggagaagagaaacaccccggaaggctgccacgtgctgctgcagagcgcacagatgcctgtgagcaccgccgtggccaccgtgatcaagaccaagcaggtggagttccagctgcaggtgggcgtgcccctgtatttcaggctgcgggcgaatcccatcaagaccatcctggacaaccagaagcggctggacagcaagggcaacatcaagaggtgcagagtgcctctgatcaaggaggccgaacagatcgcctggctgcagcggaagctgggcaatgccgccagagtggaggacgtgcaccccatcagcgagcggccccagtacttctccggcgacggaaagagcggaaagatccagaccgtgtgcttcgagggcgtgctgaccatcaacgacgcacccgccctgatcgacctcgtgcagcaggggatcggccctgccaagtccatgggctgcggactgctgtccctggcccccctg

SEQ ID NO: 90 - DNA, 561 bp, synthetic construct
  misc_feature 1..561: note = Synthetic polynucleotide
  source 1..561: mol_type = other DNA
atggaccactacctggacattagactgcgccctgacccagagttccctcctgcccagctgatgtctgtgctgtttggcaagctgcaccaggccctggtggcccagggcggtgacagaatcggagtgtctttccctgatctggacgaatctagatctagactgggagagagactgagaatccacgcgtctgccgacgacctgagagctctgctggccagaccatggctggaaggactgcgcgaccacctgcagttcggtgaacctgccgtggtgcctcacccaactccatacagacaggtgagtagagtgcaggcaaagtctaatccagagagactgagacgcagactgatgagaaggcatgacctgtccgaagaagaagccagaaagagaatcccagacacagtggccagagccctggatctgccttttgtgaccctgagaagccagtctaccggccagcacttcagactgtttattcgccacggaccactgcaggtgaccgccgaagagggaggttttacctgctacggactgagcaagggaggtttcgtgccttggttc

SEQ ID NO: 91 - DNA, 942 bp, synthetic construct
  misc_feature 1..942: note = Synthetic polynucleotide
  source 1..942: mol_type = other DNA
atggccgccagaagaggcggaatccggagaaccgacctgctgcggaggtctggccagcctcggggcagacaccgggcctccgccgccgagagcggcctgacatggatctcccctaccctgatcctggtgggcttcagccacaggggcgataggagaatgaccgagcacctgtccagactgaccctgaccctggaagtggatgcccccctggagagagcccgggtggccaccctgggcccccacctgcatggcgtgctgatggagtctatccccgccgactacgtgcagacactgcacacagtgccggtgaacccttacagccagtacgctctggcccggagcaccaccagcctggagtggaagatctccaccctgacaaatgaggcccggcagcagatcgtcggccccatcaacgacgccgccttcgccggcttccggctgcgggccagcggcatcgccacccaggtgacaagcagaagcctggagcagaaccccctgtcccagtttgccagaatcttctacgccaggcccgaaacccgcaagttcagagtggagttcctgacccccaccgccttcaagcagagcggcgagtacgtgttttggcccgatcccagactggtgttccagtccctggcccagaagtacggcgccatcgtggacggagaagagcccgaccccggcctgatcgccgagtttggccagtccgtgagactgagcgccttcagagtggccagcgccccttttgccgtgggcgccgccagggtgcccggattcaccggcagcgccaccttcaccgtgcggggagtggacaccttcgccagctacatcgccgctctgctgtggttcggcgagttcagcggatgcggcatcaaggcctccatgggaatgggcgccatccgggtgcagcctctggccccccgggagaagtgcgtgcccaagccc

SEQ ID NO: 92 - DNA, 792 bp, synthetic construct
  misc_feature 1..792: note = Synthetic polynucleotide
  source 1..792: mol_type = other DNA
atgagattcctgatcagactggtgcccgaggacaaggacagagccttcaaggtgccttacaaccaccagtactatctgcagggcctgatctacaacgccatcaagtcctccaaccccaagctggccacctacctgcacgaggtgaagggccccaagctgttcacctacagcctgttcatggccgaaaagcgggagcaccctaagggcctgccctactttctgggctacaagaagggcttcttctacttcagcacctgcgtgcccgagatcgccgaggccctggtgaacggcctgctgatgaatcccgaggtgcggctgtgggacgagagattctacctgcacgaaatcaaggtcctgcgggagcccaagaagttcaacggcagcaccttcgtgaccctgagccccatcgccgtgaccgtggtgagaaagggcaagtcctacgacgtgccccccatggaaaaggagttctacagcattatcaaggatgacctgcaggacaagtacgtgatggcctacggcgacaagccccccagtgagttcgagatggaagtgctgatcgccaagcccaagcggttccggatcaagcccggcatctatcagaccgcctggcacctggtgtttcgggcctacggcaatgacgacctgctgaaggtgggctacgaagtgggattcggggagaagaactccctgggattcggaatggtcaaggtggagggcaacaagaccaccaaggaagccgaagaacaggagaagatcaccttcaactcccgggaagagctgaaaacaggcgtg

SEQ ID NO: 93 - DNA, 708 bp, synthetic construct
  misc_feature 1..708: note = Synthetic polynucleotide
  source 1..708: mol_type = other DNA
atgttcgtgacccaggtgatcttcaacatcggcgaacggacgtaccccgacagggctcgggctatggtggccgagctgatggatggcgtccagcctggcctggtggccaccctgatgaactacatccccggcaccagcacgagccggacagagttccccaccgtgcagttcggcggcgccagcgacggcttttgcctgctgggcttcggcgacggcggcggcgccatcgtgagagatgccgtgcccctgatccacgccgccctggcaaggcggatgcctgatcggatcatccaggtggaacacaaggagcacagcctgtccgccgaggcccggccctacgtgctgagctacaccgtgcctcggatggtggtgcagaagaagcagcggcacgccgagagactgctgcacgaagccgagggaaaggctcacctggagggcctgttcctgcggagcctgcagaggcaggccgccgccgtgggcctgcccctgcccgagaacctggaggtggagttcaagggagccgtgggcgacttcgccgcaaagcacaatccaaatagcaaggtggcctaccggggactgagaggcgccgtgttcgatgtgaacgccagactgggcggcatctggaccgccggattcatgctgagcaagggctacggccagtttaacgccacccaccagctgagcggcgccgtgaacgctctgtccgaa

SEQ ID NO: 94 - DNA, 777 bp, synthetic construct
  misc_feature 1..777: note = Synthetic polynucleotide
  source 1..777: mol_type = other DNA
atgcaccagaccctgatccggatcaactggcccaagggattcaagtgcccccctgccgagttccgggaaaagctggccaagagcgagatgttcccccccgagttcttccactacggcacggaactggccgtgtgggacaagcagaccgccgaggtggagggcaagatcaagaccgtgtccaaggagaagatcatcaagacctttgacaagcccatccccctgaatggccgggccccggtcagagtgatcggcggccaggcctgggccggcgtgatcgccgaccccgagatggagggcatgctgatcccacacctggggagcatcctgaaggtggccagcagcgcggccggatgcgcagtgaagatcgaactggaacagagaaagttcggcatcagctacaccgagtaccccgtgaagtacaacctgcgggagctggtgctgaagagaagatgcgaggacgcccggtctaccgatatcgagagcctgattgccgatagaatctggggcggcgtgtccggcgagagctactatggcatcgacggcacatgcgccaagtttggcttcgaaccccccagcagagagcagctggagctgcggatcttccccatgaagaacatcggactgcacatgaagtccagcgacggactgtccaaggagtacatgagcctgattgacgccgaggtgtggatgaacgctaagctggaaggagtgtggcaggtgggcaacctgatcagcaggggctacggccggttcatcaagtctatcggcgcccagtcc

SEQ ID NO: 95 - protein, 6 aa, synthetic construct
  REGION 1..6: note = Synthetic peptide
  SITE 1..6: note = /note="Variant residues given in the sequence have no preference with respect to those in the annotations for variant positions"
  VARIANT 3: note = /replace="Phe"
  VARIANT 4: note = /replace="Tyr"
RNYFSH

SEQ ID NO: 96 - protein, 6 aa, synthetic construct
  REGION 1..6: note = Synthetic peptide
RNYFSH

SEQ ID NO: 97 - protein, 6 aa, synthetic construct
  REGION 1..6: note = Synthetic peptide
RNFYSH

SEQ ID NO: 98 - protein, 6 aa, synthetic construct
  REGION 1..6: note = Synthetic peptide
  SITE 1..6: note = /note="Variant residues given in the sequence have no preference with respect to those in the annotations for variant positions"
  VARIANT 3: note = /replace="Lys"
RNAALH

SEQ ID NO: 99 - protein, 6 aa, synthetic construct
  REGION 1..6: note = Synthetic peptide
RAFFHH

SEQ ID NO: 100 - protein, 6 aa, synthetic construct
  REGION 1..6: note = Synthetic peptide
RRAFFH

SEQ ID NO: 101 - DNA, 145 bp, synthetic construct
  misc_feature 1..145: note = Synthetic polynucleotide
  misc_feature 1..145: note = /note="Variant nucleotides given in the sequence have no preference with respect to those in the annotations for variant positions"
  variation 70: note = a, c, t, or g
  variation 145: note = /replace=" "
  source 1..145: mol_type = other DNA
ttggccactccctcthtgcgcgctcgctcgctcgctggggcctggvgaccaaaggtcgccagacggccgngctctgcccggccggccccagcgagcgagcgagcgcgcadagagggagtggccaactccatcactaggggtdccb

SEQ ID NO: 102 - DNA, 144 bp, synthetic construct
  misc_feature 1..144: note = Synthetic polynucleotide
  variation 1: note = /replace=" "
  misc_feature 1..144: note = /note="Variant nucleotides given in the sequence have no preference with respect to those in the annotations for variant positions"
  variation 89: note = a, c, t, or g
  source 1..144: mol_type = other DNA
rdtacccctagtgatggagttggccactccctctatgcgcgctcgctcgctcggtggggcctgcvgaccaaaggtcbccagacggcvgngctctgchcggccggccccaccgagcgagcgagcgcgcatagagggagtggccaa

SEQ ID NO: 103 - DNA, 22 bp, Adeno-associated virus 2
  source 1..22: mol_type = genomic DNA
ctgcgcgctcgctcgctcactg

SEQ ID NO: 104 - DNA, 22 bp, Adeno-associated virus 2
  source 1..22: mol_type = genomic DNA
cagtgagcgagcgagcgcgcag

SEQ ID NO: 105 - protein, 4 aa, synthetic construct
  REGION 1..4: note = Synthetic: linker
GSSS

SEQ ID NO: 106 - protein, 5 aa, synthetic construct
  REGION 1..5: note = Synthetic: linker
GSSSS

SEQ ID NO: 107 - protein, 6 aa, synthetic construct
  REGION 1..6: note = Synthetic: linker
GSSGSS

SEQ ID NO: 108 - skipped

SEQ ID NO: 109 - DNA, 387 bp, synthetic construct
  misc_feature 1..387: note = Synthetic: MCP nucleic acid sequence
  source 1..387: mol_type = other DNA
gcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggagtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctac

SEQ ID NO: 110 - protein, 129 aa, synthetic construct
  REGION 1..129: note = Synthetic: MCP amino acid sequence
ASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 111 - DNA, 363 bp, synthetic construct
  misc_feature 1..363: note = Synthetic: PCP nucleic acid sequence
  source 1..363: mol_type = other DNA
tccaaaacaatagtcctctccgtaggggaggcaacacggactttgaccgaaatccagtcaaccgctgaccgacaaatctttgaagagaaagtagggcctcttgtgggccgactgcgcttgactgcaagcttgcgacaaaacggcgcaaagactgcctatagggtcaaccttaaactcgaccaagccgacgtggtcgatagcggtctccctaaggttcggtatacgcaggtctggagtcatgacgtaacaatcgtagcaaacagcacagaagcctcccgaaaaagcctctacgatctgacgaaatccttggtggctacgtcacaggtggaagacctcgttgtcaaccttgtacctctgggtcga

SEQ ID NO: 112 - protein, 121 aa, synthetic construct
  REGION 1..121: note = Synthetic: PCP amino acid sequence
SKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGR

SEQ ID NO: 113 - DNA, 186 bp, synthetic construct
  misc_feature 1..186: note = Synthetic: COM nucleic acid sequence
  source 1..186: mol_type = other DNA
atgaaatcaattcgctgtaaaaactgcaacaaactgttatttaaggcggattcctttgatcacattgaaatcaggtgtccgcgttgcaaacgtcacatcataatgctgaatgcctgcgagcatcccacggagaaacattgtgggaaaagagaaaaaatcacgcattctgacgaaaccgtgcgttat

SEQ ID NO: 114 - protein, 62 aa, synthetic construct
  REGION 1..62: note = Synthetic: COM amino acid sequence
MKSIRCKNCNKLLFKADSFDHIEIRCPRCKRHIIMLNACEHPTEKHCGKREKITHSDETVRY

SEQ ID NO: 115 - DNA, 19 bp, synthetic construct
  misc_feature 1..19: note = Synthetic: MS2
  source 1..19: mol_type = other DNA
acatgaggatcacccatgt

SEQ ID NO: 116 - DNA, 128 bp, synthetic construct
  misc_feature 1..128: note = Synthetic: MS2 X3(MS2-linker-MS2-linker-MS2)
  source 1..128: mol_type = other DNA
acatgaggatcacccatgtctgcaggtcgactctagaaaacatgaggatcacccatgtctgcagtattcccgggttcattagatcctaaggtacctaattgcctagaaaacatgaggatcacccatgt

SEQ ID NO: 117 - DNA, 25 bp, synthetic construct
  misc_feature 1..25: note = Synthetic: PP7
  source 1..25: mol_type = other DNA
ggagcagacgatatggcgtcgctcc

SEQ ID NO: 118 - DNA, 146 bp, synthetic construct
  misc_feature 1..146: note = Synthetic: PP7 X3(PP7-linker-PP7-linker-PP7)
  source 1..146: mol_type = other DNA
ggagcagacgatatggcgtcgctccctgcaggtcgactctagaaaggagcagacgatatggcgtcgctccctgcagtattcccgggttcattagatcctaaggtacctaattgcctagaaaggagcagacgatatggcgtcgctcc

SEQ ID NO: 119 - DNA, 18 bp, synthetic construct
  misc_feature 1..18: note = Synthetic: com
  source 1..18: mol_type = other DNA
gaatgcctgcgagcatcc

SEQ ID NO: 120 - DNA, 125 bp, synthetic construct
  misc_feature 1..125: note = Synthetic: com X3(com-linker-com-linker-com)
  source 1..125: mol_type = other DNA
gaatgcctgcgagcatccctgcaggtcgactctagaaagaatgcctgcgagcatccctgcagtattcccgggttcattagatcctaaggtacctaattgcctagaaagaatgcctgcgagcatcc

SEQ ID NO: 121 - DNA, 1866 bp, synthetic construct
  misc_feature 1..1866: note = Synthetic: Rep2 nucleic acid sequence
  source 1..1866: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 122 - protein, 621 aa, synthetic construct
  REGION 1..621: note = Synthetic: Rep2 amino acid sequence
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 123 - DNA, 2289 bp, synthetic construct
  misc_feature 1..2289: note = Synthetic: MCP-Rep nucleic acid sequence (linker-MCP-linker-Rep)
  source 1..2289: mol_type = other DNA
atgcccggcagctccggcagtagcgcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggagtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctacggcagtagtgggtcctctgggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 124 - protein, 762 aa, synthetic construct
  REGION 1..762: note = Synthetic: MCP-Rep amino acid sequence(linker-MCP-linker-Rep)
MPGSSGSSASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIYGSSGSSGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 125 - DNA, 2289 bp, synthetic construct
  misc_feature 1..2289: note = Synthetic: MCP-Rep-Y156F nucleic acid sequence (linker-MCP-linker-Rep)
  source 1..2289: mol_type = other DNA
atgcccggcagctccggcagtagcgcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggagtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctacggcagtagtgggtcctctgggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaatttcttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 126 - protein, 762 aa, synthetic construct
  REGION 1..762: note = Synthetic: MCP-Rep-Y156F amino acid sequence (linker-MCP-linker-Rep)
MPGSSGSSASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIYGSSGSSGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNFLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 127 - DNA, 2289 bp, synthetic construct
  misc_feature 1..2289: note = Synthetic: MCP-Rep-KDE-mu nucleic acid sequence (linker-MCP-linker-Rep)
  source 1..2289: mol_type = other DNA
atgcccggcagctccggcagtagcgcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggagtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctacggcagtagtgggtcctctgggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacgcggtggtggctgcgtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 128 - protein, 762 aa, synthetic construct
  REGION 1..762: note = Synthetic: MCP-Rep-KDE-muamino acid sequence (linker-MCP-linker-Rep)
MPGSSGSSASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIYGSSGSSGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNAVVAACYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 129 - DNA, 2289 bp, synthetic construct
  misc_feature 1..2289: note = Synthetic: MCP-Rep-EKE-mu nucleic acid sequence (linker-MCP-linker-Rep)
  source 1..2289: mol_type = other DNA
atgcccggcagctccggcagtagcgcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggagtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctacggcagtagtgggtcctctgggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgcggcgggagcgagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 130 - protein, 762 aa, synthetic construct
  REGION 1..762: note = Synthetic: MCP-Rep-EKE-muamino acid sequence (linker-MCP-linker-Rep)
MPGSSGSSASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIYGSSGSSGFYEIVIKVPSDLDEHLPGISDSFVNWVAEKEWELPPDSDMDLNLIEQAPLTVAEKLQRDFLTEWRRVSKAPEALFFVQFAAGASYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 131 - DNA, 2616 bp, synthetic construct
  misc_feature 1..2616: note = Synthetic: 2XMCP-Rep nucleic acid sequence (linker-2XMCP-linker-Rep)
  source 1..2616: mol_type = other DNA
atgcccggcagctccggcagtagcgcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacggtggtggaggaggaatggcgtccaatttcacgcagttcgtcctggttgacaacggggggactggggacgttacggtcgctccgagcaactttgccaatggtattgcggagtggatttcttctaattcacggtcccaagcttacaaagtgacctgttccgtgcggcaaagttctgctcagaatagaaagtacactataaaggtcgaagtccctaagggggcctggcgatcatatctcaatatggagcttaccatcccaatatttgccactaattctgattgtgaattgattgtcaaagcaatgcaaggactcttgaaagacggaaacccaatccccagcgcaatcgcagccaactccggtatatacggcagtagtgggtcctctgggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 132 - protein, 871 aa, synthetic construct
  REGION 1..871: note = Synthetic: 2XMCP-Rep amino acid sequence (linker-2XMCP-linker-Rep)
MPGSSGSSASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGSSGSSGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 133 - DNA, 2265 bp, synthetic construct
  misc_feature 1..2265: note = Synthetic: PCP-Rep-Y156F nucleic acid sequence (linker-PCP-linker-Rep)
  source 1..2265: mol_type = other DNA
atgcccggcagctccggcagtagctccaaaacaatagtcctctccgtaggggaggcaacacggactttgaccgaaatccagtcaaccgctgaccgacaaatctttgaagagaaagtagggcctcttgtgggccgactgcgcttgactgcaagcttgcgacaaaacggcgcaaagactgcctatagggtcaaccttaaactcgaccaagccgacgtggtcgatagcggtctccctaaggttcggtatacgcaggtctggagtcatgacgtaacaatcgtagcaaacagcacagaagcctcccgaaaaagcctctacgatctgacgaaatccttggtggctacgtcacaggtggaagacctcgttgtcaaccttgtacctctgggtcgaggcagtagtgggtcctctgggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaatttcttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 134 - protein, 754 aa, synthetic construct
  REGION 1..754: note = Synthetic: PCP-Rep-Y156F amino acid sequence (linker-PCP-linker-Rep)
MPGSSGSSSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRGSSGSSGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNFLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 135 - DNA, 2088 bp, synthetic construct
  misc_feature 1..2088: note = Synthetic: COM-Rep-Y156F nucleic acid sequence (linker-COM-linker-Rep)
  source 1..2088: mol_type = other DNA
atgcccggcagctccggcagtagcatgaaatcaattcgctgtaaaaactgcaacaaactgttatttaaggcggattcctttgatcacattgaaatcaggtgtccgcgttgcaaacgtcacatcataatgctgaatgcctgcgagcatcccacggagaaacattgtgggaaaagagaaaaaatcacgcattctgacgaaaccgtgcgttatggcagtagtgggtcctctgggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaatttcttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 136 - protein, 695 aa, synthetic construct
  REGION 1..695: note = Synthetic: COM-Rep-Y156F amino acid sequence (linker-COM-linker-Rep)
MPGSSGSSMKSIRCKNCNKLLFKADSFDHIEIRCPRCKRHIIMLNACEHPTEKHCGKREKITHSDETVRYGSSGSSGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNFLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 137 - DNA, 1020 bp, synthetic construct
  misc_feature 1..1020: note = Synthetic: MCP-AAP(DJ) nucleic acid sequence (linker-MCP-linker-AAP)
  source 1..1020: mol_type = other DNA
atgggcagctccggcagtagcgcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggagtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctacggcagtagtgggtcctctctggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatga

SEQ ID NO: 138 - protein, 339 aa, synthetic construct
  REGION 1..339: note = Synthetic: MCP-AAP(DJ) amino acid sequence (linker-MCP-linker-AAP)
MGSSGSSASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIYGSSGSSLETQTQSQTLNQSENLPQPPQVWDLLQWLQAVAHQWQTITRAPTEWVIPREIGIAIPHGWATESSPPAPEPGPCPPTTTTSTSKSPTAHLEDLQMTTPTSATAPPGGILTLTDSTATFHHVTGSDSSTTTGDSGPRDSASSSSTSRSRRSRRMKAPRPSPITSPAPSRCLRTRSTSCRTFSALPTRAACLRSRRTCS

SEQ ID NO: 139 - DNA, 1020 bp, synthetic construct
  misc_feature 1..1020: note = Synthetic: AAP-MCP(DJ) nucleic acid sequence (AAP-linker-MCP-linker)
  source 1..1020: mol_type = other DNA
atgctggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcaggcagctccggcagtagcgcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggagtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctacggcagtagtgggtcctcttga

SEQ ID NO: 140 - protein, 339 aa, synthetic construct
  REGION 1..339: note = Synthetic: AAP-MCP(DJ) amino acid sequence (AAP-linker-MCP-linker)
MLETQTQSQTLNQSENLPQPPQVWDLLQWLQAVAHQWQTITRAPTEWVIPREIGIAIPHGWATESSPPAPEPGPCPPTTTTSTSKSPTAHLEDLQMTTPTSATAPPGGILTLTDSTATFHHVTGSDSSTTTGDSGPRDSASSSSTSRSRRSRRMKAPRPSPITSPAPSRCLRTRSTSCRTFSALPTRAACLRSRRTCSGSSGSSASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIYGSSGSS

SEQ ID NO: 141 - DNA, 23 bp, synthetic construct
  misc_feature 1..23: note = Synthetic: WPRE-F
  source 1..23: mol_type = other DNA
cccgtatggctttcattttctcc

SEQ ID NO: 142 - DNA, 19 bp, synthetic construct
  misc_feature 1..19: note = Synthetic: WPRE-R
  source 1..19: mol_type = other DNA
ggcaatgccccaaccagtg

SEQ ID NO: 143 - DNA, 20 bp, synthetic construct
  misc_feature 1..20: note = Synthetic: Cre-F
  source 1..20: mol_type = other DNA
ccagtagatgccactagcga

SEQ ID NO: 144 - DNA, 20 bp, synthetic construct
  misc_feature 1..20: note = Synthetic: Cre-R
  source 1..20: mol_type = other DNA
gcctggagatacagcaggta

SEQ ID NO: 145 - DNA, 20 bp, synthetic construct
  misc_feature 1..20: note = Synthetic: CAG-F
  source 1..20: mol_type = other DNA
cttctcctccgggctgtaat

SEQ ID NO: 146 - DNA, 20 bp, synthetic construct
  misc_feature 1..20: note = Synthetic: CAG-R
  source 1..20: mol_type = other DNA
ctttcacgcagccacagaaa

SEQ ID NO: 147 - DNA, 3266 bp, synthetic construct
  misc_feature 1..3266: note = Synthetic: Stuffer nucleic acid sequence
  source 1..3266: mol_type = other DNA
atatttggagggcagcttgatttcgacttcgggagggaagctgcgccatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgccgcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggccattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaaggccgccgctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcgccacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagaggcccgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcgccagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgaggccaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctaaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatcccctt

SEQ ID NO: 148 - protein, 120 aa, synthetic construct
  REGION 1..120: note = Synthetic: linker
  VARIANT 1..120: note = For every "GSSSSS" at least one "S" is present and others may be present or absent; "GSSSSS" or a variation thereof is present at least once, but all other instances may be present or absent
GSSSSSGSSSSSGSSSSSGSSSSSGSSSSSGSSSSSGSSSSSGSSSSSGSSSSSGSSSSSGSSSSSGSSSSSGSSSSSGSSSSSGSSSSSGSSSSSGSSSSSGSSSSSGSSSSSGSSSSS

(19) United States
(12) Patent Application Publication
(10) Pub. No.: US 2024/0368629 A1
(43) Pub. Date: Nov. 7, 2024

(54) RNA ADENO-ASSOCIATED VIRUS (RAAV) VECTOR AND USES THEREOF

(71) Applicant: Huidagene Therapeutics (Singapore) Pte. Ltd., Singapore (SG)

(72) Inventors: Linyu Shi, Singapore (SG); Weiya Bai, Singapore (SG)

(21) Appl. No.: 18/664,495

(22) Filed: May 15, 2024

Related U.S. Application Data
(62) Division of application No. 17/836,222, filed on Jun. 9, 2022, now Pat. No. 12,031,148.
Application No. 17/836,222 is a continuation of application No. PCT/CN2022/075366, filed on Feb. 7, 2022.

(30) Foreign Application Priority Data
Feb. 7, 2021 (WO) PCT/CN2021/075874

(51) Int. Cl. C12N 15/90; C12N 15/10; C12N 15/86

(52) U.S. Cl. CPC C12N 15/902; C12N 15/102; C12N 15/86; C12N 2310/20; C12N 2750/14143

(57) ABSTRACT

The invention described herein provides a recombinant DNA viral particle comprising a protein shell encapsulating an RNA vector genome, as well as related compositions and uses thereof.

### REFERENCE TO RELATED APPLICATION

The instant application is a divisional of U.S. application Ser. No. 17/836,222, filed on Jun. 9, 2022, which is a continuation application, filed under 35 U.S.C. 111 (a), of International Patent Application No. PCT/CN2022/075366, filed on Feb. 7, 2022, which claims foreign priority under 35 U.S.C. 365 (b), to International Patent Application No. PCT/CN2021/075874, filed on Feb. 7, 2021, the entire contents of each of the above-referenced applications, including any sequence listing and drawings, are incorporated herein by reference.

### SEQUENCE LISTING

The instant application contains a Sequence Listing XML file which has been submitted electronically in.XML format. Said XML file, created on May 13, 2024, is named “132045-00602_SL.xml” and is 261,052 bytes in size. The sequence listing contained in this XML file is part of the specification and is hereby incorporated by reference herein in its entirety.

### BACKGROUND OF THE INVENTION

Adeno-associated virus (AAV) is a small (about 20 nm in diameter) replication-defective, nonenveloped virus that infects human and other primate species. It belongs to the genus Dependoparvovirus within the family Parvoviridae. Wild type AAV can infect both dividing and non-dividing cells and may incorporate its genome into that of the host cell. Its life cycle is dependent on the presence of a helper virus, such as adenovirus (AdV), hence its name and taxonomy classification.

AAV is found in multiple vertebrate species, including human and non-human primates (NHPs). The current consensus is that AAV does not cause any human diseases, and only causes a very mild immune response. It is composed of an icosahedral protein capsid of about 20˜25 nm in diameter, and a single-stranded DNA (ssDNA) genome of about 4.7 kb that can either be the plus (sense) or minus (anti-sense) strand.

The AAV capsid comprises three types of subunit, VP1, VP2 and VP3, totaling 60 copies in a ratio of 1:1:10 (VP1: VP2: VP3). The genome is flanked by two T-shaped inverted terminal repeats (ITRs) at the ends that largely serve as the viral origins of replication and the packaging signal. The rep gene encodes four proteins required for viral replication. The Rep proteins are named after their molecular masses: Rep78, Rep68, Rep52 and Rep40. The cap gene encodes the translation from different start codons. In addition, a third gene, which encodes assembly activating protein (AAP), is encoded within the cap coding sequence in a different reading frame, and has been shown to promote virion assembly.

Currently, thirteen AAV serotypes and numerous variants have been identified, they recognize distinct cell receptors, and thereby display different tissue-type and cell-type tropism profiles. The in vivo tissue tropisms of AAV1-13 have also been well studied in many animal models.

The AAV ITR sequences comprise 145 nucleotides each. The ITR sequences can each form a hairpin for self-priming, which allows primase-independent synthesis of the second DNA strand. The ITRs were also shown to be required for both integration of the AAV DNA into the host cell genome (19th chromosome in humans) and rescue from it as well as for efficient encapsidation of the AAV DNA combined with generation of a fully assembled, DNase-resistant AAV particles.

The AAV2 ITR serves as origin of replication and is composed of two arm palindromes (namely B-B′ and C-C′) embedded in a larger stem palindrome (A-A′). The ITR can acquire two configurations (i.e., flip and flop). See FIG. 1. The flip and flop configurations have the B-B′ and the C-C′ palindrome closest to the 3′ end, respectively. The 20-nucleotide D sequence or D region is present only once at each end of the AAV genome and thus remains single-stranded.

The ITR also contains a ˜22-bp sequence-Rep-binding element (RBE)—that binds the AAV Rep78 and Rep68 proteins in a specific orientation. If the ITR is in the palindromic (hairpinned) configuration, the Rep protein also contacts a 5-base sequence at the tip of one of the short palindromes (RBE′), which activates the Rep DNA helicase and strand-specific endonuclease activities to help AAV replication and packaging (see FIG. 1).

The RBE comprises a tetranucleotide repeat (e.g., 4 repeats) with the consensus sequence of 5′-GNGC-3′. The ATP-dependent DNA helicase activities of Rep78 and Rep68 remodel the A-A′ region, generating a stem-loop that locates at the summit the terminal resolution site (trs or TRS) in a single-stranded form. In this configuration, the strand—and site-specific endonuclease catalytic domain of Rep78 and Rep68 introduces a nick at the trs. The nucleotides at the apex of the T-shaped structure correspond to an additional RBE (RBE′) that stabilizes the association between the two largest Rep proteins and the ITR.

In AAV life cycle, when AAV DNA is uncoated in the nucleus, the ITR of the incoming single-stranded genome snaps into a hairpin that provides a natural 3′-OH primer for the synthesis of the second strand. This produces a duplex molecule that has a covalently closed (hairpinned) end. The large Rep proteins then bind RBE and RBE′ within the hairpin, and the activated endonuclease cleaves one strand at a specific site within a recognition sequence called the terminal resolution site (trs). This creates a new 3′-OH primer that is used to repair the ITR to form a normal blunt-ended duplex molecule. During cleavage, a molecule of Rep78 or Rep68 is covalently attached to the 5′-end phosphate via a tyrosine-phosphate linkage. The ITR is then reconfigured into a double hairpin to produce a 3′-OH primer that directs strand displacement synthesis down the length of the genome using the cellular complexes. This displaces a single strand, which is packaged, and reforms a duplex molecule that is covalently closed at one end, beginning a new cycle of nicking, repair, and strand displacement synthesis. Each time this cycle is repeated, a new single strand is generated for packaging. Because the two ends are identical, the process occurs equally well from both ends, generating both positive and negative strands for packaging.

Since AAV is capable of transducing a wide range of species and tissues in vivo with no evidence of toxicity, and generates relatively mild innate and adaptive immune responses, it has been widely used in gene therapy.

AAV vectors are composed of the same capsid sequence and structure as found in wild-type AAVs (wtAAVs). However, AAV vectors encapsidate genomes that are devoid of all AAV protein-coding sequences and have transgene expression cassettes designed in their place. The only sequences of viral origin are the ITRs, which are needed to guide genome replication and packaging during vector production. The complete removal of viral coding sequences maximizes the packaging capacity of AAV vectors, and contributes to their low immunogenicity and cytotoxicity when delivered in vivo.

Because AAV vectors optimally accommodate genomes that are under 4.7 kb, the payload must be carefully designed to consider not only the transgene sequence but also the inclusion of regulatory elements necessary for gene expression (for example, promoter, enhancer, intron and polyadenylation signal).

A popular AAV vector production method is triple transfection of HEK293T cells, which harbor constitutively expressed AdV Ela and Elb genes, with a packaging plasmid expressing rep and cap genes, a transgene plasmid to be packaged into AAV capsids, and a helper plasmid containing other AdV genes that serve helper function, such as the E2A, E4 and VA RNA genes that are essential for replication, message RNA (mRNA) processing and translation, respectively. Fortunately, the transgene expression cassette that is built with AAV2 ITRs can be packaged into any serotype capsids by merely exchanging the capsid-coding region in the packaging plasmid or helper virus.

AAV vectors recognize and bind distinct cell receptors, and get into the cells by internalization. Intact AAV vector particles in endosomes undergo a series of pH-dependent structural changes necessary for transduction and traffic through the cytosol via the cytoskeletal network. After endosomal escape, AAV vector enters the nucleus through the nuclear pore complex, where it undergoes capsid uncoating to release the genome.

The single-stranded AAV vectors genome that is released in the nucleus is not immediately ready for gene expression until it is converted to a double-stranded form—a requirement of transcription and a rate-limiting step for transduction.

Second strand synthesis is initiated from the self-primed ITR at the 3′-end of the genome. Additionally, double-stranded genomes can be achieved by strand annealing, whereby plus-stranded and minus-stranded genomes that are packaged into separate virions anneal by Watson-Crick base pairing once in the nucleus. The double-stranded genome then undergoes circularization via intra-molecular or inter-molecular genome recombination at the ITRs. This circularization and concatemerization process stabilizes the AAV vectors genome as episomal DNA, leading to gene expression that persists in post mitotic cells (FIG. 2).

CRISPR has brought new momentum to gene therapy. CRISPR is a powerful genome editing tool, and it has shown potential in curing genetic, acquired and infectious diseases. However, delivery of the cellular components for CRISPR is still a major hurdle for its clinical translation. So far, the most successful in vivo gene editing with CRISPR uses AAV as a delivery vector.

However, conventional AAV delivery has suffered from multiple practical difficulties, including 1) off-targeting effects increased by prolonged Cas9 expression, 2) stimulation of Cas9-specific immune responses, 3) high frequency of virus integrations in the CRISPR induced double-stranded breaks. Recent studies confirm the wide spread pre-existing immunity against Cas9 in human population, which might bring an extra challenge to the edited cells if Cas9 is consistently expressing.

Thus, there is a need to improve existing gene editing tools, such as the Cas9-mediated gene editing tools.

### SUMMARY OF THE INVENTION

One aspect of the invention provides a ribonucleotide (RNA) sequence capable of being packaged into a DNA virus viral particle, the RNA sequence comprises: (1) an RNA sequence of interest (RSI), e.g., a RNA coding sequence for a gene of interest (GOI), a protein (e.g., a therapeutic protein, an antigen protein, or a gene-editing protein such as a CRISPR/Cas effector enzyme (“a Cas protein” for short), a ZFN protein, a TALEN protein)-encoding RNA, such as, a mRNA, or a non-coding, functional RNA (such as, a transfer RNA (tRNA), a ribosomal RNA (rRNA), a small interfering RNA (siRNA), a short hairpin RNA (shRNA), an antisense RNA, an antisense oligonucleotide, a micro RNA (miRNA), or an RNA component of a CRISPR-Cas (e.g., Cas9, Cas12, Cas13) system, including a guide RNA (or a gRNA), such as, a single guide RNA (or a sgRNA, a chimeric RNA, an RNA chimera), a CRISPR RNA (crRNA), and a tracr RNA), or a precursor thereof; and, (2) an RNA-packaging signal (RPS) capable of interacting, e.g., binding, directly or indirectly, to an RPS-interacting molecule that facilitates packaging of the RNA sequence into the DNA virus viral particle; optionally, a DNA sequence encoding or corresponding to the RNA sequence, or a reverse complement of the DNA sequence, has reduced, diminished, or substantially no capacity of being packaged into the DNA virus viral particle (e.g., the DNA sequence or the reverse complement thereof lacks a DNA packaging signal such as a functional AAV ITR for AAV packaging).

In certain embodiments, the DNA virus viral particle is an AAV viral particle or an oncolytic viral particle.

In certain embodiments, the RPS is located at or near the 5′ end of the RSI, at or near the 3′ end of the RSI, or internal to the RSI (e.g., inside an intron of an mRNA).

In certain embodiments, the RNA sequence comprises more than one (e.g., 1, 2, 3, or more) RPS that are identical or different.

In certain embodiments, two or more (e.g., 3) of the more than one RPS are adjacent to each other, or are in tandem, via the same or different linkers.

In certain embodiments, the RNA sequence comprises two or more RPS that are not adjacent to each other (e.g., one each located at or near one end of the RNA sequence of interest (RSI)).

In certain embodiments, the RPS comprises a transcribed modified AAV inverted terminal repeat (ITR), wherein the transcribed modified AAV ITR: (a) comprises a transcribed functional Rep-Binding Element (RBE), optionally further comprising a transcribed functional RBE′; and, (b) lacks either a transcribed terminal resolution site (TRS), or a transcribed reverse complement TRS (rcTRS), or both; optionally, the transcribed modified AAV ITR further comprises a transcribed D region sequence (D sequence or D′ sequence); and/or optionally, the RPS-interacting molecule is Rep78, Rep68, Rep52, and/or Rep40.

In certain embodiments, the transcribed modified AAV ITR is within the 3′ end 1000 nucleotides, 800 nucleotides, 500 nucleotides, 300 nucleotides, or 200 nucleotides of the RNA; optionally, the transcribed modified AAV ITR is 5′ to a poly A sequence, a polyA signal sequence (e.g., AAUAAA), or a sequence for RNA transcription termination (e.g., a histone downstream element).

In certain embodiments, the transcribed modified AAV ITR is modified based on a transcribed wild-type flip or flop ITR; optionally, the wild-type flip or flop ITR is from AAV1, AAV2, AAV3A, AAV3B, AAV4, AAV5, AAV6, AAV7, AAVrh74, AAV8, AAV9, AAV10, AAV11, AAV12, or AAV13 (optionally, the wild-type flop ITR has the nucleotide sequence of SEQ ID NO: 1).

In certain embodiments, the transcribed modified AAV ITR lacks both the transcribed TRS and the transcribed rcTRS.

In certain embodiments, the transcribed modified AAV ITR comprises the transcribed D region sequence (optionally, the modified AAV ITR has the nucleotide sequence of SEQ ID NO: 3).

In certain embodiments, the transcribed modified AAV ITR lacks the transcribed D region sequence (optionally, the modified AAV ITR has the nucleotide sequence of SEQ ID NO: 2).

In certain embodiments, the RNA sequence further comprises a second transcribed modified AAV ITR having a second transcribed functional RBE sequence but lacking either a second transcribed TRS or a second transcribed rcTRS or both; optionally, the second transcribed modified AAV ITR further comprises a second transcribed D region sequence.

In certain embodiments, the transcribed modified AAV ITR and the second transcribed modified AAV ITR are identical (or different).

In certain embodiments, the transcribed modified AAV ITR, and the second transcribed modified AAV ITR (if present), comprise a deletion from, a mutation in, or an insertion into a corresponding transcribed wild-type AAV ITR D region sequence or a corresponding transcribed wild-type TRS/rcTRS.

In certain embodiments, the second transcribed modified AAV ITR is within 5′ end 1000 nucleotides, 800 nucleotides, 500 nucleotides, 250 nucleotides, or 150 nucleotides of the RNA sequence.

In certain embodiments, the RPS comprises an MS2 sequence, an PP7 binding site, or a com binding site, and the RPS-interacting molecule comprises an RPS-interacting protein (RPSIP) capably of interacting, e.g., binding, directly or indirectly, to the RPS, such as a bacteriophage-derived MS2 coat protein (MCP) for an MS2 sequence, a PP7 bacteriophage coat protein (PCP) for an PP7 binding site, or a phage COM protein (COM) for a com binding site.

In certain embodiments, the RPSIP is associated directly or indirectly with (e.g., fused to) a protein component of the viral packaging system for the DNA virus viral particle (such as Rep78 and/or Rep68 of adeno-associated virus 2 (AAV2), or assembly-activating protein (AAP)).

In certain embodiments, the RNA sequence comprises or preferably does not comprise a transcribed DNA packaging signal, for example, a transcribed wild-type AAV ITR sequence (e.g., the RNA sequence comprises a transcribed modified AAV ITR sequence having an addition, a deletion, and/or a substitution of a nucleotide of a corresponding transcribed wild-type AAV ITR sequence to reduce the DNA packaging capability of the DNA virus viral particle).

In certain embodiments, the RNA sequence further comprises: (1) a transcribed transcription enhancer; (2) a transcribed intron sequence or exon sequence (such as one for enhancing protein expression); (3) a 5′ UTR sequence; (4) a 3′ UTR sequence; (5) a polyA sequence, or a polyadenylation (polyA) signal sequence and optionally a GU-rich region downstream of the polyA signal sequence; (6) a posttranscriptional regulatory element or sequence, such as a transcribed Woodchuck Hepatitis Virus (WHP) Posttranscriptional Regulatory Element (WPRE) sequence; and/or, (7) a transcription termination sequence (such as a histone downstream element), optionally, the RNA sequence comprises an RPS located 3′ to the posttranscriptional regulatory element or sequence, and 5′ to the polyA sequence or the polyA signal sequence.

In certain embodiments, the RNA sequence comprises, in 5′ to 3′ orientation, the RSI, the optional transcribed WPRE sequence; the RPS (such as the transcribed modified AAV ITR, the MS2 sequence, the PP7 binding site, or the com binding site); and the polyA sequence or the polyA signal sequence.

In certain embodiments, the GOI comprises a protein (e.g., a fluorescent protein, a therapeutic protein, an antigen protein, or a gene-editing protein such as a Cas protein, a ZFN protein, a TALEN protein), an enzyme (such as a Cre protein, or a CRISPR/Cas effector enzyme, e.g., Cas9, Cas 12, Cas13, or a variant thereof), a structural protein, an mRNA, a non-coding RNA (ncRNA), an siRNA, a piRNA, a short hairpin RNA or shRNA, a microRNA (miRNA) or a precursor thereof (including pre-miRNA and pri-miRNA), a ribosomal RNA (rRNA), an antisense sequence or oligonucleotide (ASO), an RNA component of a CRISPR-Cas system, including a guide RNA (or a gRNA), such as, a single guide RNA (or a sgRNA, a chimeric RNA, an RNA chimera), a CRISPR RNA (crRNA), and a tracr RNA, a guide RNA or gRNA for a CRISPR/Cas effector enzyme, an rRNA, a tRNA, a snoRNA, a snRNA, an exRNA, a scaRNA, a lncRNA, a Xist, and a HOTAIR.

In certain embodiments, the RNA sequence is a single-stranded RNA less than about 8,900 nucleotides in length, less than about 8,000 nucleotides in length, less than about 7,000 nucleotides in length, less than about 6,000 nucleotides in length, less than about 5,200 nucleotides in length, less than about 4,000 nucleotides in length, less than about 3,000 nucleotides in length, less than about 2,000 nucleotides in length, about 4,700-5,200 nucleotides in length, about 4,700-5,000 nucleotide in length, about 4,700-4,800 nucleotides in length, or about 4,700 nucleotides in length.

Another aspect of the invention provides a polynucleotide comprising a cassette encoding the RNA sequence of the invention; optionally, the polynucleotide is a DNA sequence (e.g., a DNA plasmid), optionally comprising a stuffer sequence in the backbone of the DNA plasmid, and/or optionally comprising no functional DNA packaging signal such as AAV ITR.

In certain embodiments, the polynucleotide further comprises a promoter operably linked to and driving the transcription of the RNA sequence encoded by the cassette.

In certain embodiments, the promoter is a ubiquitous promoter.

In certain embodiments, the promoter is a tissue-specific promoter.

In certain embodiments, the promoter is a constitutive promoter.

In certain embodiments, the promoter is an inducible promoter.

In certain embodiments, the polynucleotide further comprises an enhancer that enhances the transcription of the RNA sequence driven by the promoter.

Another aspect of the invention provides a recombinant DNA virus viral particle comprising an RNA genome (such as the RNA sequence of the invention or the RNA sequence transcribed from the polynucleotide of the invention) packaged within the protein shell (such as capsid) of a DNA virus (such as an AAV virus, or an oncolytic virus).

In certain embodiments, the DNA virus is AAV, and the recombinant DNA virus viral particle is a recombinant RNA adeno-associated virus (rRAAV) particle, comprising: (1) an AAV capsid; and, (2) the RNA sequence of the invention or the RNA sequence transcribed from the polynucleotide of the invention packaged within the AAV capsid.

In certain embodiments, the AAV capsid comprises a capsid from an AAV of the serotype AAV1, AAV2, AAV3A, AAV3B, AAV4, AAV5, AAV6, AAV7, AAV8, AAV9, AAV10, AAV11, AAV12, AAV13, AAV-DJ, AAV PHP.cB, Anc80L65, Anc80L65AAP, AAVrh74, or 7m8.

Another aspect of the invention provides a population of recombinant DNA virus viral particles (e.g., rRAAV particles) comprising a plurality of recombinant DNA virus viral particle (e.g., rRAAV particle) of the invention, wherein at least about 10%, 20%, 30%, 40%, 50%, 60%, 70%, 80%, 90%, 95%, 96%, 97%, 98%, 99% or more of the recombinant DNA virus viral particles (e.g., rRAAV particles) within the population have the RNA sequence of the invention or the RNA sequence transcribed from the polynucleotide of the invention packaged therein.

Another aspect of the invention provides a host cell comprising the RNA sequence of the invention, the polynucleotide of the invention, the RNA sequence transcribed from the polynucleotide of the invention, the recombinant DNA virus viral particle (e.g., rRAAV particle) of the invention, and/or the population of recombinant DNA virus viral particles (e.g., rRAAV particles) of the invention.

In certain embodiments, the host cell further comprises a viral packaging system that facilitates packaging of the RNA sequence of the invention or the RNA sequence transcribed from the polynucleotide of the invention into the DNA virus viral particle.

In certain embodiments, the viral packaging system comprises: (1) an AAV rep gene (e.g., coding sequence for Rep78, Rep68, Rep52, and/or Rep40) and an AAV cap gene (e.g., coding sequence for VP1, VP2, VP3, AAP, and/or MAAP), under the transcriptional control of one or more promoters that drive the transcription of the rep gene and cap gene, or the expression products thereof; (2) one or more coding sequences for one or more proteins required for AAV packaging, such as adenoviral E2A, E4, and VA genes, or the one or more proteins; and (3) the RPS-interacting molecule or a coding sequence thereof; optionally, the capacity of the viral packaging system of packaging a DNA sequence into the DNA virus viral particle is reduced, diminished, or substantially eliminated by, for example, (1) removing a part or all of the DNA packaging signals such as AAV ITR on the polynucleotide encoding the RNA sequence of the invention or on the polynucleotide of the invention, (2) modifying, e.g., mutating, the AAV rep gene, the AAV cap gene, and/or the one or more coding sequences for one or more proteins required for AAV packaging to reduce, diminish, or substantially eliminate the capacity of the respective translated protein to facilitate the packaging of the DNA sequence into the DNA virus viral particle (e.g., a Y156F mutation in the common sequence of Rep78 and Rep68 proteins, KDE-mu, or EKE-mu); and/or (3) enlarging the size of the polynucleotide encoding the RNA sequence of the invention or the polynucleotide of the invention.

In certain embodiments, the host cell is a mammalian cell (such as HEK293 cells) or an insect cell (such as Sf9 or Sf21 cells).

Another aspect of the invention provides a method of generating the recombinant DNA virus viral particle (e.g., rRAAV particle) of the invention or the population of recombinant DNA virus viral particles (e.g., rRAAV particles) of the invention, the method comprising: a) culturing the host cell of the invention for a sufficient time, and b) harvesting the recombinant DNA virus viral particle or the population of recombinant DNA virus viral particles.

In certain embodiments, the method further comprises isolating or purifying the recombinant DNA virus viral particle or the population of recombinant DNA virus viral particles.

Another aspect of the invention provides a method of generating a recombinant DNA virus viral particle (e.g., rRAAV particle) or a population of recombinant DNA virus viral particles, the method comprising: a) contacting a viral packaging system (e.g., a AAV packaging system) with the RNA sequence of the invention or the RNA sequence transcribed from the polynucleotide of the invention for a period of time sufficient to produce the recombinant DNA virus viral particle or the population of recombinant DNA virus viral particles, and b) harvesting the recombinant DNA virus viral particle or the population of recombinant DNA virus viral particles; and, optionally, c) isolating or purifying the harvested recombinant DNA virus viral particle or population of recombinant DNA virus viral particles.

In certain embodiments, the viral packaging system (e.g., a AAV packaging system) comprises: (1) one or more proteins for assembling the protein shell (e.g., VP1, VP2, and/or VP3 for assembling AAV capsid) of the DNA virus viral particle for packaging the RNA sequence, or one or more coding sequences thereof; (2) one or more proteins (e.g., Rep78, Rep68, Rep52, and/or Rep40 for AAV packaging) for facilitating the assembling of the protein shell and/or the packaging of the RNA sequence into the protein shell of the DNA virus viral particle, or one or more coding sequences thereof (e.g., adenoviral E2a, E4, and VA genes); and (3) the RPS-interacting molecule or a coding sequence thereof; optionally, the capacity of the viral packaging system of packaging a DNA sequence into the DNA virus viral particle is reduced, diminished, or substantially eliminated by, for example, (1) removing a part or all of the DNA packaging signals such as AAV ITR on the polynucleotide encoding the RNA sequence of the invention or on the polynucleotide of the invention, (2) modifying, e.g., mutating, the AAV rep gene, the AAV cap gene, and/or the one or more coding sequences for one or more proteins required for AAV packaging to reduce, diminish, or substantially eliminate the capacity of the respective translated protein to facilitate the packaging of the DNA sequence into the DNA virus viral particle (e.g., a Y156F mutation in the common sequence of Rep78 and Rep68 proteins, KDE-mu, or EKE-mu); and/or (3) enlarging the size of the polynucleotide encoding the RNA sequence of the invention or the polynucleotide of the invention.

Another aspect of the invention provides a system of packaging the RNA sequence of the invention or the RNA sequence transcribed from the polynucleotide of the invention into a DNA virus viral particle, comprising: (1) one or more proteins for assembling the protein shell (e.g., VP1, VP2, and/or VP3 for assembling AAV capsid) of the DNA virus viral particle for packaging the RNA sequence, or one or more coding sequences thereof; (2) one or more proteins (e.g., Rep78, Rep68, Rep52, and/or Rep40 for AAV packaging) for facilitating the assembling of the protein shell and/or the packaging of the RNA sequence into the protein shell of the DNA virus viral particle, or one or more coding sequences thereof (e.g., adenoviral E2a, E4, and VA genes); and (3) the RPS-interacting molecule or a coding sequence thereof; optionally, the capacity of the viral packaging system of packaging a DNA sequence into the DNA virus viral particle is reduced, diminished, or substantially eliminated by, for example, (1) removing a part or all of the DNA packaging signals such as AAV ITR on the polynucleotide encoding the RNA sequence of the invention or on the polynucleotide of the invention, (2) modifying, e.g., mutating, the AAV rep gene, the AAV cap gene, and/or the one or more coding sequences for one or more proteins required for AAV packaging to reduce, diminish, or substantially eliminate the capacity of the respective translated protein to facilitate the packaging of the DNA sequence into the DNA virus viral particle (e.g., a Y156F mutation in the common sequence of Rep78 and Rep68 proteins, KDE-mu, or EKE-mu); and/or (3) enlarging the size of the polynucleotide encoding the RNA sequence of the invention or the polynucleotide of the invention.

Another aspect of the invention provides a method of delivering a gene of interest (GOI) into a cell, a plant, or an animal, the method comprising contacting the cell, the plant, or the animal with the recombinant DNA virus viral particle (e.g., rRAAV particle) of the invention, the population of the recombinant DNA virus viral particles (e.g., rRAAV particles) of the invention, or the recombinant DNA virus viral particle (e.g., rRAAV particle) or the population of the recombinant DNA virus viral particles (e.g., rRAAV particles) produced by the method of the invention, wherein the GOI is encoded by the RNA sequence of the invention.

Another aspect of the invention provides a method of delivering an RNA sequence of interest (RSI) into a cell, a plant, or an animal, the method comprising contacting the cell, the plant, or the animal with the recombinant DNA virus viral particle (e.g., rRAAV particle) of the invention, the population of the recombinant DNA virus viral particles (e.g., rRAAV particles) of the invention, or the recombinant DNA virus viral particle (e.g., rRAAV particle) or the population of the recombinant DNA virus viral particles (e.g., rRAAV particles) produced by the method of the invention.

Another aspect of the invention provides a method of diagnosing, preventing, or treating a disease or disorder in a subject in need thereof, comprising administrating to the subject a therapeutically effective amount or dose of the population of the recombinant DNA virus viral particles (e.g., rRAAV particles) of the invention or produced by the method of the invention.

Another aspect of the invention provides a use of the recombinant DNA virus viral particle (e.g., rRAAV particle) of the invention, the population of the recombinant DNA virus viral particles (e.g., rRAAV particles) of the invention, or the recombinant DNA virus viral particle (e.g., rRAAV particle) or the population of the recombinant DNA virus viral particles (e.g., rRAAV particles) produced by the method of the invention in the manufacture of a medicament for diagnosing, preventing, or treating a disease or disorder in a subject in need thereof.

It should be understood that any one embodiment of the invention described herein, including those described only in the examples or claims, or only in one aspects/sections below, can be combined with any other one or more embodiments of the invention, unless explicitly disclaimed or improper.

### BRIEF DESCRIPTION OF THE DRAWINGS

FIG. 1A shows the structure and sequence of the wild type ITR of AAV2, including the A: A′ stem region sequences, the B: B′ and C: C′ T region sequences, and the unpaired D region sequence, in both the flip (SEQ ID NO: 5) and flop (SEQ ID NO: 28) configuration of 3′ ITR. The RBE, RBE′ and the TRS are also shown.

FIGS. 1B and 1C show multi-sequence alignments of 5′ (SEQ ID NOS 101, 25, 27, 29, 31, 33, 35, and 37, respectively, in order of appearance) (FIG. 1B) and 3′ (SEQ ID NOS 102, 26, 28, 30, 32, 34, 36, and 38, respectively, in order of appearance) (FIG. 1C) ITR sequences from AAV1-7.

FIG. 2 shows the life cycle of an AAV vector/viral particle, and the subject RAAV vector/viral particle.

FIG. 3 is a schematic diagram of transgene plasmids of RAAV-ITR vectors and control vectors, showing the relative position and orientation of the promoter (such as the CAG promoter or “C”), the GOI coding sequence (such as the coding sequence for the reporter gene tdTomato or “T”), the WPRE sequence (or “W”), the SV40 polyA signal sequence (or “S”), and the wild-type ITR, mutated/optimized ITR (dITR or dITR-D).

FIG. 4 is a schematic diagram showing the generation of AAV vectors and RAAV-ITR vectors with the triple-plasmid system. By co-transfecting three plasmids (e.g., a transgene plasmid, a packaging plasmid, and a helper plasmid) into a proper packaging cell like such as the HEK293 cells, recombinant AAV or RAAV viral vectors can be generated. Green ITR indicates wild type ITR, yellow ITR indicates optimized ITRs. pCAG-Transgene, pCAG-Transgene-ITR and pCAG-ITR-Transgene-ITR are transgene plasmids; pAAV-rep/cap is a packaging plasmid; and pHelper is the helper plasmid.

FIGS. 5A and 5B show representative viral vector titration process. FIG. 5A is a flowchart for RAAV titration. FIG. 5B shows primers and probes for Q-PCR.

FIGS. 6A-6C show titration of RAAV-ITR vectors. FIG. 6A shows titration of CITWS group. FIG. 6B shows titration of CTWIS group. FIG. 6C shows titration of CITWIS group.

FIGS. 7A and 7B show titration and infection of RAAV-dITR-D vectors. FIG. 7A shows titration of RAAV-dITR-D vectors. FIG. 7B shows in vitro infection of RAAV-dITR-D vectors. The same volume (5 μL) of purified RAAV-dITR-D vectors had been used to infect 2×10⁵ HEK293T cells in vitro. Fluorescence photos were taken 3 and 5 days post infection.

FIG. 8A is a schematic diagram (not to scale) showing the different plasmid constructs used to demonstrate efficient packaging of RNA into RAAV particles.

FIG. 8B shows the results of specific DNA and RNA packaging of the AAV-tdTomato and RAAV-tdTomato constructs by detecting the WPRE sequence in the packaged DNA or RNA. Efficient RNA packaging occurred when both the heterologous RNA Packaging Signal (RPS) and its cognate RPS binding protein (RBP, e.g., MCP for MS2) are both present.

FIGS. 9A-9C show reduced DNA packaging using enlarged plasmid backbone. FIG. 9A is a schematic diagram (not to scale) of the various plasmids, including the plasmid with the longer backbone sequence due to the inserted stuffer region (L-CTWM3S), used to generate the results in FIGS. 9B and 9C. FIG. 9B shows specific DNA packaging of AAV-tdTomato and RAAV-tdTomato by detecting the presence of CAG promoter sequence using CAG-specific primer pairs. FIG. 9C shows specific DNA and RNA packaging of AAV-tdTomato and RAAV-tdTomato by detecting the presence of WPRE sequence using WPRE-specific primer pairs. The results showed a surprising ˜2-fold reduction of undesired DNA packaging by using enlarged/longer plasmid backbone sequence with stuffer sequences.

FIGS. 10A and 10B show efficient packaging of the Cre transgene into RAAV using the MS2/MCP packaging system. FIG. 10A shows specific DNA packaging of AAV-Cre and RAAV-Cre by detecting the presence of CAG promoter sequence using CAG-specific primer pairs. Note that the CAG sequence is not present in the RAAV RNA sequence, and the detected RNA signal was background. FIG. 10B shows specific DNA and RNA packaging of AAV-Cre and RAAV-Cre by detecting the presence of WPRE sequence using WPRE-specific primer pairs.

FIGS. 11A-11B show that RPS/RBP improved RNA packaging of conventional AAVs. FIG. 11A shows the results of AAV genome packaging in the presence of only DNA packaging signals (i.e., ITRs). FIG. 11B shows the AAV genome packaging in presence of both DNA packaging signals (ITRs) and RNA packaging signals (MS2X3).

FIGS. 12A-12D show the results of optimizing the RAAV system and identification of the properties of optimized RAAVs. FIG. 12A represents the specific genome packaging of AAV-Cre and RAAV-Cre by detecting WPRE sequence. FIG. 12B represents the specific genome packaging of AAV-Cre and RAAV-Cre by detecting Cre sequence. FIG. 12C shows silver staining analysis of the composition of the AAV and RAAV particles. FIG. 12D shows the morphology analysis of the AAV and RAAV particles by TEM, scale bar 100 nm.

FIGS. 13A and 13B show results of reducing DNA packaging of AAV and RAAV.

FIG. 13A shows that engineered Rep reduced DNA packaging of the conventional AAV.

FIG. 13B shows reduction of DNA packaging in RAAV by using various mutant MCP fusion proteins, including double mutant MCP fusion protein DJ-MCPX2.

FIGS. 14A-14D show that the RAAV viral particles express functional transgene-encoded proteins. Samples are designated the same way in FIGS. 14A-14C. FIG. 14A shows a time course of Cre mRNA levels in infected cells. FIG. 14B shows fold change of Cre mRNA levels in infected cells from 20 hrs post infection. FIG. 14C shows a time course of Cre DNA levels in infected cells. FIG. 14D shows percentage of infected cells quantified by flow cytometry 5 days after infection, n=2 replicates.

FIGS. 15A-15D show results of DNA and mRNA analysis for the AAV or RAAV infected Ai9-MEF cells. FIG. 15A shows Ct value of the Cre mRNA. FIG. 15B shows Ct value of the Cre DNA. FIG. 15C shows Ct value of the GAPDH mRNA. FIG. 15D shows Ct value of the 36B4 DNA.

FIG. 16 shows genotype identification of Ai9-MEF cells.

FIGS. 17A-17B show transient transfer of RAAV particles. FIG. 17A shows Western blot analysis of the lifespan of Cre protein in infected cells after conventional AAV delivery.

FIG. 17B shows Western blot analysis of the lifespan of Cre protein in infected cells after RAAV delivery.

FIG. 18 shows additional functional RPS/RBP pairs—the PP7/PCP pair, and the com/COM pair-tested in the RAAV system.

FIG. 19 shows that the RAAV system is applicable for various AAV serotypes, including AAV-DJ, AAV5, AAV8, and AAV9.

FIGS. 20A and 20B shows that additional AAP and MCP fusion proteins increased RAAV yield. FIG. 20A represents the specific genome packaging of RAAV-Cre by detecting Cre sequence. FIG. 20B shows comparison of the RNA packaging efficiency of RAAVs with AAP N—or C-terminal fusions (AM or MA fusion constructs).

FIGS. 21A-21C show results of transient transfer of RAAV-Cre into the hippocampus of Ai9-Mice. FIG. 21A shows transfer of high dose of AAV-Cre into the hippocampus of Ai9-Mice. FIG. 21B shows transfer of low dose of AAV-Cre into the hippocampus of Ai9-Mice. FIG. 21C shows transfer of high dose of RAAV-Cre into the hippocampus of Ai9-Mice. FIG. 21D shows the results in a control mouse. Red signal: tdTomato; Green signal: Cre; Blue signal: DAPI (nuclei statining).

### DETAILED DESCRIPTION OF THE INVENTION

### 1. Overview

The invention described herein provides a recombinant viral particle comprising a DNA virus protein shell, and a “vector genome” comprising RNA, such as single-stranded RNA (rather than DNA). The “vector genome” may not be a typical viral RNA, in that it may have very little, if any, virus-originated sequences, other than the RNA Packaging Signal (RPS) described herein below. That is, the DNA virus normally or naturally encapsidates a DNA viral vector genome inside the protein shell, while the recombinant version of the DNA virus viral particle as described herein encapsidates instead an RNA. By “RNA” or “ribonucleic acid” it means a stretch of ribonucleotides each composed of a phosphate, a ribose, and a base (A (adenine), U (uracil), G (guanine), or C (cytosine)), each of which ribonucleotides may be modified (for example, base-modified, glycosyl-modified, phosphate-modified, e.g., oxygen-modified, fluorine-modified, sulphur-modified, pseudo-modified (e.g., pseudo-uridine-modified), methylated, capped (e.g., 5-capped)) or unmodified, and, optionally, fused directly or indirectly with a stretch of deoxyribonucleotides each composed of a phosphate, a deoxyribose, and a base (A (adenine), T (thymine), G (guanine), or C (cytosine)), each of which deoxyribonucleotides may be modified (for example, base-modified, glycosyl-modified, phosphate-modified, e.g., oxygen-modified, fluorine-modified, sulphur-modified, pseudo-modified, methylated, capped (e.g., 5-capped)) or unmodified, e.g., a RNA-DNA chimera, a DNA-RNA-DNA chimera, a RNA-DNA-RNA chimera.

A typical (non-limiting) example of such a recombinant DNA virus viral particle is adeno-associated virus (AAV), which normally/naturally encapsidates a single-stranded DNA (ssDNA) vector genome. Another non-limiting example of such DNA virus is an oncolytic DNA virus, such as an oncolytic herpes virus (e.g., herpes simplex virus or HSV), an oncolytic adenovirus, a vaccinia virus (VACV), vesicular stomatitis virus (VSV), etc.

The invention is partly based on the surprising discovery that, transcribed AAV ITR, in RNA form, can facilitate high efficiency direct packaging of transcribed RNA encompassing such transcribed AAV ITR into conventional AAV viral particles.

The invention described herein is also partly based on the surprising discovery that, other than the transcribed AAV ITR (RNA), certain artificial or heterologous RNA sequences and their cognate/corresponding/native RNA binding proteins can also serve as pairs of RNA Packaging Signals (RPS) and RPS-Interacting Proteins (RPSIPs) to replace the function of wild-type packaging signal sequences and interacting proteins useful for DNA virus packaging, thus packaging an RNA into a DNA virus protein shell that normally/naturally encapsidates a DNA vector genome.

For example, in wild-type AAV, the ITR sequences at the 5′ and 3′ ends of the DNA vector genome comprise sequence elements such as Rep-Binding Element (RBE) and RBE′ that can interact with the Rep proteins (such as Rep68 and Rep78). The Rep proteins bind the ITR and facilitate the packaging of AAV ssDNA vector genome comprising such ITR sequence elements into the AAV viral particle.

The inventors have discovered that, by providing, as an RPS, a transcribed ITR sequence, and/or an artificial or heterologous RNA sequence, such as the MS2 sequence, to an RNA sequence of interest (RSI), the resulting RNA sequence comprised of the RPS and the RSI can be efficiently packaged into an AAV viral protein shell in the presence of MCP—the bacteriophage-derived MS2 coat protein (MCP) that naturally binds MS2. The ability of the artificial RPS/RPSIP pair—e.g., MS2/MCP—to facilitate RNA packaging into a DNA virus protein shell, does not depend on the presence of, but can function independently of, the native ITR packaging signal for DNA packaging. In a sense, the heterologous MS2-MCP pair constitutes an artificial system of RPS and RPSIP pair that can effectively replace the natural ITR-Rep DNA packaging system, with the former efficiently facilitates RNA packaging. Such RNA-containing DNA virus, such as AAV, may be referred herein as R-DNA viral particle (or RAAV in the case of AAV), or recombinant R-DNA viral particle (or rRAAV in the case of AAV).

The R-DNA viral particle and RAAV viral particles of the invention can be used to deliver the RNA transcript of any transgene or gene of interest (GOI) of suitable length (e.g., within the packaging limit of the various DNA virus or AAVs) or any guide RNA to a host cell compatible with the tropism of the DNA viral protein shell or AAV viral capsid shell. As used herein, the recombinant DNA viral particles such as recombinant AAV vectors, vector genomes, and recombinant AAV viral particles or recombinant AAV particles, are referred to herein as rRAAV vectors (recombinant RNA adeno-associated virus vectors), vector genomes, and recombinant RAAV (rRAAV) viral particles or rRAAV particles, respectively (the “rRAAV vectors” and “rRAAV particles” are used exchangeably herein).

Specifically, on the one hand, just like any normal or conventional AAV vectors, the subject RAAV vectors can also be composed of any of the same capsid shells found in any wild-type AAVs carrying DNA as the viral genetic material. Thus, the subject RAAV vectors possess all the usual advantages derived from the AAV shell, such as specific/broad tropism and low immugenicity.

However, on the other hand, the genome of the subject RAAV vectors are comprised of RNAs (e.g., mRNAs), which have short lifespans, and thereby leading to a transient expression of any encoded gene product on such RNA genetic material.

Such transient expression is desired in at least some cases. For example, the RAAV vectors of the invention are advantageous for in vivo DNA gene editing, since time-restricted exposure to RAAV-encoded DNA gene editors (such as the mRNA coding sequence for a CRISPR/Cas system effector enzyme Cas9 and variants thereof fused to a base editor) may enable efficient gene editing. Such transiently expressed DNA editors also improves the safety profile of the gene therapy, by reducing off-target gene targeting, and reducing immunogenicity compared to the persistent expression of the same DNA gene editors expressed from conventional DNA-based AAV vectors.

In addition, compared to traditional DNA-based AAV vectors, the subject RAAV vectors can carry longer transgenes, because of the exclusion of at least the promoter (and also any non-transcribed enhancer sequences that may be) required for expression of the GOI encoded by a DNA-based AAV vector.

Although the subject rRAAV vectors have different sequence elements and organization compared to traditional DNA-based AAV vectors, the rRAAV viral particles have the same entry and intracellular-trafficking processes as the conventional DNA-based AAV vectors. However, they have quite different fates after entering into the host cell nucleus. After entering into the nucleus, the mRNA genome of the subject RAAV vector is released and subsequently transported to the cytoplasm, leading to translation. As is understood, mRNAs generally have short lifespans, ranging from several minutes to days, and are eventually degraded via many cellular mechanisms. However, the limited mRNA lifespan still enables the host cell to complete the protein synthesis, often without the delay due to the 2nd strand cDNA synthesis in DNA-based AAV vectors, and allowing the encoded proteins to function rapidly.

Numerous such RPS/RPSIP pairs can be used for RNA packaging into DNA virus. The inventors have demonstrated at least two additional such pairs, including the PP7 sequence and the PP7 bacteriophage coat protein (PCP), and the com sequence and the phage COM protein (COM), that efficiently package RNA comprising the heterologous RPS (i.e., PP7 and com sequences, respectively). The three pairs of RPS/RPSIP as demonstrated encompass at least two categories. Unlike MS2/MCP and PP7/PCP that are natural viral packaging systems, com/COM is not a natural viral packaging system but known to be transcription regulators that play roles in the transcription initiation of the bacteriophage Mu mom gene. Numerous transcribed modified AAV ITR sequences can also be used as RPS of the invention.

The invention described herein is also not limited to a specific serotype of DNA virus (e.g., a specific AAV serotype). The inventors have demonstrated efficient packaging of RNA sequences with suitable RPS into representative AAV viruses including AAV5, AAV8, AAV9, and AAV-DJ, using in conjunction with compatible RPSIP in each case.

The invention described herein is also based on the discovery that the efficiency of packaging undesired DNA into natural DNA virus viral particles can be decreased by several independent approaches.

In certain embodiments, the undesired DNA packaging efficiency can be reduced by increasing the overall size of the DNA vector from which the RNA of interest is transcribed. For example, in the often used triple transfection method for AAV production, the gene of interest (GOI) can be carried by a first plasmid, the required Rep and Cap proteins are encoded by the rep and cap genes on a second plasmid, while the other AAV packaging required components are provided by a third plasmid. According to this embodiment of the invention, the RNA sequence to be packaged into the DNA virus can be transcribed from the first plasmid, and the overall size of the first plasmid can be artificially increased by including a random stuffer sequence (e.g., an intron), such as a stuffer sequence that is at least about 1 kb, 2 kb, 3 kb, 4 kb, 5 kb or more in length, or a stuffer sequence that increases the overall size of the first plasmid by 1 kb, 2 kb, 3 kb, 4 kb, 5 kb or more, e.g., to about 6 kb, 7 kb, 8 kb, 9 kb, 10 kb or more, etc.

In certain other embodiments, the undesired DNA packaging efficiency can be reduced by inhibiting the function of a canonical element that facilitates DNA packaging. Such a canonical element for DNA packaging may include a DNA sequence (such as an element of the AAV ITR sequence that facilitates DNA packaging, including the trs sequence, the RBE or RBE′ sequence, or the entire ITR sequence of an AAV); and/or a protein element participating in the DNA packaging, such as, a protein that interacts with the DNA sequence (such as a mutant Rep68 or Rep 78 protein that lacks or has diminished trs-endonuclease activity).

Thus, one aspect of the invention provides a ribonucleotide (RNA) sequence capable of being packaged into a DNA virus viral particle, such as a DNA virus that naturally packages DNA, wherein the RNA sequence comprises: (1) an RNA sequence of interest (RSI); and, (2) an RNA-packaging signal (RPS) capable of interacting, e.g., binding, directly or indirectly to an RPS-interacting molecule (e.g., an RPS-interacting protein or RPSIP) that facilitates packaging of the RNA sequence into the DNA virus viral particle.

Such an RNA sequence can comprise any RSI (RNA), which may be encoded by “a gene of interest” or “GOI” (DNA).

As used herein, “a gene of interest” or “GOI” includes any coding sequence for a protein or polypeptide, including intron and exon sequences, and/or coding sequence for any non-translated RNA or non-coding RNA (ncRNA, such as siRNA, piRNA, short hairpin RNA or shRNA, microRNA or miRNA or precursors thereof including pre-miRNA and pri-miRNA, antisense sequence or oligonucleotide (ASO), guide RNA or gRNA for CRISPR/Cas, rRNA, tRNA, snoRNA, snRNA, exRNA, scaRNA, lncRNA, Xist, and HOTAIR, etc.).

Similarly, representative (non-limiting) RSI includes, for example, a protein (e.g., a therapeutic protein, an antigen protein, or a gene-editing protein such as a CRISPR/Cas effector enzyme (“a Cas protein” for short), a ZFN protein, a TALEN protein)-encoding RNA, such as an mRNA, or a non-coding, functional RNA (such as a transfer RNA (tRNA), a ribosomal RNA (rRNA), a transfer-messenger RNA (tmRNA), a small interfering RNA (siRNA), a short hairpin RNA (shRNA), an antisense RNA or oligonucleotide (ASO), a micro RNA (miRNA), an RNA aptamer, or an RNA component of a CRISPR-Cas (e.g., Cas9, Cas12, Cas13) system, such as, a single guide RNA (or an sgRNA, a chimeric RNA, an RNA chimera), a CRISPR RNA (crRNA) and a tracr RNA), or a precursor thereof, or an RNA component of a RISC complex or RNAi pathway (such as shRNA, miRNA, or siRNA), a regulatory RNA, Piwi-interacting RNAs (piRNAs), small nucleolar RNAs (snoRNAs), a long non-coding RNA (lncRNA) (including intergenic lincRNA, intronic ncRNA, and sense/antisense lncRNA), a long intervening/intergenic noncoding RNA (lincRNA), an enhancer RNA, a bacterial small RNA (sRNA), snRNA, exRNA, scaRNA, Xist, and HOTAIR, and a precursor thereof.

The RNA sequence of the invention or GOI can comprise one coding sequence, or more than one (e.g., 2, 3, 4, 5, 6, 7, 8, 9, 10 or more) coding sequences. The length of the coding sequence, or the combined length of all coding sequences, may be no more than the maximum length of RNA that can be packaged into a particular or chosen DNA virus viral particle (e.g., AAV viral particle), which can differ from one specific DNA virus (e.g., AAV) viral particle from another.

In certain embodiments, a DNA sequence encoding or corresponding to the RNA sequence of the invention, or a reverse complement of the DNA sequence, has reduced, diminished, or substantially no capacity of being packaged into the DNA virus viral particle. For example, the DNA sequence may encode the RNA sequence of the invention (e.g., the DNA sequence has the reverse complement sequence of the RNA sequence of the invention). The DNA sequence may also correspond to the RNA sequence of the invention, in that the DNA sequence has otherwise identical nucleotide sequence as the RNA sequence of the invention, except that the DNA sequence has T's, instead of the U's in the RNA sequence of the invention. Regardless, the DNA sequence or the reverse complement thereof may lack a functional DNA packaging signal for packaging into the DNA virus viral particle, such as an AAV ITR for AAV packaging, such that the DNA sequence or the reverse complement thereof (DNA) has reduced, diminished, or substantially no capacity of being packaged into the DNA virus viral particle.

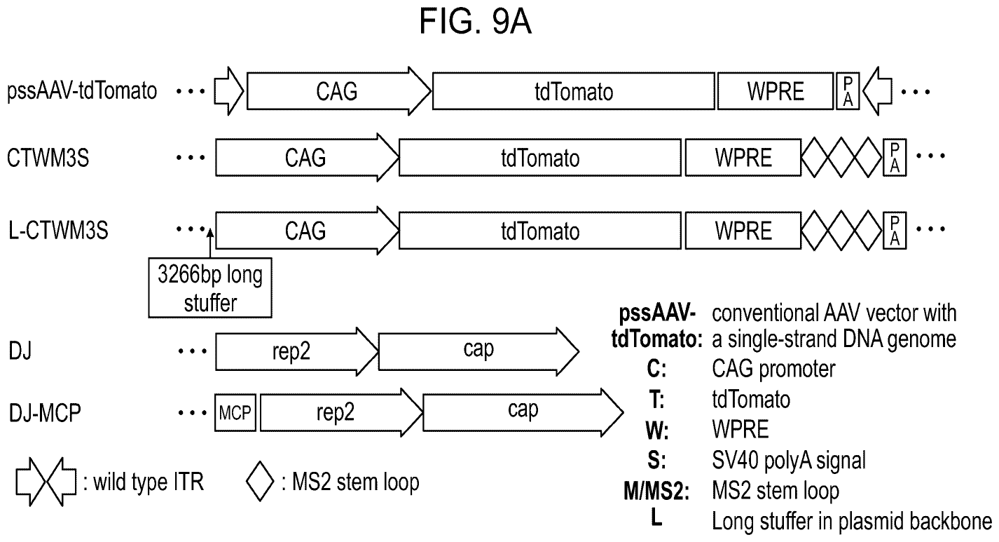

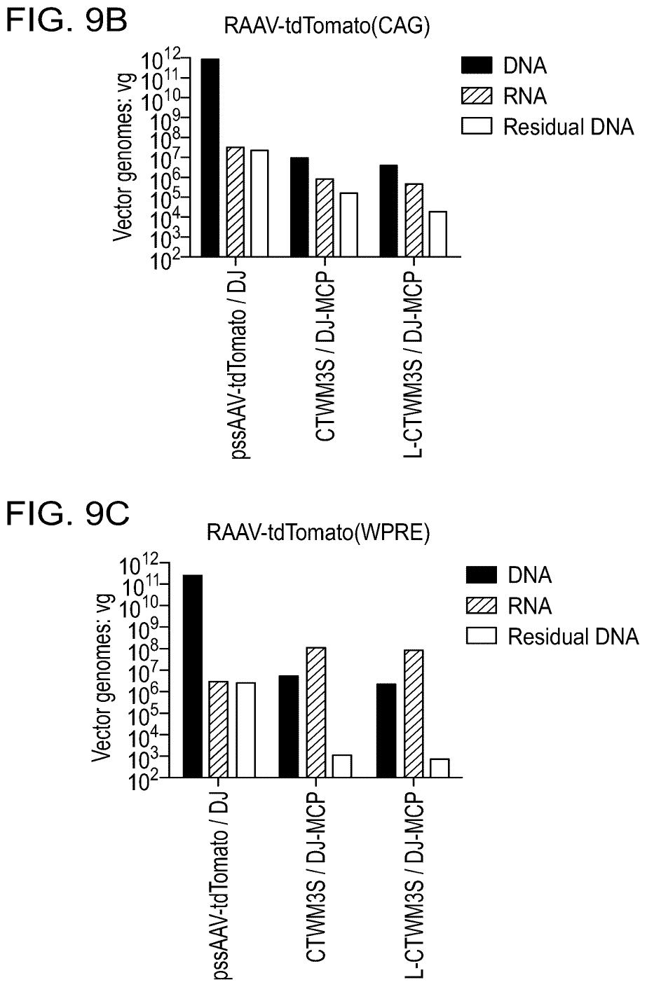

In certain embodiments, the RNA sequence of the invention is transcribed from a DNA construct, such as transcribed from a DNA plasmid encoding the RNA sequence, wherein the DNA construct/plasmid comprises a stuffer sequence (e.g., an intron sequence) in its backbone sequence to enhance packaging of the RNA sequence of the invention, and/or to reduce undesired packaging of DNA into the DNA virus viral particle. For example, the RNA sequence of the invention can be transcribed from a DNA construct/plasmid, and the overall size of the DNA construct/plasmid can be artificially increased by including a random DNA stuffer sequence, such as a stuffer sequence that is at least about 1 kb, 2 kb, 3 kb, 4 kb, 5 kb or more in length, or a stuffer sequence that increases the overall size of the DNA construct/plasmid by 1 kb, 2 kb, 3 kb, 4 kb, 5 kb or more, e.g., to about 6 kb, 7 kb, 8 kb, 9 kb, 10 kb or more, etc. The stuffer sequence can be located upstream (e.g., immediately upstream) of the transcription unit comprising the coding sequence for the RNA sequence of the invention (see FIG. 9A, in which a long stuffer sequence of >3 kb is inserted immediately upstream of a CAG promoter that drives the transcription of an exemplary RNA sequence of the invention). In certain embodiments, the stuffer sequence is inserted immediately upstream of a promoter operably linked to the codon sequence for the RNA sequence of the invention. Optionally, in some embodiment, the coding sequence for the RNA sequence of the invention is devoid of a functional natural DNA packaging signals for the DNA virus viral particle, such as devoid of a functional ITR sequence that supports packaging into an AAV viral particle.

In certain embodiments, the RNA sequence of the invention is capable of being packaged into a DNA virus viral particle that is an AAV viral particle. Any AAV virus can be used to package the RNA sequence of the invention, including, but not limited to, AAV1, AAV2, AAV3A, AAV3B, AAV4, AAV5, AAV6, AAV7, AAV8, AAV9, AAV10, AAV 11, AAV 12, AAV 13, AAVrh10, AAVrh74, AAVhu32, AAVhu37, AAV-DJ, AAV PHP.cB, Anc80L65, Anc80L65AAP, AAVrh74, or 7m8.

In certain embodiments, the RNA sequence of the invention is capable of being packaged into a DNA virus viral particle that is an oncolytic viral particle. Exemplary (non-limiting) oncolytic viral particles include: oncolytic herpes virus (e.g., herpes simplex virus or HSV), an oncolytic adenovirus, a vaccinia virus (VACV), vesicular stomatitis virus (VSV), etc.

The location of the RPS in the RNA sequence of the invention can be flexible. In certain embodiments, the RPS is located at or near the 5′ end of the RNA sequence of the invention, at or near the 3′ end of the RNA sequence of the invention, or internal to the RNA sequence of the invention. In certain embodiments, the RPS is located at or near the 5′ end of the RNA sequence of interest (RSI), at or near the 3′ end of the RNA sequence of interest (RSI), or internal to the RNA sequence of interest (e.g., inside an intron of an mRNA).

There can be one or more RPS in the RNA sequence of the invention. In certain embodiments, the RNA sequence of the invention comprises more than one (e.g., 1, 2, 3, or more) RPS that are identical or substantially identical. In certain embodiments, the RNA sequence of the invention comprises more than one (e.g., 1, 2, 3, or more) RPS, and at least two of which are different from each other.

In cases where more than one RPS are present on the RNA sequence of the invention, at least two of the more than one RPS are adjacent to each other, such as in tandem, with an optional linker sequence in between. The linker between any two adjacent RPS sequences may be the same or different. The linker sequence may be a randomized RNA sequence with no substantial secondary structure, no known functional sequences or elements, and/or may be less than 50% in GC content. The length of the linker may be any where between 1-1 kb, 1-500 bases, 1-200 bases, 1 to about 100 bases, 1 to about 60 bases, about 5 to about 55 bases, about 10 to about 30 bases, or about 15-25 bases.

In certain embodiments, the RNA sequence of the invention comprises 3 RPS sequences adjacent to one another, separated by two linker sequences, each independently about 20 or about 50 bases. For example, the first two of three identical RPS sequences may be separated by a linker of 20 bases, and/or the last two of the RPS sequences may be separated by a linker of 51 bases.

In certain embodiments, the RNA sequence of the invention comprises more than one RPS (e.g., 1, 2, 3, 4, or 5 RPS), wherein at least two of the more than one RPS are not adjacent to each other. For example, one of the RPS may be located at the 5′ end of the RNA sequence of the invention, while another RPS may be located at the 3′ end of the RNA sequence of the invention, and an optional 3rd RPS may be located inside an intron of an mRNA as the RSI within the RNA sequence of the invention. A 4^{th} and/or a 5^{th} RPS may be located close or adjacent to any one the first, second, or third RPS.

In certain embodiments, the RNA sequence of the invention comprises at least two (e.g., two or more) RPS sequences that are not adjacent to each other, e.g., one each located at or near one end of the RNA sequence of interest (RSI).

In certain embodiments, the RPS comprises a transcribed modified AAV inverted terminal repeat (ITR), wherein the transcribed modified AAV ITR (a) comprises a transcribed functional Rep-Binding Element (RBE), optionally further comprising a transcribed functional RBE′; and, (b) lacks either a transcribed terminal resolution site (TRS), or a transcribed reverse complement TRS (rcTRS), or both. In certain embodiments, the transcribed modified AAV ITR further comprises a transcribed D region sequence (D sequence or D′ sequence). In certain embodiments, the RPS-interacting molecule is Rep78, Rep68, Rep52, and/or Rep40.

As used herein, “AAV viral particle” includes viral particles comprising any wild-type capsids of adeno-associated virus (AAV) (belonging to the genus Dependoparvovirus, which in turn belongs to the family Parvoviridae), as well as engineered or variants thereof having modified sequence and/or tissue or host tropism.

As used herein, “intron” refers to a non-coding segment of a DNA or an RNA, which are normally removed a transcribed RNA through splicing. However, the RNA sequence of the invention may comprise an intron sequence, such as an intron sequence from a heterologous gene (“heterologous” with respect to the gene of interest or GOI, which is to be expressed as a transgene delivered to a host cell by the rRAAV viral particle of the invention), in order to enhance the expression of the GOI. Such intron sequence in the RNA sequence of the invention may or may not be removed by splicing. In addition, such intron sequence may further comprise a transcribed enhancer or a part thereof, since certain enhancers can be located within an intron of a coding DNA.

As used herein, “exon” refers to a coding segment of a DNA or an RNA, which exon is to be translated into a protein sequence. However, in certain embodiments, an exon sequence within the RNA sequence of the invention may encode part of or the entirety of the GOI to be expressed as a transgene delivered to a host cell by the rRAAV viral particle of the invention. In other embodiments, an exon sequence within the RNA sequence of the invention may belong to a heterologous gene (with respect to the GOI), and the presence of such exon may enhance the expression of the GOI.

As used herein, “coding sequence” includes a polynucleotide sequence of a DNA or an RNA which encodes a product that can be (a) a protein or a polypeptide, or (2) other than a protein or a polypeptide (e.g., ncRNA, such as siRNA, piRNA, short hairpin RNA or shRNA, microRNA or miRNA or precursors thereof including pre-miRNA and pri-miRNA, antisense sequence or oligonucleotide (ASO), guide RNA or gRNA for CRISPR/Cas, rRNA, RNA, snoRNA, snRNA, exRNA, scaRNA, lncRNA, Xist, and HOTAIR, etc.).

The ribonucleotide coding sequence for the gene of interest may be further processed inside the cell, once the RNA content of the RAAV viral particle is separated from the AAV capsid and released into the cell. Processing of the coding sequence can produce one or more RNA products, such as siRNA, miRNA, and/or mRNA, which may be further translated into protein product(s), or be incorporated into other cellular machinery such as the RISC complex or a CRISPR/Cas effector enzyme (such as a Class 2, type II, V, or VI effector enzyme).

As used herein, the term “transcribed,” and grammatical variations thereof, refers to a nucleotide sequence comprising ribonucleic acid (RNA) nucleotides that have been transcribed from a DNA template (e.g., double-stranded DNA and/or single-stranded DNA). The transcribed RNA molecule can corresponds to either a plus strand or a minus strand of an AAV ssDNA, wherein the transcribed plus strand RNA was transcribed from the minus strand of the DNA template and the transcribed minus strand RNA was transcribed from the plus strand of the DNA template. In certain embodiments, the transcribed RNA molecule can either be transcribed from the sense or antisense strand of a double stranded DNA template. For example, when the dsDNA sequence is represented by the sequence of only one strand (such as SEQ ID NO: 1), a transcribed RNA using the dsDNA as template may have the same sequence as the sense strand or the antisense strand, as the case may be. That is, RNA transcribed from double-stranded DNA shown as SEQ ID NO: 1 may have the same sequence as SEQ ID NO: 1 or its reverse complement, except that the T's in DNA are replaced by U's in the transcribed RNA.

The transcribed modified AAV inverted terminal repeat (ITR) sequence of the invention is an RNA sequence (as opposed to the single-stranded DNA sequence in the conventional AAV viral genome encapsidated within the AAV viral particle). As the wild-type AAV ITR DNA sequence, the transcribed modified AAV ITR sequence (RNA) also supports binding of the RNA sequence of the invention to the AAV Rep protein, and is thus capable of supporting the direct packaging of the RNA sequence of the invention into the AAV viral particle. In certain embodiments, the transcribed modified ITR sequence comprises a transcribed Rep-binding element (RBE) (e.g., a transcribed functional RBE), and optionally a transcribed RBE′ (e.g., a transcribed functional RBE′), for Rep binding. In certain embodiments, the transcribed modified ITR sequence supports or facilitates packaging or encapsidation of the RNA sequence into an AAV viral particle.

In certain embodiments, the modified ITR comprises a wild-type RBE.

In certain embodiments, the modified ITR comprises a functional RBE that retains at least about 60%, 70%, 80%, 90%, 95%, 100% or more of the ability of wild-type RBE for supporting AAV packaging, such as Rep binding. In certain embodiments, the functional RBE comprises up to about 30%, 25%, 20%, 15%, 10%, or 5% of sequence variation compared to the wild-type RBE, due to, for example, insertion, deletion, substitution, and/or other mutation of one or more nucleotides of the RBE.

In certain embodiments, the modified AAV ITR DNA template, from which the transcribed modified AAV ITR is transcribed, is defective as an ITR, in that it lacks one or more functions of the corresponding wild-type AAV ITR, such as being able to be cleaved at the TRS (transcribed terminal resolution site, see below). This can be due to, for example, the lack of a functional TRS. In one embodiment, the wild-type TRS is completely deleted such that the modified ITR has no TRS. In one embodiment, the wild-type TRS is mutated by deleting, inserting, substituting, and/or mutating one or more nucleotides such that it can no longer to recognized and cleaved by Rep during AAV replication.

In certain embodiments, the modified AAV ITR DNA template retains the RBE or a functional variant thereof as described herein, and optionally the RBE′ or a functional variant thereof. In certain embodiments, the RBE and/or RBE′ is/are functional with respect to binding to AAV Rep78/68.

The transcribed modified AAV inverted terminal repeat (ITR) of the invention further lacks either a transcribed terminal resolution site (TRS), or a transcribed reverse complement TRS (rcTRS), or both. In certain embodiments, the TRS is at the 5′ end of the modified AAV ITR. In certain embodiments, the TRS is between the D region sequence and the RBE.

In certain embodiments, the transcribed modified AAV ITR lacks both the transcribed TRS and the transcribed rcTRS.

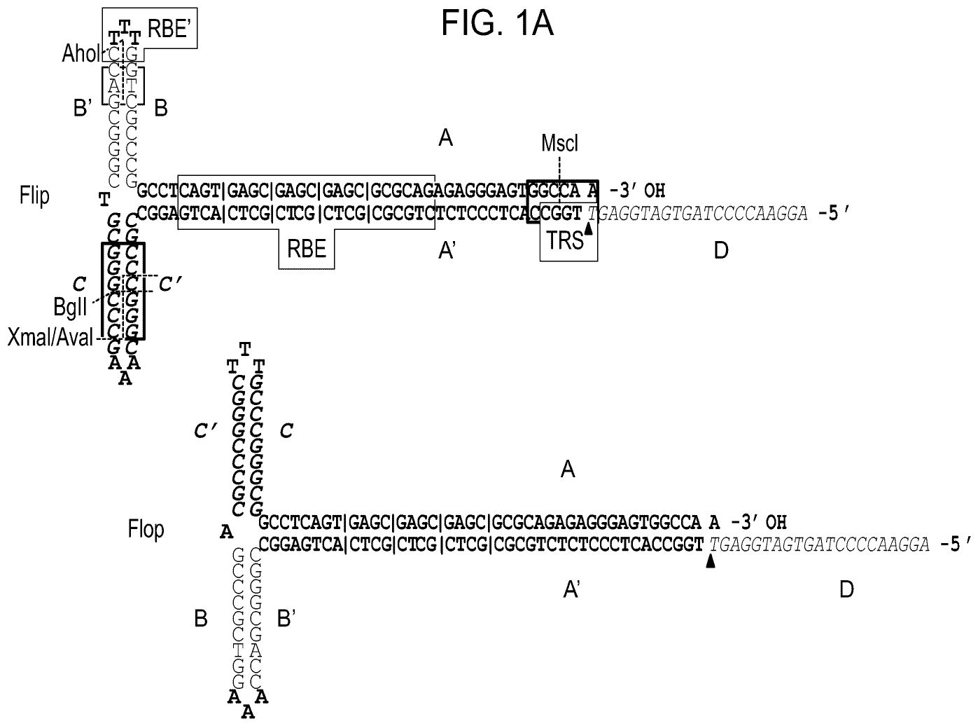

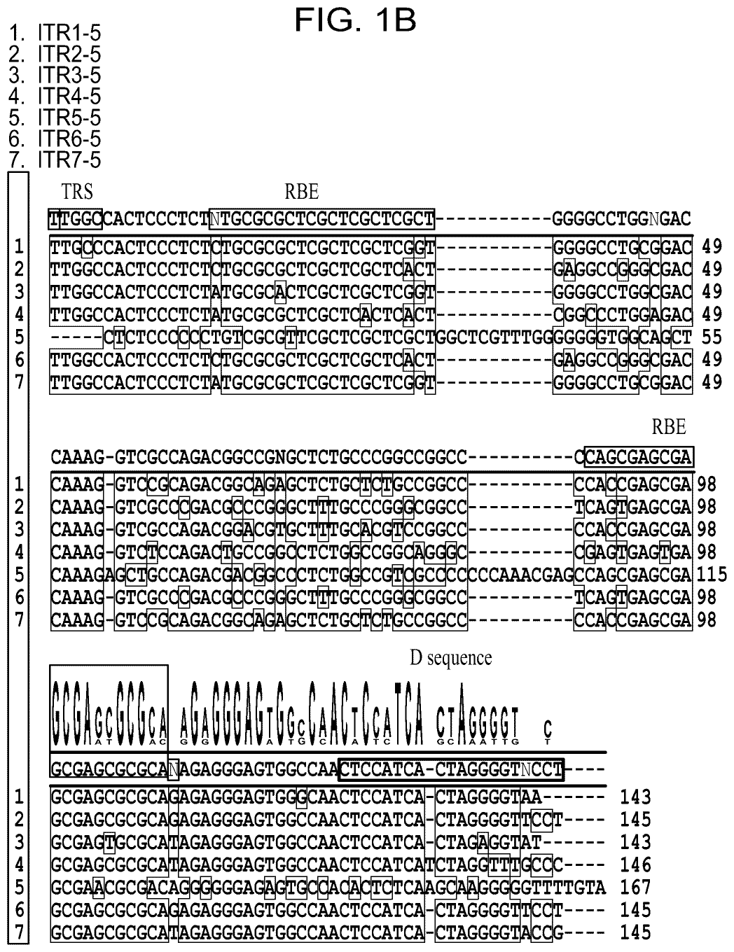

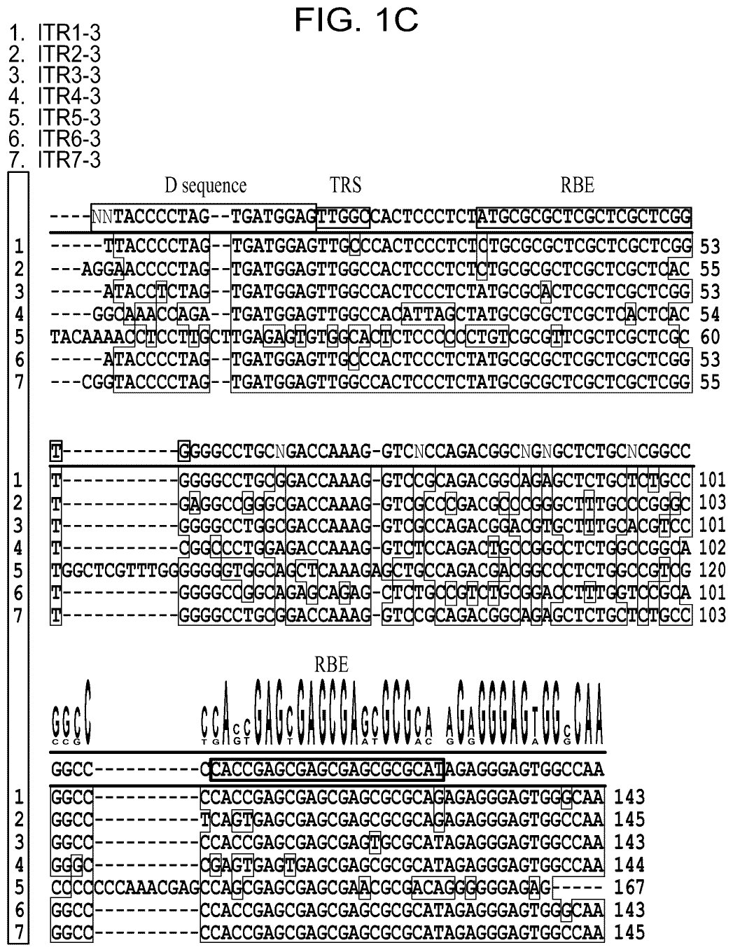

As used herein, “terminal resolution site” or “TRS” refers to the single-stranded DNA sequence in the single-stranded AAV vector genome (plus or minus strand) that is recognized and nicked by the AAV Rep proteins during AAV replication. As used herein, “reverse complement TRS (rcTRS)” refers to the single-stranded DNA sequence in the single-stranded AAV vector genome (plus or minus strand) that is reverse complement sequence of the TRS. The rcTRS pairs with the TRS to form a double stranded DNA region at one end of the A region stem. See FIGS. 1A-1C.

In AAV2 ITR, the TRS comprises the sequence of TTGGC, with the Rep cleavage site in between the two T's; while the rcTRS comprises the sequence of GCCAA. One TRS is located at the juncture of the D and A region sequences, and is at the most 5′ end of the A region sequence (e.g., between the D region sequence and the RBE). See FIGS. 1B and 1C for the TRS and rcTRS in 5′ and 3′ ITR multi-sequence alignment of representative AAV's.

As used herein, a “transcribed TRS” is a single-stranded RNA sequence resulting from transcribing the TRS DNA template. For AAV2 TRS comprising TTGGC, the transcribed TRS comprises GCCAA.

As used herein, a “transcribed rcTRS” is a single-stranded RNA sequence resulting from transcribing the rcTRS DNA template. For AAV2 rcTRS comprising GCCAA, the transcribed rcTRS comprises UUGGC.

Thus, a transcribed modified AAV ITR “lacks a transcribed AAV2 TRS,” if it does not have the GCCAA sequence at the location the GCCAA sequence normally appears in a corresponding transcribed wild-type AAV2 ITR, e.g., due to complete deletion of the GCCAA sequence, or due to insertion, deletion, substitution, and/or other mutation of one or more nucleotides within the GCCAA sequence. This can result from transcribing a modified AAV ITR having a complete deletion of the TRS (TTGGC), or due to insertion, deletion, substitution, and/or other mutation of one or more nucleotides within the wild-type TRS.

Thus, in certain embodiments, the RNA sequence of the invention or the transcribed modified AAV ITR lacks a transcribed functional TRS.

Similarly, a transcribed modified AAV ITR “lacks a transcribed AAV2 rcTRS,” if it does not have the UUGGC sequence at the location the UUGGC sequence normally appears in a corresponding transcribed wild-type AAV2 ITR, e.g., due to complete deletion of the GCCAA sequence, or due to insertion, deletion, substitution, and/or other mutation of one or more nucleotides within the GCCAA sequence. This can result from transcribing a modified AAV ITR having a complete deletion of the rcTRS, or due to insertion, deletion, substitution, and/or other mutation of one or more nucleotides within the wild-type rcTRS.

In certain embodiments, the transcribed modified AAV ITR further comprises a transcribed D region sequence (D or D′ sequence in a wild-type AAV ITR) or a mutant D region sequence (e.g., one with one or more nucleotide insertion, deletion, substitution, and/or other mutation) that substantially retains the function of a wild-type D region sequence. In other embodiment, the transcribed modified AAV ITR does not comprises a transcribed D region sequence, or does not comprise a mutant D region sequence (e.g., one with one or more nucleotide insertion, deletion, substitution, and/or other mutation) that substantially retains the function of a wild-type D region sequence.

In certain embodiments, the transcribed modified AAV ITR comprises the transcribed (functional) D region sequence. Optionally, the modified AAV ITR DNA template has the nucleotide sequence of SEQ ID NO: 3. Optionally, the transcribed modified AAV ITR comprises an RNA equivalent of SEQ ID NO: 3 (i.e., the RNA equivalent has the same base sequence as the DNA sequence of SEQ ID NO: 3). Optionally, the transcribed modified AAV ITR comprises an RNA equivalent of the reverse complement of SEQ ID NO: 3 (i.e., the RNA equivalent has the same base sequence as the DNA sequence of the reverse complement of SEQ ID NO: 3).

In certain embodiments, the transcribed modified AAV ITR lacks the transcribed (functional) D region sequence. Optionally, the modified AAV ITR DNA template has the nucleotide sequence of SEQ ID NO: 2. Optionally, the transcribed modified AAV ITR comprises an RNA equivalent of SEQ ID NO: 2 (i.e., the RNA equivalent has the same base sequence as the DNA sequence of SEQ ID NO: 2). Optionally, the transcribed modified AAV ITR comprises an RNA equivalent of the reverse complement of SEQ ID NO: 2 (i.e., the RNA equivalent has the same base sequence as the DNA sequence of the reverse complement of SEQ ID NO: 2).

As used herein, “D region sequence” refers to either the D sequence or its reverse complement D′ sequence. Location of the D region sequence depends on whether the ITR takes the “flip” or the “flop” configuration. See FIGS. 1A-1C. For example, in wild-type AAV2 ITR (see FIG. 2 of Srivastava et al., J. Viol. 45 (2): 555-564, 1983, incorporated herein by reference), the plus strand ssDNA sequence comprises, from 5′ to 3′, palindromic sequence segments named A, B, B′, C, C′, A′, D, . . . , D′, A, C, C, B, B′, and A′, in which A: A′, B: B′, C: C′ and D: D′ are reverse complement sequences of each other and can form base-paired stem sequences (though the D and D′ sequences may not actually base-pair with each other in the ssDNA AAV vector genome). The 5′ ITR of the plus strand has the B: B′ stem closer to one end (5′ end) of the sequence than the C: C′ stem, and is known as the flip ITR. The 3′ ITR of the plus strand has the C: C′ stem closer to one end (3′ end) of the sequence than the B: B′ stem, and is known as the flop ITR.

The transcribed modified AAV ITR sequence of the invention may lack a functional transcribed D region sequence (D or D′ sequence) by, for example, deletion, insertion, substitution, and/or other mutation of one or more nucleotides of the transcribed wild-type D region sequence.

In certain embodiments, the RNA or transcribed modified AAV ITR sequence of the invention comprises a mutated transcribed D region sequence and/or a mutated transcribed TRS sequence. In certain embodiments, the RNA or transcribed modified AAV ITR sequence of the invention comprises no transcribed D region sequence and/or no transcribed TRS/rcTRS sequence.

In certain embodiments, the transcribed modified AAV ITR is modified based on a transcribed wild-type flip ITR or a wild-type flop ITR.

In certain embodiments, the wild-type flip ITR or the wild-type flop ITR is from AAV1, AAV2, AAV3A, AAV3B, AAV4, AAV5, AAV6, AAV7, AAV8, AAV9, AAV10, AAV 11, AAV 12, AAV 13, AAVrh10, AAVrh74, AAVhu32, AAVhu37, AAV PHP.cB, Anc80L65, Anc80L65AAP, AAVrh74, or 7m8. Optionally, the wild-type flop ITR has the nucleotide sequence of SEQ ID NO: 1.

In certain embodiments, the transcribed D region sequence is present, and is not within the 3′ end 50 nucleotides (e.g., 40 nt, 30 nt, 25 nt, or 20 nt) of the RNA.

In certain embodiments, the transcribed D region sequence is present, and is within the 3′ end 50 nucleotides (e.g., 40 nt, 30 nt, 25 nt, or 20 nt) of the RNA.

In certain embodiments, the transcribed modified AAV ITR is within the 3′ end 1000 nucleotides of the RNA. In certain embodiments, the transcribed modified AAV ITR is within the 3′ end 800 nucleotides of the RNA. In certain embodiments, the transcribed modified AAV ITR is within the 3′ end 500 nucleotides of the RNA. In certain embodiments, the transcribed modified AAV ITR is within the 3′ end 300 nucleotides of the RNA. In certain embodiments, the transcribed modified AAV ITR is within the 3′ end 200 nucleotides of the RNA.

In certain embodiments, the transcribed modified AAV ITR is 5′ to a polyA sequence, a polyA signal sequence (e.g., AAUAAA), or a sequence for RNA transcription termination (e.g., a histone downstream element).

As used herein, “polyA sequence” or “polyA tail” refers to a string of adenine ribonucleotides or adenosine monophosphates (e.g., a string of RNA with each base therewithin an adenine). Such a polyA tail is important for the nuclear export, translation and stability of mRNA. The length of the polyA sequence can vary in different mRNA or the RNA sequence of the invention, and can be about 250 nucleotides of polyA, about 230 nucleotides of polyA, about 200 nucleotides of polyA, about 180 nucleotides of poly A, about 160 nucleotides of polyA, about 140 nucleotides of polyA, about 120 nucleotides of polyA, about 100 nucleotides of polyA, or less.

As used herein, “polyA signal sequence” refers to an RNA sequence (such as AAUAAA) that is located downstream of the most 3′ exon, and is recognized by an RNA cleavage complex that cleaves off the 3′ terminal sequence of a newly transcribed RNA by RNA polymerase (such as Pol II) such that polyadenylation can occur. Polyadenylate polymerase then adds and extends the poly(A) tail by adding adenosine monophosphate units from ATP to the nascent cleaved 3′ end of the RNA. The initial RNA cleavage is typically catalyzed by the enzyme CPSF (cleavage/polyadenylation specificity factor), and occurs about 10-30 nucleotides downstream of its binding site—the polyA signal sequence, which is often AAUAAA on the transcribed RNA. The sequence at/or immediately 5′ to the site of RNA cleavage is frequently (but not always) CA. The polyA signal sequence recognized by the RNA cleavage complex varies between different groups of eukaryotes, with most human polyadenylation sites containing the AAUAAA sequence, though this sequence is less common in plants and fungi mRNA. In addition, other variants that bind more weakly to CPSF exist. All such sequence motifs recognized by the RNA cleavage complex to enable RNA cleavage and the subsequent polyadenylation are within the scope of the polyA signal sequence.

Also as used herein, “a transcribed GU-rich region downstream of the polyA site” refers to a sequence that may be used by other proteins (such as the cleavage stimulation factor or CstF) to enhance binding specificity of CPSF to the poly A signal sequence (e.g., AAUAAA).

In certain embodiments, the RNA sequence of the invention further comprises a recognition sequence for CFI (cleavage factor I), such as a set of UGUAA sequences in mammals, that can recruit CPSF even if the AAUAAA polyA signal sequence is missing.

As used herein, “a sequence for RNA transcription termination” includes an RNA sequence motif present at or near the 3′ end of a transcribed RNA (such as a transcribed RNA without a polyA tail) that terminates transcription. Almost all eukaryotic mRNAs are polyadenylated, with the exception of metazoan replication-dependent histone mRNAs, in which mRNA processing occurs at a site of highly conserved stem-loop structure and a purine rich region around 20 nucleotides downstream. These are the few (if not the only) eukaryotic mRNAs that lack a poly(A) tail, ending instead in a stem-loop structure followed by a purine-rich sequence, termed histone downstream element (HDE) or histone 3′ UTR stem-loop. HDE directs where the RNA is cleaved during/after transcription, so that the 3′ end of the histone mRNA is formed. HDE is involved in nucleocytoplasmic transport of the histone mRNAs, and in the regulation of stability and of translation efficiency in the cytoplasm.

In certain embodiments, the RNA sequence of the invention further comprises a second transcribed modified AAV ITR of the invention. In certain embodiments, the second transcribed modified AAV ITR has a transcribed functional RBE sequence but lacks either a second transcribed TRS or a second transcribed rcTRS or both; optionally, the second transcribed modified AAV ITR further comprises or lacks a second transcribed D region sequence. In certain embodiments, the second transcribed modified AAV ITR comprises a second transcribed mutated D region sequence and/or a second transcribed mutated TRS sequence.

In certain embodiments, for the RNA sequence of the invention having two transcribed modified AAV ITR, the transcribed modified AAV ITR and the second transcribed modified AAV ITR are identical.

In certain embodiments, for the RNA sequence of the invention having two transcribed modified AAV ITR, the transcribed modified AAV ITR and the second transcribed modified AAV ITR are different.

In certain embodiments, the transcribed modified AAV ITR, the second transcribed modified AAV ITR (if present), comprise a deletion from, a mutation in, or an insertion into a corresponding transcribed wild-type AAV ITR D region sequence or a corresponding transcribed wild-type TRS/rcTRS.

In certain embodiments, for the RNA sequence of the invention having two transcribed modified AAV ITR, the second transcribed modified AAV ITR is within 5′ end 1000 nucleotides, 800 nucleotides, 500 nucleotides, 250 nucleotides, or 150 nucleotides of the RNA sequence.

In certain embodiments, the RPS comprises an MS2 sequence, an PP7 binding site, or a com binding site, and the RPS-interacting molecule comprises an RPS-interacting protein (RPSIP) capably of interacting, e.g., recognizing and binding, directly or indirectly, to the RPS, such as a bacteriophage-derived MS2 coat protein (MCP) for an MS2 sequence, a PP7 bacteriophage coat protein (PCP) for an PP7 binding site, or a phage COM protein (COM) for a com binding site. Sequences of these RPS/RPSIP pair are described in the sequence section of the specification.

Any of the one or more RPS sequences described herein above, including any of the transcribed modified ITR sequences, and any of the MS2 sequence, PP7 binding site, and/or com binding site, alone or in combination, can facilitate the packaging of the RNA sequence of the invention into the DNA virus viral particle, in the presence of a suitable/compatible cognate RPSIP.

In certain embodiments, the RPSIP is, or is associated directly or indirectly with, a protein component of the viral packaging system for the DNA virus viral particle. For example, in some embodiments, the RPSIP is a protein component of the viral packaging system for the DNA virus, such as, Rep78, Rep68, Rep52, and/or Rep40 for AAV. For example, in some embodiments, the RPSIP may be directly fused to a protein component of the viral packaging system for the DNA virus. Exemplary protein components of the viral packaging system for AAV include any of the Rep proteins (such as Rep78 and/or Rep68 of adeno-associated virus 2 (AAV2)), and/or any of the assembly-activating protein (AAP).

In certain embodiments, the fusion is an N-terminal fusion wherein the RPSIP (such as MCP, PCP, or COM) is fused N-terminal to a Rep68/78 protein, and/or to an AAP.

In certain embodiments, the fusion is an N-terminal fusion wherein the RPSIP (such as MCP, PCP, or COM) is fused C-terminal to a Rep68/78 protein, and/or to an AAP.

In certain embodiments, the fusion is a direct fusion with no linker sequences in-between.

In certain embodiments, the fusion is through one or more linker sequence, such as a flexible peptide linker that may include a Gly and Ser rich linker or GS linker. Representative GS linkers include 1, 2, 3, 4, 5 or more repeats of Gly or Ser, such as GS, GSS, GSSS (SEQ ID NO: 105), GSSSS (SEQ ID NO: 106), and repeats thereof (e.g., (GSₚ)ₙ (SEQ ID NO: 148), wherein p is an integer between 1-5, and n is an integer between 1-20. One typical such GS linker is GS3 (SEQ ID NO: 105) linker or GS4 (SEQ ID NO: 106) linker. In certain embodiments, p is 3 or 4, and n is 1.

In certain embodiments, the RNA sequence of the invention can comprise, but preferably does not comprise, a transcribed DNA packaging signal, for example, a transcribed wild-type AAV ITR sequence. For example, the RNA sequence of the invention may comprise a transcribed modified AAV ITR sequence having an addition, a deletion, and/or a substitution of a nucleotide of a corresponding transcribed wild-type AAV ITR sequence to reduce the DNA packaging capability of the DNA virus viral particle.

In certain embodiments, the RNA sequence of the invention further comprises one or more of: (1) a coding sequence for a protein (such as an mRNA encoding a therapeutic protein or a CRISPR/Cas effector enzyme including any of the Cas effectors described herein below, e.g., Cas9, or a variant thereof, optionally fused to a base editor), a non-coding RNA (ncRNA), or a functional RNA (such as a tRNA, a ribosomal RNA (rRNA), an RNAi reagent or precursor thereof, siRNA, shRNA, miRNA or precursors thereof including pre-miRNA and pri-miRNA, antisense RNA (ASO), piRNA, an RNA component of CRISPR-Cas system such as a guide RNA (or gRNA), a single guide RNA (or sgRNA, chimeric RNA, RNA chimera), a CRISPR RNA (crRNA), or a tracr RNA), snoRNA, snRNA, cxRNA, scaRNA, lncRNA, Xist, and HOTAIR, etc.); (2) a transcribed transcription enhancer; (3) a transcribed intron sequence or exon sequence (such as one for enhancing protein expression); (4) a 5′ UTR sequence; (5) a 3′ UTR sequence; (6) a polyA sequence, or a (transcribed) polyadenylation (polyA) signal sequence, and optionally a transcribed polyA site and a transcribed GU-rich region downstream of the polyA site; (7) a posttranscriptional regulatory element or sequence, such as a transcribed Woodchuck Hepatitis Virus (WHP) Posttranscriptional Regulatory Element (WPRE) sequence; and/or, (8) a transcription termination sequence (such as a histone downstream element).

In certain embodiments, the RNA sequence of the invention comprises an RPS located 3′ to the posttranscriptional regulatory element or sequence, and 5′ to the polyA sequence or the polyA signal sequence.

For example, in certain embodiments, the RNA sequence of the invention comprises, in 5′ to 3′ orientation, the RSI; the optional transcribed WPRE sequence (that may or may not be present); the RPS (such as the transcribed modified AAV ITR, the MS2 sequence, the PP7 binding site, or the com binding site); and the polyA sequence or the polyA signal sequence.

In certain embodiments, the RNA sequence of the invention encodes, or the GOI comprises, a protein (e.g., a fluorescent protein, a therapeutic protein, an antigen protein, or a gene-editing protein such as a Cas protein, a ZFN protein, a TALEN protein), an enzyme (such as a Cre protein, or a CRISPR/Cas effector enzyme, e.g., Cas9, Cas12, Cas13, or a variant thereof), a structural protein, an mRNA, a non-coding RNA (ncRNA), an siRNA, a piRNA, a short hairpin RNA or shRNA, a microRNA (miRNA) or a precursor thereof (including pre-miRNA and pri-miRNA), a ribosomal RNA (rRNA), an antisense sequence or oligonucleotide (ASO), an RNA component of a CRISPR-Cas system, including a guide RNA (or a gRNA), such as a single guide RNA (or an sgRNA, a chimeric RNA, an RNA chimera), a CRISPR RNA (crRNA), and a tracr RNA, a guide RNA or gRNA for a CRISPR/Cas effector enzyme, an rRNA, a tRNA, a snoRNA, a snRNA, an exRNA, a scaRNA, a lncRNA, a Xist, and a HOTAIR.

The overall length of the RNA sequence of the invention depends on the packaging capacity of the AAV viral particle. Most AAV viral particles have a packaging capacity of about 4,700-5,200 nucleotides, but certain AAV viral particles such as AAV5 particles can package up to 8,900 nucleotides.

Thus, in certain embodiments, the RNA sequence of the invention to be packaged into an AAV viral particle is a single-stranded RNA (ssRNA) less than about 8,900 nucleotides in length.

In certain embodiments, the RNA sequence is a ssRNA less than about 8,000 nucleotides in length. In certain embodiments, the RNA sequence is a ssRNA less than about 7,000 nucleotides in length. In certain embodiments, the RNA sequence is a ssRNA less than about 6,000 nucleotides in length. In certain embodiments, the RNA sequence is a ssRNA less than about 5,200 nucleotides in length. In certain embodiments, the RNA sequence is a ssRNA less than about 4,000 nucleotides in length. In certain embodiments, the RNA sequence is a ssRNA less than about 3,000 nucleotides in length. In certain embodiments, the RNA sequence is a ssRNA less than about 2,000 nucleotides in length.

In certain embodiments, the RNA sequence is a ssRNA about 4,700-5,200 nucleotides in length. In certain embodiments, the RNA sequence is a ssRNA about 4,700-5,000 nucleotide in length. In certain embodiments, the RNA sequence is a ssRNA about 4,700-4,800 nucleotides in length. In certain embodiments, the RNA sequence is a ssRNA about 4,700 nucleotides in length.

Another aspect of the invention provides a polynucleotide comprising a (transcription) cassette encoding the RNA sequence of the invention; optionally, the polynucleotide is a DNA sequence (e.g., a DNA plasmid), optionally comprising a stuffer sequence in the backbone of the DNA plasmid, and/or optionally comprising no functional DNA packaging signal such as AAV ITR.

In certain embodiments, the polynucleotide comprising the cassette is a DNA vector encoding the RNA sequence of the invention. Such DNA vector and/or the cassette thereof can be used to transcribe and produce the RNA sequence of the invention for further packaging into, e.g., an AAV viral particle.

In certain embodiments, the polynucleotide further comprises a promoter operably linked to and driving the transcription of the RNA sequence of the invention encoded by the cassette to produce the RNA sequence of the invention.

In certain embodiments, the promoter is a ubiquitous promoter.

In certain embodiments, the promoter is a tissue-specific promoter.

In certain embodiments, the promoter is a constitutive promoter.

In certain embodiments, the promoter is an inducible promoter.

In certain embodiments, the polynucleotide further comprises an enhancer that enhances the transcription of the RNA sequence driven by the promoter.

Another aspect of the invention provides a recombinant DNA virus viral particle comprising an RNA genome (such as the RNA sequence of the invention, or the RNA sequence transcribed from the polynucleotide of the invention) packaged within the protein shell (such as capsid) of a DNA virus (such as an AAV virus, or an oncolytic virus).

In certain embodiments, the DNA virus is AAV, and the recombinant DNA virus viral particle is a recombinant RNA adeno-associated virus (rRAAV) particle, comprising: (1) an AAV capsid; and, (2) the RNA sequence of the invention, or the RNA sequence transcribed from the polynucleotide of the invention, packaged within the AAV capsid.

In certain embodiments, the AAV capsid comprises a capsid from an AAV of the serotype AAV1, AAV2, AAV3A, AAV3B, AAV4, AAV5, AAV6, AAV7, AAVrh74, AAV8, AAV9, AAV10, AAV11, AAV12, AAV13, AAV-DJ, AAV PHP.cB, Anc80L65, Anc80L65AAP, or 7m8.

A related aspect of the invention provides a population of recombinant DNA virus viral particles (e.g., rRAAV particles) comprising a plurality of recombinant DNA virus viral particle (e.g., rRAAV particle) of the invention, wherein at least about 10%, 20%, 30%, 40%, 50%, 60%, 70%, 80%, 90%, 95%, 96%, 97%, 98%, 99% or more of the recombinant DNA virus viral particles (e.g., rRAAV particles) within the population have encapsidated RNA sequence of the invention, or the RNA sequence transcribed from the polynucleotide of the invention packaged therein.

In certain embodiments, the population of recombinant viral particles (e.g., rRAAV particles) comprises at least 1×10⁴ viral particles, at least 2×10⁴ viral particles, at least 5×10⁴ viral particles, at least 1×10⁵ viral particles, at least 2×10⁵ viral particles, at least 5×10⁵ viral particles, at least 1×10⁶ viral particles, at least 2×10⁶ viral particles, at least 5×10⁶ viral particles, at least 1×10⁷ viral particles, at least 2×10⁷ viral particles, at least 5×10⁷ viral particles, at least 1×10⁸ viral particles, at least 2×10⁸ viral particles, at least 5×10⁸ viral particles, at least 1×10⁹ viral particles, at least 2×10⁹ viral particles, at least 5×10⁹ viral particles, at least 1×10¹⁰ viral particles, at least 2×10¹⁰ viral particles, at least 5×10¹⁰ viral particles, at least 1×10¹¹ viral particles, at least 2×10¹¹ viral particles, at least 5×10¹¹ viral particles, at least 1×10¹² viral particles, at least 2×10¹² viral particles, at least 5×10¹² viral particles, at least 1×10¹³ viral particles, at least 2×10¹³ viral particles, at least 5×10¹³ viral particles, at least 1×10¹⁴ viral particles, at least 2×10¹⁴ viral particles, at least 5×10¹⁴ viral particles, at least 1×10¹⁵ viral particles, at least 2×10¹⁵ viral particles, at least 5×10¹⁵ viral particles, at least 1×10¹⁶ viral particles, at least 2×10¹⁶ viral particles, or at least 5× 10¹⁶ viral particles.

In certain embodiments, at most 50%, 45%, 40%, 35%, 30%, 25%, 20%, 15%, 10%, 5%, 3%, 2%, 1%, 0.1%, 0.01% or less of the population of recombinant viral particles encapsidate non-RNA (e.g., DNA) within the viral particles.

Another aspect of the invention provides a host cell comprising the RNA sequence of the invention, the polynucleotide of the invention, the RNA sequence transcribed from the polynucleotide of the invention, the recombinant DNA virus viral particle (e.g., rRAAV particle) of the invention, and/or the population of recombinant DNA virus viral particle (e.g., rAAV particle) of the invention.

In certain embodiments, the host cell further comprises a viral packaging system that facilitates packaging of the RNA sequence of the invention, or the RNA sequence transcribed from the polynucleotide of the invention into the DNA virus viral particle.

In certain embodiments, the viral packaging system comprises: (1) an AAV rep gene (e.g., coding sequence for Rep78, Rep68, Rep52, and/or Rep40) and an AAV cap gene (e.g., coding sequence for VP1, VP2, and/or VP3, AAP, and/or MAAP), under the transcriptional control of one or more promoters that drive the transcription of the rep gene and cap gene, or the expression products thereof; (2) one or more coding sequences for one or more proteins required for AAV packaging, such as adenoviral E2A, E4, and VA genes, or the one or more proteins; and (3) the RPS-interacting molecule or a coding sequence thereof.

In certain embodiments, the capacity of the viral packaging system of packaging a DNA sequence into the DNA virus viral particle is reduced, diminished, or substantially eliminated by, for example, (1) removing a part or all of the DNA packaging signals such as AAV ITR on the polynucleotide encoding the RNA sequence of the invention or on the polynucleotide of the invention, (2) modifying, e.g., mutating, the AAV rep gene, the AAV cap gene, and/or the one or more coding sequences for one or more proteins required for AAV packaging to reduce, diminish, or substantially eliminate the capacity of the respective translated protein in order to facilitate the packaging of the DNA sequence into the DNA virus viral particle (e.g., a Y156F mutation in the common sequence of Rep78 and Rep68 proteins, KDE-mu, or EKE-mu); and/or (3) enlarging the size of the polynucleotide encoding the RNA sequence of the invention or the polynucleotide of the invention. In an embodiment, enlarging the size of the polynucleotide encoding the RNA sequence of the invention or the polynucleotide of the invention is made by inserting a stuffer sequence (e.g., an intron) into (e.g., the backbone of) the polynucleotide (e.g., a DNA plasmid).

In certain embodiments, the AAV rep gene, the AAV cap gene, and/or the proteins required for AAV packaging comprises a mutation that diminishes or reduces capacity to facilitate packaging of DNA into the DNA virus viral particle.

In certain embodiments, the Rep68/Rep 78 protein required for DNA packaging comprises a mutation that compromises or diminishes its trs-endonuclease activity. The trs-endonuclease activity is believed to be required to resolve AAV replication (DNA) intermediates at the trs sequence or site, such that individual units of AAV ssDNA can be resolved before packaging into the AAV capsid.

In certain embodiments, the trs-endonuclease mutation comprise a Y156F mutation in the common sequence of Rep78 and Rep68 proteins.

In certain embodiments, the Rep78/Rep68 proteins comprise a KDE-mu mutation (see sequence below in the sequence section).

In certain embodiments, Rep78/Rep68 proteins comprise a EKE-mu mutation (see sequence below in the sequence section).

In certain embodiments, Rep78/Rep68 proteins comprise two or more mutations selected form the Y156F mutation, the KDE-mu mutation, and the EKE-mu mutation.

In certain embodiments, the Rep68/Rep78 are from any one of the AAVs with serotype of AAV1, AAV2, AAV3A, AAV3B, AAV4, AAV5, AAV6, AAV7, AAV8, AAV9, AAV10, AAV11, AAV12, AAV13, AAV-DJ, AAV PHP.eB, Anc80L65, Anc80L65AAP, AAVrh74, or 7m8, and has a corresponding trs-endonuclease mutation of the Y156F mutation, the KDE-mu mutation, and/or the EKE-mu mutation.

In certain embodiments, the host cell further comprises: (1) a coding sequence for an AAV rep gene and an AAV cap gene, under the transcriptional control of one or more promoters that drive the transcription of the rep gene and cap gene; and, (2) coding sequences for proteins required for AAV packaging, such as adenoviral E2A, E4, and VA genes.

In certain embodiments, the host cell is a mammalian cell, such as a HEK293 cell or a variant thereof (e.g., HEK293T cell), or an insect cell, such as Sf9 or Sf21 cells.

Another aspect of the invention provides a method of generating the recombinant DNA virus viral particle (e.g., rRAAV particle) or the population of recombinant DNA virus viral particles (e.g., rRAAV particles) of the invention, the method comprising: a) culturing the host cell of the invention for a sufficient time, and b) harvesting the recombinant DNA virus viral particle or the population of recombinant DNA virus viral particles.

In certain embodiments, the method further comprises isolating or purifying the recombinant DNA virus viral particle or the population of recombinant DNA virus viral particles.

Another aspect of the invention provides a method of generating a recombinant DNA virus viral particle (e.g., rRAAV particle) or a population of recombinant DNA virus viral particles, the method comprising: a) contacting a viral packaging system (e.g., an AAV packaging system) with the RNA sequence of the invention or the RNA sequence transcribed from the polynucleotide of the invention, for a period of time sufficient to produce the recombinant DNA virus viral particle of the invention, or the population of recombinant DNA virus viral particles of the invention, and b) harvesting the recombinant DNA virus viral particle of the invention, or the population of recombinant DNA virus viral particles of the invention; and, optionally, c) isolating or purifying the harvested recombinant DNA virus viral particle of the invention, or the population of recombinant DNA virus viral particles of the invention.

In certain embodiments, the viral packaging system (e.g., a AAV packaging system) comprises: (1) one or more proteins for assembling the protein shell (e.g., VP1, VP2, and/or VP3 for assembling AAV capsid) of the DNA virus viral particle for packaging the RNA sequence, or one or more coding sequences thereof; (2) one or more proteins (e.g., Rep78, Rep68, Rep52, and/or Rep40 for AAV packaging) for facilitating the assembling of the protein shell and/or the packaging of the RNA sequence into the protein shell of the DNA virus viral particle, or one or more coding sequences thereof (e.g., adenoviral E2a, E4, and VA genes); and (3) the RPS-interacting molecule or a coding sequence thereof. Optionally, the capacity of the viral packaging system of packaging a DNA sequence into the DNA virus viral particle is reduced, diminished, or substantially eliminated by, for example, (1) removing a part or all of the DNA packaging signals such as AAV ITR on the polynucleotide encoding the RNA sequence of the invention, or on the polynucleotide of the invention, (2) modifying, e.g., mutating, the AAV rep gene, the AAV cap gene, and/or the one or more coding sequences for one or more proteins required for AAV packaging to reduce, diminish, or substantially eliminate the capacity of the respective translated protein to facilitate the packaging of the DNA sequence into the DNA virus viral particle (e.g., a Y156F mutation in the common sequence of Rep78 and Rep68 proteins, KDE-mu, or EKE-mu); and/or (3) enlarging the size of the polynucleotide encoding the RNA sequence of the invention or the polynucleotide of the invention.

Another aspect of the invention provides a system of packaging the RNA sequence of the invention or the RNA sequence transcribed from the polynucleotide of the invention into a DNA virus viral particle, the system comprising: (1) one or more proteins for assembling the protein shell (e.g., VP1, VP2, and/or VP3 for assembling AAV capsid) of the DNA virus viral particle for packaging the RNA sequence, or one or more coding sequences thereof; (2) one or more proteins (e.g., Rep78, Rep68, Rep52, and/or Rep40 for AAV packaging) for facilitating the assembling of the protein shell and/or the packaging of the RNA sequence of the invention into the protein shell of the DNA virus viral particle, or one or more coding sequences thereof (e.g., adenoviral E2a, E4, and VA genes); and (3) the RPS-interacting molecule or a coding sequence thereof. Optionally, the capacity of the viral packaging system of packaging a DNA sequence into the DNA virus viral particle is reduced, diminished, or substantially eliminated by, for example, (1) removing a part or all of the DNA packaging signals such as AAV ITR on the polynucleotide encoding the RNA sequence of the invention or on the polynucleotide of the invention, (2) modifying, e.g., mutating, the AAV rep gene, the AAV cap gene, and/or the one or more coding sequences for one or more proteins required for AAV packaging to reduce, diminish, or substantially eliminate the capacity of the respective translated protein to facilitate the packaging of the DNA sequence into the DNA virus viral particle (e.g., a Y156F mutation in the common sequence of Rep78 and Rep68 proteins, KDE-mu, or EKE-mu); and/or (3) enlarging the size of the polynucleotide encoding the RNA sequence of the invention or the polynucleotide of the invention.

Another aspect of the invention provides a method of delivering an RNA sequence of interest (RSI) into a cell, a plant, or an animal, the method comprising contacting the cell, the plant, or the animal with the recombinant DNA virus viral particle (e.g., rRAAV particle) of the invention, the population of recombinant DNA virus viral particles (e.g., rRAAV particles) of the invention, or the recombinant DNA virus viral particle (e.g., rRAAV particle) or the population of recombinant DNA virus viral particles (e.g., rRAAV particles) produced by the method of the invention, wherein the GOI is optionally encoded by the RNA sequence of the invention.

Another aspect of the invention provides a method of diagnosing, preventing, or treating a disease or disorder in a subject in need thereof, comprising administrating to the subject a therapeutically effective amount or dose of the population of the recombinant DNA virus viral particles (e.g., rRAAV particles) of the invention, or produced by the method of the invention.

Another aspect of the invention provides a use of the recombinant DNA virus viral particle (e.g., rRAAV particle) of the invention, the population of the recombinant DNA virus viral particles (e.g., rRAAV particles) of the invention, or the recombinant DNA virus viral particle (e.g., rRAAV particle) or the population of the recombinant DNA virus viral particles (e.g., rRAAV particles) produced by the method of the invention, in the manufacture of a medicament for diagnosing, preventing, or treating a disease or disorder in a subject in need thereof.

Another aspect of the invention provides a fusion protein or a conjugate, comprising an RPSIP of the invention fused or conjugated to a protein component of the viral packaging system for the DNA virus, wherein the RPSIP interacts with/binds to an RPS on the RNA sequence of the invention to facilitate the packaging of the RNA sequence into the DNA virus.

In certain embodiments, the RPS is MS2, and the RPSIP is MCP.

In certain embodiments, the RPS is PP7 binding site, and the RPSIP is PCP. In certain embodiments, the RPS is com, and the RPSIP is phage COM protein.

In certain embodiments, the fusion or conjugate comprises more than one RPSIP, each independently binds to one or more RPS on the RNA sequence of the invention. In certain embodiments, at least two of the more than one RPSIP are identical. In certain embodiments, at least two of the more than one RPSIP are different.

In certain embodiments, the fusion or conjugate comprises two MCP in tandem.

In certain embodiments, the protein component of the viral packaging system for the DNA virus comprises a Rep protein of an AAV, such as a Rep68 or a Rep78 of the AAV.

In certain embodiments, the Rep protein comprises one or more mutations that compromises or diminishes trs-endonuclease activity. In certain embodiments, the mutations comprise the Y156F mutation, the KDE-mu mutation, and/or the EKE-mu mutation.

In certain embodiments, the protein component of the viral packaging system for the DNA virus comprises an assembly-activating protein (AAP).

In certain embodiments, the RPSIP is fused to the protein component of the viral packaging system for the DNA virus (e.g., a Rep protien or an AAP) directly.

In certain embodiments, the RPSIP is fused to the protein component of the viral packaging system for the DNA virus (e.g., a Rep protien or an AAP) through a peptide linker.

In certain embodiments, the peptide linker is a flexible linker, such as a Gly and Ser containing linker. In certain embodiments, the Gly and Ser containing linker comprises 1-20 repeats (e.g., 1-5 or 1-3 repeats) of GSn, wherein n is 1, 2, 3, 4, or 5. In certain embodiments, the GSn linker is GS2, GS3 (SEQ ID NO: 105), or GS4 (SEQ ID NO: 106), with 1-4 (e.g., 2) repeats. In certain embodiments, the linker is GSSGSS (SEQ ID NO: 107).

In certain embodiments, the fusion protein comprises MCP and Rep, wherein the Rep optionally comprises a Y156F mutation, a KDE-mu mutation, and/or a EKE-mu mutation. In certain embodiments, MCP is fused N-terminal to Rep (MCP-Rep). In certain embodiments, the Rep fused to MCP comprises a Y156F mutation, a KDE-mu mutation, and/or a EKE-mu mutation. In certain embodiments, the MCP-Rep fusion is linked by a GS, linker, such as GSSGSS (SEQ ID NO: 107). In certain embodiments, the MCP-Rep comprises two MCP in tandem (e.g., without any linker between the two MCP moieties). In certain embodiments, the MCP is C-terminal to another GSn linker, such as GSSGSS (SEQ ID NO: 107).

In certain embodiments, the fusion protein comprises PCP and Rep, wherein the Rep optionally comprises a Y156F mutation, a KDE-mu mutation, and/or a EKE-mu mutation. In certain embodiments, PCP is fused N-terminal to Rep (PCP-Rep). In certain embodiments, the Rep fused to PCP comprises a Y156F mutation, a KDE-mu mutation, and/or a EKE-mu mutation. In certain embodiments, the PCP-Rep fusion is linked by a GSn linker, such as GSSGSS (SEQ ID NO: 107). In certain embodiments, the PCP-Rep comprises two PCP in tandem (e.g., without any linker between the two PCP moieties). In certain embodiments, the PCP is C-terminal to another GSn linker, such as GSSGSS (SEQ ID NO: 107).

In certain embodiments, the fusion protein comprises COM and Rep, wherein the Rep optionally comprises a Y156F mutation, a KDE-mu mutation, and/or a EKE-mu mutation. In certain embodiments, COM is fused N-terminal to Rep (COM-Rep). In certain embodiments, the Rep fused to COM comprises a Y156F mutation, a KDE-mu mutation, and/or a EKE-mu mutation. In certain embodiments, the COM-Rep fusion is linked by a GSn linker, such as GSSGSS (SEQ ID NO: 107). In certain embodiments, the COM-Rep comprises two COM in tandem (e.g., without any linker between the two COM moictics). In certain embodiments, the COM is C-terminal to another GSn linker, such as GSSGSS (SEQ ID NO: 107).

In certain embodiments, the fusion protein comprises MCP and AAP. In certain embodiments, MCP is fused N-terminal to AAP (MCP-AAP, or MA). In certain embodiments, MCP is fused C-terminal to AAP (AAP-MCP, or AM). In certain embodiments, the MCP-AAP or AAP-MCP fusion is linked by a GSn linker, such as GSSGSS (SEQ ID NO: 107). In certain embodiments, the MCP-AAP fusion is C-terminal to another GSn linker, such as GSSGSS (SEQ ID NO: 107). In certain embodiments, the AAP-MCP fusion is N-terminal to another GSn linker, such as GSSGSS (SEQ ID NO: 107).

Another aspect of the invention provides a polynucleotide encoding any one of the fusions between the RPSIP of the invention and the protein component of the viral packaging system for the DNA virus (e.g., AAP or a Rep protein).

With the general aspects of the invention described herein above, the following sections provides additional details for specific elements of the invention described herein. Each specific element is contemplated to be able to combined with any one or more additional elements of the invention, even though all possible combinations or permutations of the elements are not explicitly recited.

### 2. AAV Serotypes

AAV particles packaging ribopolynucleotides of the invention may comprise or be derived from any natural or recombinant AAV serotypes.

According to the present disclosure, the AAV particles may utilize or be based on a serotype selected from any of the following serotypes, and variants thereof, including but not limited to: AAV1, AAV10, AAV106.1/hu.37, AAV11, AAV114.3/hu.40, AAV 12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2, AAV2.5T, AAV2-15/rh.62, AAV223.1, AAV223.2, AAV223.4, AAV 223.5, AAV223.6, AAV223.7, AAV2-3/rh.61, AAV24.1, AAV2-4/rh.50, AAV2-5/rh.51, AAV27.3, AAV29.3/bb.1, AAV29.5/bb.2, AAV2G9, AAV-2-pre-miRNA-101, AAV3, AAV3.1/hu.6, AAV3.1/hu.9, AAV3-11/rh.53, AAV3-3, AAV33.12/hu. 17, AAV33.4/hu. 15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-1b, AAV42-2, AAV42-3a,

AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r 11.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAV A3.3, AAV A3.4, AAV A3.5, AAV A3.7, AAV-b, AAVCI, AAVC2, AAVC5, AAVCh.5, AAVCh.5R1, AAVcy.2, AAVcy.3, AAVcy.4, AAVcy.5, AAVCy.5RI, AAVCy.5R2, AAVCy.5R3, AAVCy.5R4, AAVcy.6, AAV-DJ, AAV-DJ8, AAVF3, AAVF5, AAV-h, AAVH-1/hu.l, AAVH2, AAVH-5/hu.3, AAVH6, AAVhE1.1, AAVhER1.14, AAVhErl.16, AA VhErl. 18, AAVhER1.23, AA VhErl.35, AA VhErl.36, AAVhErl.5, AAVhErl.7, AAVhEr1.8, AAVhEr2.16, AA VhEr2.29, AA VhEr2.30, AA VhEr2.31, AAVhEr2.36, AA VhEr2.4, AAVhEr3.1, AAVhu. 1, AAVhu. 10, AAVhu.11, AA Vhu. 1, AAVhu. 12, AAVhu. 13, AAVhu.14/9, AAVhu. 15, AAVhu.16, AAVhu. 17, AAVhu. 18, AAVhu. 19, AA Vhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44RI, AAVhu.44R2, AAVhu.44R3, AAVhu.45, AAVhu.46, AAVhu.47, AAVhu.48, AAVhu.48RI, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu.7, AAVhu.8, AAVhu.9, AAVhu.t 19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC8, AAVpi. 1, AAVpi.2, AAVpi.3, AAVrh.10, AAVrh.12, AAVrh.13, AAVrh. 13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64RI, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, BNP61 AAV, BNP62 AAV, BNP63 AAV, bovine AAV, caprine AAV, Japanese AAV 10, true type AAV (ttAAV), UPENN AAV 10, AAV-LK16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, and/or AAV SM 10-8.

In certain embodiments, the AAV serotype may comprise a mutation in the AAV9 sequence, such as the sequence described by Pulicherla et al. (Molecular Therapy 19 (6): 1070-1078, 2011), such as AAV9.9, AAV9.11, AAV9.13, AAV9.16, AAV9.24, AAV9.45, AAV9.47, AAV9.61, AAV9.68, AAV9.84.

In certain embodiments, the AAV serotype may comprise a sequence described in U.S. Pat. No. 6,156,303, such as AAV3B (SEQ ID NOs: 1 and 10 of U.S. Pat. No. 6,156,303), AAV6 (SEQ ID NOS: 2, 7 and 11 of U.S. Pat. No. 6,156,303), AAV2 (SEQ ID NOs: 3 and 8 of U.S. Pat. No. 6,156,303), AAV3A (SEQ ID NOs: 4 and 9, of U.S. Pat. No. 6,156,303), or derivatives thereof.

In certain embodiments, the serotype may be AAV-DJ or a variant thereof, such as AAVDJ8 (or AAV-DJ8), as described by Grimm et al. (Journal of Virology 82 (12): 5887-5911, 2008). The amino acid sequence of AAV-DJ8 may comprise two or more mutations in order to remove the heparin binding domain (HBD). As a non-limiting example, the AAV-DJ sequence described as SEQ ID NO: 1 in U.S. Pat. No. 7,588,772 may comprise two mutations: (1) R587Q (Arg at amino acid 587 is changed to glutamine Gln), and (2) R590T. As another non-limiting example, the AAV-DJ sequence may comprise three mutations: (1) K406R, (2) R587Q, and (3) R590T.

In certain embodiments, the AAV serotype may comprise a sequence as described in WO2015/121501, such as true type AAV (ttAAV) (SEQ ID NO: 2 of WO2015/121501), the so-called UPenn AAV10 (SEQ ID NO: 8 of WO2015/121501), or the so-called Japanese AAV10 (SEQ ID NO: 9 of WO2015/121501), or variants thereof.

AAV capsid serotype selection or use may be from a variety of species. In certain embodiments, the AAV may be an avian AAV (aAAV). The aAAV serotype may comprise a sequence described in U.S. Pat. No. 9,238,800, such as aAAV (SEQ ID NOs: 1, 2, 4, 6, 8, 10, 12, and 14 of U.S. Pat. No. 9,238,800), or variants thereof.

In certain embodiments, the AAV may be a bovine AAV (bAAV). The bAAV serotype may comprise a sequence described in U.S. Pat. No. 9,193,769, such as bAAV (SEQ ID NOS: 1 and 6 of U.S. Pat. No. 9,193,769), or variants thereof. The bAAV serotype may comprise a sequence as described in U.S. Pat. No. 7,427,396, such as bAAV (SEQ ID NOs: 5 and 6 of U.S. Pat. No. 7,427,396), or variants thereof.

In certain embodiments, the AAV may be a caprine AAV. The caprine AAV serotype may comprise a sequence described in U.S. Pat. No. 7,427,396, such as caprine AAV (SEQ ID NO: 3 of U.S. Pat. No. 7,427,396), or variants thereof.

In certain embodiments, the AAV may be engineered as a hybrid AAV from two or more parental serotypes.

In certain embodiments, the AAV may be AAV2G9, which comprises sequences from AAV2 and AAV9. The AAV2G9 AAV serotype may comprise a sequence described in US 2016-0017005 A1. (incorporated herein by reference).

In certain embodiments, the AAV may be a serotype generated by the AAV9 capsid library with mutations in amino acids 390-627 (VPI numbering) as described by Pulicherla et al. (Molecular Therapy 19 (6): 1070-1078, 2011, incorporated herein by reference). The serotype and corresponding nucleotide and amino acid substitutions may be, but is not limited to: AAV9.1 (G1594C; D532H), AAV6.2 (T1418A and T1436X; V473D and 1479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F411I), AAV9.9 (G1203A, G1785T, W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V606I), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T5821), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (C1531A, T1609A; L511I, L537M), AAV9.55 (T1605A; F535L), AAV9.58 (C1475T, C1579A; T492I, H527N), AAV.59 (T1336C; Y446H), AAV9.61 (A1493T; N4981), AAV9.64 (C1531A, A1617T; L511I), AAV9.65 (C1335T, T1530C, C1568A; A523D), AAV9.68 (C1510A; P504T), AAV9.80 (G1441A;G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K528I), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In certain embodiments, the AAV may be a serotype comprising at least one AAV capsid CD8+ T-cell epitope. As a non-limiting example, the serotype may be AAV1, AAV2 or AAV 8.

In certain embodiments, the AAV may be a variant, such as PHP.A or PHP.B as described in Deverman (Nature Biotechnology. 34 (2): 204-209, 2016, incorporated herein by reference).

In certain embodiments, the AAV may be a serotype generated by Cre-recombination-based AAV targeted evolution (CREATE) described by Deverman et al., (Nature Biotechnology 34 (2): 204-209, 2016, incorporated herein by reference). In certain embodiments, the AAV serotypes generated in this manner have improved CNS transduction and/or neuronal and astrocytic tropism, as compared to other AAV serotypes.

In some embodiments, the AAV serotypes may be an AAV9 derivative with a 7-amino acid insertion between amino acids 588-589. Non-limiting examples of these 7-amino acid insertions include PHP.A, PHP.B, PHP.B2, PHP.B3, PHP.N, PHP.S, G2A12, G2A15, G2A3, G2B4, and G2B5.

In certain embodiments, the AAV may be a serotype selected from any of those found in SEQ ID NOs: 4,734-5,302 and in Table 2 of WO2018/002719A1 (incorporated herein by reference). In certain embodiments, the AAV may be encoded by a sequence, fragment or variant as described in SEQ ID NOs: 4,734-5,302 of WO2018/002719A1 (incorporated herein by reference).

In certain embodiments, the AAV VP1 capsid sequence is one of the following: AAV1, AAV2, AAV3A, AAV3B, AAV4, AAV5, AAV6, AAV7, AAV8, AAV9, AAV10, AAV11, AAV12, AAV13, AAV-DJ, AAV PHP.eB, Anc80L65, Anc80L65AAP, or 7m8.

Protein sequences of the above representative VPI capsids are provided below.

(AAV1; SEQ ID NO: 6)
MAADGYLPDWLEDNLSEGIREWWDLKPGAPKPKANQQKQDDGRGLVLPGYKYLGPFNGLDKGEPVNAA
DAAALEHDKAYDQQLKAGDNPYLRYNHADAEFQERLQEDTSFGGNLGRAVFQAKKRVLEPLGLVEEGA
KTAPGKKRPVEQSPQEPDSSSGIGKTGQQPAKKRLNFGQTGDSESVPDPQPLGEPPATPAAVGPTTMA
SGGGAPMADNNEGADGVGNASGNWHCDSTWLGDRVITTSTRTWALPTYNNHLYKQISSASTGASNDNH
YFGYSTPWGYFDFNRFHCHFSPRDWQRLINNNWGFRPKRLNFKLFNIQVKEVTTNDGVTTIANNLTST
VQVFSDSEYQLPYVLGSAHQGCLPPFPADVFMIPQYGYLTLNNGSQAVGRSSFYCLEYFPSQMLRTGN
NFTFSYTFEEVPFHSSYAHSQSLDRLMNPLIDQYLYYLNRTQNQSGSAQNKDLLFSRGSPAGMSVQPK
NWLPGPCYRQQRVSKTKTDNNNSNFTWTGASKYNLNGRESIINPGTAMASHKDDEDKFFPMSGVMIFG
KESAGASNTALDNVMITDEEEIKATNPVATERFGTVAVNFQSSSTDPATGDVHAMGALPGMVWQDRDV
YLQGPIWAKIPHTDGHFHPSPLMGGFGLKNPPPQILIKNTPVPANPPAEFSATKFASFITQYSTGQVS
VEIEWELQKENSKRWNPEVQYTSNYAKSANVDFTVDNNGLYTEPRPIGTRYLTRPL
(AAV2; SEQ ID NO: 7)
MAADGYLPDWLEDTLSEGIRQWWKLKPGPPPPKPAERHKDDSRGLVLPGYKYLGPFNGLDKGEPVNEA
DAAALEHDKAYDRQLDSGDNPYLKYNHADAEFQERLKEDTSFGGNLGRAVFQAKKRVLEPLGLVEEPV
KTAPGKKRPVEHSPVEPDSSSGTGKAGQQPARKRLNFGQTGDADSVPDPQPLGQPPAAPSGLGTNTMA
TGSGAPMADNNEGADGVGNSSGNWHCDSTWMGDRVITTSTRTWALPTYNNHLYKQISSQSGASNDNHY
FGYSTPWGYFDFNRFHCHESPRDWQRLINNNWGFRPKRLNFKLENIQVKEVTQNDGTTTIANNLTSTV
QVFTDSEYQLPYVLGSAHQGCLPPFPADVFMVPQYGYLTLNNGSQAVGRSSFYCLEYFPSQMLRTGNN
FTFSYTFEDVPFHSSYAHSQSLDRLMNPLIDQYLYYLSRTNTPSGTTTQSRLQFSQAGASDIRDQSRN
WLPGPCYRQQRVSKTSADNNNSEYSWTGATKYHLNGRDSLVNPGPAMASHKDDEEKFFPQSGVLIFGK
QGSEKTNVDIEKVMITDEEEIRTTNPVATEQYGSVSTNLQRGNRQAATADVNTQGVLPGMVWQDRDVY
LQGPIWAKIPHTDGHFHPSPLMGGFGLKHPPPQILIKNTPVPANPSTTFSAAKFASFITQYSTGQVSV
EIEWELQKENSKRWNPEIQYTSNYNKSVNVDFTVDTNGVYSEPRPIGTRYLTRNL
(AAV3A; SEQ ID NO: 8)
MAADGYLPDWLEDNLSEGIREWWALKPGVPQPKANQQHQDNRRGLVLPGYKYLGPGNGLDKGEPVNEA
DAAALEHDKAYDQQLKAGDNPYLKYNHADAEFQERLQEDTSFGGNLGRAVFQAKKRILEPLGLVEEAA
KTAPGKKGAVDQSPQEPDSSSGVGKSGKQPARKRLNFGQTGDSESVPDPQPLGEPPAAPTSLGSNTMA
SGGGAPMADNNEGADGVGNSSGNWHCDSQWLGDRVITTSTRTWALPTYNNHLYKQISSQSGASNDNHY
FGYSTPWGYFDFNRFHCHFSPRDWQRLINNNWGFRPKKLSFKLFNIQVRGVTQNDGTTTIANNLTSTV
QVFTDSEYQLPYVLGSAHQGCLPPFPADVFMVPQYGYLTLNNGSQAVGRSSFYCLEYFPSQMLRTGNN
FQFSYTFEDVPFHSSYAHSQSLDRLMNPLIDQYLYYLNRTQGTTSGTTNQSRLLFSQAGPQSMSLQAR
NWLPGPCYRQQRLSKTANDNNNSNFPWTAASKYHLNGRDSLVNPGPAMASHKDDEEKFFPMHGNLIFG
KEGTTASNAELDNVMITDEEEIRTTNPVATEQYGTVANNLQSSNTAPTTGTVNHQGALPGMVWQDRDV
YLQGPIWAKIPHTDGHFHPSPLMGGFGLKHPPPQIMIKNTPVPANPPTTFSPAKFASFITQYSTGQVS
VEIEWELQKENSKRWNPEIQYTSNYNKSVNVDFTVDTNGVYSEPRPIGTRYLTRNL
(AAV3B; SEQ ID NO: 9)
MAADGYLPDWLEDNLSEGIREWWALKPGVPQPKANQQHQDNRRGLVLPGYKYLGPGNGLDKGEPVNEA
DAAALEHDKAYDQQLKAGDNPYLKYNHADAEFQERLQEDTSFGGNLGRAVFQAKKRILEPLGLVEEAA
KTAPGKKRPVDQSPQEPDSSSGVGKSGKQPARKRLNFGQTGDSESVPDPQPLGEPPAAPTSLGSNTMA
SGGGAPMADNNEGADGVGNSSGNWHCDSQWLGDRVITTSTRTWALPTYNNHLYKQISSQSGASNDNHY
FGYSTPWGYFDFNRFHCHFSPRDWQRLINNNWGFRPKKLSFKLFNIQVKEVTQNDGTTTIANNLTSTV
QVFTDSEYQLPYVLGSAHQGCLPPFPADVFMVPQYGYLTLNNGSQAVGRSSFYCLEYFPSQMLRTGNN
FQFSYTFEDVPFHSSYAHSQSLDRLMNPLIDQYLYYLNRTQGTTSGTTNQSRLLFSQAGPQSMSLQAR
NWLPGPCYRQQRLSKTANDNNNSNFPWTAASKYHLNGRDSLVNPGPAMASHKDDEEKFFPMHGNLIFG
KEGTTASNAELDNVMITDEEEIRTTNPVATEQYGTVANNLQSSNTAPTTRTVNDQGALPGMVWQDRDV
YLQGPIWAKIPHTDGHFHPSPLMGGFGLKHPPPQIMIKNTPVPANPPTTFSPAKFASFITQYSTGQVS
VEIEWELQKENSKRWNPEIQYTSNYNKSVNVDFTVDTNGVYSEPRPIGTRYLTRNL
(AAV4; SEQ ID NO: 10)
MTDGYLPDWLEDNLSEGVREWWALQPGAPKPKANQQHQDNARGLVLPGYKYLGPGNGLDKGEPVNAAD
AAALEHDKAYDQQLKAGDNPYLKYNHADAEFQQRLQGDTSFGGNLGRAVFQAKKRVLEPLGLVEQAGE
TAPGKKRPLIESPQQPDSSTGIGKKGKQPAKKKLVFEDETGAGDGPPEGSTSGAMSDDSEMRAAAGGA
AVEGGQGADGVGNASGDWHCDSTWSEGHVTTTSTRTWVLPTYNNHLYKRLGESLQSNTYNGFSTPWGY
FDFNRFHCHFSPRDWQRLINNNWGMRPKAMRVKIFNIQVKEVTTSNGETTVANNLTSTVQIFADSSYE
LPYVMDAGQEGSLPPFPNDVFMVPQYGYCGLVTGNTSQQQTDRNAFYCLEYFPSQMLRTGNNFEITYS
FEKVPFHSMYAHSQSLDRLMNPLIDQYLWGLQSTTTGTTLNAGTATTNFTKLRPTNFSNFKKNWLPGP
SIKQQGFSKTANQNYKIPATGSDSLIKYETHSTLDGRWSALTPGPPMATAGPADSKFSNSQLIFAGPK
QNGNTATVPGTLIFTSEEELAATNATDTDMWGNLPGGDQSNSNLPTVDRLTALGAVPGMVWQNRDIYY
QGPIWAKIPHTDGHFHPSPLIGGFGLKHPPPQIFIKNTPVPANPATTFSSTPVNSFITQYSTGQVSVQ
IDWEIQKERSKRWNPEVQFTSNYGQQNSLLWAPDAAGKYTEPRAIGTRYLTHHL
(AAV5; SEQ ID NO: 11)
MSFVDHPPDWLEEVGEGLREFLGLEAGPPKPKPNQQHQDQARGLVLPGYNYLGPGNGLDRGEPVNRAD
EVAREHDISYNEQLEAGDNPYLKYNHADAEFQEKLADDTSFGGNLGKAVFQAKKRVLEPFGLVEEGAK
TAPTGKRIDDHFPKRKKARTEEDSKPSTSSDAEAGPSGSQQLQIPAQPASSLGADTMSAGGGGPLGDN
NQGADGVGNASGDWHCDSTWMGDRVVTKSTRTWVLPSYNNHQYREIKSGSVDGSNANAYFGYSTPWGY
FDFNRFHSHWSPRDWQRLINNYWGFRPRSLRVKIFNIQVKEVTVQDSTTTIANNLTSTVQVFTDDDYQ
LPYVVGNGTEGCLPAFPPQVFTLPQYGYATLNRDNTENPTERSSFFCLEYFPSKMLRTGNNFEFTYNF
EEVPFHSSFAPSQNLFKLANPLVDQYLYRFVSTNNTGGVQFNKNLAGRYANTYKNWFPGPMGRTQGWN
LGSGVNRASVSAFATTNRMELEGASYQVPPQPNGMTNNLQGSNTYALENTMIFNSQPANPGTTATYLE
GNMLITSESETQPVNRVAYNVGGQMATNNQSSTTAPATGTYNLQEIVPGSVWMERDVYLQGPIWAKIP
ETGAHFHPSPAMGGFGLKHPPPMMLIKNTPVPGNITSFSDVPVSSFITQYSTGQVTVEMEWELKKENS
KRWNPEIQYTNNYNDPQFVDFAPDSTGEYRTTRPIGTRYLTRPL
(AAV6; SEQ ID NO: 12)
MAADGYLPDWLEDNLSEGIREWWDLKPGAPKPKANQQKQDDGRGLVLPGYKYLGPFNGLDKGEPVNAA
DAAALEHDKAYDQQLKAGDNPYLRYNHADAEFQERLQEDTSFGGNLGRAVFQAKKRVLEPFGLVEEGA
KTAPGKKRPVEQSPQEPDSSSGIGKTGQQPAKKRLNFGQTGDSESVPDPQPLGEPPATPAAVGPTTMA
SGGGAPMADNNEGADGVGNASGNWHCDSTWLGDRVITTSTRTWALPTYNNHLYKQISSASTGASNDNH
YFGYSTPWGYFDFNRFHCHFSPRDWQRLINNNWGFRPKRLNFKLFNIQVKEVTTNDGVTTIANNLTST
VQVFSDSEYQLPYVLGSAHQGCLPPFPADVFMIPQYGYLTLNNGSQAVGRSSFYCLEYFPSQMLRTGN
NFTFSYTFEDVPFHSSYAHSQSLDRLMNPLIDQYLYYLNRTQNQSGSAQNKDLLFSRGSPAGMSVQPK
NWLPGPCYRQQRVSKTKTDNNNSNFTWTGASKYNLNGRESIINPGTAMASHKDDKDKFFPMSGVMIFG
KESAGASNTALDNVMITDEEEIKATNPVATERFGTVAVNLQSSSTDPATGDVHVMGALPGMVWQDRDV
YLQGPIWAKIPHTDGHFHPSPLMGGFGLKHPPPQILIKNTPVPANPPAEFSATKFASFITQYSTGQVS
VEIEWELQKENSKRWNPEVQYTSNYAKSANVDFTVDNNGLYTEPRPIGTRYLTRPL
(AAV7; SEQ ID NO: 13)
MAADGYLPDWLEDNLSEGIREWWDLKPGAPKPKANQQKQDNGRGLVLPGYKYLGPFNGLDKGEPVNAA
DAAALEHDKAYDQQLKAGDNPYLRYNHADAEFQERLQEDTSFGGNLGRAVFQAKKRVLEPLGLVEEGA
KTAPAKKRPVEPSPQRSPDSSTGIGKKGQQPARKRLNFGQTGDSESVPDPQPLGEPPAAPSSVGSGTV
AAGGGAPMADNNEGADGVGNASGNWHCDSTWLGDRVITTSTRTWALPTYNNHLYKQISSETAGSTNDN
TYFGYSTPWGYFDFNRFHCHFSPRDWQRLINNNWGFRPKKLRFKLFNIQVKEVTTNDGVTTIANNLTS
TIQVFSDSEYQLPYVLGSAHQGCLPPFPADVFMIPQYGYLTLNNGSQSVGRSSFYCLEYFPSQMLRTG
NNFEFSYSFEDVPFHSSYAHSQSLDRLMNPLIDQYLYYLARTQSNPGGTAGNRELQFYQGGPSTMAEQ
AKNWLPGPCFRQQRVSKTLDQNNNSNFAWTGATKYHLNGRNSLVNPGVAMATHKDDEDRFFPSSGVLI
FGKTGATNKTTLENVLMTNEEEIRPTNPVATEEYGIVSSNLQAANTAAQTQVVNNQGALPGMVWQNRD
VYLQGPIWAKIPHTDGNFHPSPLMGGFGLKHPPPQILIKNTPVPANPPEVFTPAKFASFITQYSTGQV
SVEIEWELQKENSKRWNPEIQYTSNFEKQTGVDFAVDSQGVYSEPRPIGTRYLTRNL
(AAV8; SEQ ID NO: 14)
MAADGYLPDWLEDNLSEGIREWWALKPGAPKPKANQQKQDDGRGLVLPGYKYLGPFNGLDKGEPVNAA
DAAALEHDKAYDQQLQAGDNPYLRYNHADAEFQERLQEDTSFGGNLGRAVFQAKKRVLEPLGLVEEGA
KTAPGKKRPVEPSPQRSPDSSTGIGKKGQQPARKRLNFGQTGDSESVPDPQPLGEPPAAPSGVGPNTM
AAGGGAPMADNNEGADGVGSSSGNWHCDSTWLGDRVITTSTRTWALPTYNNHLYKQISNGTSGGATND
NTYFGYSTPWGYFDFNRFHCHFSPRDWQRLINNNWGFRPKRLSFKLFNIQVKEVTQNEGTKTIANNLT
STIQVFTDSEYQLPYVLGSAHQGCLPPFPADVFMIPQYGYLTLNNGSQAVGRSSFYCLEYFPSQMLRT
GNNFQFTYTFEDVPFHSSYAHSQSLDRLMNPLIDQYLYYLSRTQTTGGTANTQTLGFSQGGPNTMANQ
AKNWLPGPCYRQQRVSTTTGQNNNSNFAWTAGTKYHLNGRNSLANPGIAMATHKDDEERFFPSNGILI
FGKQNAARDNADYSDVMLTSEEEIKTTNPVATEEYGIVADNLQQQNTAPQIGTVNSQGALPGMVWQNR
DVYLQGPIWAKIPHTDGNFHPSPLMGGFGLKHPPPQILIKNTPVPADPPTTFNQSKLNSFITQYSTGQ
VSVEIEWELQKENSKRWNPEIQYTSNYYKSTSVDFAVNTEGVYSEPRPIGTRYLTRNL
(AAV9; SEQ ID NO: 15)
MAADGYLPDWLEDNLSEGIREWWALKPGAPQPKANQQHQDNARGLVLPGYKYLGPGNGLDKGEPVNAA
DAAALEHDKAYDQQLKAGDNPYLKYNHADAEFQERLKEDTSFGGNLGRAVFQAKKRLLEPLGLVEEAA
KTAPGKKRPVEQSPQEPDSSAGIGKSGAQPAKKRLNFGQTGDTESVPDPQPIGEPPAAPSGVGSLTMA
SGGGAPVADNNEGADGVGSSSGNWHCDSQWLGDRVITTSTRTWALPTYNNHLYKQISNSTSGGSSNDN
AYFGYSTPWGYFDFNRFHCHFSPRDWQRLINNNWGFRPKRLNFKLFNIQVKEVTDNNGVKTIANNLTS
TVQVFTDSDYQLPYVLGSAHEGCLPPFPADVFMIPQYGYLTLNDGSQAVGRSSFYCLEYFPSQMLRTG
NNFQFSYEFENVPFHSSYAHSQSLDRLMNPLIDQYLYYLSKTINGSGQNQQTLKFSVAGPSNMAVQGR
NYIPGPSYRQQRVSTTVTQNNNSEFAWPGASSWALNGRNSLMNPGPAMASHKEGEDRFFPLSGSLIFG
KQGTGRDNVDADKVMITNEEEIKTTNPVATESYGQVATNHQSAQAQAQTGWVQNQGILPGMVWQDRDV
YLQGPIWAKIPHTDGNEHPSPLMGGFGMKHPPPQILIKNTPVPADPPTAFNKDKLNSFITQYSTGQVS
VEIEWELQKENSKRWNPEIQYTSNYYKSNNVEFAVNTEGVYSEPRPIGTRYLTRNL
(AAV10; SEQ ID NO: 16)
MAADGYLPDWLEDNLSEGIREWWDLKPGAPKPKANQQKQDDGRGLVLPGYKYLGPFNGLDKGEPVNAA
DAAALEHDKAYDQQLKAGDNPYLRYNHADAEFQERLQEDTSFGGNLGRAVFQAKKRVLEPLGLVEEAA
KTAPGKKRPVEPSPQRSPDSSTGIGKKGQQPAKKRLNFGQTGESESVPDPQPIGEPPAGPSGLGSGTM
AAGGGAPMADNNEGADGVGSSSGNWHCDSTWLGDRVITTSTRTWALPTYNNHLYKQISNGTSGGSTND
NTYFGYSTPWGYFDFNRFHCHFSPRDWQRLINNNWGFRPKRLSFKLFNIQVKEVTQNEGTKTIANNLT
STIQVFTDSEYQLPYVLGSAHQGCLPPFPADVFMIPQYGYLTLNNGSQAVGRSSFYCLEYFPSQMLRT
GNNFEFSYTFEDVPFHSSYAHSQSLDRLMNPLIDQYLYYLSRTQSTGGTQGTQQLLFSQAGPANMSAQ
AKNWLPGPCYRQQRVSTTLSQNNNSNFAWTGATKYHLNGRDSLVNPGVAMATHKDDEERFFPSSGVLM
FGKQGAGRDNVDYSSVMLTSEEEIKTTNPVATEQYGVVADNLQQANTGPIVGNVNSQGALPGMVWQNR
DVYLQGPIWAKIPHTDGNFHPSPLMGGFGLKHPPPQILIKNTPVPADPPTTFSQAKLASFITQYSTGQ
VSVEIEWELQKENSKRWNPEIQYTSNYYKSTNVDFAVNTEGTYSEPRPIGTRYLTRNL
(AAV11; SEQ ID NO: 17)
MAADGYLPDWLEDNLSEGIREWWDLKPGAPKPKANQQKQDDGRGLVLPGYKYLGPFNGLDKGEPVNAA
DAAALEHDKAYDQQLKAGDNPYLRYNHADAEFQERLQEDTSFGGNLGRAVFQAKKRVLEPLGLVEEGA
KTAPGKKRPLESPQEPDSSSGIGKKGKQPARKRLNFEEDTGAGDGPPEGSDTSAMSSDIEMRAAPGGN
AVDAGQGSDGVGNASGDWHCDSTWSEGKVTTTSTRTWVLPTYNNHLYLRLGTTSSSNTYNGFSTPWGY
FDFNRFHCHFSPRDWQRLINNNWGLRPKAMRVKIFNIQVKEVTTSNGETTVANNLTSTVQIFADSSYE
LPYVMDAGQEGSLPPFPNDVFMVPQYGYCGIVTGENQNQTDRNAFYCLEYFPSQMLRTGNNFEMAYNF
EKVPFHSMYAHSQSLDRLMNPLLDQYLWHLQSTTSGETLNQGNAATTFGKIRSGDFAFYRKNWLPGPC
VKQQRFSKTASQNYKIPASGGNALLKYDTHYTLNNRWSNIAPGPPMATAGPSDGDFSNAQLIFPGPSV
TGNTTTSANNLLFTSEEEIAATNPRDTDMFGQIADNNQNATTAPITGNVTAMGVLPGMVWQNRDIYYQ
GPIWAKIPHADGHFHPSPLIGGFGLKHPPPQIFIKNTPVPANPATTFTAARVDSFITQYSTGQVAVQI
EWEIEKERSKRWNPEVQFTSNYGNQSSMLWAPDTTGKYTEPRVIGSRYLTNHL
(AAV12; SEQ ID NO: 18)
MAADGYLPDWLEDNLSEGIREWWALKPGAPQPKANQQHQDNGRGLVLPGYKYLGPFNGLDKGEPVNEA
DAAALEHDKAYDKQLEQGDNPYLKYNHADAEFQQRLATDTSFGGNLGRAVFQAKKRILEPLGLVEEGV
KTAPGKKRPLEKTPNRPTNPDSGKAPAKKKQKDGEPADSARRTLDFEDSGAGDGPPEGSSSGEMSHDA
EMRAAPGGNAVEAGQGADGVGNASGDWHCDSTWSEGRVTTTSTRTWVLPTYNNHLYLRIGTTANSNTY
NGFSTPWGYFDFNRFHCHFSPRDWQRLINNNWGLRPKSMRVKIFNIQVKEVTTSNGETTVANNLTSTV
QIFADSTYELPYVMDAGQEGSFPPFPNDVFMVPQYGYCGWTGKNQNQTDRNAFYCLEYFPSQMLRTG
NNFEVSYQFEKVPFHSMYAHSQSLDRMMNPLLDQYLWHLQSTTTGNSLNQGTATTTYGKITTGDFAYY
RKNWLPGACIKQQKFSKNANQNYKIPASGGDALLKYDTHTTLNGRWSNMAPGPPMATAGAGDSDFSNS
QLIFAGPNPSGNTTTSSNNLLFTSEEEIATTNPRDTDMFGQIADNNQNATTAPHIANLDAMGIVPGMV
WQNRDIYYQGPIWAKVPHTDGHFHPSPLMGGFGLKHPPPQIFIKNTPVPANPNTTFSAARINSFLTQY
STGQVAVQIDWEIQKEHSKRWNPEVQFTSNYGTQNSMLWAPDNAGNYHELRAIGSRFLTHHL
(AAV13; SEQ ID NO: 19)
MTDGYLPDWLEDNLSEGVREWWALQPGAPKPKANQQHQDNARGLVLPGYKYLGPGNGLDKGEPVNAAD
AAALEHDKAYDQQLKAGDNPYLKYNHADAEFQERLQEDTSFGGNLGRAVFQAKKRILEPLGLVEEAAK
TAPGKKRPVEQSPAEPDSSSGIGKSGQQPARKRLNFGQTGDTESVPDPQPLGQPPAAPSGVGSTTMAS
GGGAPMADNNEGADGVGNSSGNWHCDSQWLGDRVITTSTRTWALPTYNNHLYKQISSQSGATNDNHYF
GYSTPWGYFDFNRFHCHFSPRDWQRLINNNWGFRPKRLNFKLFNIQVKEVTQNDGTTTIANNLTSTVQ
VFTDSEYQLPYVLGSAHQGCLPPFPADVFMVPQYGYLTLNNGSQAVGRSSFYCLEYFPSQMLRTGNNF
QFSYTFEDVPFHSSYAHSQSLDRLMNPLIDQYLYYLNRTQTASGTQQSRLLFSQAGPTSMSLQAKNWL
PGPCYRQQRLSKQANDNNNSNFPWTGATKYHLNGRDSLVNPGPAMASHKDDKEKFFPMHGTLIFGKEG
TNANNADLENVMITDEEEIRTTNPVATEQYGTVSNNLQNSNAGPTTGTVNHQGALPGMVWQDRDVYLQ
GPIWAKIPHTDGHFHPSPLMGGFGLKHPPPQIMIKNTPVPANPPTNFSAAKFASFITQYSTGQVSVEI
EWELQKENSKRWNPEIQYTSNYNKSVNVDFTVDTNGVYSEPRPIGTRYLTRNL
(AAV-DJ; SEQ ID NO: 20)
MAADGYLPDWLEDTLSEGIRQWWKLKPGPPPPKPAERHKDDSRGLVLPGYKYLGPFNGLDKGEPVNEA
DAAALEHDKAYDRQLDSGDNPYLKYNHADAEFQERLKEDTSFGGNLGRAVFQAKKRLLEPLGLVEEAA
KTAPGKKRPVEHSPVEPDSSSGTGKAGQQPARKRLNFGQTGDADSVPDPQPIGEPPAAPSGVGSLTMA
AGGGAPMADNNEGADGVGNSSGNWHCDSTWMGDRVITTSTRTWALPTYNNHLYKQISNSTSGGSSNDN
AYFGYSTPWGYFDFNRFHCHFSPRDWQRLINNNWGFRPKRLSFKLFNIQVKEVTQNEGTKTIANNLTS
TIQVFTDSEYQLPYVLGSAHQGCLPPFPADVFMIPQYGYLTLNNGSQAVGRSSFYCLEYFPSQMLRTG
NNFQFTYTFEDVPFHSSYAHSQSLDRLMNPLIDQYLYYLSRTQTTGGTTNTQTLGFSQGGPNTMANQA
KNWLPGPCYRQQRVSKTSADNNNSEYSWTGATKYHLNGRDSLVNPGPAMASHKDDEEKFFPQSGVLIF
GKQGSEKTNVDIEKVMITDEEEIRTTNPVATEQYGSVSTNLQRGNRQAATADVNTQGVLPGMVWQDRD
VYLQGPIWAKIPHTDGHFHPSPLMGGFGLKHPPPQILIKNTPVPADPPTTFNQSKLNSFITQYSTGQV
SVEIEWELQKENSKRWNPEIQYTSNYYKSTSVDFAVNTEGVYSEPRPIGTRYLTRNL
(AAV PHP.eB; SEQ ID NO: 21)
MAADGYLPDWLEDNLSEGIREWWALKPGAPQPKANQQHQDNARGLVLPGYKYLGPGNGLDKGEPVNAA
DAAALEHDKAYDQQLKAGDNPYLKYNHADAEFQERLKEDTSFGGNLGRAVFQAKKRLLEPLGLVEEAA
KTAPGKKRPVEQSPQEPDSSAGIGKSGAQPAKKRLNFGQTGDTESVPDPQPIGEPPAAPSGVGSLTMA
SGGGAPVADNNEGADGVGSSSGNWHCDSQWLGDRVITTSTRTWALPTYNNHLYKQISNSTSGGSSNDN
AYFGYSTPWGYFDFNRFHCHFSPRDWQRLINNNWGFRPKRLNFKLFNIQVKEVTDNNGVKTIANNLTS
TVQVFTDSDYQLPYVLGSAHEGCLPPFPADVFMIPQYGYLTLNDGSQAVGRSSFYCLEYFPSQMLRTG
NNFQFSYEFENVPFHSSYAHSQSLDRLMNPLIDQYLYYLSRTINGSGQNQQTLKFSVAGPSNMAVQGR
NYIPGPSYRQQRVSTTVTQNNNSEFAWPGASSWALNGRNSLMNPGPAMASHKEGEDRFFPLSGSLIFG
KQGTGRDNVDADKVMITNEEEIKTTNPVATESYGQVATNHQSDGTLAVPFKAQAQTGWVQNQGILPGM
VWQDRDVYLQGPIWAKIPHTDGNFHPSPLMGGFGMKHPPPQILIKNTPVPADPPTAFNKDKLNSFITQ
YSTGQVSVEIEWELQKENSKRWNPEIQYTSNYYKSNNVEFAVNTEGVYSEPRPIGTRYLTRNL
(Anc80L65; SEQ ID NO: 22)
MAADGYLPDWLEDNLSEGIREWWDLKPGAPKPKANQQKQDDGRGLVLPGYKYLGPFNGLDKGEPVNAA
DAAALEHDKAYDQQLKAGDNPYLRYNHADAEFQERLQEDTSFGGNLGRAVFQAKKRVLEPLGLVEEGA
KTAPGKKRPVEQSPQEPDSSSGIGKKGQQPARKRLNFGQTGDSESVPDPQPLGEPPAAPSGVGSNTMA
AGGGAPMADNNEGADGVGNASGNWHCDSTWLGDRVITTSTRTWALPTYNNHLYKQISSQSGGSTNDNT
YFGYSTPWGYFDFNRFHCHFSPRDWQRLINNNWGFRPKKLNFKLFNIQVKEVTTNDGTTTIANNLTST
VQVFTDSEYQLPYVLGSAHQGCLPPFPADVFMIPQYGYLTLNNGSQAVGRSSFYCLEYFPSQMLRTGN
NFQFSYTFEDVPFHSSYAHSQSLDRLMNPLIDQYLYYLSRTQTTSGTAGNRTLQFSQAGPSSMANQAK
NWLPGPCYRQQRVSKTTNQNNNSNFAWTGATKYHLNGRDSLVNPGPAMATHKDDEDKFFPMSGVLIFG
KQGAGNSNVDLDNVMITNEEEIKTTNPVATEEYGTVATNLQSANTAPATGTVNSQGALPGMVWQDRDV
YLQGPIWAKIPHTDGHFHPSPLMGGFGLKHPPPQILIKNTPVPANPPTTFSPAKFASFITQYSTGQVS
VEIEWELQKENSKRWNPEIQYTSNYNKSTNVDFAVDTNGVYSEPRPIGTRYLTRNL
(Anc80L65AAP; SEQ ID NO: 23)
MAADGYLPDWLEDNLSEGIREWWDLKPGAPKPKANQQKQDDGRGLVLPGYKYLGPFNGLDKGEPVNAA
DAAALEHDKAYDQQLKAGDNPYLRYNHADAEFQERLQEDTSFGGNLGRAVFQAKKRVLEPLGLVEEGA
KTAPGKKRPVEQSPQEPDSSSGIGKKGQQPARKRLNFGQTGDSESVPDPQPLGEPPAAPSGVGSNTMA
AGGGAPMADNNEGADGVGNASGNWHCDSTWLGDRVITTSTRTWALPTYNNHLYKQISSQSGGSTNDNT
YFGYSTPWGYFDFNRFHCHFSPRDWQRLINNNWGFRPKKLNFKLFNIQVKEVTTNDGTTTIANNLTST
VQVFTDSEYQLPYVLGSAHQGCLPPFPADVFMIPQYGYLTLNNGSQAVGRSSFYCLEYFPSQMLRTGN
NFQFSYTFEDVPFHSSYAHSQSLDRLMNPLIDQYLYYLSRTQTTSGTAGNRTLQFSQAGPSSMANQAK
NWLPGPCYRQQRVSKTTNQNNNSNFAWTGATKYHLNGRDSLVNPGPAMATHKDDEDKFFPMSGVLIFG
KQGAGNSNVDLDNVMITNEEEIKTTNPVATEEYGTVATNLQSANTAPATGTVNSQGALPGMVWQDRDV
YLQGPIWAKIPHTDGHFHPSPLMGGFGLKHPPPQILIKNTPVPANPPTTFSPAKFASFITQYSTGQVS
VEIEWELQKENSKRWNPEIQYTSNYNKSTNVDFAVDTNGVYSEPRPIGTRYLTRNL
(7m8; SEQ ID NO: 24)
MAADGYLPDWLEDTLSEGIRQWWKLKPGPPPPKPAERHKDDSRGLVLPGYKYLGPFNGLDKGEPVNEA
DAAALEHDKAYDRQLDSGDNPYLKYNHADAEFQERLKEDTSFGGNLGRAVFQAKKRVLEPLGLVEEPV
KTAPGKKRPVEHSPVEPDSSSGTGKAGQQPARKRLNFGQTGDADSVPDPQPLGQPPAAPSGLGTNTMA
TGSGAPMADNNEGADGVGNSSGNWHCDSTWMGDRVTTTSTRTWALPTYNNHLYKQISSQSGASNDNHY
FGYSTPWGYFDFNRFHCHFSPRDWQRLINNNWGFRPKRLNFKLFNIQVKEVTQNDGTTTIANNLTSTV
QVFTDSEYQLPYVLGSAHQGCLPPFPADVFMVPQYGYLTLNNGSQAVGRSSFYCLEYFPSQMLRTGNN
FTFSYTFEDVPFHSSYAHSQSLDRLMNPLIDQYLYYLSRTNTPSGTTTQSRLQFSQAGASDIRDQSRN
WLPGPCYRQQRVSKTSADNNNSEYSWTGATKYHLNGRDSLVNPGPAMASHKDDEEKFFPQSGVLIFGK
QGSEKTNVDIEKVMITDEEEIRTTNPVATEQYGSVSTNLQRGNLALGETTRPARQAATADVNTQGVLP
GMVWQDRDVYLQGPIWAKIPHTDGHFHPSPLMGGFGLKHPPPQILIKNTPVPANPSTTFSAAKFASFI
TQYSTGQVSVEIEWELQKENSKRWNPEIQYTSNYNKSINVDFTVDTNGVYSEPRPIGTRYLTRNL

### 3. Modified AAV ITR

Any transcribed AAV ITR sequences (RNA) can be modified according to the disclosure herein, by engineering the encoding modified AAV ITR DNA template to, e.g., eliminate or inactivate the TRS or equivalent thereof, and/or to eliminate the D region sequence thereof. The transcribed modified AAV ITR, resulting from transcribing such modified AAV ITR DNA template, retains the ability to facilitate the packaging of the RNA sequence of the invention into an AAV viral particle.

During AAV DNA replication, the ITRs are nicked by the virus-encoded Rep proteins at the terminal resolution site (TRS). This origin function requires three DNA sequence elements, namely the Rep binding element (RBE), a small palindrome that comprises a single tip of an internal hairpin within the terminal repeat (RBE′), and the TRS. Rep is tethered to the RBE (DNA) in a specific orientation during TRS nicking. This orientation appears to align Rep on the AAV ITR, allowing specific nucleotide contacts with the RBE′ and directing nicking to the TRS. Alterations in the polarity or position of the RBE relative to the TRS greatly inhibit Rep nicking. Substitutions within the RBE′ also reduce Rep specific activity, but only to a lesser extent. Rep interactions with the RBE and RBE′ during TRS nicking are functionally distinct, in that the Rep contact with the RBE is necessary for both the DNA helicase activity and the TRS cleavage. Meanwhile, Rep interaction with RBE′ is required primarily for ITR unwinding and formation of the TRS stem-loop structure, but is not required for TRS cleavage.

At least one transcribed modified ITR sequence (RNA) of the invention is present on the RNA sequence of the invention. The transcribed modified ITR sequence of the invention is preferably located closer to the 3′ end of the RNA sequence of the invention.

In certain embodiments, the RNA sequence of the invention comprises two transcribed modified ITR sequences.

In certain embodiments, the two transcribed modified ITR sequences may be derived from the same AAV serotype.

In another embodiment, the two transcribed modified ITR sequences may be derived from two different AAVs of different serotypes.

In certain embodiments, the transcribed modified ITR sequence(s) include(s) an insertion, deletion, and/or a mutation.

In some embodiments, the rRAAV RNA sequence of the invention comprises one transcribed modified/mutated ITR sequence and one transcribed wild-type ITR sequence.

In some embodiments, the transcribed modified ITR sequence(s) is/are based on a wild-type ITR in either the flip orientation or the flop orientation.

The subject transcribed modified ITR sequences, or their coding DNA sequences, can be readily prepared based on wild-type ITR sequences known in the art.

Representative (non-limiting) wild-type ITR sequences (DNA) including at least the following sequences listed in Table 1. A multi-sequence alignment for the 5′ ITR sequences, and a multi-sequence alignment for the 3′ ITR sequences of AAV1-AAV7 are shown in FIGS. 1B and 1C, respectively, including the consensus sequences, the TRS, the RBE, and the D region sequences.

TABLE 1
Representative Wild-type AAV Inverted Terminal
Repeat (ITR) Sequences

| AAV ITR | DNA Sequences |
|---|---|
| AAV1 5′ ITR | TTGCCCACTCCCTCTCTGCGCGCTCGCTCGCTCGGTGGGGCCTGCGGAC |
| | CAAAGGTCCGCAGACGGCAGAGCTCTGCTCTGCCGGCCCCACCGAGCGA |
| | GCGAGCGCGCAGAGAGGGAGTGGGCAACTCCATCACTAGGGGTAA |
| | (SEQ ID NO: 25) |
| AAV1 3′ ITR | TTACCCCTAGTGATGGAGTTGCCCACTCCCTCTCTGCGCGCTCGCTCGC |
| | TCGGTGGGGCCTGCGGACCAAAGGTCCGCAGACGGCAGAGCTCTGCTCT |
| | GCCGGCCCCACCGAGCGAGCGAGCGCGCAGAGAGGGAGTGGGCAA |
| | (SEQ ID NO: 26) |
| AAV2 5′ ITR | TTGGCCACTCCCTCTCTGCGCGCTCGCTCGCTCACTGAGGCCGGGCGAC |
| | CAAAGGTCGCCCGACGCCCGGGCTTTGCCCGGGCGGCCTCAGTGAGCGA |
| | GCGAGCGCGCAGAGAGGGAGTGGCCAACTCCATCACTAGGGGTTCCT |
| | (SEQ ID NO: 27) |
| AAV2 3′ ITR | AGGAACCCCTAGTGATGGAGTTGGCCACTCCCTCTCTGCGCGCTCGCTC |
| | GCTCACTGAGGCCGGGCGACCAAAGGTCGCCCGACGCCCGGGCTTTGCC |
| | CGGGCGGCCTCAGTGAGCGAGCGAGCGCGCAGAGAGGGAGTGGCCAA |
| | (SEQ ID NO: 28) |
| AAV3 5′ ITR | TTGGCCACTCCCTCTATGCGCACTCGCTCGCTCGGTGGGGCCTGGCGAC |
| | CAAAGGTCGCCAGACGGACGTGCTTTGCACGTCCGGCCCCACCGAGCGA |
| | GCGAGTGCGCATAGAGGGAGTGGCCAACTCCATCACTAGAGGTAT |
| | (SEQ ID NO: 29) |
| AAV3 3′ ITR | ATACCTCTAGTGATGGAGTTGGCCACTCCCTCTATGCGCACTCGCTCGC |
| | TCGGTGGGGCCTGGCGACCAAAGGTCGCCAGACGGACGTGCTTTGCACG |
| | TCCGGCCCCACCGAGCGAGCGAGTGCGCATAGAGGGAGTGGCCAA |
| | (SEQ ID NO: 30) |
| AAV4 5′ ITR | TTGGCCACTCCCTCTATGCGCGCTCGCTCACTCACTCGGCCCTGGAGAC |
| | CAAAGGTCTCCAGACTGCCGGCCTCTGGCCGGCAGGGCCGAGTGAGTGA |
| | GCGAGCGCGCATAGAGGGAGTGGCCAACTCCATCATCTAGGTTTGCCC |
| | (SEQ ID NO: 31) |
| AAV4 3′ ITR | GGCAAACCAGATGATGGAGTTGGCCACATTAGCTATGCGCGCTCGCTCA |
| | CTCACTCGGCCCTGGAGACCAAAGGTCTCCAGACTGCCGGCCTCTGGCC |
| | GGCAGGGCCGAGTGAGTGAGCGAGCGCGCATAGAGGGAGTGGCCAA |
| | (SEQ ID NO: 32) |
| AAV5 5′ ITR | CTCTCCCCCCTGTCGCGTTCGCTCGCTCGCTGGCTCGTTTGGGGGGGTG |
| | GCAGCTCAAAGAGCTGCCAGACGACGGCCCTCTGGCCGTCGCCCCCCCA |
| | AACGAGCCAGCGAGCGAGCGAACGCGACAGGGGGGAGAGTGCCACACTC |
| | TCAAGCAAGGGGGTTTTGTA (Seq ID NO: 33) |
| AAV5 3′ ITR | TACAAAACCTCCTTGCTTGAGAGTGTGGCACTCTCCCCCCTGTCGCGTT |
| | CGCTCGCTCGCTGGCTCGTTTGGGGGGGTGGCAGCTCAAAGAGCTGCCA |
| | GACGACGGCCCTCTGGCCGTCGCCCCCCCAAACGAGCCAGCGAGCGAGC |
| | GAACGCGACAGGGGGGAGAG (Seq ID NO: 34) |
| AAV6 5′ ITR | TTGGCCACTCCCTCTCTGCGCGCTCGCTCGCTCACTGAGGCCGGGCGAC |
| | CAAAGGTCGCCCGACGCCCGGGCTTTGCCCGGGCGGCCTCAGTGAGCGA |
| | GCGAGCGCGCAGAGAGGGAGTGGCCAACTCCATCACTAGGGGTTCCT |
| | (SEQ ID NO: 35) |
| AAV6 3′ ITR | ATACCCCTAGTGATGGAGTTGCCCACTCCCTCTATGCGCGCTCGCTCGC |
| | TCGGTGGGGCCGGCAGAGCAGAGCTCTGCCGTCTGCGGACCTTTGGTCC |
| | GCAGGCCCCACCGAGCGAGCGAGCGCGCATAGAGGGAGTGGGCAA |
| | (SEQ ID NO: 36) |
| AAV7 5′ ITR | TTGGCCACTCCCTCTATGCGCGCTCGCTCGCTCGGTGGGGCCTGCGGAC |
| | CAAAGGTCCGCAGACGGCAGAGCTCTGCTCTGCCGGCCCCACCGAGCGA |
| | GCGAGCGCGCATAGAGGGAGTGGCCAACTCCATCACTAGGGGTACCG |
| | (SEQ ID NO: 37) |
| AAV7 3′ ITR | CGGTACCCCTAGTGATGGAGTTGGCCACTCCCTCTATGCGCGCTCGCTC |
| | GCTCGGTGGGGCCTGCGGACCAAAGGTCCGCAGACGGCAGAGCTCTGCT |
| | CTGCCGGCCCCACCGAGCGAGCGAGCGCGCATAGAGGGAGTGGCCAA |
| | (SEQ ID NO: 38) |

As used herein, “RBE sequence” or “RBE” refers to the AAV ITR sequences within the A: A′ palindromic stem sequences that, when base-paired, form a stem (usually a double stranded region of about 21-23, or about 22 bp) and facilitate ITR binding to AAV Rep proteins (Rep78 and Rep68). A representative RBE sequence is shown in FIG. 1A, in both the flip and flop configurations of the wild-type AAV2 ITR.

Wild-type ITR sequences of the numerous AAV serotypes known in the art are readily available, each can be aligned with the other AAV ITRs as in FIGS. 1B and 1C. The results of the alignment can be used to identify the RBE sequences for any AAV ITR.

A “transcribed (functional) RBE” refers to a transcribed RNA corresponding to the RBE DNA template, which is either wild-type RBE, or a functional variant thereof with one or more nucleotide insertions, deletions, substitutions, and/or other mutations, so long as the functional variant RBE substantially retains the ability to bind to Rep (e.g., retains at least about 60%, 70%, 80%, 90%, 95%, or enhanced binding to Rep of the same serotype). In certain embodiments, the RBE DNA template or the transcribed RBE RNA differs from the wild-type sequence by no more than 10, 9, 8, 7, 6, 5, 4, 3, 2, 1 nucleotide(s). In certain embodiments, the functional RBE comprises up to about 30%, 25%, 20%, 15%, 10%, or 5% of sequence variation compared to the wild-type RBE, due to, for example, insertion, deletion, substitution, and/or other mutation of one or more nucleotides of the RBE.

In certain embodiments, the nucleotide sequence difference does not result in loss of paired base pair (e.g., a GC pair in the wild-type RBE can be changed to CG, AT/AU or TA/UA in the variant RBE without losing the original paired base pair).

In certain embodiments, the transcribed modified ITR sequence (RNA) retains a transcribed Rep-binding element (transcribed RBE) or a functional variant thereof, to facilitate Rep-mediated packaging. For example, the RBE DNA sequence for wild-type AAV2 ITR is SEQ ID NO: 5.

In certain embodiments, the transcribed modified ITR sequence (RNA) further retains a transcribed Rep-binding element' (transcribed RBE') sequence. For example, in FIG. 1A, the CTTTG DNA sequence forming the hairpin or loop structure in the B: B' segment of the flip ITR is the RBE' sequence.

In certain embodiments, the transcribed modified ITR sequence lacks a transcribed TRS, and/or a transcribed rcTRS, or both.

In certain embodiments, the RNA sequence of the invention lacks a transcribed (functional) TRS sequence, due to the fact that its corresponding DNA sequence lacks certain sequence elements of the wild-type TRS, such that wild-type TRS function is lost in the DNA (e.g., the sequence or internal strand normally occupied by the wild-type TRS sequence between the A: A′ segment and D region sequence, which is normally recognized and cleaved by endonuclease during AAV replication, is not cleaved if present in the ssDNA vector genome of AAV ITR).

For example, in some embodiments, the reverse complement of the TRS may be deleted or mutated, as in the dITR and dITR-D sequence used in the examples.

Alternatively or in addition, the TRS normally between the A: A′ segment and D region sequence may lack one or more nucleotides, or have one or more nucleotide substitutions or mutations (such as lacking or substituting/mutating 4 nucleotides in the dITR sequence used in the examples).

In certain embodiments, the entire or nearly the entire TRS/rcTRS in the wild-type sequence is deleted such that the resulting RNA transcript lacks a functional TRS sequence.

In certain embodiments, a part of the wild-type TRS/rcTRS sequence is altered/mutated by, for example, having an insertion, deletion, substitution, and/or other mutation in the wild-type sequence, such that the mutated TRS/rcTRS produces a corresponding RNA transcript that lacks a transcribed functional TRS. For example, in certain embodiments, 12, 3, 4, or 5 consecutive or non-consecutive TRS nucleotides and/or rcTRS nucleotides can be deleted or substituted in a mutated sequence.

In certain embodiments, the transcribed modified ITR sequence is transcribed from a modified ITR lacking a D region sequence, or at least a functional D region sequence (D sequence or D′ sequence, depending on the flip or flop configuration). For example, in some embodiments, the entire D region sequence is deleted such that the resulting RNA transcript lacks a transcribed functional D region sequence. In other embodiments, at least a portion of the D region sequence is mutated (e.g., having deletion, insertion, substitution, and/or other mutation) such that the resulting RNA transcript lacks a transcribed functional D region sequence. In certain embodiments, the mutated D region sequence has no more than 10, 9, 8, 7, 6, 5, 4, 3, 2, or 1 nucleotide of the wild-type sequence.

In certain embodiments, the modified ITR sequence (DNA template) lacks 1, 2, 3, 4, 5, 6, 7, 8, 9, or 10 most 5′ end nucleotides of the wild-type ITR sequence. For example, the dITR sequence (SEQ ID NO: 2) and the dITR-D sequence (SEQ ID NO: 3) both lack the most 5′ end 8 nucleotides compared to the wild-type ITR sequence (SEQ ID NO: 1).

Corresponding DNA sequences encoding any of the above described transcribed RNA coding sequence (DNA coding sequence for the GOI), transcribed modified AAV ITR (modified AAV ITR), transcribed functional RBE (functional RBE), transcribed functional D region sequence (functional D region sequence), and transcribed functional TRS sequence (functional TRS sequence) are expressly contemplated as within the scope of the invention.

4. Introns, Exons, UTRs, Enhancers, and other elements

The RNA sequence of the invention to be encapsidated in the rRAAV viral particles of the invention may further comprise additional optional sequence elements (such as expression control elements) that may enhance or regulate the expression of the GOI.

Expression control elements present within the RNA sequence of the invention facilitate proper heterologous polynucleotide (e.g., GOI) transcription and/or translation, including, e.g., splicing signal for introns, maintenance of the correct reading frame of the gene to permit in-frame translation of mRNA and, stop codons etc.

Typically, expression control elements, some within the RNA sequence of the invention, and others present on the DNA encoding the RNA sequence of the invention, are nucleic acid sequence(s), such as promoters and enhancers that influence expression of an operably linked heterologous polynucleotide (e.g., GOI). Such elements typically act in cis but may also act in trans. Expression control can be effected at the level of transcription, translation, splicing, message stability, etc. Typically, an expression control element that modulates transcription is juxtaposed near the 5′ end (i.e., “upstream”) of the transcribed polynucleotide. Expression control elements can also be located at the 3′ end (i.e., “downstream”) of the transcribed sequence or within the transcript (e.g., in an intron). Expression control elements can be located at a distance away from the transcribed gene of interest sequence (e.g., 100 to 500, 500 to 1000, 2,000 to 5,000, or more nucleotides from the gene of interest polynucleotide). Nevertheless, owing to the polynucleotide length limitations for viral vectors, such as AAV vectors, such expression control elements will typically be within 1-1,000, 1-500, 1-250, or 1-100 nucleotides from the transcribed gene of interest sequence.

Some non-limiting expression control elements that may be present on the RNA sequence of the invention, or DNA encoding the RNA sequence of the invention, are described in further details herein below.

### Introns

Introns are known to possess a posttranscriptional regulatory element that efficiently induces transport of mRNA out of the nucleus and enhances mRNA stability.

In certain embodiments, the rRAAV can include one or more introns or a fragment thereof. In some embodiments, the one or more introns are fragments of the gene of interest. In some embodiments, the one or more introns are heterologous to the gene of interest.

Introns have been reported to affect the levels of gene expression. This effect is known as Intron Mediated Enhancement (IME) of gene expression (Lu et al., Mol Genet Genomics 279:563-572, 2008). In some embodiments, the levels of gene expression are increases by about 1.5-fold, about 2-fold, about 2.5-fold, about 3-fold, about 3.5 fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, or about 10-fold when compared to gene expression from a sequence without the one or more introns.

Non-limiting introns include SV40 intron, beta globin intron, and short chimeric intron (CIB). Other introns include the ColE2-RNA-OUT, OIPR, and R6K-RNA-OUT introns described in Lu et al., Hum Gene Ther. 2017;28 (1): 125-134 (incorporated by reference); the human hemoglobin subunit beta (HBB2) synthetic intron (Snyder et al., Hum Gene Ther, 8 (1997), pp. 1891-1900, incorporated by reference).

In some embodiments, the one or more introns may be less than 25 nucleotides, less than 50 nucleotides, less than 100 nucleotides, less than 150 nucleotides, less than 200 nucleotides, less than 250 nucleotides, less than 300 nucleotides, less than 350 nucleotides, less than 400 nucleotides, less than 450 nucleotides, or less than 500 nucleotides.

In some embodiments, the one or more introns may be more than 25 nucleotides, more than 50 nucleotides, more than 100 nucleotides, more than 150 nucleotides, more than 200 nucleotides, more than 250 nucleotides, more than 300 nucleotides, more than 350 nucleotides, more than 400 nucleotides, more than 450 nucleotides, or more than 500 nucleotides.

In some embodiments, the one or more introns may be about 50 to about 100 nucleotides, about 50 to about 200 nucleotides, about 50 to about 300 nucleotides, about 50 to about 400 nucleotides, about 50 to about 500 nucleotides, about 100 to about 200 nucleotides, about 100 to about 300 nucleotides, about 100 to about 400 nucleotides, about 100 to about 500 nucleotides, about 200 to about 300 nucleotides, about 200 to about 400 nucleotides, about 200 to about 500 nucleotides, about 300 to about 400 nucleotides, about 300 to about 500 nucleotides, or about 400 to about 500 nucleotides.

### Enhancers

The term “enhancer” as used herein can refer to a sequence that is located adjacent to the gene of interest. Enhancer elements are typically located upstream of a promoter element in the DNA encoding the RNA sequence of the invention, but can also be located downstream of or within an intron sequence (e.g., a gene of interest) and remain functional.

Thus the enhancer or part thereof may be present in the transcribed RNA sequence of the invention.

Non-limiting examples of suitable enhancers include a CMV enhancer.

In certain embodiments, an enhancer element can be located 100 base pairs, 200 base pairs, or 300 or more base pairs upstream or downstream of a gene of interest (e.g., in the RNA sequence of the invention or a DNA coding sequence therefor). Enhancer elements typically increase expressed of a gene of interest above increased expression afforded by a promoter element.

### Untranslated Regions (UTRs)

As used herein, “Untranslated Regions” (“UTRs”) refer to RNA that are not translated after transcription. For example, the 5′ UTR is upstream of the start code of the gene of interest and the 3′ UTR is downstream of the stop codon of the gene of interest. In some embodiments, the 5′ and/or 3′ UTRs may have an insertion, deletion, or modification to enhance stability of the transcribed gene of interest. For Example, the 5′ UTR may comprise a translation initiation sequence such as, but not limited to, a Kozak sequence and an internal ribosome entry site (IRES). Kozak sequences have the consensus CCR (A/G) CCAUGG (SEQ ID NO: 108), where R is a purine (adenine or guanine) three bases upstream of the start codon (AUG), which is followed by another ‘G’.

3′ UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA (U/A) (U/A) nonamers. Molecules containing this type of AREs include GM-CSF and TNF-a. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo. Any of these 5′ and/or 3′ UTR sequences can be present in the RNA sequence of the invention.

In some embodiments, the 5′ UTR and/or 3′UTR may comprise heterologous sequence to the gene of interest. In some embodiments, the 5′ UTR and/or 3′ UTR are native to the gene of interest.

In certain embodiments, a 5′ UTR and/or a 3′ UTR from an mRNA normally expressed in a specific tissue or organ, such as lung, liver, pancreas, endothelial cells, CNS, neurons, astrocytes, skeletal muscle, cardiac muscle, smooth muscle, blood, hematopoietic cells may be used in the RNA sequence of the invention comprising a GOI targeted to one or more of these tissues.

### Polyadenylation Sequence

In certain embodiments, the RNA sequence of the invention comprise a transcribed modified AAV ITR that is 5′ to a polyA sequence, a polyA signal sequence (e.g., AAUAAA), or a sequence for RNA transcription termination (e.g., a histone downstream element).

The “polyA sequence,” “polyA tail,” “polyA signal sequence,” and “a sequence for RNA transcription termination” are defined herein above.

In certain embodiments, the RNA sequence of the invention comprises a poly A tail. Such RNA sequence can be packaged into the rRAAV viral particles of the invention and be delivered directly into a target cell, and the GOI encoded by the RNA sequence of the invention can be directly translated.

In certain embodiments, the RNA sequence of the invention comprises a polyA signal sequence and optionally a transcribed GU-rich region downstream of the polyA site. Such RNA sequence can be packaged into the rRAAV viral particles of the invention and be delivered directly into a target cell. Once inside the target cell, the polyA signal sequence may be recognized and further processed by the cytosolic polyA addition enzymes to produce a polyA tail, before the GOI encoded by the RNA sequence of the invention is translated.

Representative polyA signal sequence and surrounding sequences include human growth hormone (hGH) polyA sequence (see Liu et al., Gene Ther 20:308-317, 2013, incorporated by reference), bovine growth hormone polyadenylation signal (bGHpA) (Goodwin and Rottman, J Biol Chem. 1992 Aug. 15; 267 (23): 16330-4, incorporated by reference), SV40 early or late polyadenylation signal, and the synthetic polyA signal used in Choi et al. (Mol Brain. 2014; 7:17, incorporated herein by reference).

### Transcription Enhancer

As used herein, a “transcription enhancer” refer to cis-acting nucleotide sequences that can increase the transcription of the gene of interest. In some embodiments, the transcription enhancer can be located in the intron or partially in an exon region of the transcribed RAAV RNA sequence of the invention.

### WPRE

In certain embodiments, the RNA sequence of the invention comprises a transcribed WPRE sequence, encoded by the WPRE sequence on the encoding DNA.

Woodchuck Hepatitis Virus (WHP) Posttranscriptional Regulatory Element (WPRE) is a 600-bp or so DNA sequence that, when transcribed, creates a tertiary structure enhancing expression.

WPRE is commonly used in molecular biology to increase expression of genes delivered by viral vectors. It is a tripartite regulatory element with gamma, alpha, and beta components. The alpha component is 80 bp long: GCCACGGCGGAACTCATCGCCGCC

TGCCTTGCCCGCTGCTGGACAGGGGCTCGGCTGTTGGGCACTGACAATTCC GTGGT (SEQ ID NO: 39). When used alone, the alpha component is only 9% as active as the full tripartite WPRE sequence, which is 100% identical to base pairs 1093-1684 of the Woodchuck hepatitis B virus (WHV8) genome.

In certain embodiments, the transcribed WPRE sequence or part thereof (such as the gamma, alpha, and beta elements, preferably in the given order) is present in a 3′ UTR region of a GOI on the subject RNA sequence encapsidated in the rRAAV viral particle of the invention, to substantially increase stability and protein yield of the RNA sequence of the invention.

In certain embodiments, the WPRE sequence is a shorted WPRE (WPRE2) containing a minimal gamma element and a partial alpha-beta element (see Kalev-Zylinska, J Neurosci. 2007, 27:10456-10467, incorporated by reference).

In certain embodiments, the WPRE sequence is a shorted WPRE (WPRE3) containing minimal gamma and alpha elements (see Choi et al., Mol Brain 7, 17 (2014), incorporated by reference).

In certain embodiments, the RNA sequence of the invention comprises a WPRE sequence and a GOI lacking introns.

### Promoters

The term “promoter” as used herein is defined as a DNA sequence recognized by the synthetic machinery of the cell, or introduced synthetic machinery, required to initiate the specific transcription of a polynucleotide sequence.

Thus the RNA sequence of the invention does not comprise a promoter. On the other hand, a DNA encoding the RNA sequence of the invention (such as an expression cassette or expression vector encoding the RNA sequence of the invention) comprises a promoter for transcribing the RNA sequence of the invention.

As used herein, the term “promoter/regulatory sequence” means a nucleic acid sequence which is required for expression of a gene product operably linked to the promoter/regulatory sequence. In some instances, this sequence may be the core promoter sequence. In other instances, this sequence may also include an enhancer sequence and other regulatory elements which are required for expression of the gene product. The promoter/regulatory sequence may, for example, be one which expresses the gene product (e.g., the RNA sequence of the invention) in a tissue or cell type specific manner.

As used herein, the term “operable linkage” or “operably linked” refers to a physical or functional juxtaposition of the components so described as to permit them to function in their intended manner. In the example of an expression control element in operable linkage with a heterologous polynucleotide, the relationship is such that the control element modulates expression of the heterologous polynucleotide. More specifically, for example, two DNA sequences operably linked means that the two DNAs are arranged (cis or trans) in such a relationship that at least one of the DNA sequences is able to exert a physiological effect upon the other sequence.

In certain embodiments, the promoter is a constitutive promoter.

As used herein, a “constitutive” promoter is a nucleotide sequence which, when operably linked with a polynucleotide which encodes or specifies a gene product, causes the gene product to be produced in a cell under most or all physiological conditions of the cell.

In certain embodiments, a promoter that can be used to constitutively drive the expression of the RNA sequence of the invention from a DNA encoding the same can include: a β glucuronidase (GUSB) promoter, a cytomegalovirus (CMV) immediate-early (Ie) enhancer and/or promoter, a chicken β-actin (CBA) promoter or derivative thereof such as a CAG promoter, CB promoter, a (human) elongation factor 1α-subunit (EF1α) promoter, and a ubiquitin C (UBC) promoter.

In certain embodiments, the promoter is an inducible promoter.

As used herein, an “inducible” promoter is a nucleotide sequence which, when operably linked with a polynucleotide which encodes or specifies a gene product, causes the gene product to be produced in a cell substantially only when an inducer which corresponds to the promoter is present in the cell.

In certain embodiments, the promoter is a tissue-specific promoter, a species specific promoter, or a cell cycle-specific promoter. See Parr et al., Nat. Med.3:1145-9, 1997 (entire contents incorporated herein by reference).

As used herein, a “tissue—or cell-type-specific” promoter is a nucleotide sequence which, when operably linked with a polynucleotide encodes or specified by a gene, causes the gene product to be produced in a specific cell type or a specific tissue preferentially, due to, for example, the cell/tissue is a cell type or tissue type in which the promoter is normally active.

Tissue—or cell type-specific promoters may include neuronal tissue specific promoter; CNS—or PNS-specific promoter such as astrocyte, oligodendrocyte, or neuronal promotor; hematopietic lineage specific promoter such as B cell promoter, T cell promoter, NK cell promoter, monocyte promoter, leukocyte promoter, macrophage promoter; endothelial cell promoter; pancreatic promoter; liver/hepatic cell promoter; lung tissue promoter, etc.

Representative tissue-specific promoters include prion promoter, neuron-specific enolase (NSE), neurofilament light (NFL) promoter, neurofilament heavy (NFH) promoter, platelet-derived growth factor (PDGF), platelet-derived growth factor B-chain (PDGF-β), synapsin (Syn), synapsin 1 (Syn1), methyl-CpG binding protein 2 (MeCP2), Ca2+/calmodulin-dependent protein kinase II (CaMKII), metabotropic glutamate receptor 2 (mGluR2), neurofilament light (NFL) or heavy (NFH), β-globin minigene nβ2, preproenkephalin (PPE), enkephalin (Enk) and excitatory amino acid transporter 2 (EAAT2) promoters.

Astrocyte-specific promoters include glial fibrillary acidic protein (GFAP) and EAAT2 promoters.

Oligodendrocyte-specific promoters include the myelin basic protein (MBP) promoter.

In some embodiments, the promoter is heterologous to the gene of interest. In some embodiments, the promoter is the natural promoter of the gene of interest. In some embodiments, the heterologous promoter includes an insertion, deletion, substitution, and/or other mutation. In some embodiments, the natural promoter includes an insertion, deletion, substitution, and/or other mutation.

In certain embodiments, the promoter is a Pol II promoter. In certain embodiments, the promoter is a Pol III promoter, such as U6 promoter.

### 5. Vectors (Plasmids or Bacmids)

As used herein, a “vector” generally refers to a composition of matter which comprises an isolated nucleic acid (DNA or RNA) and which can be used to deliver the isolated nucleic acid to the interior of a cell.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids, bacmids (e.g., naked or contained in liposomes) and viruses (e.g., lentiviruses, retroviruses, adenoviruses, and adeno-associated viruses) that incorporate the recombinant polynucleotide. An rRAAV RNA sequence of the invention comprising a GOI is a vector for delivering the GOI into a target/host cell through a rRAAV viral particle encapsidating the vector.

In certain embodiments, the rRAAV RNA sequence of the invention is encoded by a DNA expression vector, such as a plasmid or bacmid (e.g., one that can be maintained or replicated like a baculovirus inside an insect cell). Such DNA expression vector can transcribe the RNA sequence of the invention within a suitable host cell, such as a mammalian packaging cell (e.g., HEK293T cells) or an insect packaging cell (e.g., Sf9 cells), such that the subject rRAAV viral particles can be produced in the presence of other elements necessary for rRAAV packaging (such as rep and cap coding sequences).

Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

In some embodiments, the RAAV is transcribed from a plasmid or bacmid. The plasmid or bacmid can include the gene of interest sequence. In some embodiments, the promoter is operably linked to the gene of interest and is located upstream of the gene of interest. In some embodiments, promoter is not in the transcribed RAAV.

6. AAV particles and populations of AAV particles

In certain embodiments, the invention provides an isolated rRAAV viral particle comprising any one of the RNA sequence of the invention encapsidated within any one of the AAV capsid or viral particle described herein.

In some embodiments, the AAV capsid or viral particle is of a serotype or a combination of one or more serotypes described herein.

In the rRAAV vectors or RNA sequence of the present invention, the rRAAV genome (RNA) may be either a single stranded (ss) nucleic acid or a double stranded (ds), self-complementary (sc) nucleic acid.

A related aspect of the invention provides a population of recombinant viral particles (e.g., rRAAV particles) comprising a plurality of recombinant viral particle (e.g., rRAAV particle) of the invention, wherein at least about 10%, 20%, 30%, 40%, 50%, 60%, 70%, 80%, 90%, 95%, 96%, 97%, 98%, 99% or more of the recombinant viral particles (e.g., rRAAV particles) within the population have encapsidated RNA sequence of the invention.

In some embodiments, the population of rRAAV particles contain a plurality of rRAAV viral particle of the invention, wherein about 40%, 50%, 60%, 70%, 80%, 90%, 95%, 96%, 97%, 98%, 99% or more of the rRAAV particles within the population have encapsidated RNA sequence of the invention.

In certain embodiments, the population of recombinant viral particles (e.g., rRAAV particles) comprises at least 1×10⁴ viral particles, at least 2×10⁴ viral particles, at least 5×10⁴ viral particles, at least 1×10⁵ viral particles, at least 2×10⁵ viral particles, at least 5×10⁵ viral particles, at least 1×10⁶ viral particles, at least 2×10⁶ viral particles, at least 5×10⁶ viral particles, at least 1×10⁷ viral particles, at least 2×10⁷ viral particles, at least 5×10⁷ viral particles, at least 1×10⁸ viral particles, at least 2×10⁸ viral particles, at least 5×10⁸ viral particles, at least 1×10⁹ viral particles, at least 2×10⁹ viral particles, at least 5×10⁹ viral particles, at least 1×10¹⁰ viral particles, at least 2×10¹⁰ viral particles, at least 5×10¹⁰ viral particles, at least 1×10¹¹ viral particles, at least 2×10¹¹ viral particles, at least 5×10¹¹ viral particles, at least 1×10¹² viral particles, at least 2×10¹² viral particles, at least 5×10¹² viral particles, at least 1×10¹³ viral particles, at least 2×10¹³ viral particles, at least 5×10¹³ viral particles, at least 1×10¹⁴ viral particles, at least 2×10¹⁴ viral particles, at least 5×10¹⁴ viral particles, at least 1×10¹⁵ viral particles, at least 2×10¹⁵ viral particles, at least 5×10¹⁵ viral particles, at least 1×10¹⁶ viral particles, at least 2×10¹⁶ viral particles, or at least 5× 10¹⁶ viral particles.

In certain embodiments, at most 50%, 45%, 40%, 35%, 30%, 25%, 20%, 15%, 10%, 5%, 3%, 2%, 1%, 0.1%, 0.01% or less of the population of recombinant viral particles encapsidate non-RNA (e.g., DNA) within the viral particles.

### 7. Host Cells and AAV Production

General principles of rAAV production are known in the art. See review in, for example, Carter (Current Opinions in Biotechnology, 1533-539, 1992); and Muzyczka, Curr. Topics in Microbial, and Immunol 158:97-129, 1992, both incorporated herein by reference). Various approaches are described in Ratschin et al (Mol. Cell. Biol. 4:2072, 1984; Hermonat et al. (Proc. Natl. Acad. Sci. USA 81:6466, 1984); Tratschin et al. (Mol. Cell. Biol. 5:3251, 1985); Mclaughlin et al. (J. Virol 62:1963, 1988); and Lebkowski et al. (Mol. Cell. Biol 7:349, 1988), Samulski et al. (J. Virol 63:3822-3828, 1989); U.S. Pat. No. 5,173,414; WO 95/13365 and U.S. Pat. No. 5,658,776; WO 95/13392; WO 96/17947; PCT/US98/18600; WO 97/09441; WO 97/08298; WO 97/21825; WO 97/06243; WO 99/11764; Perrin et al. (Vaccine 13:1244-1250, 1995; Paul et al. (Human Gene Therapy 4:609-615, 1993); Clark et al. (Gene Therapy 3:1124-1132, 1996; U.S. Pat. Nos. 5,786,211; 5,871,982; and 6,258,595.

AAV vector serotypes can be matched to target cell types. For example, Table 2 of WO2018002719A1 lists exemplary cell types that can be transduced by the indicated AAV serotypes (incorporated herein by reference).

Packaging cells are used to form virus particles that are capable of infecting a host cell. Such cells include HEK293 and Sf9 cells, which can be used to package AAV and adenovirus.

Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions can be supplied in trans by the packaging cell line, usually as a result of expression of these viral functions/proteins (such as the rep and cap genes for AAV) either as transgenes integrated into the packaging cell, or as transgenes on a second viral vector or expression vector introduced into the packaging cell.

For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, recombinant AAVs may be produced using the triple transfection method (described in detail in U.S. Pat. No. 6,001,650). Typically, the recombinant AAVs are produced by transfecting a host cell with an recombinant AAV vector (comprising a gene of interest) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some embodiments, the subject rRAAV is produced using a baculovirus expression system packaged in insect cells such as Sf9 cells. See, for example, WO2007046703, WO2007148971, WO2009014445, WO2009104964, WO2013036118, WO2011112089, WO2016083560, WO2015137802, and WO2019016349, all incorporated herein by reference.

The vector titers are usually expressed as viral genomes per ml (vg/ml). In certain embodiments, viral titers is above 1×10⁹, above 5×10¹⁰, above 1×10¹¹, above 5×10¹¹, above 1×10¹², above 5×10¹², or above 1×10¹³ vg/ml.

### 8. Gene of Interest (GOI) or RNA Sequence of Interest (RSI)

The rRAAV viral particles of the invention can be used to deliver any gene of interest (GOI) or RNA sequence of Interest (RSI) to a host cell, for any purpose, so long as the GOI is an RNA within the packaging limit of the chosen AAV viral capsid or AAV viral particle shell, such as about 4,700 nucleotides overall length for most AAV viral particles, up to about 8,900 nucleotides for certain large capacity AAV viral particles such as AAV5.

In certain embodiments, representative (non-limiting) RNA sequence of interest (RSI) includes, for example, a protein-encoding RNA, an mRNA, a non-coding RNA (ncRNA), a tRNA, a ribosomal RNA (rRNA), a transfer-messenger RNA (tmRNA), an antisense oligonucleotide (ASO), an RNA aptamer, an RNA component of CRISPR-Cas system such as a single guide RNA (or sgRNA, chimeric RNA, RNA chimera), CRISPR RNA (crRNA), tracr RNA, or an RNA component of a RISC complex or RNAi pathway (such as shRNA, miRNA, or siRNA), a regulatory RNA, Piwi-interacting RNAs (piRNAs), small nucleolar RNAs (snoRNAs), a long non-coding RNA (lncRNA) (including intergenic lincRNA, intronic ncRNA, and sense/antisense lncRNA), a long intervening/intergenic noncoding RNA (lincRNA), an enhancer RNA, a bacterial small RNA (sRNA), snRNA, exRNA, scaRNA, Xist, and HOTAIR, and a precursor thereof.

In certain embodiments, the RNA sequence of the invention comprises a coding sequence for a protein or polypeptide.

In certain embodiments, protein or polypeptide is a wild-type protein or functional equivalent or variant thereof (such as an enzyme or a structural protein) that can be used to replace a defective protein in a target cell, tissue, or organism.

In certain embodiments, protein or polypeptide is a wild-type protein or functional equivalent or variant thereof (such as an enzyme or a structural protein) that can be used to antagonize the detrimental effect of a compound (small molecule compound, or macromolecules such as lipids, fatty acids, protein, nucleic acid, etc) in a target cell, tissue, or organism.

For example, in certain embodiments, the RNA sequence of the invention comprises a coding sequence for an effector enzyme of CRISPR/Cas system.

In certain embodiments, the CRISPR-Cas system is a Class 1 system, and the effector enzyme is a type I, III, or IV effector enzyme.

In certain embodiments, the CRISPR-Cas system is a Class 2 system, and the effector enzyme is a type II, V, or VI effector enzyme.

For example, in some embodiments, the effector enzyme is a Class 2, type II enzyme such as Cas9, including Streptococcus pyogenes (SpCas9) or SaCas9 (see WO 2014/093622 (PCT/US2013/074667), incorporated by reference).

In certain embodiments, the Cas effector enzyme is a Class 2, type V Cas protein, including Cas12a (formerly known as Cpf1, such as Francisella novicida Cas 12a), C2cl, and C2c3.

In certain embodiments, the Cas effector enzyme is a Class 2, type VI Cas protein, including Cas13a (also known as C2c2), Cas13b, Cas13c, Cas13d, Cas13e, and Cas13f. These Cas proteins use their crRNA to recognize target RNA sequences, rather than target DNA sequences in Cas9 and Cas12a.

In certain embodiments, the Cas effector enzyme is any one of the Cas effector enzymes described in WO2020/028555 (entire content incorporated herein by reference), including any of Cas9, Cas12 (e.g., Cas12a, Cas12b, Cas12c, Cas12d, etc.), Cas13 (e.g., Cas13a, Cas13b (such as Cas13b-t1, Cas13b-t2, Cas13b-t3), Cas13c, Cas13d, etc.), Cas14, CasX, and CasY.

In certain embodiments, the Cas effector enzyme is fused to a DNA and/or RNA base editor, such as Cytosine or Adenine base editors (CBEs or ABEs). In certain embodiments, the base editor preferantially edits DNA bases and optionally have reduced or substantially no off-target RNA base editing capability. In certain embodiments, the base editor preferantially edits RNA bases and optionally have reduced or substantially no off-target DNA base editing capability. In certain embodiments, the base editor edits both DNA and RNA bases.

In certain embodiments, the base editor is a first, second (BE2), third (BE3), or fourth generation (BE4) base editor. In certain embodiments, the base editor is a dual base editor.

In certain embodiments, the base editor is an RNA adenosine deaminase (ADAR), such as ADARI, ADAR2, or ADARDD including ADAR2DD (E488Q).

In any of the above embodiments, the RNA sequence of the invention can further comprise a guide RNA sequence designed to be loaded into the encoded CRISPR/Cas effector enzyme for binding to a target polynucleotide sequence complementary to the guide RNA. Such gRNA sequence can be processed by cellular nucleases and be released/separated from the RNA sequence of the invention after the RNA sequence of the invention has been delivered by the rRAAV viral particles of the invention to a target host cell. For example, the gRNA can be present in an unpaired 5′ or 3′ flanking region sequence of a pri-miRNA hairpin structure that is part of the RNA sequence of the invention, and, upon processing of the pri-miRNA by cellular enzymes such as Drosha, is released/separated from the primary pri-miRNA transcript.

In certain embodiments, the RNA sequence of the invention comprises a coding sequence for an effector enzyme of CRISPR/Cas system, and further comprising a coding sequence for the DNA or RNA base-editing enzyme or domain, such that a fusion of a Cas effector enzyme and the DNA/RNA base-editing enzyme/domain is encoded by the RNA sequence. In certain embodiments, the Cas effector enzyme is defective in nuclease activity, such that it is able to bind to a target polynucleotide sequence through the guide RNA it binds, but is unable to cleave the DNA/RNA target polynucleotide.

In certain embodiments, the RNA sequence of the invention comprises a coding sequence for a variant or derivative of the effector enzyme of CRISPR/Cas system, wherein the variant comprises deletions (such as N and/or C terminal deletions, e.g., N-terminal deletion of no more than 210 residues, and/or a C-terminal deletion of no more than 180 residues for Cas13e or Cas13f), insertions, or substitutions of a wild-type CRISPR/Cas system effector enzyme but substantially retains the ability of the wild-type effector enzyme to bind to the gRNA, and/or to cleave the target polynucleotide. In certain embodiments, the variant lacks activity to cleave a target polynucleotide.

In certain embodiments, the RNA base-editing domain encoded by the RNA sequence of the invention is an adenosine deaminase, such as a double-stranded RNA-specific adenosine deaminase (e.g., ADARI or ADAR2); apolipoprotein B mRNA editing enzyme; catalytic polypeptide-like (APOBEC); or activation-induced cytidine deaminase (AID).

In certain embodiments, the RNA base-editing domain encoded by the RNA sequence of the invention comprises an adenosine deaminase and/or a cytidine deaminase, such as a cytidine deaminase acting on RNA (CDAR), such as a double-stranded RNA-specific adenosine deaminase (ADAR) (e.g., ADARI or ADAR2), apolipoprotein B mRNA editing enzyme, catalytic polypeptide-like (APOBEC, such as APOBEC1, APOBEC2, APOBEC3A, APOBEC3B, APOBEC3C, APOBEC3D, APOBEC3E, APOBEC3F, APOBEC3G, APOBEC3H, and APOBEC4), activation-induced cytidine deaminase (AID), a cytidine deaminase 1 (CDA1), or a mutant thereof.

In certain embodiments, the ADAR has E488Q/T375G double mutation or is ADAR2DD.

In certain embodiments, the base-editing domain is further fused to an RNA-binding domain, such as MS2.

In certain embodiments, the variant or derivative of the encoded CRISPR/Cas effector enzyme further comprises an RNA methyltransferase, a RNA demethylase, an RNA splicing modifier, a localization factor, or a translation modification factor.

In certain embodiments, the Cas effector enzyme, the variant/derivative, or a functional fragment thereof comprises a nuclear localization signal (NLS) sequence or a nuclear export signal (NES).

In certain embodiments, the Cas effector enzyme, the variant/derivative thereof, or the functional fragment thereof, is fused to a heterologous functional domain. In certain embodiments, the heterologous functional domain comprises: a nuclear localization signal (NLS), a reporter protein or a detection label (e.g., GST, HRP, CAT, GFP, HcRed, DsRed, CFP, YFP, BFP), a localization signal, a protein targeting moiety, a DNA binding domain (e.g., MBP, Lex A DBD, Gal4 DBD), an epitope tag (e.g., His, myc, V5, FLAG, HA, VSV-G, Trx, etc), a transcription activation domain (e.g., VP64 or VPR), a transcription inhibition domain (e.g., KRAB moiety or SID moiety), a nuclease (e.g., FokI), a deamination domain (e.g., ADAR1, ADAR2, APOBEC, AID, or TAD), a methylase, a demethylase, a transcription release factor, an HDAC, a polypeptide having ssRNA cleavage activity, a polypeptide having dsRNA cleavage activity, a polypeptide having ssDNA cleavage activity, a polypeptide having dsDNA cleavage activity, a DNA or RNA ligase, or any combination thereof. In certain embodiments, the heterologous functional domain is fused N-terminally, C-terminally, or internally in the fusion protein.

In certain embodiments, the RNA sequence of the invention comprises a coding sequence for a CasPR (CRISPR-associated Protein for Class 1 pre-crRNA processing) fusion protein, comprising a CasPR (or a homolog, an ortholog, a paralog, a variant, a derivative, or a functional fragment thereof) fused to a heterologous functional domain; or a functional variant thereof.

In certain embodiments, the CasPR is Cas5d, Cas6, or Csf5.

In certain embodiments, the CasPR is MtCas6 (I-A) (Sequence 1), MmCas6 (I-B) (Sequence 2), SpCas5d (I-C1) (Sequence 3), BhCas5d (I-C2) (Sequence 4), SaCas6 (I-D) (Sequence 5), EcCas6e (I-E) (Sequence 6), PaCas6f (I-F) (Sequence 7), MtCas6 (III-A) (Sequence 8), PfCas6 (III-B) (Sequence 9), PaCsf5 (IV-A1) (Sequence 10), or MtCsf5 (IV-A2) (Sequence 11). All sequences incorporated herein by reference.

| Sequence No. | Description |
|---|---|
| 1 | MtCas6 Amino Acid Sequence |
| 2 | MmCas6 Amino Acid Sequence |
| 3 | SpCas5d Amino Acid Sequence |
| 4 | BhCas5d Amino Acid Sequence |
| 5 | SaCas6 Amino Acid Sequence |
| 6 | EcCas6e Amino Acid Sequence |
| 7 | PaCas6f Amino Acid Sequence |
| 8 | MtCas6 Amino Acid Sequence |
| 9 | PfCas6 Amino Acid Sequence |
| 10 | PaCsf5 Amino Acid Sequence |
| 11 | MtCsf5 Amino Acid Sequence |
| 12 | MtCas6 Direct Repeat (DR) Sequence |
| 13 | MmCas6 Direct Repeat (DR) Sequence |
| 14 | SpCas5d Direct Repeat (DR) Sequence |
| 15 | BhCas5d Direct Repeat (DR) Sequence |
| 16 | SaCas6 Direct Repeat (DR) Sequence |
| 17 | EcCas6e Direct Repeat (DR) Sequence |
| 18 | PaCas6f Direct Repeat (DR) Sequence |
| 19 | MtCas6 Direct Repeat (DR) Sequence |
| 20 | PfCas6 Direct Repeat (DR) Sequence |
| 21 | PaCsf5 Direct Repeat (DR) Sequence |
| 22 | MtCsf5 Direct Repeat (DR) Sequence |
| 23 | MtCas6 Direct Repeat (DR) Transcript Sequence |
| 24 | MmCas6 Direct Repeat (DR) Transcript Sequence |
| 25 | SpCas5d Direct Repeat (DR) Transcript Sequence |
| 26 | BhCas5d Direct Repeat (DR) Transcript Sequence |
| 27 | SaCas6 Direct Repeat (DR) Transcript Sequence |
| 28 | EcCas6e Direct Repeat (DR) Transcript Sequence |
| 29 | PaCas6f Direct Repeat (DR) Transcript Sequence |
| 30 | MtCas6 Direct Repeat (DR) Transcript Sequence |
| 31 | PfCas6 Direct Repeat (DR) Transcript Sequence |
| 32 | PaCsf5 Direct Repeat (DR) Transcript Sequence |
| 33 | MtCsf5 Direct Repeat (DR) Transcript Sequence |

(Sequence 1)
(SEQ ID NO: 40)
MPLIFKIGYNVIPLQDVILPTPSSKVLKYLIQSGKLIPSLKDLITSRDKY
KPIFISHLGFNQRRIFQTNGNLKTITKGSRLSSIIAFSTQANVLSEVADE
GIFETVYGKFHIMIESIEIVEVEKLKEEVEKHMNDNIRVRFVSPTLLSSK
VLLPPSLSERYKKIHAGYSTLPSVGLIVAYAYNVYCNLIGKKEVEVRAFK
FGILSNALSRIIGYDLHPVTVAIGEDSKGNLRKARGVMGWIEFDIPDERL
KRRALNYLLTSSYLGIGRSRGIGFGEIRLEFRKIEEKEG (Sequence
1)
(Sequence 2)
(SEQ ID NO: 41)
MDLEYMHISYPNILLNMRDGSKLRGYFAKKYIDEEIVHNHRDNAFVYKYP
QIQFKIIDRSPLIIGIGSLGINFLESKRIFFEKELIISNDTNDITEVNVH
KDMDHFGTTDKILKYQFKTPWMALNAKNSEIYKNSDEIDREEFLKRVLIG
NILSMSKSLGYTIEEKLKVKINLKEVPVKFKNQNMVGFRGEFYINFDIPQ
YLGIGRNVSRGFGTVVKV
(Sequence 3)
(SEQ ID NO: 42)
MYRSRDFYVRVSGQRALFTNPATKGGSERSSYSVPTRQALNGIVDAIYYK
PTFTNIVTEVKVINQIQTELQGVRALLHDYSADLSYVSYLSDVVYLIKFH
FVWNEDRKDLNSDRLPAKHEAIMERSIRKGGRRDVFLGTRECLGLLDDIS
QEEYETTVSYYNGVNIDLGIMFHSFAYPKDKKTPLKSYFTKTVMKNGVIT
FKAQSECDIVNTLSSYAFKAPEEIKSVNDECMEYDAMEKGEN
(Sequence 4)
(SEQ ID NO: 43)
MRNEVQFELFGDYALFTDPLTKIGGEKLSYSVPTYQALKGIAESIYWKPT
IVFVIDELRVMKPIQMESKGVRPIEYGGGNTLAHYTYLKDVHYQVKAHFE
FNLHRPDLAFDRNEGKHYSILQRSLKAGGRRDIFLGARECQGYVAPCEFG
SGDGFYDGQGKYHLGTMVHGFNYPDETGQHQLDVRLWSAVMENGYIQFPR
PEDCPIVRPVKEMEPKIFNPDNVQSAEQLLHDLGGE
(Sequence 5)
(SEQ ID NO: 44)
MPNDPYSLYSIVIELGAAEKGFPTGILGRSLHSQVLQWFKQDNPFLATEL
HQSQISPFSISPLMGKRHAKLTKAGDRLFFRICLLRGDLLQPLLNGIEQT
VNQSVCLDKFRFRLCQTHILPGSHPLAGASHYSLISQTPVSSKITLDFKS
STSFKVDRKIIQVFPLGEHVFNSLLRRWNNFAPEDLHFSQVDWSIPIAAF
DVKTIPIHLKKVEIGAQGWVTYIFPNTEQAKIASVLSEFAFFSGVGRKTT
MGMGQVQVRS
(Sequence 6)
(SEQ ID NO: 45)
MYLSKVIlARAWSRDLYQLHQGLWHLFPNRPDAARDFLFHVEKRNTPEGC
HVLLQSAQMPVSTAVATVIKTKQVEFQLQVGVPLYFRLRANPIKTILDNQ
KRLDSKGNIKRCRVPLIKEAEQIAWLQRKLGNAARVEDVHPISERPQYFS
GDGKSGKIQTVCFEGVLTINDAPALIDLVQQGIGPAKSMGCGLLSLAPL
(Sequence 7)
(SEQ ID NO: 46)
MDHYLDIRLRPDPEFPPAQLMSVLFGKLHQALVAQGGDRIGVSFPDLDES
RSRLGERLRIHASADDLRALLARPWLEGLRDHLQFGEPAVVPHPTPYRQV
SRVQAKSNPERLRRRLMRRHDLSEEEARKRIPDTVARALDLPFVTLRSQS
TGQHFRLFIRHGPLQVTAEEGGFTCYGLSKGGFVPWE
(Sequence 8)
(SEQ ID NO: 47)
MAARRGGIRRTDLLRRSGQPRGRHRASAAESGLTWISPTLILVGFSHRGD
RRMTEHLSRLTLTLEVDAPLERARVATLGPHLHGVLMESIPADYVQTLHT
VPVNPYSQYALARSTTSLEWKISTLTNEARQQIVGPINDAAFAGFRLRAS
GIATQVTSRSLEQNPLSQFARIFYARPETRKFRVEFLTPTAFKQSGEYVF
WPDPRLVFQSLAQKYGAIVDGEEPDPGLIAEFGQSVRLSAFRVASAPFAV
GAARVPGFTGSATFTVRGVDTFASYIAALLWFGEFSGCGIKASMGMGAIR
VQPLAPREKCVPKP
(Sequence 9)
(SEQ ID NO: 48)
MRFLIRLVPEDKDRAFKVPYNHQYYLQGLIYNAIKSSNPKLATYLHEVKG
PKLFTYSLFMAEKREHPKGLPYFLGYKKGFFYFSTCVPEIAEALVNGLLM
NPEVRLWDERFYLHEIKVLREPKKFNGSTFVTLSPIAVTVVRKGKSYDVP
PMEKEFYSIIKDDLQDKYVMAYGDKPPSEFEMEVLIAKPKRFRIKPGIYQ
TAWHLVFRAYGNDDLLKVGYEVGFGEKNSLGFGMVKVEGNKTTKEAEEQE
KITFNSREELKTGV
(Sequence 10)
(SEQ ID NO: 49)
MFVTQVIFNIGERTYPDRARAMVAELMDGVQPGLVATLMNYIPGTSTSRT
EFPTVQFGGASDGFCLLGFGDGGGAIVRDAVPLIHAALARRMPDRIIQVE
HKEHSLSAEARPYVLSYTVPRMVVQKKQRHAERLLHEAEGKAHLEGLFLR
SLQRQAAAVGLPLPENLEVEFKGAVGDFAAKHNPNSKVAYRGLRGAVFDV
NARLGGIWTAGFMLSKGYGQFNATHQLSGAVNALSE
(Sequence 11)
(SEQ ID NO: 50)
MHQTLIRINWPKGFKCPPAEFREKLAKSEMFPPEFFHYGTELAVWDKQTA
EVEGKIKTVSKEKIIKTFDKPIPLNGRAPVRVIGGQAWAGVIADPEMEGM
LIPHLGSILKVASSAAGCAVKIELEQRKFGISYTEYPVKYNLRELVLKRR
CEDARSTDIESLIADRIWGGVSGESYYGIDGTCAKFGFEPPSREQLELRI
FPMKNIGLHMKSSDGLSKEYMSLIDAEVWMNAKLEGVWQVGNLISRGYGR
FIKSIGAQS
Sequence 12:
(SEQ ID NO: 51)
GATAATCTCTTATAGAATTGAAAG
Sequence 13:
(SEQ ID NO: 52)
CTAAAAGAATAACTTGCAAAATAACAAGCATTGAAAC
Sequence 14:
(SEQ ID NO: 53)
GTCTCACCCTTCATGGGTGAGTGGATTGAAAT
Sequence 15:
(SEQ ID NO: 54)
GTCGCACTCTTCATGGGTGCGTGGATTGAAAT
Sequence 16:
(SEQ ID NO: 55)
GTTTCAGTCCCGTAGTCGGGATTTAGTGGTTGGAAAG
Sequence 17:
(SEQ ID NO: 56)
GAGTTCCCCGCGCCAGCGGGGATAAACCG
Sequence 18:
(SEQ ID NO: 57)
GTTCACTGCCGTATAGGCAGCTAAGAAA
Sequence 19:
(SEQ ID NO: 58)
GTCGTCAGACCCAAAACCCCGAGAGGGGACGGAAAC
Sequence 20:
(SEQ ID NO: 59)
GTTACAATAAGACTAAAATAGAATTGAAAG
Sequence 21:
(SEQ ID NO: 60)
GTATTTCCCGCGTGCGCGGGGGTGAGCGG
Sequence 22:
(SEQ ID NO: 61)
TATTGGATACACCCACTCATTGGTGGGTGGTTAGAAC
Sequence 23:
(SEQ ID NO: 62)
GAUAAUCUCUUAUAGAAUUGAAAG
Sequence 24:
(SEQ ID NO: 63)
CUAAAAGAAUAACUUGCAAAAUAACAAGCAUUGAAAC
Sequence 25:
(SEQ ID NO: 64)
GUCUCACCCUUCAUGGGUGAGUGGAUUGAAAU
Sequence 26:
(SEQ ID NO: 65)
GUCGCACUCUUCAUGGGUGCGUGGAUUGAAAU
Sequence 27:
(SEQ ID NO: 66)
GUUUCAGUCCCGUAGUCGGGAUUUAGUGGUUGGAAAG
Sequence 28:
(SEQ ID NO: 67)
GAGUUCCCCGCGCCAGCGGGGAUAAACCG
Sequence 29:
(SEQ ID NO: 68)
GUUCACUGCCGUAUAGGCAGCUAAGAAA
Sequence 30:
(SEQ ID NO: 69)
GUCGUCAGACCCAAAACCCCGAGAGGGGACGGAAAC
Sequence 31:
(SEQ ID NO: 70)
GUUACAAUAAGACUAAAAUAGAAUUGAAAG
Sequence 32:
(SEQ ID NO: 71)
GUAUUUCCCGCGUGCGCGGGGGUGAGCGG
Sequence 33:
(SEQ ID NO: 72)
UAUUGGAUACACCCACUCAUUGGUGGGUGGUUAGAAC

In certain embodiments, the heterologous functional domain fused to the CasPR comprises: a nuclear localization signal (NLS), a reporter protein or a detection label (e.g., GST, HRP, CAT, GFP, HcRed, DsRed, CFP, YFP, BFP), a localization signal, a protein targeting moiety, a DNA binding domain (e.g., MBP, Lex A DBD, Gal4 DBD), an epitope tag (e.g., His, myc, V5, FLAG, HA, VSV-G, Trx, etc), a transcription activation domain (e.g., VP64 or VPR), a transcription inhibition domain (e.g., KRAB moiety or SID moiety), a nuclease (e.g., FokI), a deamination domain (e.g., ADAR1, ADAR2, APOBEC, AID, or TAD), a methylase, a demethylase, a transcription release factor, an HDAC, a polypeptide having ssRNA cleavage activity, a polypeptide having dsRNA cleavage activity, a polypeptide having ssDNA cleavage activity, a polypeptide having dsDNA cleavage activity, a DNA or RNA ligase, or any combination thereof. In certain embodiments, the heterologous functional domain comprises an RNA base editor. In certain embodiments, the RNA base editor edits A→G single base change. In certain embodiments, the RNA base editor edits C→U single base change. In certain embodiments, the RNA base editor comprises ADAR2DD or a derivative thereof. In certain embodiments, the ADAR2DD derivative comprises the E488Q/T375A double mutations.

In certain embodiments, the fusion protein has the amino acid sequence of any one of Sequences 45-55.

MtCas6 (I-A):
(Sequence 45)
(SEQ ID NO: 73)
ATGCCCAAGAAGAAGCGGAAGGTGATGCCTCTGATCTTCAAGATCGGCTATAACGTGATCCC
CCTGCAGGACGTGATCCTGCCCACCCCTTCCAGCAAGGTGCTGAAGTACCTGATCCAGAGCG
GCAAGCTGATCCCCAGCCTGAAGGACCTGATCACCAGCCGGGACAAGTACAAGCCAATCTTC
ATCTCCCACCTGGGCTTCAACCAGCGGAGGATTTTCCAGACCAACGGCAATCTGAAAACCAT
CACCAAGGGCAGTAGACTGAGCTCCATCATCGCCTTCAGCACCCAGGCCAACGTGCTGTCCG
AGGTGGCCGATGAAGGGATCTTCGAAACCGTGTACGGAAAGTTCCACATCATGATCGAAAGC
ATCGAGATCGTGGAGGTGGAAAAGCTGAAGGAGGAGGTGGAGAAGCACATGAACGACAACAT
CAGAGTGAGATTCGTGTCTCCCACACTGCTGAGCTCCAAGGTGCTGCTGCCCCCCAGCCTGT
CCGAAAGATACAAGAAGATCCACGCCGGGTACAGCACCCTGCCCAGCGTGGGCCTGATCGTG
GCCTACGCCTACAACGTGTACTGCAATCTGATCGGCAAGAAGGAAGTGGAAGTGCGGGCCTT
CAAGTTTGGAATCCTGAGCAACGCCCTGTCCAGAATCATCGGCTACGACCTGCACCCTGTGA
CCGTGGCCATCGGCGAGGACAGCAAGGGGAATCTGAGAAAGGCTCGGGGCGTGATGGGCTGG
ATCGAGTTCGACATCCCCGACGAAAGACTGAAGCGGCGGGCCCTGAACTATCTGCTGACCAG
CAGCTACCTGGGCATCGGGAGATCTCGGGGCATCGGCTTCGGCGAGATCCGGCTGGAGTTCC
GGAAGATTGAAGAGAAGGAGGGACCCAAGAAGAAGCGGAAGGTGGGTGGAGGCGGAGGTTCT
GGGGGAGGAGGTAGTGGCGGTGGTGGTTCAGGAGGCGGCGGAAGCCAGCTGCATTTACCGCA
GGTTTTAGCTGACGCTGTCTCACGCCTGGTCATAGGTAAGTTTGGTGACCTGACCGACAACT
TCTCCTCCCCTCACGCTCGCAGAATAGGTCTGGCTGGAGTCGTCATGACAACAGGCACAGAT
GTTAAAGATGCCAAGGTGATATGTGTTTCTACAGGAGCAAAATGTATTAATGGTGAATACCT
AAGTGATCGTGGCCTTGCATTAAATGACTGCCATGCAGAAATAGTATCTCGGAGATCCTTGC
TCAGATTTCTTTATACACAACTTGAGCTTTACTTAAATAACGAGGATGATCAAAAAAGATCC
ATCTTTCAGAAATCAGAGCGAGGGGGGTTTAGGCTGAAGGAGAATATACAGTTTCATCTGTA
CATCAGCACCTCTCCCTGTGGAGATGCCAGAATCTTCTCACCACATGAGGCAATCCTGGAAG
AACCAGCAGATAGACACCCAAATCGTAAAGCAAGAGGACAGCTACGGACCAAAATAGAGGCT
GGTCAGGGGACGATTCCAGTGCGCAACAATGCGAGCATCCAAACGTGGGACGGGGTGCTGCA
AGGGGAGCGGCTGCTCACCATGTCCTGCAGTGACAAGATTGCACGCTGGAACGTGGTGGGCA
TCCAGGGATCACTGCTCAGCATTTTCGTGGAGCCCATTTACTTCTCGAGCATCATCCTGGGC
AGCCTTTACCACGGGGACCACCTTTCCAGGGCCATGTACCAGCGGATCTCCAACATAGAGGA
CCTGCCACCTCTCTACACCCTCAACAAGCCTTTGCTCACAGGCATCAGCAATGCAGAAGCAC
GGCAGCCAGGGAAGGCCCCCATATTCAGTGTCAACTGGACGGTAGGCGACTCCGCTATTGAG
GTCATCAACGCCACGACTGGGAAGGGAGAGCTGGGCCGCGCGTCCCGCCTGTGTAAGCACGC
GTTGTACTGTCGCTGGATGCGTGTGCACGGCAAGGTTCCCTCCCACTTACTACGCTCCAAGA
TTACCAAGCCCAACGTGTACCATGAGACAAAGCTGGCGGCAAAGGAGTACCAGGCCGCCAAG
GCGCGTCTGTTCACAGCCTTCATCAAGGCGGGGCTGGGGGCCTGGGTGGAGAAGCCCACCGA
GCAGGACCAGTTCTCACTCACGTAA
MmCas6 (I-B):
(Sequence 46)
(SEQ ID NO: 74)
ATGCCCAAGAAGAAGCGGAAGGTGATGGACCTGGAGTACATGCACATCTCCTACCCTAACAT
CCTGCTGAACATGCGGGACGGCAGCAAGCTGCGGGGCTACTTCGCCAAGAAGTACATCGACG
AAGAGATTGTGCACAACCACAGAGACAACGCCTTTGTGTACAAGTACCCCCAGATCCAGTTT
AAGATCATCGATAGAAGCCCCCTGATCATCGGCATTGGCTCTCTGGGCATCAATTTCCTGGA
GAGCAAGCGGATCTTCTTCGAGAAGGAACTGATTATCAGCAACGACACCAACGACATCACCG
AGGTGAACGTGCACAAGGACATGGATCACTTCGGCACGACCGACAAGATCCTGAAGTACCAG
TTCAAGACCCCTTGGATGGCACTGAACGCCAAGAATAGCGAGATCTACAAGAACTCTGACGA
GATCGACCGGGAGGAGTTCCTGAAGAGAGTGCTGATTGGGAATATCCTGAGCATGTCTAAGA
GCCTGGGCTATACCATCGAAGAAAAGCTGAAGGTGAAGATTAACCTGAAGGAAGTGCCCGTG
AAGTTCAAGAACCAGAACATGGTGGGCTTTCGGGGCGAGTTCTACATCAACTTCGACATCCC
TCAGTATCTGGGCATCGGCCGGAATGTGTCCCGGGGATTCGGCACAGTGGTGAAGGTGCCCA
AGAAGAAGCGGAAGGTGGGTGGAGGCGGAGGTTCTGGGGGAGGAGGTAGTGGCGGTGGTGGT
TCAGGAGGCGGCGGAAGCCAGCTGCATTTACCGCAGGTTTTAGCTGACGCTGTCTCACGCCT
GGTCATAGGTAAGTTTGGTGACCTGACCGACAACTTCTCCTCCCCTCACGCTCGCAGAATAG
GTCTGGCTGGAGTCGTCATGACAACAGGCACAGATGTTAAAGATGCCAAGGTGATATGTGTT
TCTACAGGAGCAAAATGTATTAATGGTGAATACCTAAGTGATCGTGGCCTTGCATTAAATGA
CTGCCATGCAGAAATAGTATCTCGGAGATCCTTGCTCAGATTTCTTTATACACAACTTGAGC
TTTACTTAAATAACGAGGATGATCAAAAAAGATCCATCTTTCAGAAATCAGAGCGAGGGGGG
TTTAGGCTGAAGGAGAATATACAGTTTCATCTGTACATCAGCACCTCTCCCTGTGGAGATGC
CAGAATCTTCTCACCACATGAGGCAATCCTGGAAGAACCAGCAGATAGACACCCAAATCGTA
AAGCAAGAGGACAGCTACGGACCAAAATAGAGGCTGGTCAGGGGACGATTCCAGTGCGCAAC
AATGCGAGCATCCAAACGTGGGACGGGGTGCTGCAAGGGGAGCGGCTGCTCACCATGTCCTG
CAGTGACAAGATTGCACGCTGGAACGTGGTGGGCATCCAGGGATCACTGCTCAGCATTTTCG
TGGAGCCCATTTACTTCTCGAGCATCATCCTGGGCAGCCTTTACCACGGGGACCACCTTTCC
AGGGCCATGTACCAGCGGATCTCCAACATAGAGGACCTGCCACCTCTCTACACCCTCAACAA
GCCTTTGCTCACAGGCATCAGCAATGCAGAAGCACGGCAGCCAGGGAAGGCCCCCATATTCA
GTGTCAACTGGACGGTAGGCGACTCCGCTATTGAGGTCATCAACGCCACGACTGGGAAGGGA
GAGCTGGGCCGCGCGTCCCGCCTGTGTAAGCACGCGTTGTACTGTCGCTGGATGCGTGTGCA
CGGCAAGGTTCCCTCCCACTTACTACGCTCCAAGATTACCAAGCCCAACGTGTACCATGAGA
CAAAGCTGGCGGCAAAGGAGTACCAGGCCGCCAAGGCGCGTCTGTTCACAGCCTTCATCAAG
GCGGGGCTGGGGGCCTGGGTGGAGAAGCCCACCGAGCAGGACCAGTTCTCACTCACGTAA
SpCas5d (I-C1):
(Sequence 47)
(SEQ ID NO: 75)
ATGCCCAAGAAGAAGCGGAAGGTGATGAGAAATGAAGTGCAGTTCGAGCTGTTCGGCGACTA
CGCCCTGTTCACCGACCCCCTGACCAAGATCGGCGGCGAAAAGCTGAGCTACAGCGTGCCTA
CCTACCAGGCCCTGAAGGGCATCGCCGAGAGCATCTACTGGAAGCCCACCATCGTGTTCGTG
ATCGACGAACTGCGGGTCATGAAGCCCATTCAGATGGAGTCTAAGGGCGTGAGGCCCATCGA
GTACGGCGGCGGCAACACCCTGGCCCACTACACCTACCTGAAGGATGTGCACTACCAGGTGA
AGGCCCACTTCGAGTTCAACCTGCACCGGCCCGACCTGGCCTTCGATAGAAACGAGGGCAAG
CACTACTCCATCCTGCAGAGAAGCCTGAAGGCCGGCGGCAGAAGAGATATTTTCCTGGGCGC
CCGGGAGTGCCAGGGCTACGTGGCCCCCTGCGAGTTCGGCAGCGGCGACGGCTTCTACGACG
GCCAGGGCAAGTACCACCTGGGAACCATGGTGCACGGTTTCAACTACCCCGACGAAACCGGA
CAGCACCAGCTGGATGTGAGACTGTGGTCTGCCGTCATGGAAAACGGCTACATCCAGTTCCC
CCGCCCTGAGGACTGCCCCATCGTGCGGCCTGTGAAGGAGATGGAACCCAAGATCTTCAACC
CCGACAACGTGCAGTCCGCCGAACAGCTGCTGCACGACCTGGGCGGCGAACCCAAGAAGAAG
CGGAAGGTGGGTGGAGGCGGAGGTTCTGGGGGAGGAGGTAGTGGCGGTGGTGGTTCAGGAGG
CGGCGGAAGCCAGCTGCATTTACCGCAGGTTTTAGCTGACGCTGTCTCACGCCTGGTCATAG
GTAAGTTTGGTGACCTGACCGACAACTTCTCCTCCCCTCACGCTCGCAGAATAGGTCTGGCT
GGAGTCGTCATGACAACAGGCACAGATGTTAAAGATGCCAAGGTGATATGTGTTTCTACAGG
AGCAAAATGTATTAATGGTGAATACCTAAGTGATCGTGGCCTTGCATTAAATGACTGCCATG
CAGAAATAGTATCTCGGAGATCCTTGCTCAGATTTCTTTATACACAACTTGAGCTTTACTTA
AATAACGAGGATGATCAAAAAAGATCCATCTTTCAGAAATCAGAGCGAGGGGGGTTTAGGCT
GAAGGAGAATATACAGTTTCATCTGTACATCAGCACCTCTCCCTGTGGAGATGCCAGAATCT
TCTCACCACATGAGGCAATCCTGGAAGAACCAGCAGATAGACACCCAAATCGTAAAGCAAGA
GGACAGCTACGGACCAAAATAGAGGCTGGTCAGGGGACGATTCCAGTGCGCAACAATGCGAG
CATCCAAACGTGGGACGGGGTGCTGCAAGGGGAGCGGCTGCTCACCATGTCCTGCAGTGACA
AGATTGCACGCTGGAACGTGGTGGGCATCCAGGGATCACTGCTCAGCATTTTCGTGGAGCCC
ATTTACTTCTCGAGCATCATCCTGGGCAGCCTTTACCACGGGGACCACCTTTCCAGGGCCAT
GTACCAGCGGATCTCCAACATAGAGGACCTGCCACCTCTCTACACCCTCAACAAGCCTTTGC
TCACAGGCATCAGCAATGCAGAAGCACGGCAGCCAGGGAAGGCCCCCATATTCAGTGTCAAC
TGGACGGTAGGCGACTCCGCTATTGAGGTCATCAACGCCACGACTGGGAAGGGAGAGCTGGG
CCGCGCGTCCCGCCTGTGTAAGCACGCGTTGTACTGTCGCTGGATGCGTGTGCACGGCAAGG
TTCCCTCCCACTTACTACGCTCCAAGATTACCAAGCCCAACGTGTACCATGAGACAAAGCTG
GCGGCAAAGGAGTACCAGGCCGCCAAGGCGCGTCTGTTCACAGCCTTCATCAAGGCGGGGCT
GGGGGCCTGGGTGGAGAAGCCCACCGAGCAGGACCAGTTCTCACTCACGTAA
BhCas5d (I-C2):
(Sequence 48)
(SEQ ID NO: 76)
ATGCCCAAGAAGAAGCGGAAGGTGATGTACAGAAGCCGGGACTTCTACGTGAGAGTGTCCGG
CCAGCGGGCCCTGTTCACCAACCCCGCCACCAAGGGCGGCTCCGAACGGAGCTCCTACTCCG
TGCCTACCCGGCAGGCCCTGAACGGGATTGTGGACGCCATCTACTACAAGCCCACGTTCACC
AACATCGTGACCGAGGTGAAGGTGATTAACCAGATCCAGACCGAACTGCAGGGCGTGCGGGC
CCTGCTGCATGACTACAGCGCCGACCTGAGCTACGTGTCCTACCTGAGCGACGTGGTGTACC
TGATTAAGTTTCATTTCGTGTGGAACGAGGATAGAAAGGACCTGAATAGCGACCGGCTGCCA
GCCAAGCATGAGGCCATCATGGAGCGGTCTATCCGGAAGGGCGGCAGACGGGACGTGTTCCT
GGGCACCAGAGAATGCCTGGGCCTGCTGGACGACATCAGCCAGGAAGAATACGAAACCACAG
TGAGCTATTACAATGGGGTGAACATCGACCTGGGCATCATGTTCCACAGCTTCGCTTACCCC
AAGGACAAGAAAACCCCCCTGAAGTCCTACTTCACAAAGACCGTGATGAAGAACGGCGTGAT
CACCTTCAAGGCCCAGTCCGAATGCGATATTGTGAACACCCTGAGCTCCTACGCCTTCAAGG
CCCCCGAGGAGATCAAGAGCGTGAACGACGAGTGCATGGAGTACGACGCCATGGAGAAGGGC
GAAAACCCCAAGAAGAAGCGGAAGGTGGGTGGAGGCGGAGGTTCTGGGGGAGGAGGTAGTGG
CGGTGGTGGTTCAGGAGGCGGCGGAAGCCAGCTGCATTTACCGCAGGTTTTAGCTGACGCTG
TCTCACGCCTGGTCATAGGTAAGTTTGGTGACCTGACCGACAACTTCTCCTCCCCTCACGCT
CGCAGAATAGGTCTGGCTGGAGTCGTCATGACAACAGGCACAGATGTTAAAGATGCCAAGGT
GATATGTGTTTCTACAGGAGCAAAATGTATTAATGGTGAATACCTAAGTGATCGTGGCCTTG
CATTAAATGACTGCCATGCAGAAATAGTATCTCGGAGATCCTTGCTCAGATTTCTTTATACA
CAACTTGAGCTTTACTTAAATAACGAGGATGATCAAAAAAGATCCATCTTTCAGAAATCAGA
GCGAGGGGGGTTTAGGCTGAAGGAGAATATACAGTTTCATCTGTACATCAGCACCTCTCCCT
GTGGAGATGCCAGAATCTTCTCACCACATGAGGCAATCCTGGAAGAACCAGCAGATAGACAC
CCAAATCGTAAAGCAAGAGGACAGCTACGGACCAAAATAGAGGCTGGTCAGGGGACGATTCC
AGTGCGCAACAATGCGAGCATCCAAACGTGGGACGGGGTGCTGCAAGGGGAGCGGCTGCTCA
CCATGTCCTGCAGTGACAAGATTGCACGCTGGAACGTGGTGGGCATCCAGGGATCACTGCTC
AGCATTTTCGTGGAGCCCATTTACTTCTCGAGCATCATCCTGGGCAGCCTTTACCACGGGGA
CCACCTTTCCAGGGCCATGTACCAGCGGATCTCCAACATAGAGGACCTGCCACCTCTCTACA
CCCTCAACAAGCCTTTGCTCACAGGCATCAGCAATGCAGAAGCACGGCAGCCAGGGAAGGCC
CCCATATTCAGTGTCAACTGGACGGTAGGCGACTCCGCTATTGAGGTCATCAACGCCACGAC
TGGGAAGGGAGAGCTGGGCCGCGCGTCCCGCCTGTGTAAGCACGCGTTGTACTGTCGCTGGA
TGCGTGTGCACGGCAAGGTTCCCTCCCACTTACTACGCTCCAAGATTACCAAGCCCAACGTG
TACCATGAGACAAAGCTGGCGGCAAAGGAGTACCAGGCCGCCAAGGCGCGTCTGTTCACAGC
CTTCATCAAGGCGGGGCTGGGGGCCTGGGTGGAGAAGCCCACCGAGCAGGACCAGTTCTCAC
TCACGTAA
SaCas6 (I-D):
(Sequence 49)
(SEQ ID NO: 77)
ATGCCCAAGAAGAAGCGGAAGGTGATGCCCAACGATCCCTACAGCCTGTACTCCATCGTGAT
CGAACTGGGCGCCGCCGAAAAGGGATTCCCCACAGGCATCCTGGGCAGAAGCCTGCATAGCC
AGGTGCTGCAGTGGTTCAAGCAGGATAACCCCTTCCTGGCCACCGAGCTGCACCAGAGCCAG
ATCTCCCCCTTCTCCATCTCTCCACTGATGGGCAAGCGGCACGCCAAGCTGACCAAGGCCGG
CGACCGGCTGTTCTTTCGGATCTGCCTGCTGAGAGGAGATCTGCTGCAGCCCCTGCTGAACG
GCATTGAGCAGACCGTGAACCAGAGCGTGTGCCTGGACAAGTTCCGGTTCCGGCTGTGCCAG
ACCCACATCCTGCCCGGCAGCCACCCTCTGGCTGGCGCCTCCCACTATAGCCTGATCAGCCA
GACCCCAGTGAGCTCCAAGATTACCCTGGACTTCAAGAGTTCTACCTCCTTCAAGGTGGACC
GGAAGATCATCCAAGTGTTCCCTCTGGGCGAACACGTGTTCAACAGCCTGCTCAGACGCTGG
AATAACTTCGCCCCCGAGGACCTGCACTTCTCTCAGGTGGACTGGAGCATCCCCATCGCCGC
ATTCGACGTGAAAACCATCCCCATCCACCTGAAGAAGGTCGAGATCGGCGCACAGGGCTGGG
TGACCTACATCTTCCCCAACACAGAACAGGCCAAGATCGCCTCCGTGCTGAGCGAATTCGCC
TTCTTCAGCGGAGTGGGACGGAAAACCACCATGGGCATGGGCCAGGTGCAGGTGCGGTCCCC
CAAGAAGAAGCGGAAGGTGGGTGGAGGCGGAGGTTCTGGGGGAGGAGGTAGTGGCGGTGGTG
GTTCAGGAGGCGGCGGAAGCCAGCTGCATTTACCGCAGGTTTTAGCTGACGCTGTCTCACGC
CTGGTCATAGGTAAGTTTGGTGACCTGACCGACAACTTCTCCTCCCCTCACGCTCGCAGAAT
AGGTCTGGCTGGAGTCGTCATGACAACAGGCACAGATGTTAAAGATGCCAAGGTGATATGTG
TTTCTACAGGAGCAAAATGTATTAATGGTGAATACCTAAGTGATCGTGGCCTTGCATTAAAT
GACTGCCATGCAGAAATAGTATCTCGGAGATCCTTGCTCAGATTTCTTTATACACAACTTGA
GCTTTACTTAAATAACGAGGATGATCAAAAAAGATCCATCTTTCAGAAATCAGAGCGAGGGG
GGTTTAGGCTGAAGGAGAATATACAGTTTCATCTGTACATCAGCACCTCTCCCTGTGGAGAT
GCCAGAATCTTCTCACCACATGAGGCAATCCTGGAAGAACCAGCAGATAGACACCCAAATCG
TAAAGCAAGAGGACAGCTACGGACCAAAATAGAGGCTGGTCAGGGGACGATTCCAGTGCGCA
ACAATGCGAGCATCCAAACGTGGGACGGGGTGCTGCAAGGGGAGCGGCTGCTCACCATGTCC
TGCAGTGACAAGATTGCACGCTGGAACGTGGTGGGCATCCAGGGATCACTGCTCAGCATTTT
CGTGGAGCCCATTTACTTCTCGAGCATCATCCTGGGCAGCCTTTACCACGGGGACCACCTTT
CCAGGGCCATGTACCAGCGGATCTCCAACATAGAGGACCTGCCACCTCTCTACACCCTCAAC
AAGCCTTTGCTCACAGGCATCAGCAATGCAGAAGCACGGCAGCCAGGGAAGGCCCCCATATT
CAGTGTCAACTGGACGGTAGGCGACTCCGCTATTGAGGTCATCAACGCCACGACTGGGAAGG
GAGAGCTGGGCCGCGCGTCCCGCCTGTGTAAGCACGCGTTGTACTGTCGCTGGATGCGTGTG
CACGGCAAGGTTCCCTCCCACTTACTACGCTCCAAGATTACCAAGCCCAACGTGTACCATGA
GACAAAGCTGGCGGCAAAGGAGTACCAGGCCGCCAAGGCGCGTCTGTTCACAGCCTTCATCA
AGGCGGGGCTGGGGGCCTGGGTGGAGAAGCCCACCGAGCAGGACCAGTTCTCACTCACGTAA
EcCas6e (I-E):
(Sequence 50)
(SEQ ID NO: 78)
ATGCCTAAGAAGAAGCGGAAGGTGTACCTGAGCAAGGTGATCATCGCCAGAGCCTGGAGCAG
AGACCTGTACCAGCTGCACCAGGGCCTGTGGCACCTGTTCCCCAACCGGCCCGACGCCGCCC
GGGATTTCCTGTTCCACGTGGAGAAGAGAAACACCCCGGAAGGCTGCCACGTGCTGCTGCAG
AGCGCACAGATGCCTGTGAGCACCGCCGTGGCCACCGTGATCAAGACCAAGCAGGTGGAGTT
CCAGCTGCAGGTGGGCGTGCCCCTGTATTTCAGGCTGCGGGCGAATCCCATCAAGACCATCC
TGGACAACCAGAAGCGGCTGGACAGCAAGGGCAACATCAAGAGGTGCAGAGTGCCTCTGATC
AAGGAGGCCGAACAGATCGCCTGGCTGCAGCGGAAGCTGGGCAATGCCGCCAGAGTGGAGGA
CGTGCACCCCATCAGCGAGCGGCCCCAGTACTTCTCCGGCGACGGAAAGAGCGGAAAGATCC
AGACCGTGTGCTTCGAGGGCGTGCTGACCATCAACGACGCACCCGCCCTGATCGACCTCGTG
CAGCAGGGGATCGGCCCTGCCAAGTCCATGGGCTGCGGACTGCTGTCCCTGGCCCCCCTGCC
CAAGAAGAAGCGGAAGGTGGGTGGAGGCGGAGGTTCTGGGGGAGGAGGTAGTGGCGGTGGTG
GTTCAGGAGGCGGCGGAAGCCAGCTGCATTTACCGCAGGTTTTAGCTGACGCTGTCTCACGC
CTGGTCATAGGTAAGTTTGGTGACCTGACCGACAACTTCTCCTCCCCTCACGCTCGCAGAAT
AGGTCTGGCTGGAGTCGTCATGACAACAGGCACAGATGTTAAAGATGCCAAGGTGATATGTG
TTTCTACAGGAGCAAAATGTATTAATGGTGAATACCTAAGTGATCGTGGCCTTGCATTAAAT
GACTGCCATGCAGAAATAGTATCTCGGAGATCCTTGCTCAGATTTCTTTATACACAACTTGA
GCTTTACTTAAATAACGAGGATGATCAAAAAAGATCCATCTTTCAGAAATCAGAGCGAGGGG
GGTTTAGGCTGAAGGAGAATATACAGTTTCATCTGTACATCAGCACCTCTCCCTGTGGAGAT
GCCAGAATCTTCTCACCACATGAGGCAATCCTGGAAGAACCAGCAGATAGACACCCAAATCG
TAAAGCAAGAGGACAGCTACGGACCAAAATAGAGGCTGGTCAGGGGACGATTCCAGTGCGCA
ACAATGCGAGCATCCAAACGTGGGACGGGGTGCTGCAAGGGGAGCGGCTGCTCACCATGTCC
TGCAGTGACAAGATTGCACGCTGGAACGTGGTGGGCATCCAGGGATCACTGCTCAGCATTTT
CGTGGAGCCCATTTACTTCTCGAGCATCATCCTGGGCAGCCTTTACCACGGGGACCACCTTT
CCAGGGCCATGTACCAGCGGATCTCCAACATAGAGGACCTGCCACCTCTCTACACCCTCAAC
AAGCCTTTGCTCACAGGCATCAGCAATGCAGAAGCACGGCAGCCAGGGAAGGCCCCCATATT
CAGTGTCAACTGGACGGTAGGCGACTCCGCTATTGAGGTCATCAACGCCACGACTGGGAAGG
GAGAGCTGGGCCGCGCGTCCCGCCTGTGTAAGCACGCGTTGTACTGTCGCTGGATGCGTGTG
CACGGCAAGGTTCCCTCCCACTTACTACGCTCCAAGATTACCAAGCCCAACGTGTACCATGA
GACAAAGCTGGCGGCAAAGGAGTACCAGGCCGCCAAGGCGCGTCTGTTCACAGCCTTCATCA
AGGCGGGGCTGGGGGCCTGGGTGGAGAAGCCCACCGAGCAGGACCAGTTCTCACTCACGTAA
PaCas6f (I-F):
(Sequence 51)
(SEQ ID NO: 79)
ATGCCTAAGAAGAAGAGAAAGGTGGACCACTACCTGGACATTAGACTGCGCCCTGACCCAGA
GTTCCCTCCTGCCCAGCTGATGTCTGTGCTGTTTGGCAAGCTGCACCAGGCCCTGGTGGCCC
AGGGCGGTGACAGAATCGGAGTGTCTTTCCCTGATCTGGACGAATCTAGATCTAGACTGGGA
GAGAGACTGAGAATCCACGCGTCTGCCGACGACCTGAGAGCTCTGCTGGCCAGACCATGGCT
GGAAGGACTGCGCGACCACCTGCAGTTCGGTGAACCTGCCGTGGTGCCTCACCCAACTCCAT
ACAGACAGGTGAGTAGAGTGCAGGCAAAGTCTAATCCAGAGAGACTGAGACGCAGACTGATG
AGAAGGCATGACCTGTCCGAAGAAGAAGCCAGAAAGAGAATCCCAGACACAGTGGCCAGAGC
CCTGGATCTGCCTTTTGTGACCCTGAGAAGCCAGTCTACCGGCCAGCACTTCAGACTGTTTA
TTCGCCACGGACCACTGCAGGTGACCGCCGAAGAGGGAGGTTTTACCTGCTACGGACTGAGC
AAGGGAGGTTTCGTGCCTTGGTTCCCCAAGAAGAAGCGGAAGGTGGGTGGAGGCGGAGGTTC
TGGGGGAGGAGGTAGTGGCGGTGGTGGTTCAGGAGGCGGCGGAAGCCAGCTGCATTTACCGC
AGGTTTTAGCTGACGCTGTCTCACGCCTGGTCATAGGTAAGTTTGGTGACCTGACCGACAAC
TTCTCCTCCCCTCACGCTCGCAGAATAGGTCTGGCTGGAGTCGTCATGACAACAGGCACAGA
TGTTAAAGATGCCAAGGTGATATGTGTTTCTACAGGAGCAAAATGTATTAATGGTGAATACC
TAAGTGATCGTGGCCTTGCATTAAATGACTGCCATGCAGAAATAGTATCTCGGAGATCCTTG
CTCAGATTTCTTTATACACAACTTGAGCTTTACTTAAATAACGAGGATGATCAAAAAAGATC
CATCTTTCAGAAATCAGAGCGAGGGGGGTTTAGGCTGAAGGAGAATATACAGTTTCATCTGT
ACATCAGCACCTCTCCCTGTGGAGATGCCAGAATCTTCTCACCACATGAGGCAATCCTGGAA
GAACCAGCAGATAGACACCCAAATCGTAAAGCAAGAGGACAGCTACGGACCAAAATAGAGGC
TGGTCAGGGGACGATTCCAGTGCGCAACAATGCGAGCATCCAAACGTGGGACGGGGTGCTGC
AAGGGGAGCGGCTGCTCACCATGTCCTGCAGTGACAAGATTGCACGCTGGAACGTGGTGGGC
ATCCAGGGATCACTGCTCAGCATTTTCGTGGAGCCCATTTACTTCTCGAGCATCATCCTGGG
CAGCCTTTACCACGGGGACCACCTTTCCAGGGCCATGTACCAGCGGATCTCCAACATAGAGG
ACCTGCCACCTCTCTACACCCTCAACAAGCCTTTGCTCACAGGCATCAGCAATGCAGAAGCA
CGGCAGCCAGGGAAGGCCCCCATATTCAGTGTCAACTGGACGGTAGGCGACTCCGCTATTGA
GGTCATCAACGCCACGACTGGGAAGGGAGAGCTGGGCCGCGCGTCCCGCCTGTGTAAGCACG
CGTTGTACTGTCGCTGGATGCGTGTGCACGGCAAGGTTCCCTCCCACTTACTACGCTCCAAG
ATTACCAAGCCCAACGTGTACCATGAGACAAAGCTGGCGGCAAAGGAGTACCAGGCCGCCAA
GGCGCGTCTGTTCACAGCCTTCATCAAGGCGGGGCTGGGGGCCTGGGTGGAGAAGCCCACCG
AGCAGGACCAGTTCTCACTCACGTAA
MtCas6 (III-A):
(Sequence 80)
(SEQ ID NO: 52)
ATGCCCAAGAAGAAGCGGAAGGTGATGGCCGCCAGAAGAGGCGGAATCCGGAGAACCGACCT
GCTGCGGAGGTCTGGCCAGCCTCGGGGCAGACACCGGGCCTCCGCCGCCGAGAGCGGCCTGA
CATGGATCTCCCCTACCCTGATCCTGGTGGGCTTCAGCCACAGGGGCGATAGGAGAATGACC
GAGCACCTGTCCAGACTGACCCTGACCCTGGAAGTGGATGCCCCCCTGGAGAGAGCCCGGGT
GGCCACCCTGGGCCCCCACCTGCATGGCGTGCTGATGGAGTCTATCCCCGCCGACTACGTGC
AGACACTGCACACAGTGCCGGTGAACCCTTACAGCCAGTACGCTCTGGCCCGGAGCACCACC
AGCCTGGAGTGGAAGATCTCCACCCTGACAAATGAGGCCCGGCAGCAGATCGTCGGCCCCAT
CAACGACGCCGCCTTCGCCGGCTTCCGGCTGCGGGCCAGCGGCATCGCCACCCAGGTGACAA
GCAGAAGCCTGGAGCAGAACCCCCTGTCCCAGTTTGCCAGAATCTTCTACGCCAGGCCCGAA
ACCCGCAAGTTCAGAGTGGAGTTCCTGACCCCCACCGCCTTCAAGCAGAGCGGCGAGTACGT
GTTTTGGCCCGATCCCAGACTGGTGTTCCAGTCCCTGGCCCAGAAGTACGGCGCCATCGTGG
ACGGAGAAGAGCCCGACCCCGGCCTGATCGCCGAGTTTGGCCAGTCCGTGAGACTGAGCGCC
TTCAGAGTGGCCAGCGCCCCTTTTGCCGTGGGCGCCGCCAGGGTGCCCGGATTCACCGGCAG
CGCCACCTTCACCGTGCGGGGAGTGGACACCTTCGCCAGCTACATCGCCGCTCTGCTGTGGT
TCGGCGAGTTCAGCGGATGCGGCATCAAGGCCTCCATGGGAATGGGCGCCATCCGGGTGCAG
CCTCTGGCCCCCCGGGAGAAGTGCGTGCCCAAGCCCCCCAAGAAGAAGCGGAAGGTGGGTGG
AGGCGGAGGTTCTGGGGGAGGAGGTAGTGGCGGTGGTGGTTCAGGAGGCGGCGGAAGCCAGC
TGCATTTACCGCAGGTTTTAGCTGACGCTGTCTCACGCCTGGTCATAGGTAAGTTTGGTGAC
CTGACCGACAACTTCTCCTCCCCTCACGCTCGCAGAATAGGTCTGGCTGGAGTCGTCATGAC
AACAGGCACAGATGTTAAAGATGCCAAGGTGATATGTGTTTCTACAGGAGCAAAATGTATTA
ATGGTGAATACCTAAGTGATCGTGGCCTTGCATTAAATGACTGCCATGCAGAAATAGTATCT
CGGAGATCCTTGCTCAGATTTCTTTATACACAACTTGAGCTTTACTTAAATAACGAGGATGA
TCAAAAAAGATCCATCTTTCAGAAATCAGAGCGAGGGGGGTTTAGGCTGAAGGAGAATATAC
AGTTTCATCTGTACATCAGCACCTCTCCCTGTGGAGATGCCAGAATCTTCTCACCACATGAG
GCAATCCTGGAAGAACCAGCAGATAGACACCCAAATCGTAAAGCAAGAGGACAGCTACGGAC
CAAAATAGAGGCTGGTCAGGGGACGATTCCAGTGCGCAACAATGCGAGCATCCAAACGTGGG
ACGGGGTGCTGCAAGGGGAGCGGCTGCTCACCATGTCCTGCAGTGACAAGATTGCACGCTGG
AACGTGGTGGGCATCCAGGGATCACTGCTCAGCATTTTCGTGGAGCCCATTTACTTCTCGAG
CATCATCCTGGGCAGCCTTTACCACGGGGACCACCTTTCCAGGGCCATGTACCAGCGGATCT
CCAACATAGAGGACCTGCCACCTCTCTACACCCTCAACAAGCCTTTGCTCACAGGCATCAGC
AATGCAGAAGCACGGCAGCCAGGGAAGGCCCCCATATTCAGTGTCAACTGGACGGTAGGCGA
CTCCGCTATTGAGGTCATCAACGCCACGACTGGGAAGGGAGAGCTGGGCCGCGCGTCCCGCC
TGTGTAAGCACGCGTTGTACTGTCGCTGGATGCGTGTGCACGGCAAGGTTCCCTCCCACTTA
CTACGCTCCAAGATTACCAAGCCCAACGTGTACCATGAGACAAAGCTGGCGGCAAAGGAGTA
CCAGGCCGCCAAGGCGCGTCTGTTCACAGCCTTCATCAAGGCGGGGCTGGGGGCCTGGGTGG
AGAAGCCCACCGAGCAGGACCAGTTCTCACTCACGTAA
PfCas6 (III-B):
(Sequence 53)
(SEQ ID NO: 81)
ATGCCCAAGAAGAAGCGGAAGGTGATGAGATTCCTGATCAGACTGGTGCCCGAGGACAAGGA
CAGAGCCTTCAAGGTGCCTTACAACCACCAGTACTATCTGCAGGGCCTGATCTACAACGCCA
TCAAGTCCTCCAACCCCAAGCTGGCCACCTACCTGCACGAGGTGAAGGGCCCCAAGCTGTTC
ACCTACAGCCTGTTCATGGCCGAAAAGCGGGAGCACCCTAAGGGCCTGCCCTACTTTCTGGG
CTACAAGAAGGGCTTCTTCTACTTCAGCACCTGCGTGCCCGAGATCGCCGAGGCCCTGGTGA
ACGGCCTGCTGATGAATCCCGAGGTGCGGCTGTGGGACGAGAGATTCTACCTGCACGAAATC
AAGGTCCTGCGGGAGCCCAAGAAGTTCAACGGCAGCACCTTCGTGACCCTGAGCCCCATCGC
CGTGACCGTGGTGAGAAAGGGCAAGTCCTACGACGTGCCCCCCATGGAAAAGGAGTTCTACA
GCATTATCAAGGATGACCTGCAGGACAAGTACGTGATGGCCTACGGCGACAAGCCCCCCAGT
GAGTTCGAGATGGAAGTGCTGATCGCCAAGCCCAAGCGGTTCCGGATCAAGCCCGGCATCTA
TCAGACCGCCTGGCACCTGGTGTTTCGGGCCTACGGCAATGACGACCTGCTGAAGGTGGGCT
ACGAAGTGGGATTCGGGGAGAAGAACTCCCTGGGATTCGGAATGGTCAAGGTGGAGGGCAAC
AAGACCACCAAGGAAGCCGAAGAACAGGAGAAGATCACCTTCAACTCCCGGGAAGAGCTGAA
AACAGGCGTGCCCAAGAAGAAGCGGAAGGTGGGTGGAGGCGGAGGTTCTGGGGGAGGAGGTA
GTGGCGGTGGTGGTTCAGGAGGCGGCGGAAGCCAGCTGCATTTACCGCAGGTTTTAGCTGAC
GCTGTCTCACGCCTGGTCATAGGTAAGTTTGGTGACCTGACCGACAACTTCTCCTCCCCTCA
CGCTCGCAGAATAGGTCTGGCTGGAGTCGTCATGACAACAGGCACAGATGTTAAAGATGCCA
AGGTGATATGTGTTTCTACAGGAGCAAAATGTATTAATGGTGAATACCTAAGTGATCGTGGC
CTTGCATTAAATGACTGCCATGCAGAAATAGTATCTCGGAGATCCTTGCTCAGATTTCTTTA
TACACAACTTGAGCTTTACTTAAATAACGAGGATGATCAAAAAAGATCCATCTTTCAGAAAT
CAGAGCGAGGGGGGTTTAGGCTGAAGGAGAATATACAGTTTCATCTGTACATCAGCACCTCT
CCCTGTGGAGATGCCAGAATCTTCTCACCACATGAGGCAATCCTGGAAGAACCAGCAGATAG
ACACCCAAATCGTAAAGCAAGAGGACAGCTACGGACCAAAATAGAGGCTGGTCAGGGGACGA
TTCCAGTGCGCAACAATGCGAGCATCCAAACGTGGGACGGGGTGCTGCAAGGGGAGCGGCTG
CTCACCATGTCCTGCAGTGACAAGATTGCACGCTGGAACGTGGTGGGCATCCAGGGATCACT
GCTCAGCATTTTCGTGGAGCCCATTTACTTCTCGAGCATCATCCTGGGCAGCCTTTACCACG
GGGACCACCTTTCCAGGGCCATGTACCAGCGGATCTCCAACATAGAGGACCTGCCACCTCTC
TACACCCTCAACAAGCCTTTGCTCACAGGCATCAGCAATGCAGAAGCACGGCAGCCAGGGAA
GGCCCCCATATTCAGTGTCAACTGGACGGTAGGCGACTCCGCTATTGAGGTCATCAACGCCA
CGACTGGGAAGGGAGAGCTGGGCCGCGCGTCCCGCCTGTGTAAGCACGCGTTGTACTGTCGC
TGGATGCGTGTGCACGGCAAGGTTCCCTCCCACTTACTACGCTCCAAGATTACCAAGCCCAA
CGTGTACCATGAGACAAAGCTGGCGGCAAAGGAGTACCAGGCCGCCAAGGCGCGTCTGTTCA
CAGCCTTCATCAAGGCGGGGCTGGGGGCCTGGGTGGAGAAGCCCACCGAGCAGGACCAGTTC
TCACTCACGTAA
PaCsf5 (IV-A1):
(Sequence 54)
(SEQ ID NO: 82)
ATGCCTAAGAAGAAGCGGAAGGTGTTCGTGACCCAGGTGATCTTCAACATCGGCGAACGGAC
GTACCCCGACAGGGCTCGGGCTATGGTGGCCGAGCTGATGGATGGCGTCCAGCCTGGCCTGG
TGGCCACCCTGATGAACTACATCCCCGGCACCAGCACGAGCCGGACAGAGTTCCCCACCGTG
CAGTTCGGCGGCGCCAGCGACGGCTTTTGCCTGCTGGGCTTCGGCGACGGCGGCGGCGCCAT
CGTGAGAGATGCCGTGCCCCTGATCCACGCCGCCCTGGCAAGGCGGATGCCTGATCGGATCA
TCCAGGTGGAACACAAGGAGCACAGCCTGTCCGCCGAGGCCCGGCCCTACGTGCTGAGCTAC
ACCGTGCCTCGGATGGTGGTGCAGAAGAAGCAGCGGCACGCCGAGAGACTGCTGCACGAAGC
CGAGGGAAAGGCTCACCTGGAGGGCCTGTTCCTGCGGAGCCTGCAGAGGCAGGCCGCCGCCG
TGGGCCTGCCCCTGCCCGAGAACCTGGAGGTGGAGTTCAAGGGAGCCGTGGGCGACTTCGCC
GCAAAGCACAATCCAAATAGCAAGGTGGCCTACCGGGGACTGAGAGGCGCCGTGTTCGATGT
GAACGCCAGACTGGGCGGCATCTGGACCGCCGGATTCATGCTGAGCAAGGGCTACGGCCAGT
TTAACGCCACCCACCAGCTGAGCGGCGCCGTGAACGCTCTGTCCGAACCCAAGAAGAAGCGG
AAGGTGGGTGGAGGCGGAGGTTCTGGGGGAGGAGGTAGTGGCGGTGGTGGTTCAGGAGGCGG
CGGAAGCCAGCTGCATTTACCGCAGGTTTTAGCTGACGCTGTCTCACGCCTGGTCATAGGTA
AGTTTGGTGACCTGACCGACAACTTCTCCTCCCCTCACGCTCGCAGAATAGGTCTGGCTGGA
GTCGTCATGACAACAGGCACAGATGTTAAAGATGCCAAGGTGATATGTGTTTCTACAGGAGC
AAAATGTATTAATGGTGAATACCTAAGTGATCGTGGCCTTGCATTAAATGACTGCCATGCAG
AAATAGTATCTCGGAGATCCTTGCTCAGATTTCTTTATACACAACTTGAGCTTTACTTAAAT
AACGAGGATGATCAAAAAAGATCCATCTTTCAGAAATCAGAGCGAGGGGGGTTTAGGCTGAA
GGAGAATATACAGTTTCATCTGTACATCAGCACCTCTCCCTGTGGAGATGCCAGAATCTTCT
CACCACATGAGGCAATCCTGGAAGAACCAGCAGATAGACACCCAAATCGTAAAGCAAGAGGA
CAGCTACGGACCAAAATAGAGGCTGGTCAGGGGACGATTCCAGTGCGCAACAATGCGAGCAT
CCAAACGTGGGACGGGGTGCTGCAAGGGGAGCGGCTGCTCACCATGTCCTGCAGTGACAAGA
TTGCACGCTGGAACGTGGTGGGCATCCAGGGATCACTGCTCAGCATTTTCGTGGAGCCCATT
TACTTCTCGAGCATCATCCTGGGCAGCCTTTACCACGGGGACCACCTTTCCAGGGCCATGTA
CCAGCGGATCTCCAACATAGAGGACCTGCCACCTCTCTACACCCTCAACAAGCCTTTGCTCA
CAGGCATCAGCAATGCAGAAGCACGGCAGCCAGGGAAGGCCCCCATATTCAGTGTCAACTGG
ACGGTAGGCGACTCCGCTATTGAGGTCATCAACGCCACGACTGGGAAGGGAGAGCTGGGCCG
CGCGTCCCGCCTGTGTAAGCACGCGTTGTACTGTCGCTGGATGCGTGTGCACGGCAAGGTTC
CCTCCCACTTACTACGCTCCAAGATTACCAAGCCCAACGTGTACCATGAGACAAAGCTGGCG
GCAAAGGAGTACCAGGCCGCCAAGGCGCGTCTGTTCACAGCCTTCATCAAGGCGGGGCTGGG
GGCCTGGGTGGAGAAGCCCACCGAGCAGGACCAGTTCTCACTCACGTAA
MtCsf5 (IV-A2):
(Sequence 55)
(SEQ ID NO: 83)
ATGCCCAAGAAGAAGAGAAAGGTGCACCAGACCCTGATCCGGATCAACTGGCCCAAGGGATT
CAAGTGCCCCCCTGCCGAGTTCCGGGAAAAGCTGGCCAAGAGCGAGATGTTCCCCCCCGAGT
TCTTCCACTACGGCACGGAACTGGCCGTGTGGGACAAGCAGACCGCCGAGGTGGAGGGCAAG
ATCAAGACCGTGTCCAAGGAGAAGATCATCAAGACCTTTGACAAGCCCATCCCCCTGAATGG
CCGGGCCCCGGTCAGAGTGATCGGCGGCCAGGCCTGGGCCGGCGTGATCGCCGACCCCGAGA
TGGAGGGCATGCTGATCCCACACCTGGGGAGCATCCTGAAGGTGGCCAGCAGCGCGGCCGGA
TGCGCAGTGAAGATCGAACTGGAACAGAGAAAGTTCGGCATCAGCTACACCGAGTACCCCGT
GAAGTACAACCTGCGGGAGCTGGTGCTGAAGAGAAGATGCGAGGACGCCCGGTCTACCGATA
TCGAGAGCCTGATTGCCGATAGAATCTGGGGCGGCGTGTCCGGCGAGAGCTACTATGGCATC
GACGGCACATGCGCCAAGTTTGGCTTCGAACCCCCCAGCAGAGAGCAGCTGGAGCTGCGGAT
CTTCCCCATGAAGAACATCGGACTGCACATGAAGTCCAGCGACGGACTGTCCAAGGAGTACA
TGAGCCTGATTGACGCCGAGGTGTGGATGAACGCTAAGCTGGAAGGAGTGTGGCAGGTGGGC
AACCTGATCAGCAGGGGCTACGGCCGGTTCATCAAGTCTATCGGCGCCCAGTCCCCCAAGAA
GAAGCGGAAGGTGGGTGGAGGCGGAGGTTCTGGGGGAGGAGGTAGTGGCGGTGGTGGTTCAG
GAGGCGGCGGAAGCCAGCTGCATTTACCGCAGGTTTTAGCTGACGCTGTCTCACGCCTGGTC
ATAGGTAAGTTTGGTGACCTGACCGACAACTTCTCCTCCCCTCACGCTCGCAGAATAGGTCT
GGCTGGAGTCGTCATGACAACAGGCACAGATGTTAAAGATGCCAAGGTGATATGTGTTTCTA
CAGGAGCAAAATGTATTAATGGTGAATACCTAAGTGATCGTGGCCTTGCATTAAATGACTGC
CATGCAGAAATAGTATCTCGGAGATCCTTGCTCAGATTTCTTTATACACAACTTGAGCTTTA
CTTAAATAACGAGGATGATCAAAAAAGATCCATCTTTCAGAAATCAGAGCGAGGGGGGTTTA
GGCTGAAGGAGAATATACAGTTTCATCTGTACATCAGCACCTCTCCCTGTGGAGATGCCAGA
ATCTTCTCACCACATGAGGCAATCCTGGAAGAACCAGCAGATAGACACCCAAATCGTAAAGC
AAGAGGACAGCTACGGACCAAAATAGAGGCTGGTCAGGGGACGATTCCAGTGCGCAACAATG
CGAGCATCCAAACGTGGGACGGGGTGCTGCAAGGGGAGCGGCTGCTCACCATGTCCTGCAGT
GACAAGATTGCACGCTGGAACGTGGTGGGCATCCAGGGATCACTGCTCAGCATTTTCGTGGA
GCCCATTTACTTCTCGAGCATCATCCTGGGCAGCCTTTACCACGGGGACCACCTTTCCAGGG
CCATGTACCAGCGGATCTCCAACATAGAGGACCTGCCACCTCTCTACACCCTCAACAAGCCT
TTGCTCACAGGCATCAGCAATGCAGAAGCACGGCAGCCAGGGAAGGCCCCCATATTCAGTGT
CAACTGGACGGTAGGCGACTCCGCTATTGAGGTCATCAACGCCACGACTGGGAAGGGAGAGC
TGGGCCGCGCGTCCCGCCTGTGTAAGCACGCGTTGTACTGTCGCTGGATGCGTGTGCACGGC
AAGGTTCCCTCCCACTTACTACGCTCCAAGATTACCAAGCCCAACGTGTACCATGAGACAAA
GCTGGCGGCAAAGGAGTACCAGGCCGCCAAGGCGCGTCTGTTCACAGCCTTCATCAAGGCGG
GGCTGGGGGCCTGGGTGGAGAAGCCCACCGAGCAGGACCAGTTCTCACTCACGTAA

In certain embodiments, the heterologous functional domain is fused N-terminally, C-terminally, or internally in the fusion protein.

In certain embodiments, the functional variant of the CasPR fusion comprises a protein having the amino acid sequence of: (1) any one of Sequences 1-11, except for 1, 2, 3, 4, 5, 6, 7, 8, 9, or 10 amino acid substitutions, additions, or deletions, wherein the protein maintains the ability of one of Sequences 1-11 for binding to a direct repeat sequence of a Class 1, type I, III, or IV CRISPR system (e.g., any one of Sequences 12-33); or, (2) at least 80%, 85%, 90%, 91%, 92%, 93%, 94%, 95%, 96%, 97%, 98%, 99% or more sequence identity with any one of Sequences 1-11, wherein the protein maintains the ability of one of the CasPRs of Sequences 1-11 for binding to a direct repeat sequence of a Class 1, type I, III, or IV CRISPR system (e.g., any one of Sequences 12-33); optionally, with the proviso that the protein is not any one of Sequences 1-11. All sequences incorporated herein by reference.

In certain embodiments, the RNA sequence of the invention further encodes a CasPR guide RNA comprising a guide sequence capable of hybridizing to a target RNA, and a direct repeat (DR) sequence 3′ (or 5′) to the guide sequence. In certain embodiments, the DR sequence has substantially the same secondary structure as the secondary structure of any one of Sequences 12-33. In certain embodiments, the DR sequence is encoded by any one of Sequences 12-33, or a functional portion thereof that binds to a cognate wild-type CasPR. In certain embodiments, the target RNA is encoded by a eukaryotic DNA. In certain embodiments, the eukaryotic DNA is a non-human mammalian DNA, a non-human primate DNA, a human DNA, a plant DNA, an insect DNA, a bird DNA, a reptile DNA, a rodent DNA, a fish DNA, a worm/nematode DNA, a yeast DNA. In certain embodiments, the target RNA is an mRNA.

In certain embodiments, the CasPR guide sequence is between 15-120 nucleotides, between 20-100 nucleotides, between 25-80 nucleotides, between 15-55 nucleotides, between 25-35 nucleotides, or about 30 nucleotides.

In certain embodiments, the CasPR guide sequence is 90-100% complementary to the target RNA. In certain embodiments, the CasPR guide RNA results from processing of a pre-crRNA transcript by the CasPR, and wherein the pre-crRNA comprises two or more guide RNAs having different guide sequences for different target RNAs.

In certain embodiments, the variant or derivative of the CasPR fusion comprises conserved amino acid substitutions of one or more residues of any one of Sequences 1-11; optionally, the variant or derivative of the CasPR fusion comprises only conserved amino acid substitutions. In certain embodiments, the derivative of the CasPR fusion is capable of binding to the CasPR guide sequence hybridized to the target RNA, but has no RNase catalytic activity due to a mutation in the RNase catalytic site of the CasPR. In certain embodiments, the derivative of the CasPR fusion has an N-terminal deletion of no more than 5, 10, 15, 20, 25, 30, 35, 40, 45, or 50 residues, and/or a C-terminal deletion of no more than 5, 10, 15, 20, 25, 30, 35, 40, 45, or 50 residues.

In certain embodiments, the CasPR is a Cas5d, a Cas6, or a Csf5, such as MtCas6 (I-A), MmCas6 (I-B), SpCas5d (I-C1), BhCas5d (I-C2), SaCas6 (I-D), EcCas6e (I-E), PaCas6f (I-F), MtCas6 (III-A), PfCas6 (III-B), PaCsf5 (IV-A1), or MtCsf5 (IV-A2).

In certain embodiments, the heterologous functional domain of the CasPR fusion comprises an RNA base-editing domain. In certain embodiments, the RNA base-editing domain comprises an adenosine deaminase and/or a cytidine deaminase, such as a cytidine deaminase acting on RNA (CDAR), such as a double-stranded RNA-specific adenosine deaminase (ADAR) (e.g., ADAR1 or ADAR2), apolipoprotein B mRNA editing enzyme, catalytic polypeptide-like (APOBEC, such as APOBEC1, APOBEC2, APOBEC3A, APOBEC3B, APOBEC3C, APOBEC3D, APOBEC3E, APOBEC3F, APOBEC3G, APOBEC3H, and APOBEC4), activation-induced cytidine deaminase (AID), a cytidine deaminase 1 (CDA1), or a mutant thereof. In certain embodiments, the ADAR comprises E488Q/T375G double mutation or comprises ADAR2DD. In certain embodiments, the base-editing domain is further fused to an RNA-binding domain, such as MS2.

In certain embodiments, the derivative of the CasPR fusion further comprises an RNA methyltransferase, a RNA demethylase, an RNA splicing modifier, a localization factor, or a translation modification factor.

In certain embodiments, the CasPR, the homolog, the ortholog, the paralog, the variant, the derivative, or the functional fragment comprises a nuclear localization signal (NLS) sequence or a nuclear export signal (NES).

In certain embodiments, targeting of the target RNA results in a modification of the target RNA. In certain embodiments, the modification of the target RNA is a cleavage of the target RNA. In certain embodiments, the modification of the target RNA is deamination of an adenosine (A) to an inosine (I), and/or deamination of a cytidine (C) to a uracil (U).

In certain embodiments, the RNA sequence of the invention encodes a codon-optimized polynucleotide encoding a wild-type CasPR (e.g., Cas5d, Cas6, or Csf5), a homolog thereof, an ortholog thereof, a paralog thereof, a variant or derivative thereof, or a functional fragment thereof, wherein the polynucleotide is codon-optimized for mammalian (e.g., human) expression, optionally, the wild-type CasPR has the amino acid sequence of any one of Sequences 1-11. In certain embodiments, the codon-optimized polynucleotide has the amino acid sequence of any one of Sequences 34-44. In certain embodiments, the codon-optimized polynucleotide further comprises sequence encoding a heterologous functional domain. In certain embodiments, the heterologous functional domain comprises an RNA base editor.

MtCas6 (I-A):
(Sequence 34)
(SEQ ID NO: 84)
ATGCCTCTGATCTTCAAGATCGGCTATAACGTGATCCCCCTGCAGGACGTGATCCTGCCCAC
CCCTTCCAGCAAGGTGCTGAAGTACCTGATCCAGAGCGGCAAGCTGATCCCCAGCCTGAAGG
ACCTGATCACCAGCCGGGACAAGTACAAGCCAATCTTCATCTCCCACCTGGGCTTCAACCAG
CGGAGGATTTTCCAGACCAACGGCAATCTGAAAACCATCACCAAGGGCAGTAGACTGAGCTC
CATCATCGCCTTCAGCACCCAGGCCAACGTGCTGTCCGAGGTGGCCGATGAAGGGATCTTCG
AAACCGTGTACGGAAAGTTCCACATCATGATCGAAAGCATCGAGATCGTGGAGGTGGAAAAG
CTGAAGGAGGAGGTGGAGAAGCACATGAACGACAACATCAGAGTGAGATTCGTGTCTCCCAC
ACTGCTGAGCTCCAAGGTGCTGCTGCCCCCCAGCCTGTCCGAAAGATACAAGAAGATCCACG
CCGGGTACAGCACCCTGCCCAGCGTGGGCCTGATCGTGGCCTACGCCTACAACGTGTACTGC
AATCTGATCGGCAAGAAGGAAGTGGAAGTGCGGGCCTTCAAGTTTGGAATCCTGAGCAACGC
CCTGTCCAGAATCATCGGCTACGACCTGCACCCTGTGACCGTGGCCATCGGCGAGGACAGCA
AGGGGAATCTGAGAAAGGCTCGGGGCGTGATGGGCTGGATCGAGTTCGACATCCCCGACGAA
AGACTGAAGCGGCGGGCCCTGAACTATCTGCTGACCAGCAGCTACCTGGGCATCGGGAGATC
TCGGGGCATCGGCTTCGGCGAGATCCGGCTGGAGTTCCGGAAGATTGAAGAGAAGGAGGGA
MmCas6 (I-B):
(Sequence 35)
(SEQ ID NO: 85)
ATGGACCTGGAGTACATGCACATCTCCTACCCTAACATCCTGCTGAACATGCGGGACGGCAG
CAAGCTGCGGGGCTACTTCGCCAAGAAGTACATCGACGAAGAGATTGTGCACAACCACAGAG
ACAACGCCTTTGTGTACAAGTACCCCCAGATCCAGTTTAAGATCATCGATAGAAGCCCCCTG
ATCATCGGCATTGGCTCTCTGGGCATCAATTTCCTGGAGAGCAAGCGGATCTTCTTCGAGAA
GGAACTGATTATCAGCAACGACACCAACGACATCACCGAGGTGAACGTGCACAAGGACATGG
ATCACTTCGGCACGACCGACAAGATCCTGAAGTACCAGTTCAAGACCCCTTGGATGGCACTG
AACGCCAAGAATAGCGAGATCTACAAGAACTCTGACGAGATCGACCGGGAGGAGTTCCTGAA
GAGAGTGCTGATTGGGAATATCCTGAGCATGTCTAAGAGCCTGGGCTATACCATCGAAGAAA
AGCTGAAGGTGAAGATTAACCTGAAGGAAGTGCCCGTGAAGTTCAAGAACCAGAACATGGTG
GGCTTTCGGGGCGAGTTCTACATCAACTTCGACATCCCTCAGTATCTGGGCATCGGCCGGAA
TGTGTCCCGGGGATTCGGCACAGTGGTGAAGGTG
SpCas5d (I-C1):
(Sequence 36)
(SEQ ID NO: 86)
ATGAGAAATGAAGTGCAGTTCGAGCTGTTCGGCGACTACGCCCTGTTCACCGACCCCCTGAC
CAAGATCGGCGGCGAAAAGCTGAGCTACAGCGTGCCTACCTACCAGGCCCTGAAGGGCATCG
CCGAGAGCATCTACTGGAAGCCCACCATCGTGTTCGTGATCGACGAACTGCGGGTCATGAAG
CCCATTCAGATGGAGTCTAAGGGCGTGAGGCCCATCGAGTACGGCGGCGGCAACACCCTGGC
CCACTACACCTACCTGAAGGATGTGCACTACCAGGTGAAGGCCCACTTCGAGTTCAACCTGC
ACCGGCCCGACCTGGCCTTCGATAGAAACGAGGGCAAGCACTACTCCATCCTGCAGAGAAGC
CTGAAGGCCGGCGGCAGAAGAGATATTTTCCTGGGCGCCCGGGAGTGCCAGGGCTACGTGGC
CCCCTGCGAGTTCGGCAGCGGCGACGGCTTCTACGACGGCCAGGGCAAGTACCACCTGGGAA
CCATGGTGCACGGTTTCAACTACCCCGACGAAACCGGACAGCACCAGCTGGATGTGAGACTG
TGGTCTGCCGTCATGGAAAACGGCTACATCCAGTTCCCCCGCCCTGAGGACTGCCCCATCGT
GCGGCCTGTGAAGGAGATGGAACCCAAGATCTTCAACCCCGACAACGTGCAGTCCGCCGAAC
AGCTGCTGCACGACCTGGGCGGCGAA
BhCas5d (I-C2):
(Sequence 37)
(SEQ ID NO: 87)
ATGTACAGAAGCCGGGACTTCTACGTGAGAGTGTCCGGCCAGCGGGCCCTGTTCACCAACCC
CGCCACCAAGGGCGGCTCCGAACGGAGCTCCTACTCCGTGCCTACCCGGCAGGCCCTGAACG
GGATTGTGGACGCCATCTACTACAAGCCCACGTTCACCAACATCGTGACCGAGGTGAAGGTG
ATTAACCAGATCCAGACCGAACTGCAGGGCGTGCGGGCCCTGCTGCATGACTACAGCGCCGA
CCTGAGCTACGTGTCCTACCTGAGCGACGTGGTGTACCTGATTAAGTTTCATTTCGTGTGGA
ACGAGGATAGAAAGGACCTGAATAGCGACCGGCTGCCAGCCAAGCATGAGGCCATCATGGAG
CGGTCTATCCGGAAGGGCGGCAGACGGGACGTGTTCCTGGGCACCAGAGAATGCCTGGGCCT
GCTGGACGACATCAGCCAGGAAGAATACGAAACCACAGTGAGCTATTACAATGGGGTGAACA
TCGACCTGGGCATCATGTTCCACAGCTTCGCTTACCCCAAGGACAAGAAAACCCCCCTGAAG
TCCTACTTCACAAAGACCGTGATGAAGAACGGCGTGATCACCTTCAAGGCCCAGTCCGAATG
CGATATTGTGAACACCCTGAGCTCCTACGCCTTCAAGGCCCCCGAGGAGATCAAGAGCGTGA
ACGACGAGTGCATGGAGTACGACGCCATGGAGAAGGGCGAAAAC
SaCas6 (I-D):
(Sequence 38)
(SEQ ID NO: 88)
ATGCCCAACGATCCCTACAGCCTGTACTCCATCGTGATCGAACTGGGCGCCGCCGAAAAGGG
ATTCCCCACAGGCATCCTGGGCAGAAGCCTGCATAGCCAGGTGCTGCAGTGGTTCAAGCAGG
ATAACCCCTTCCTGGCCACCGAGCTGCACCAGAGCCAGATCTCCCCCTTCTCCATCTCTCCA
CTGATGGGCAAGCGGCACGCCAAGCTGACCAAGGCCGGCGACCGGCTGTTCTTTCGGATCTG
CCTGCTGAGAGGAGATCTGCTGCAGCCCCTGCTGAACGGCATTGAGCAGACCGTGAACCAGA
GCGTGTGCCTGGACAAGTTCCGGTTCCGGCTGTGCCAGACCCACATCCTGCCCGGCAGCCAC
CCTCTGGCTGGCGCCTCCCACTATAGCCTGATCAGCCAGACCCCAGTGAGCTCCAAGATTAC
CCTGGACTTCAAGAGTTCTACCTCCTTCAAGGTGGACCGGAAGATCATCCAAGTGTTCCCTC
TGGGCGAACACGTGTTCAACAGCCTGCTCAGACGCTGGAATAACTTCGCCCCCGAGGACCTG
CACTTCTCTCAGGTGGACTGGAGCATCCCCATCGCCGCATTCGACGTGAAAACCATCCCCAT
CCACCTGAAGAAGGTCGAGATCGGCGCACAGGGCTGGGTGACCTACATCTTCCCCAACACAG
AACAGGCCAAGATCGCCTCCGTGCTGAGCGAATTCGCCTTCTTCAGCGGAGTGGGACGGAAA
ACCACCATGGGCATGGGCCAGGTGCAGGTGCGGTCC
EcCas6e (I-E):
(Sequence 39)
(SEQ ID NO: 89)
ATGTACCTGAGCAAGGTGATCATCGCCAGAGCCTGGAGCAGAGACCTGTACCAGCTGCACCA
GGGCCTGTGGCACCTGTTCCCCAACCGGCCCGACGCCGCCCGGGATTTCCTGTTCCACGTGG
AGAAGAGAAACACCCCGGAAGGCTGCCACGTGCTGCTGCAGAGCGCACAGATGCCTGTGAGC
ACCGCCGTGGCCACCGTGATCAAGACCAAGCAGGTGGAGTTCCAGCTGCAGGTGGGCGTGCC
CCTGTATTTCAGGCTGCGGGCGAATCCCATCAAGACCATCCTGGACAACCAGAAGCGGCTGG
ACAGCAAGGGCAACATCAAGAGGTGCAGAGTGCCTCTGATCAAGGAGGCCGAACAGATCGCC
TGGCTGCAGCGGAAGCTGGGCAATGCCGCCAGAGTGGAGGACGTGCACCCCATCAGCGAGCG
GCCCCAGTACTTCTCCGGCGACGGAAAGAGCGGAAAGATCCAGACCGTGTGCTTCGAGGGCG
TGCTGACCATCAACGACGCACCCGCCCTGATCGACCTCGTGCAGCAGGGGATCGGCCCTGCC
AAGTCCATGGGCTGCGGACTGCTGTCCCTGGCCCCCCTG
PaCas6f (I-F):
(Sequence 40)
(SEQ ID NO: 90)
ATGGACCACTACCTGGACATTAGACTGCGCCCTGACCCAGAGTTCCCTCCTGCCCAGCTGAT
GTCTGTGCTGTTTGGCAAGCTGCACCAGGCCCTGGTGGCCCAGGGCGGTGACAGAATCGGAG
TGTCTTTCCCTGATCTGGACGAATCTAGATCTAGACTGGGAGAGAGACTGAGAATCCACGCG
TCTGCCGACGACCTGAGAGCTCTGCTGGCCAGACCATGGCTGGAAGGACTGCGCGACCACCT
GCAGTTCGGTGAACCTGCCGTGGTGCCTCACCCAACTCCATACAGACAGGTGAGTAGAGTGC
AGGCAAAGTCTAATCCAGAGAGACTGAGACGCAGACTGATGAGAAGGCATGACCTGTCCGAA
GAAGAAGCCAGAAAGAGAATCCCAGACACAGTGGCCAGAGCCCTGGATCTGCCTTTTGTGAC
CCTGAGAAGCCAGTCTACCGGCCAGCACTTCAGACTGTTTATTCGCCACGGACCACTGCAGG
TGACCGCCGAAGAGGGAGGTTTTACCTGCTACGGACTGAGCAAGGGAGGTTTCGTGCCTTGG
TTC
MtCas6 (III-A):
(Sequence 41)
(SEQ ID NO: 91)
ATGGCCGCCAGAAGAGGCGGAATCCGGAGAACCGACCTGCTGCGGAGGTCTGGCCAGCCTCG
GGGCAGACACCGGGCCTCCGCCGCCGAGAGCGGCCTGACATGGATCTCCCCTACCCTGATCC
TGGTGGGCTTCAGCCACAGGGGCGATAGGAGAATGACCGAGCACCTGTCCAGACTGACCCTG
ACCCTGGAAGTGGATGCCCCCCTGGAGAGAGCCCGGGTGGCCACCCTGGGCCCCCACCTGCA
TGGCGTGCTGATGGAGTCTATCCCCGCCGACTACGTGCAGACACTGCACACAGTGCCGGTGA
ACCCTTACAGCCAGTACGCTCTGGCCCGGAGCACCACCAGCCTGGAGTGGAAGATCTCCACC
CTGACAAATGAGGCCCGGCAGCAGATCGTCGGCCCCATCAACGACGCCGCCTTCGCCGGCTT
CCGGCTGCGGGCCAGCGGCATCGCCACCCAGGTGACAAGCAGAAGCCTGGAGCAGAACCCCC
TGTCCCAGTTTGCCAGAATCTTCTACGCCAGGCCCGAAACCCGCAAGTTCAGAGTGGAGTTC
CTGACCCCCACCGCCTTCAAGCAGAGCGGCGAGTACGTGTTTTGGCCCGATCCCAGACTGGT
GTTCCAGTCCCTGGCCCAGAAGTACGGCGCCATCGTGGACGGAGAAGAGCCCGACCCCGGCC
TGATCGCCGAGTTTGGCCAGTCCGTGAGACTGAGCGCCTTCAGAGTGGCCAGCGCCCCTTTT
GCCGTGGGCGCCGCCAGGGTGCCCGGATTCACCGGCAGCGCCACCTTCACCGTGCGGGGAGT
GGACACCTTCGCCAGCTACATCGCCGCTCTGCTGTGGTTCGGCGAGTTCAGCGGATGCGGCA
TCAAGGCCTCCATGGGAATGGGCGCCATCCGGGTGCAGCCTCTGGCCCCCCGGGAGAAGTGC
GTGCCCAAGCCC
PfCas6 (III-B):
(Sequence 42)
(SEQ ID NO: 92)
ATGAGATTCCTGATCAGACTGGTGCCCGAGGACAAGGACAGAGCCTTCAAGGTGCCTTACAA
CCACCAGTACTATCTGCAGGGCCTGATCTACAACGCCATCAAGTCCTCCAACCCCAAGCTGG
CCACCTACCTGCACGAGGTGAAGGGCCCCAAGCTGTTCACCTACAGCCTGTTCATGGCCGAA
AAGCGGGAGCACCCTAAGGGCCTGCCCTACTTTCTGGGCTACAAGAAGGGCTTCTTCTACTT
CAGCACCTGCGTGCCCGAGATCGCCGAGGCCCTGGTGAACGGCCTGCTGATGAATCCCGAGG
TGCGGCTGTGGGACGAGAGATTCTACCTGCACGAAATCAAGGTCCTGCGGGAGCCCAAGAAG
TTCAACGGCAGCACCTTCGTGACCCTGAGCCCCATCGCCGTGACCGTGGTGAGAAAGGGCAA
GTCCTACGACGTGCCCCCCATGGAAAAGGAGTTCTACAGCATTATCAAGGATGACCTGCAGG
ACAAGTACGTGATGGCCTACGGCGACAAGCCCCCCAGTGAGTTCGAGATGGAAGTGCTGATC
GCCAAGCCCAAGCGGTTCCGGATCAAGCCCGGCATCTATCAGACCGCCTGGCACCTGGTGTT
TCGGGCCTACGGCAATGACGACCTGCTGAAGGTGGGCTACGAAGTGGGATTCGGGGAGAAGA
ACTCCCTGGGATTCGGAATGGTCAAGGTGGAGGGCAACAAGACCACCAAGGAAGCCGAAGAA
CAGGAGAAGATCACCTTCAACTCCCGGGAAGAGCTGAAAACAGGCGTG
PaCsf5 (IV-A1):
(Sequence 43)
(SEQ ID NO: 93)
ATGTTCGTGACCCAGGTGATCTTCAACATCGGCGAACGGACGTACCCCGACAGGGCTCGGGC
TATGGTGGCCGAGCTGATGGATGGCGTCCAGCCTGGCCTGGTGGCCACCCTGATGAACTACA
TCCCCGGCACCAGCACGAGCCGGACAGAGTTCCCCACCGTGCAGTTCGGCGGCGCCAGCGAC
GGCTTTTGCCTGCTGGGCTTCGGCGACGGCGGCGGCGCCATCGTGAGAGATGCCGTGCCCCT
GATCCACGCCGCCCTGGCAAGGCGGATGCCTGATCGGATCATCCAGGTGGAACACAAGGAGC
ACAGCCTGTCCGCCGAGGCCCGGCCCTACGTGCTGAGCTACACCGTGCCTCGGATGGTGGTG
CAGAAGAAGCAGCGGCACGCCGAGAGACTGCTGCACGAAGCCGAGGGAAAGGCTCACCTGGA
GGGCCTGTTCCTGCGGAGCCTGCAGAGGCAGGCCGCCGCCGTGGGCCTGCCCCTGCCCGAGA
ACCTGGAGGTGGAGTTCAAGGGAGCCGTGGGCGACTTCGCCGCAAAGCACAATCCAAATAGC
AAGGTGGCCTACCGGGGACTGAGAGGCGCCGTGTTCGATGTGAACGCCAGACTGGGCGGCAT
CTGGACCGCCGGATTCATGCTGAGCAAGGGCTACGGCCAGTTTAACGCCACCCACCAGCTGA
GCGGCGCCGTGAACGCTCTGTCCGAA
MtCsf5 (IV-A2):
(Sequence 44)
(SEQ ID NO: 94)
ATGCACCAGACCCTGATCCGGATCAACTGGCCCAAGGGATTCAAGTGCCCCCCTGCCGAGTT
CCGGGAAAAGCTGGCCAAGAGCGAGATGTTCCCCCCCGAGTTCTTCCACTACGGCACGGAAC
TGGCCGTGTGGGACAAGCAGACCGCCGAGGTGGAGGGCAAGATCAAGACCGTGTCCAAGGAG
AAGATCATCAAGACCTTTGACAAGCCCATCCCCCTGAATGGCCGGGCCCCGGTCAGAGTGAT
CGGCGGCCAGGCCTGGGCCGGCGTGATCGCCGACCCCGAGATGGAGGGCATGCTGATCCCAC
ACCTGGGGAGCATCCTGAAGGTGGCCAGCAGCGCGGCCGGATGCGCAGTGAAGATCGAACTG
GAACAGAGAAAGTTCGGCATCAGCTACACCGAGTACCCCGTGAAGTACAACCTGCGGGAGCT
GGTGCTGAAGAGAAGATGCGAGGACGCCCGGTCTACCGATATCGAGAGCCTGATTGCCGATA
GAATCTGGGGCGGCGTGTCCGGCGAGAGCTACTATGGCATCGACGGCACATGCGCCAAGTTT
GGCTTCGAACCCCCCAGCAGAGAGCAGCTGGAGCTGCGGATCTTCCCCATGAAGAACATCGG
ACTGCACATGAAGTCCAGCGACGGACTGTCCAAGGAGTACATGAGCCTGATTGACGCCGAGG
TGTGGATGAACGCTAAGCTGGAAGGAGTGTGGCAGGTGGGCAACCTGATCAGCAGGGGCTAC
GGCCGGTTCATCAAGTCTATCGGCGCCCAGTCC

In certain embodiments, the RNA sequence of the invention encodes a non-naturally occurring polynucleotide comprising a derivative of any one of Sequences 12-33, wherein the derivative (i) has one or more (e.g., 1, 2, 3, 4, 5, 6, 7, 8, 9 or 10) nucleotides additions, deletions, substitutions, and/or other mutations compared to any one of Sequences 12-33; (ii) has at least 20%, 30%, 40%, 50%, 60%, 70%, 80%, 90%, 95%, or 97% sequence identity to any one of Sequences 12-33; (iii) hybridize under stringent conditions with any one of Sequences 12-33, or any of (i) and (ii); or (iv) is a complement of any of (i)-(iii), provided that the derivative is not any one of Sequences 12-33, and that the derivative encodes an RNA (or is an RNA) that has maintained substantially the same secondary structure (e.g., stems, loops, bulges, single-stranded regions) as any of the RNA encoded by Sequences 12-33. In certain embodiments, the derivative functions as a DR sequence for any one of the CasPR, the ortholog thereof, the paralog thereof, the variant thereof, the derivative thereof, or the functional fragment thereof, of the invention.

In certain embodiments, the RNA sequence of the invention comprises a coding sequence for an engineered Clustered Regularly Interspaced Short Palindromic Repeat (CRISPR)-Cas13 effector enzyme, wherein the engineered Cas13: (1) comprises a mutation in a region spacially close to an endonuclease catalytic domain of the corresponding wild-type Cas13 effector enzyme; (2) substantially preserves guide sequence-specific endonuclease cleavage activity of the wild-type Cas 13 towards a target RNA complementary to the guide sequence; and, (3) substantially lacks guide sequence-independent collateral endonuclease cleavage activity of the wild-type Cas 13 towards a non-target RNA that does not bind to the guide sequence.

In certain embodiments, the Cas 13 is a Cas13a, a Cas13b, a Cas13c, a Cas13d (including CasRx), a Cas13c, or a Cas13f.

In certain embodiments, the Cas 13e has the amino acid sequence of SEQ ID NO: 4 of PCT/CN2020/119559 (incorporated herein by reference).

In certain embodiments, the region includes residues within 120, 110, 100, 90, or 80 amino acids from any residues of the endonuclease catalytic domain (e.g., an RXXXXH domain) in the primary sequence of the Cas13.

In certain embodiments, the region includes residues more than 100, 110, 120, or 130 residues away from any residues of the endonuclease catalytic domain in the primary sequence of the Cas 13, but are spacially within 1-10 or 5 ångström of a residue of the endonuclease catalytic domain.

In certain embodiments, the endonuclease catalytic domain is a HEPN domain, optionally a HEPN domain comprising an RXXXXH motif. In certain embodiments, the RXXXXH motif comprises a R {N/H/K} X1X2X3H sequence. In certain embodiments, in the R {N/H/K} X1X2X3H sequence, X1 is R, S, D, E, Q, N, G, or Y; X2 is I, S, T, V, or L; and X3 is L, F, N, Y, V, I, S, D, E, or A. In certain embodiments, the RXXXXH motif is an N-terminal RXXXXH motif comprising an RNXXXH sequence, such as an RN {Y/F} {F/Y} SH sequence (SEQ ID NO: 95). In certain embodiments, the N-terminal RXXXXH motif has a RNYFSH sequence (SEQ ID NO: 96). In certain embodiments, the N-terminal RXXXXH motif has a RNFYSH sequence (SEQ ID NO: 97). In certain embodiments, the RXXXXH motif is a C-terminal RXXXXH motif comprising an

R {N/A/R} {A/K/S/F} {A/L/F} {F/H/L} H sequence. In certain embodiments, the C-terminal RXXXXH motif has a RN (A/K) ALH sequence (SEQ ID NO: 98). In certain embodiments, the C-terminal RXXXXH motif has a RAFFHH (SEQ ID NO: 99) or RRAFFH sequence (SEQ ID NO: 100).

In certain embodiments, the region comprises, consists essentially of, or consists of residues corresponding to residues between residues 2-187, 227-242, or 634-755 of SEQ ID NO: 4 of PCT/CN2020/119559 (incorporated by reference). In certain embodiments, the region comprises, consists essentially of, or consists of residues corresponding to residues between residues 35-51, 52-67, 156-171, 666-682, or 712-727 of SEQ ID NO: 4 of PCT/CN2020/119559 (incorporated by reference).

In certain embodiments, the mutation comprises, consists essentially of, or consists of substitutions, within a stretch of 15-20 consecutive amino acids within the region, one or more charged or polar residues to a charge-neutal short chain aliphatic residue (such as A). In certain embodiments, the stretch is about 16 or 17 residues. In certain embodiments, substantially all, except for up to 1, 2, or 3, charged and polar residues within the stretch are substituted. In certain embodiments, a total of about 7, 8, 9, or 10 charged and polar residues within the stretch are substituted. In certain embodiments, the N- and C-terminal 2 residues of the stretch are substituted to amino acids the coding sequences of which contain a restriction enzyme recognition sequence. In certain embodiments, the N-terminal two residues are VF, and the C-terminal 2 residues are ED, and the restriction enzyme is Bpil. In certain embodiments, the one or more charged or polar residues comprise N, Q, R, K, H, D, E, Y, S, and T residues. In certain embodiments, the one or more charged or polar residues comprise R, K, H, N, Y, and/or Q residues. In certain embodiments, one or more Y residue(s) within the stretch is substituted. In certain embodiments, the one or more Y residues(s) correspond to Y672, Y676, and/or Y751 of wild-type Cas13c.1 (SEQ ID NO: 4 of PCT/CN2020/119559 (incorporated by reference)). In certain embodiments, the stretch is residues 35-51, 52-67, 156-171, 666-682, or 712-727 of SEQ ID NO: 4 of

PCT/CN2020/119559 (incorporated by reference). In certain embodiments, the mutation comprises Ala substitution(s) corresponding to any one or more of SEQ ID NOs: 37-39, 45, and 48 of PCT/CN2020/119559 (incorporated by reference). In certain embodiments, the charge-neutal short chain aliphatic residue is Ala (A). In certain embodiments, the mutation comprises, consists essentially of, or consists of substitutions within 2, 3, 4, or 5 the stretches of 15-20 consecutive amino acids within the region.

In certain embodiments, the engineered Cas13 preserves at least about 50%, 60%, 70%, 80%, 85%, 90%, 95%, 96%, 97%, 98%, or 99% of the guide sequence-specific endonuclease cleavage activity of the wild-type Cas 13 towards the target RNA.

In certain embodiments, the engineered Cas13 lacks at least about 80%, 85%, 90%, 95%, 96%, 97%, 98%, 99%, or 100% of the guide sequence-independent colleteral endonuclease cleavage activity of the wild-type Cas 13 towards the non-target RNA.

In certain embodiments, the engineered Cas 13 preserves at least about 80-90% of the guide sequence-specific endonuclease cleavage activity of the wild-type Cas13 towards the target RNA, and lacks at least about 95-100% of the guide sequence-independent colleteral endonuclease cleavage activity of the wild-type Cas 13 towards the non-target RNA.

In certain embodiments, the engineered Cas13 of the invention has an amino acid sequence at least 80%, 85%, 90%, 95%, 96%, 97%, 98%, 99%, 99.1%, 99.2%, 99.3%, 99.4%, 99.5%, 99.6%, 99.7%, 99.8%, or 99.86% identical to any one of SEQ ID NOs: 6-10 of PCT/CN2020/119559 (incorporated by reference), excluding any one or more of the regions defined by SEQ ID NOs: 16, 20, 24, 28, and 32 of PCT/CN2020/119559

### INCORPORATED BY REFERENCE

In certain embodiments, the amino acid sequence contains up to 1, 2, 3, 4, or 5 differences in each of one or more regions defined by SEQ ID NO: 16, 20, 24, 28, and 32 of PCT/CN2020/119559 (incorporated by reference), as compared to SEQ ID NOs: 17, 21, 25, 29, and 33 of PCT/CN2020/119559 (incorporated by reference), respectively.

In certain embodiments, he engineered Cas13 of the invention has the amino acid sequence of any one of SEQ ID NOs: 6-10 of PCT/CN2020/119559 (incorporated by reference). In certain embodiments, the engineered Cas13 of the invention has the amino acid sequence of SEQ ID NO: 9 or 10 of PCT/CN2020/119559 (incorporated by reference).

In certain embodiments, the engineered Cas13 of the invention further comprises a nuclear localization signal (NLS) sequence or a nuclear export signal (NES). In certain embodiments, the engineered Cas13 comprises an N—and/or a C-terminal NLS.

In certain embodiments, the RNA sequence of the invention encoding the engineered CRISPR/Cas13 effector enzyme of the invention is codon-optimized for expression in a eukaryote, a mammal, such as a human or a non-human mammal, a plant, an insect, a bird, a reptile, a rodent (e.g., mouse, rat), a fish, a worm/nematode, or a yeast.

In certain embodiments, the RNA sequence of the invention comprises a coding sequence for the engineered Clustered Regularly Interspaced Short Palindromic Repeat (CRISPR)-Cas13 effector enzyme, the coding sequence having (i) one or more (e.g., 1, 2, 3, 4, 5, 6, 7, 8, 9 or 10) nucleotides additions, deletions, substitutions, and/or other mutations compared to the wild-type sequence; (ii) at least 50%, 60%, 70%, 80%, 90%, 95%, or 97% sequence identity to the wild-type sequence; (iii) hybridize under stringent conditions with the wild-type sequence, or any of (i) and (ii); or (iv) is a complement of any of (i)-(iii).

In certain embodiments, the RNA sequence of the invention comprises a coding sequence for a non-coding RNA (ncRNA), such as siRNA, piRNA, short hairpin RNA or shRNA, microRNA or miRNA or precursors thereof including pre-miRNA and pri-miRNA, antisense sequence or oligonucleotide (ASO), guide RNA or gRNA for CRISPR/Cas, rRNA, tRNA, snoRNA, snRNA, exRNA, scaRNA, lncRNA, Xist, and HOTAIR, etc.
9. Method of Use

The rRAAV viral particles and RNA sequences of the invention can be used to deliver any GOI/RSI to any suitable target cell, tissue, or organism for any use for gene therapy.

In certain embodiments, the rRAAV viral particles and RNA sequences of the invention can be used in a method of treatment, in which a defective or loss of function disease gene can be replaced by a functional version of the gene to restore the lost function. For example, in certain embodiments, a wild-type coding sequence, or a variant coding sequence encoding a variant protein of the wild-type protein and having preserved at least one desired functions of the wild-type protein can be delivered to the target cell/tissue/organ, to express the encoded wild-type of variant thereof, in order to compensate for the loss of function of the disease gene.

In certain other embodiments, the rRAAV viral particles and RNA sequences of the invention can be used in a method of treatment, in which a defective or gain of function disease gene can be knocked out, knocked down, or otherwise down-regulated by a gene targeting agent to alleviate the detrimental effect of the disease gene. The gene targeting agent can be a CRISPR/Cas effector enzyme (such as an engineered Cas9 or Cas13 effector enzyme as described herein), optionally with a guide RNA that is provided simultaneously (or separately), that together target the disease gene. In certain embodiments, the gene targeting agent can be a Cas effector enzyme linked to a DNA or RNA base editor for DNA-RNA base editing. In certain embodiments, the gene targeting agent is an siRNA, shRNA, microRNA, or antisense RNA.

In certain embodiments, the invention provides a method of modifying a target RNA in a target cell, the method comprising contacting the target cell with an rRAAV viral particle or RNA sequence of the invention encoding a CasPR or engineered CRISPR/Cas effector enzyme described herein (or ortholog, paralog, variant, derivative, or functional fragment thereof), wherein a guide sequence for the CasPR/Cas effector enzyme is complementary to at least 15 nucleotides of the target RNA, and wherein the CasPR/engineered Cas effector enzyme associates with the guide sequence to form a complex that binds to and modified the target RNA.

In certain embodiments, the invention provides a method of treating a condition or disease in a subject in need thereof, the method comprising administering to the subject a composition comprising the an rRAAV viral particle or RNA sequence of the invention encoding a CasPR or engineered CRISPR/Cas effector enzyme described herein (or ortholog, paralog, variant, derivative, or functional fragment thereof), wherein a guide sequence for the CasPR/Cas effector enzyme is complementary to at least 15 nucleotides of the target RNA, and wherein the CasPR/engineered Cas effector enzyme associates with the guide sequence to form a complex that binds to and modified the target RNA, thereby treating the condition or disease in the subject.

In certain embodiments, the target RNA is modified by cleavage by the CasPR or engineered Cas effector enzyme complex. In certain embodiments, the target RNA is modified by deamination by a derivative comprising a double-stranded RNA-specific adenosine and/or cytidine deaminase. In certain embodiments, the target RNA is an mRNA, a tRNA, an rRNA, a non-coding RNA, an lncRNA, or a nuclear RNA. In certain embodiments, the target RNA is within a cell. In certain embodiments, the cell is a cancer cell. In certain embodiments, the cell is infected with an infectious agent. In certain embodiments, the infectious agent is a virus, a prion, a protozoan, a fungus, or a parasite. In certain embodiments, the cell is a neuronal cell (e.g., astrocyte, glial cell (e.g., Muller glia cell, oligodendrocyte, ependymal cell, Schwan cell, NG2 cell, or satellite cell)).

In certain embodiments, the condition or disease is a cancer or an infectious disease. In certain embodiments, the cancer is Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, or urinary bladder cancer. In certain embodiments, the method is an in vitro method, an in vivo method, or an ex vivo method. In certain embodiments, upon binding of the complex to the target RNA, the engineered Cas13 does not exhibit substantial (or detectable) collateral RNase activity.

In certain embodiments, the condition or disease is a neurological condition such as glaucoma, age-related RGC loss, optic nerve injury, retinal ischemia, Leber's hereditary optic neuropathy, a neurological condition associated with degeneration of RGC neurons, a neurological condition associated with degeneration of functional neurons in the striatum of a subject in need thereof, Parkinson's disease, Alzheimer's disease, Huntington's disease, Schizophrenia, depression, drug addiction, movement disorder such as chorea, choreoathetosis, and dyskinesias, bipolar disorder, Autism spectrum disorder (ASD), or dysfunction.

In certain embodiments, the method of the invention causes one or more of: (i) in vitro or in vivo induction of cellular senescence; (ii) in vitro or in vivo cell cycle arrest; (iii) in vitro or in vivo cell growth inhibition and/or cell growth inhibition; (iv) in vitro or in vitro induction of anergy; (v) in vitro or in vitro induction of apoptosis; and (vi) in vitro or in vitro induction of necrosis.

### Further Embodiments of the Invention

1. A polynucleotide sequence, including, but not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof, capable of being packaged into a DNA virus viral particle, said polynucleotide sequence comprises:
   (1) a polynucleotide sequence of interest (PSI), e.g., a RNA coding sequence for a gene of interest (GOI), a protein (e.g., a therapeutic protein, an antigen protein, or a gene-editing protein such as a CRISPR/Cas effector enzyme (“a Cas protein” for short), a ZFN protein, a TALEN protein)-encoding RNA, such as, a mRNA, or a non-coding, functional RNA (such as, a transfer RNA (tRNA), a ribosomal RNA (rRNA), a small interfering RNA (siRNA), a short hairpin RNA (shRNA), an antisense RNA, an antisense oligonucleotide, a micro RNA (miRNA), or an RNA component of a CRISPR-Cas (e.g., Cas9, Cas12, Cas13) system, including a guide RNA (or a gRNA), such as, a single guide RNA (or a sgRNA, a chimeric RNA, an RNA chimera), a CRISPR RNA (crRNA), and a tracr RNA), or a precursor thereof; and,
   (2) a polynucleotide-packaging signal (PPS) capable of interacting, e.g., binding, directly or indirectly, to an PPS-interacting molecule that facilitates packaging of the polynucleotide sequence into the DNA virus viral particle;
optionally, a DNA sequence encoding or corresponding to said polynucleotide sequence, or a reverse complement of said DNA sequence, has reduced, diminished, or substantially no capacity of being packaged into the DNA virus viral particle (e.g., the DNA sequence or the reverse complement thereof lacks a DNA packaging signal such as a functional AAV ITR for AAV packaging).
2. The polynucleotide sequence of any preceding embodiment, wherein the DNA virus viral particle is an AAV viral particle or an oncolytic viral particle.
3. The polynucleotide sequence of any preceding embodiment, wherein the PPS is located at or near the 5′ end of the PSI, at or near the 3′ end of the PSI, or internal to the PSI (e.g., inside an intron of an mRNA).
4. The polynucleotide sequence of any preceding embodiment, comprising more than one (e.g., 1, 2, 3, or more) PPS that are identical or different.
5. The polynucleotide sequence of any preceding embodiment, wherein two or more (e.g., 3) of said more than one PPS are adjacent to each other, or are in tandem, via the same or different linkers.
6. The polynucleotide sequence of any preceding embodiment, comprising two or more PPS that are not adjacent to each other (e.g., one each located at or near one end of the polynucleotide sequence of interest (PSI)).
7. The polynucleotide sequence of any preceding embodiment, wherein the PPS comprises a transcribed modified AAV inverted terminal repeat (ITR), wherein said transcribed modified AAV ITR:
   (a) comprises a transcribed functional Rep-Binding Element (RBE), optionally further comprising a transcribed functional RBE′; and,
   (b) lacks either a transcribed terminal resolution site (TRS), or a transcribed reverse complement TRS (rcTRS), or both;
optionally, said transcribed modified AAV ITR further comprises a transcribed D region sequence (D sequence or D′ sequence); and/or optionally, the PPS-interacting molecule is Rep78, Rep68, Rep52, and/or Rep40.
8. The polynucleotide sequence of any preceding embodiment, wherein the transcribed modified AAV ITR is within the 3′ end 1000 nucleotides, 800 nucleotides, 500 nucleotides, 300 nucleotides, or 200 nucleotides of the RNA; optionally, the transcribed modified AAV ITR is 5′ to a polyA sequence, a polyA signal sequence (e.g., AAUAAA), or a sequence for RNA transcription termination (e.g., a histone downstream element).
9. The polynucleotide sequence of any preceding embodiment, wherein the transcribed modified AAV ITR is modified based on a transcribed wild-type flip or flop ITR; optionally, said wild-type flip or flop ITR is from AAV1, AAV2, AAV3A, AAV3B, AAV4, AAV5, AAV6, AAV7, AAVrh74, AAV8, AAV9, AAV10, AAV11, AAV12, or AAV13 (optionally, said wild-type flop ITR has the nucleotide sequence of SEQ ID NO: 1).
10. The polynucleotide sequence of any preceding embodiment, wherein the transcribed modified AAV ITR lacks both the transcribed TRS and the transcribed rcTRS.
11. The polynucleotide sequence of any preceding embodiment, wherein the transcribed modified AAV ITR comprises the transcribed D region sequence (optionally, said modified AAV ITR has the nucleotide sequence of SEQ ID NO: 3).
12. The polynucleotide sequence of any preceding embodiment, wherein the transcribed modified AAV ITR lacks the transcribed D region sequence (optionally, said modified AAV ITR has the nucleotide sequence of SEQ ID NO: 2).
13. The polynucleotide sequence of any preceding embodiment, further comprising a second transcribed modified AAV ITR having a second transcribed functional RBE sequence but lacking either a second transcribed TRS or a second transcribed rcTRS or both; optionally, said second transcribed modified AAV ITR further comprises a second transcribed D region sequence.
14. The polynucleotide sequence of any preceding embodiment, wherein the transcribed modified AAV ITR and the second transcribed modified AAV ITR are identical (or different).
15. The polynucleotide sequence of any preceding embodiment, wherein the transcribed modified AAV ITR, and the second transcribed modified AAV ITR (if present), comprise a deletion from, a mutation in, or an insertion into a corresponding transcribed wild-type AAV ITR D region sequence or a corresponding transcribed wild-type TRS/rcTRS.
16. The polynucleotide sequence of any preceding embodiment, wherein the second transcribed modified AAV ITR is within 5′ end 1000 nucleotides, 800 nucleotides, 500 nucleotides, 250 nucleotides, or 150 nucleotides of the polynucleotide sequence.
17. The polynucleotide sequence of any preceding embodiment, wherein the PPS comprises an MS2 sequence, an PP7 binding site, or a com binding site, and the PPS-interacting molecule comprises an PPS-interacting protein (PPSIP) capably of interacting, e.g., binding, directly or indirectly, to the PPS, such as a bacteriophage-derived MS2 coat protein (MCP) for an MS2 sequence, a PP7 bacteriophage coat protein (PCP) for an PP7 binding site, or a phage COM protein (COM) for a com binding site.
18. The polynucleotide sequence of any preceding embodiment, wherein the PPSIP is associated directly or indirectly with (e.g., fused to) a protein component of the viral packaging system for the DNA virus viral particle (such as Rep78 and/or Rep68 of adeno-associated virus 2 (AAV2), or assembly-activating protein (AAP)).
19. The polynucleotide sequence of any preceding embodiment, wherein the polynucleotide sequence comprises or preferably does not comprise a transcribed DNA packaging signal, for example, a transcribed wild-type AAV ITR sequence (e.g., the polynucleotide sequence comprises a transcribed modified AAV ITR sequence having an addition, a deletion, and/or a substitution of a nucleotide of a corresponding transcribed wild-type AAV ITR sequence to reduce the DNA packaging capability of the DNA virus viral particle).
20. The polynucleotide sequence of any preceding embodiment, further comprising:
   (1) a transcribed transcription enhancer;
   (2) a transcribed intron sequence or exon sequence (such as one for enhancing protein expression);
   (3) a 5′ UTR sequence;
   (4) a 3′ UTR sequence;
   (5) a polyA sequence, or a polyadenylation (polyA) signal sequence and optionally a GU-rich region downstream of the polyA signal sequence;
   (6) a posttranscriptional regulatory element or sequence, such as a transcribed Woodchuck Hepatitis Virus (WHP) Posttranscriptional Regulatory Element (WPRE) sequence; and/or,
   (7) a transcription termination sequence (such as a histone downstream element), optionally, the polynucleotide sequence comprises an PPS located 3′ to the posttranscriptional regulatory element or sequence, and 5′ to the polyA sequence or the polyA signal sequence.
21. The polynucleotide sequence of any preceding embodiment, comprising, in 5′ to 3′ orientation, the PSI, the optional transcribed WPRE sequence; the PPS (such as the transcribed modified AAV ITR, the MS2 sequence, the PP7 binding site, or the com binding site); and the polyA sequence or the polyA signal sequence.
22. The polynucleotide sequence of any preceding embodiment, wherein the GOI comprises a protein (e.g., a fluorescent protein, a therapeutic protein, an antigen protein, or a gene-editing protein such as a Cas protein, a ZFN protein, a TALEN protein), an enzyme (such as a Cre protein, or a CRISPR/Cas effector enzyme, e.g., Cas9, Cas12, Cas13, or a variant thereof), a structural protein, an mRNA, a non-coding RNA (ncRNA), an siRNA, a piRNA, a short hairpin RNA or shRNA, a microRNA (miRNA) or a precursor thereof (including pre-miRNA and pri-miRNA), a ribosomal RNA (rRNA), an antisense sequence or oligonucleotide (ASO), an RNA component of a CRISPR-Cas system, including a guide RNA (or a gRNA), such as, a single guide RNA (or a sgRNA, a chimeric RNA, an RNA chimera), a CRISPR RNA (crRNA), and a tracr RNA, a guide RNA or gRNA for a CRISPR/Cas effector enzyme, an rRNA, a tRNA, a snoRNA, a snRNA, an exRNA, a scaRNA, a lncRNA, a Xist, and a HOTAIR.
23. The polynucleotide sequence of any preceding embodiment, which is a single-stranded RNA less than about 8,900 nucleotides in length, less than about 8,000 nucleotides in length, less than about 7,000 nucleotides in length, less than about 6,000 nucleotides in length, less than about 5,200 nucleotides in length, less than about 4,000 nucleotides in length, less than about 3,000 nucleotides in length, less than about 2,000 nucleotides in length, about 4,700-5,200 nucleotides in length, about 4,700-5,000 nucleotide in length, about 4,700-4,800 nucleotides in length, or about 4,700 nucleotides in length.
24. A polynucleotide comprising a cassette encoding the polynucleotide sequence of any preceding embodiment; optionally, the polynucleotide is a DNA sequence (e.g., a DNA plasmid), optionally comprising a stuffer sequence in the backbone of the DNA plasmid, and/or optionally comprising no functional DNA packaging signal such as AAV ITR.
25. The polynucleotide of any preceding embodiment, further comprising a promoter operably linked to and driving the transcription of the polynucleotide sequence encoded by the cassette.
26. The polynucleotide of any preceding embodiment, wherein the promoter is a ubiquitous promoter.
27. The polynucleotide of any preceding embodiment, wherein the promoter is a tissue-specific promoter.
28. The polynucleotide of any preceding embodiment, wherein the promoter is a constitutive promoter.
29. The polynucleotide of any preceding embodiment, wherein the promoter is an inducible promoter.
30. The polynucleotide of any preceding embodiment, further comprising an enhancer that enhances the transcription of the polynucleotide sequence driven by the promoter.
31. A recombinant DNA virus viral particle comprising an polynucleotide genome (such as the polynucleotide sequence of any preceding embodiment or the polynucleotide sequence transcribed from the polynucleotide of any preceding embodiment) packaged within the protein shell (such as capsid) of a DNA virus (such as an AAV virus, or an oncolytic virus).
32. The recombinant DNA virus viral particle of any preceding embodiment, wherein the DNA virus is AAV, and the recombinant DNA virus viral particle is a recombinant polynucleotide adeno-associated virus (rPAAV) particle, comprising:
   (1) an AAV capsid; and,
   (2) the polynucleotide sequence of any preceding embodiment or the polynucleotide sequence transcribed from the polynucleotide of any preceding embodiment packaged within said AAV capsid.
33. The recombinant DNA virus viral particle of any preceding embodiment, wherein the AAV capsid comprises a capsid from an AAV of the serotype AAV1, AAV2, AAV3A, AAV3B, AAV4, AAV5, AAV6, AAV7, AAV8, AAV9, AAV10, AAV11, AAV12, AAV13, AAV-DJ, AAV PHP.eB, Anc80L65, Anc80L65AAP, AAVrh74, or 7m8.
34. A population of recombinant DNA virus viral particles (e.g., rPAAV particles) comprising a plurality of recombinant DNA virus viral particle (e.g., rPAAV particle) of any preceding embodiment, wherein at least about 10%, 20%, 30%, 40%, 50%, 60%, 70%, 80%, 90%, 95%, 96%, 97%, 98%, 99% or more of the recombinant DNA virus viral particles (e.g., rPAAV particles) within said population have the polynucleotide sequence of any preceding embodiment or the polynucleotide sequence transcribed from the polynucleotide of any preceding embodiment packaged therein.
35. A host cell comprising the polynucleotide sequence of any preceding embodiment, the polynucleotide of any preceding embodiment, the polynucleotide sequence transcribed from the polynucleotide of any preceding embodiment, the recombinant DNA virus viral particle (e.g., rPAAV particle) of any preceding embodiment, and/or the population of recombinant DNA virus viral particles (e.g., rPAAV particles) of any preceding embodiment.
36. The host cell of any preceding embodiment, further comprising a viral packaging system that facilitates packaging of the polynucleotide sequence of any preceding embodiment or the polynucleotide sequence transcribed from the polynucleotide of any preceding embodiment into the DNA virus viral particle.
37. The host cell of any preceding embodiment, wherein the viral packaging system comprises:
   (1) an AAV rep gene (e.g., coding sequence for Rep78, Rep68, Rep52, and/or Rep40) and an AAV cap gene (e.g., coding sequence for VP1, VP2, VP3, AAP, and/or MAAP), under the transcriptional control of one or more promoters that drive the transcription of said rep gene and cap gene, or the expression products thereof;
   (2) one or more coding sequences for one or more proteins required for AAV packaging, such as adenoviral E2A, E4, and VA genes, or said one or more proteins; and
   (3) the PPS-interacting molecule or a coding sequence thereof;
optionally, the capacity of the viral packaging system of packaging a DNA sequence into the DNA virus viral particle is reduced, diminished, or substantially eliminated by, for example, (1) removing a part or all of the DNA packaging signals such as AAV ITR on the polynucleotide encoding the polynucleotide sequence of any preceding embodiment or on the polynucleotide of any preceding embodiment, (2) modifying, e.g., mutating, said AAV rep gene, said AAV cap gene, and/or said one or more coding sequences for one or more proteins required for AAV packaging to reduce, diminish, or substantially eliminate the capacity of the respective translated protein to facilitate the packaging of the DNA sequence into the DNA virus viral particle (e.g., a Y156F mutation in the common sequence of Rep78 and Rep68 proteins, KDE-mu, or EKE-mu); and/or (3) enlarging the size of the polynucleotide encoding the polynucleotide sequence of any preceding embodiment or the polynucleotide of any preceding embodiment.
38. The host cell of any preceding embodiment, which is a mammalian cell (such as HEK293 cells) or an insect cell (such as Sf9 or Sf21 cells).
39. A method of generating the recombinant DNA virus viral particle (e.g., rPAAV particle) of any preceding embodiment or the population of recombinant DNA virus viral particles (e.g., rPAAV particles) of any preceding embodiment, the method comprising:
   a) culturing the host cell of any preceding embodiment for a sufficient time, and
   b) harvesting the recombinant DNA virus viral particle or the population of recombinant DNA virus viral particles.
40. The method of any preceding embodiment, further comprising isolating or purifying said recombinant DNA virus viral particle or said population of recombinant DNA virus viral particles.
41. A method of generating a recombinant DNA virus viral particle (e.g., rPAAV particle) or a population of recombinant DNA virus viral particles, the method comprising:
   a) contacting a viral packaging system (e.g., a AAV packaging system) with the polynucleotide sequence of any preceding embodiment or the polynucleotide sequence transcribed from the polynucleotide of any preceding embodiment for a period of time sufficient to produce the recombinant DNA virus viral particle or the population of recombinant DNA virus viral particles, and
   b) harvesting the recombinant DNA virus viral particle or the population of recombinant DNA virus viral particles; and, optionally,
   c) isolating or purifying the harvested recombinant DNA virus viral particle or population of recombinant DNA virus viral particles.
42. The method of any preceding embodiment, wherein the viral packaging system (e.g., a AAV packaging system) comprises:
   (1) one or more proteins for assembling the protein shell (e.g., VP1, VP2, and/or VP3 for assembling AAV capsid) of the DNA virus viral particle for packaging the polynucleotide sequence, or one or more coding sequences thereof;
   (2) one or more proteins (e.g., Rep78, Rep68, Rep52, and/or Rep40 for AAV packaging) for facilitating the assembling of the protein shell and/or the packaging of the polynucleotide sequence into the protein shell of the DNA virus viral particle, or one or more coding sequences thereof (e.g., adenoviral E2a, E4, and VA genes); and
(3) the PPS-interacting molecule or a coding sequence thereof;
optionally, the capacity of the viral packaging system of packaging a DNA sequence into the DNA virus viral particle is reduced, diminished, or substantially eliminated by, for example, (1) removing a part or all of the DNA packaging signals such as AAV ITR on the polynucleotide encoding the polynucleotide sequence of any preceding embodiment or on the polynucleotide of any preceding embodiment, (2) modifying, e.g., mutating, said AAV rep gene, said AAV cap gene, and/or said one or more coding sequences for one or more proteins required for AAV packaging to reduce, diminish, or substantially eliminate the capacity of the respective translated protein to facilitate the packaging of the DNA sequence into the DNA virus viral particle (e.g., a Y156F mutation in the common sequence of Rep78 and Rep68 proteins, KDE-mu, or EKE-mu); and/or (3) enlarging the size of the polynucleotide encoding the polynucleotide sequence of any preceding embodiment or the polynucleotide of any preceding embodiment.
43. A system of packaging the polynucleotide sequence of any preceding embodiment or the polynucleotide sequence transcribed from the polynucleotide of any preceding embodiment into a DNA virus viral particle, comprising:
   (1) one or more proteins for assembling the protein shell (e.g., VP1, VP2, and/or VP3 for assembling AAV capsid) of the DNA virus viral particle for packaging the polynucleotide sequence, or one or more coding sequences thereof;
   (2) one or more proteins (e.g., Rep78, Rep68, Rep52, and/or Rep40 for AAV packaging) for facilitating the assembling of the protein shell and/or the packaging of the polynucleotide sequence into the protein shell of the DNA virus viral particle, or one or more coding sequences thereof (e.g., adenoviral E2a, E4, and VA genes); and
   (3) the PPS-interacting molecule or a coding sequence thereof;
optionally, the capacity of the viral packaging system of packaging a DNA sequence into the DNA virus viral particle is reduced, diminished, or substantially eliminated by, for example, (1) removing a part or all of the DNA packaging signals such as AAV ITR on the polynucleotide encoding the polynucleotide sequence of any preceding embodiment or on the polynucleotide of any preceding embodiment, (2) modifying, e.g., mutating, said AAV rep gene, said AAV cap gene, and/or said one or more coding sequences for one or more proteins required for AAV packaging to reduce, diminish, or substantially eliminate the capacity of the respective translated protein to facilitate the packaging of the DNA sequence into the DNA virus viral particle (e.g., a Y156F mutation in the common sequence of Rep78 and Rep68 proteins, KDE-mu, or EKE-mu); and/or (3) enlarging the size of the polynucleotide encoding the polynucleotide sequence of any preceding embodiment or the polynucleotide of any preceding embodiment.
44. A method of delivering a gene of interest (GOI) into a cell, a plant, or an animal, the method comprising contacting the cell, the plant, or the animal with the recombinant DNA virus viral particle (e.g., rPAAV particle) of any preceding embodiment, the population of the recombinant DNA virus viral particles (e.g., rPAAV particles) of any preceding embodiment, or the recombinant DNA virus viral particle (e.g., rPAAV particle) or the population of the recombinant DNA virus viral particles (e.g., rPAAV particles) produced by the method of any preceding embodiment, wherein said GOI is encoded by said polynucleotide sequence (of any preceding embodiment).
45. A method of delivering an polynucleotide sequence of interest (PSI) into a cell, a plant, or an animal, the method comprising contacting the cell, the plant, or the animal with the recombinant DNA virus viral particle (e.g., rPAAV particle) of any preceding embodiment, the population of the recombinant DNA virus viral particles (e.g., rPAAV particles) of any preceding embodiment, or the recombinant DNA virus viral particle (e.g., rPAAV particle) or the population of the recombinant DNA virus viral particles (e.g., rPAAV particles) produced by the method of any preceding embodiment.
46. A method of diagnosing, preventing, or treating a disease or disorder in a subject in need thereof, comprising administrating to the subject a therapeutically effective amount or dose of the population of the recombinant DNA virus viral particles (e.g., rPAAV particles) of any preceding embodiment or produced by the method of any preceding embodiment.
47. Use of the recombinant DNA virus viral particle (e.g., rPAAV particle) of any preceding embodiment, the population of the recombinant DNA virus viral particles (e.g., rPAAV particles) of any preceding embodiment, or the recombinant DNA virus viral particle (e.g., rPAAV particle) or the population of the recombinant DNA virus viral particles (e.g., rPAAV particles) produced by the method of any preceding embodiment in the manufacture of a medicament for diagnosing, preventing, or treating a disease or disorder in a subject in need thereof.

### EXAMPLES

The examples herein below are provided to illustrate several exemplary embodiments of the invention, and are not limiting in any respect.

### Example 1 Efficient Packaging of RNA into RAAV Viral Particles

This example demonstrates that RNA vector genome can be efficiently packaged into AAV viral capsids, especially with the modified/recombinant RNA designed for direct packaging into AAV capsids.

First, it was surprisingly shown that the AAV packaging signal-ITR (DNA), when transcribed (as RNA), was able to facilitate the packaging of the RNA sequence of the invention (e.g., the rRAAV vector genome RNA) into AAV particles, especially when it is presented in certain configurations (e.g., when the transcribed modified AAV ITR sequence is close to the 3′ end of the transcribed RNA sequence of the invention).

Specifically, wild-type and modified AAV ITR sequences (DNA) from the ends of the AAV vector genome were moved into their respective transgene expression cassettes, to ensure that all the transgene transcripts (RNA's) contain a candidate packaging signal. In order to block the production of conventional AAV vectors with ssDNA genomes during RAAV production, optimized ITRs (dITR and dITR-D) were used instead of wild type ITR (Table 2).

TABLE 2
Nucleic Acid Sequences of Tested ITRs

| ITR Names | Nucleic Acid (DNA) Sequences |
|---|---|
| wild type | TTGGCCACTCCCTCTCTGCGCGCTCGCTCGCTCACTGAGGCCGCCCGGG |
| ITR2 | CAAAGCCCGGGCGTCGGGCGACCTTTGGTCGCCCGGCCTCAGTGAGCGA |
| (Flop) | GCGAGCGCGCAGAGAGGGAGTGGCCAACTCCATCACTAGGGGTTCT |
| | (SEQ ID NO: 1) |
| dITR | TCCCTCTCTGCGCGCTCGCTCGCTCACTGAGGCCGCCCGGGCAAAGCCC |
| | GGGCGTCGGGCGACCTTTGGTCGCCCGGCCTCAGTGAGCGAGCGAGCGC |
| | GCAGAGAGGGAGTGG (SEQ ID NO: 2) |
| dITR-D | TCCCTCTCTGCGCGCTCGCTCGCTCACTGAGGCCGCCCGGGCAAAGCCC |
| | GGGCGTCGGGCGACCTTTGGTCGCCCGGCCTCAGTGAGCGAGCGAGCGC |
| | GCAGAGAGGGAGTGGACTAGCTCCATCACTAGGGGTTCCT |
| | (SEQ ID NO: 3) |

Specifically, in the wild-type AAV2 ITR (ITR2) sequence in the Flop configuration, the TRS (“TTGGC”) is at the 5′ end of the ITR, and its reverse complement sequence GCCAA is double underlined. This sequence can be cloned into the coding plasmid in either direction (i.e., either the sequence shown as SEQ ID NO: 1, or its reverse complement sequence, can be used as template to transcribe the RNA sequence of the invention). In the experiments herein, the wild-type AAV2 ITR sequence was cloned in an orientation such that the transcribed RNA had the same sequence as SEQ ID NO: 1 (or SEQ ID NO: 2 or 3 below) except that T's were replaced by U's in the transcribed RNA. Regardless, upon transcription of either this sequence or its reverse transcript, the resulting transcribed RNA of the wild-type ITR2 comprises the palindromic transcribed RBE (shaded in grey). In the experiment herein, the transcribed RNA comprises a transcribed wild-type AAV2 ITR that is equivalent to SEQ ID NO: 1, except that all T's were replaced by U's. If the reverse complement sequence of SEQ ID NO: 1 were used as the DNA template, the transcribed RNA would comprise a transcribed TRS (UUGGC) encoded by GCCAA. The transcribed TRS is located between the transcribed RBE and the transcribed D sequence.

One of the modified ITR sequence is “delta ITR” (or “dITR” for short), which is defective because the dITR lacks both the D region sequence (bold italic), the TRS at the 5′ end, and the reverse complement TRS sequence (“GCCAA”) except for the first G. Upon transcription of this sequence, the transcribed RNA of the dITR also comprises the palindromic transcribed RBE (shaded in grey), and a transcribed defective ITR that lacks a transcribed TRS (UUGGC) encoded by GCCAA. In this experiment, however, the reverse complement sequence of SEQ ID NO: 2 served as the DNA template, such that the transcribed RNA comprises a transcribed modified AAV2 ITR (transcribed dITR) having the same sequence as SEQ ID NO: 2, except that all T's were replaced by U's.

Another one of the modified ITR sequence is “dITR-D,” which is also defective because it retains its D sequence (“CTCCATCACTAGGGGTTCCT,” SEQ ID NO: 4) but lacks the 5′ end TRS (TTGGC). In addition, only the first G in the reverse complement TRS (GCCAA) is retained in dITR-D, and the remaining CCAA sequence is replaced by an unrelated ACTAG sequence. In this experiment, the reverse complement sequence of SEQ ID NO: 3 served as the DNA template, such that the transcribed RNA comprises a transcribed modified AAV2 ITR (transcribed dITR-D) having the same sequence as SEQ ID NO: 3, except that all T's were replaced by U's.

Note that both the dITR and dITR-D sequences retain the shaded palindromic RBE sequence SEQ ID NOS 103-104, respectively (CTGCGCGCTCGCTCGCTCACTG . . . CAGTGAGCGAGCGAGCGCGCAG), and their respective transcribed modified ITR's also have the RBE sequence.

Such optimized ITR coding sequences (DNA) were inserted into two positions of the tdTomato expression cassette-one located in-between the promoter and the tdTomato coding sequence, and the other located in-between the Woodchuck Hepatitis Virus (WHP) Posttranscriptional Regulatory Element (WPRE) and the SV40 polyA signal.

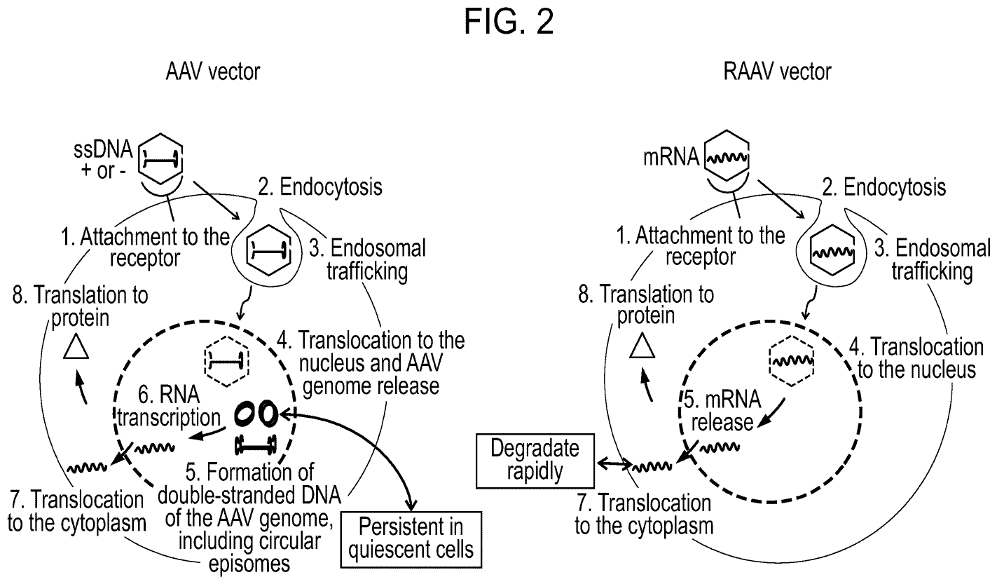

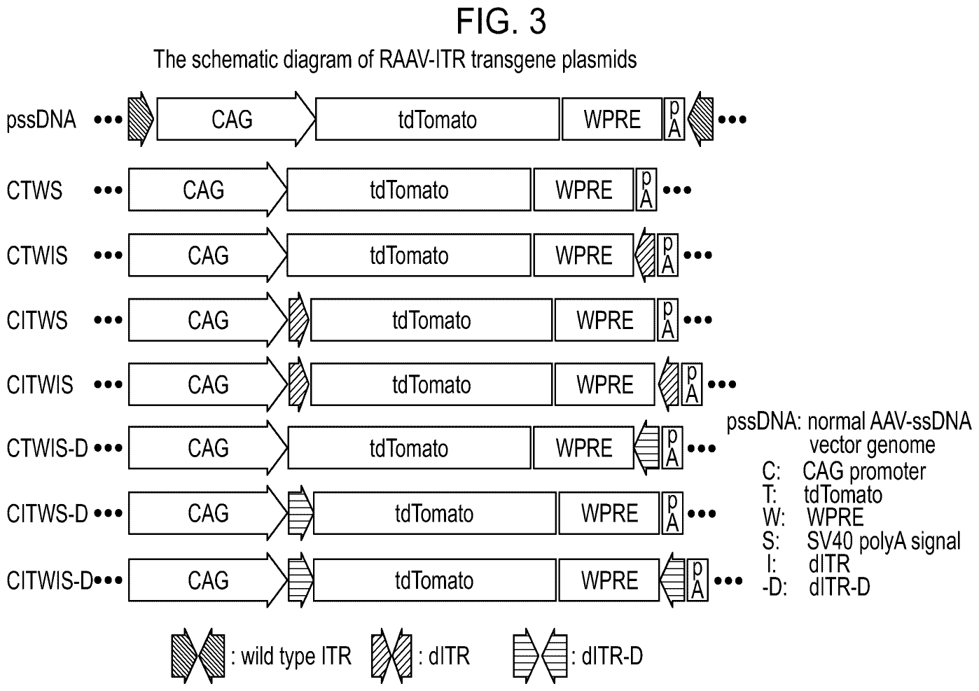

Based on the sequences, numbers and positions of the optimized-ITRs used, a series of the various ITR-based RAAV vectors were constructed (see FIG. 3).

A conventional AAV vector with a ssDNA vector genome and no ITR sequences at either end (“CTWS,” which stands for the sequence elements CAG promoter, tdTomato transgene, WPRE sequence, and SV40 polyA signal sequence) were used as a control. For this experiment, AAV serotype DJ was chosen because of its excellent transduction efficiency in cultured cells used. AAV-DJ is a synthetic serotype with a chimeric capsid of AAV-2, 8, and 9. It contains a heparin-binding domain in its capsid, which may efficiently transduce a broad range of cell types and escape from immune neutralization (Grimm et al., J. Virol. 82:5887-5911, 2008).

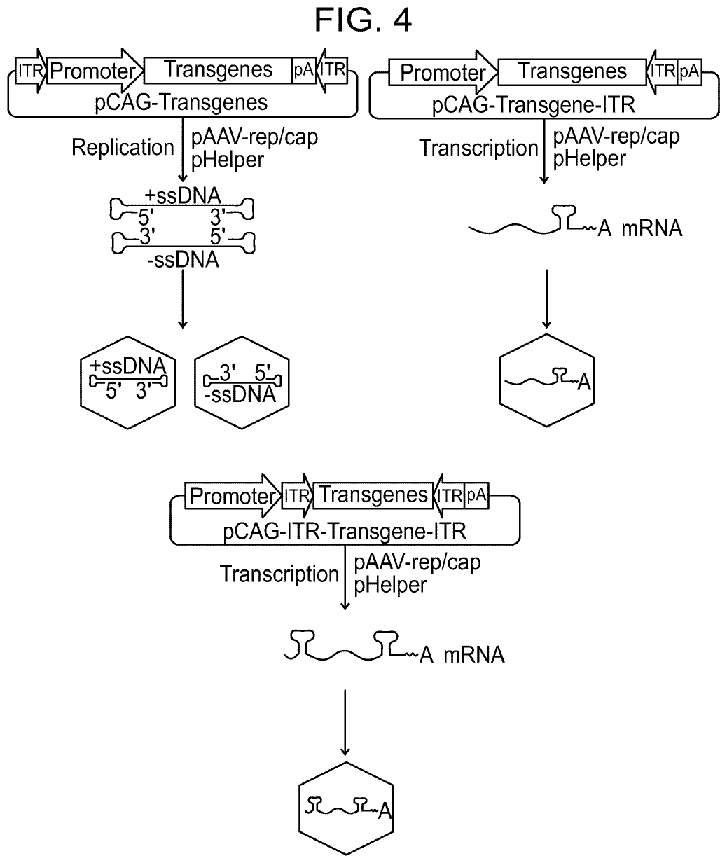

Both the various RAAV-ITR viral particles and the control viral particles were generated by using the triple-plasmid transfection system (FIG. 4).

In particular, the RAAV vectors were generated by co-transfecting tansgene plasmid, packaging plasmid and helper plasmid (weight ratio was 1:1:2) into HEK293T cells. The HEK293T cells were cultured in competent DMEM medium, and the cells were plated 24 hrs before transfection. Before transfection, the culture medium was replaced with fresh DMEM containing 2% FBS. PEI-MAX was used as the transfection reagent. The supernatant was then collected at Day 2 and Day 5 post transfection, and transfected cells were harvested on Day 5. RAAV vectors were purified by using iodixanol density gradient ultracentrifugation.

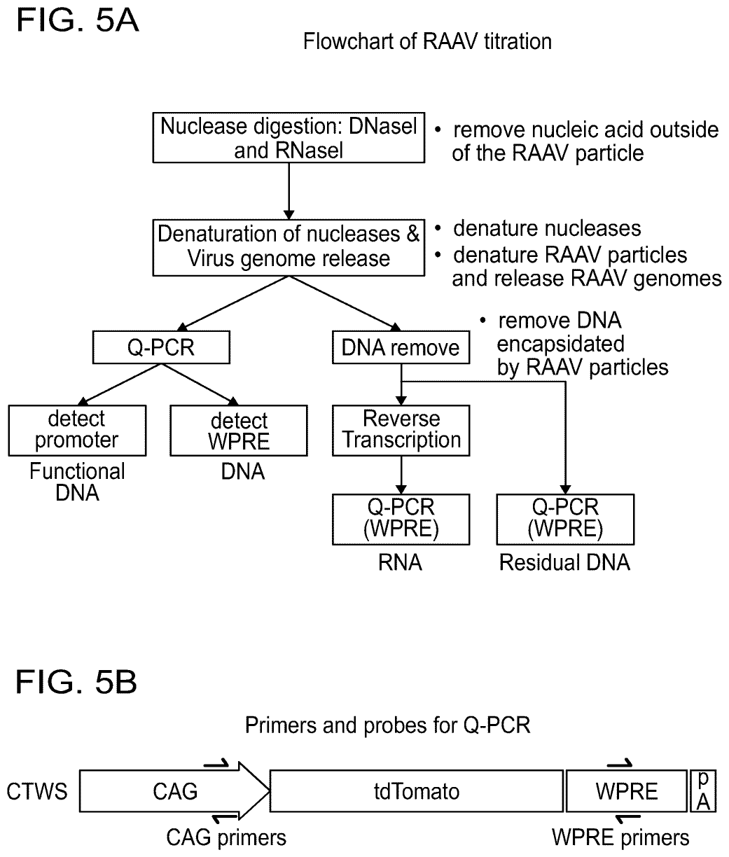

Viral titers (DNA titer and RNA titer) were determined by Q-PCR and Reverse transcription-PCR (RT-PCT), respectively, using the procedure in FIG. 5A.

Briefly, the harvested and purified RAAV viral particles were first subjected to DNase I and RNase I treatment at 37° C. for 2 hours to remove all nucleic acids outside the protein shells of the viral particles. Next, the nucleases were denatured at 100° C. for about 30 min, before the RAAV viral particles were denatured and ruptured to release the RAAV nucleic acid contents for further analysis.

Q-PCR was used to analyze the nuclease-resistant products, in order to titrate the DNA vector genome encapsidated within the RAAV viral particles, Specifically, a primer pair specific for the promoter sequence was used in one set of Q-PCR to detect/quantitate any functional DNA, and a primer pair specific for the WPRE sequence was used in another set of Q-PCR to detect/quantitate any DNA vector genome encapsidated in the RAAV viral particles. Sec FIG. 5B.

Meanwhile, in another sample, any RAAV-encapsidated DNA was first removed by DNA removal through Dnase I digestion, before the remaining RNA was subjected to reverse-transcription, and the resulting cDNA was used as Q-PCR templates for detection/quantitation of WPRE sequences transcribed into RNA. To detect/quantitate any residue DNA that may be present after incomplete DNA removal, a sample after the DNA removal step was directly amplified using Q-PCR to detect any WPRE (DNA) sequences that might be present in that sample. See FIG. 5A.

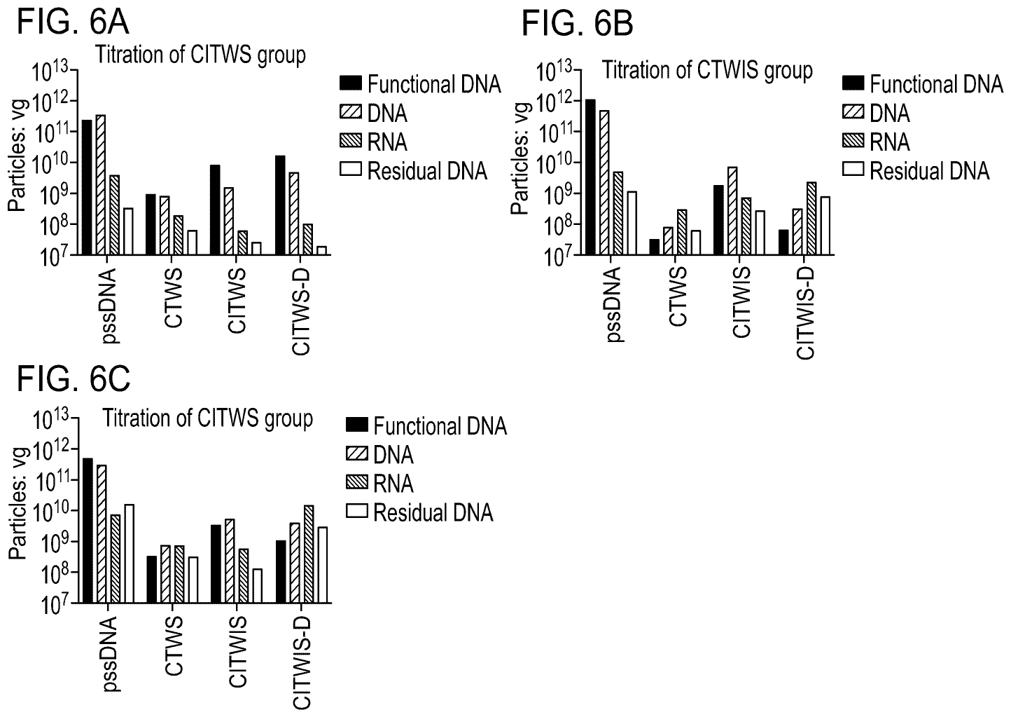

To test the packaging efficiency of the CITWS construct (see FIG. 6A), when the conventional pssDNA construct (with wild-type ITR sequences on both ends) was used to generate viral particles, the vast majority of the viral particles contained functional DNA vector genome with promoter sequences and the WPRE sequences. Very occasionally (two orders of magnitude, or about 1% of the time), RNA vector genome was also packaged into viral particles (see the bar labelled as “RNA” which is about 2 orders of magnitude lower than the bar labelled as “DNA” and “Functional DNA”). Residual DNA is one order of magnitude less than the packaged RNA vector genome.

Meanwhile, removing the ITR sequences from both ends of the AAV vector genome essentially abolished packaging—the CTWS construct (ITR-frec) in FIG. 6A produced 2-2.5 orders of magnitude less of packaged DNA, and even less RNA.

Adding back only one optimized ITR sequence (either the dITR or dITR-D sequence), between the 3′ of the promoter and 5′ to the GOI coding sequence, did not appear to enhance RNA packaging compared to the CTWS control, though DNA packaging seemed to have slightly improved. See FIG. 6A.

Interestingly, a very different result was achieved in FIG. 6B, in which the CTWIS constructs were tested. Specifically, essentially the same results were obtained regarding packaging the pssDNA constructs (compared FIGS. 6A and 6B)-most packaged viral particles contained DNA (99% or more) and negligible amount (1% or less) of RNA. However, including an optimized ITR sequence (dITR) between the 3′ end of the WPRE sequence and the 5′ end of the polyA sequence significantly reduced or even reversed the packaging efficiency difference between DNA and RNA. This effect is even more prominent when the dITR-D sequence was used, when the vast majority of the packaged nucleic acids are RNA (1-2 orders of magnitude over packaged DNA).

Essentially the same results were obtained if one additional (i.e., a second) optimized ITR sequence was inserted between the promoter and the GOI coding sequence in the CITWIS constructs. Sec FIG. 6C.

These results demonstrated that, optimized ITRs (dITR and dITR-D) impaired the replication of the conventional AAV vectors, thereby leading to a reduction of DNA packaging into the RAAV viral particle.

Compared to the control vector CTWS (ITR-free) and the RAAV-dITR vectors, RAAV vectors with the dITR-D optimized ITR seem to have a better ability to encapsidate the transcribed mRNAs directly into RAAV particles, especially when the dITR-D ITR is located downstream of the mRNA coding sequence and WPRE sequence (for example, just 5′ to the polyA signal). See FIGS. 6A-6C. In contrast, a dITR-D sequence located upstream of the mRNA coding sequence (e.g., right after the promoter sequence in the expression construct) hardly facilitated direct mRNA packaging into the RAAV viral particles. Meanwhile, if another dITR-D sequence was inserted downstream of the mRNA coding sequence (e.g., right 5′ to the polyA sequence), packaging of the resulting RAAV mRNAs was similarly highly increased (FIG. 6C).

In conclusion, CITWIS-D, which harbors dITR-D signals at both ends of its mRNA genome, has the best ability to encapsidate specific mRNAs, despite the fact that its yield (mRNA-harbouring particles) is 20-fold lower than the yield of conventional AAV vectors with ssDNA vector genomes (pssAAV group). Unlike the conventional AAV vectors, RAAV vector CITWIS-D have an impaired DNA packaging, with its DNA-carrying particles only taking up about 20% or less of the RAAV vector stock, and the percentage of the particles harbouring functional DNAs is even lower (e.g., less than 10%) (FIGS. 6A-6C).

Since the AAV packaging capacity is limited (<4700 nt), the undesired RAAV DNA packaging could be reduced by enlarging the size of the transgene plasmids, and functional DNA packaging could be further reduced by increasing the length of the transgene cassette, for example, by inserting cis-acting elements (such as, enhancer, intron, etc.) or non-functional stuffer sequence into the cassette.

### Example 2 The RAAV Viral Particles are Functional

This example demonstrates that the subject RAAV-dITR-D vectors are infectious and can be used as gene delivery vectors.

The same volume of purified RAAV-dITR-D (CITWIS-D) vectors were used to infect 2×10⁵ HEK293T cells in vitro, at the same MOI of about 50,000 (the MOI of the CITWIS-D vectors was calculated based on the sum of the number of DNA-particles and mRNA-particles).

Specifically, HEK293T cells were plated into 24-well plates about 24 hrs before infection. RAAV vectors were then mixed completely with 1 mL of DMEM (containing 2% FBS). The culture medium of the cells was then removed, and the cells were incubated with mixed RAAV vectors overnight. Fluorescence photos were taken 3 and 5 days post infection.

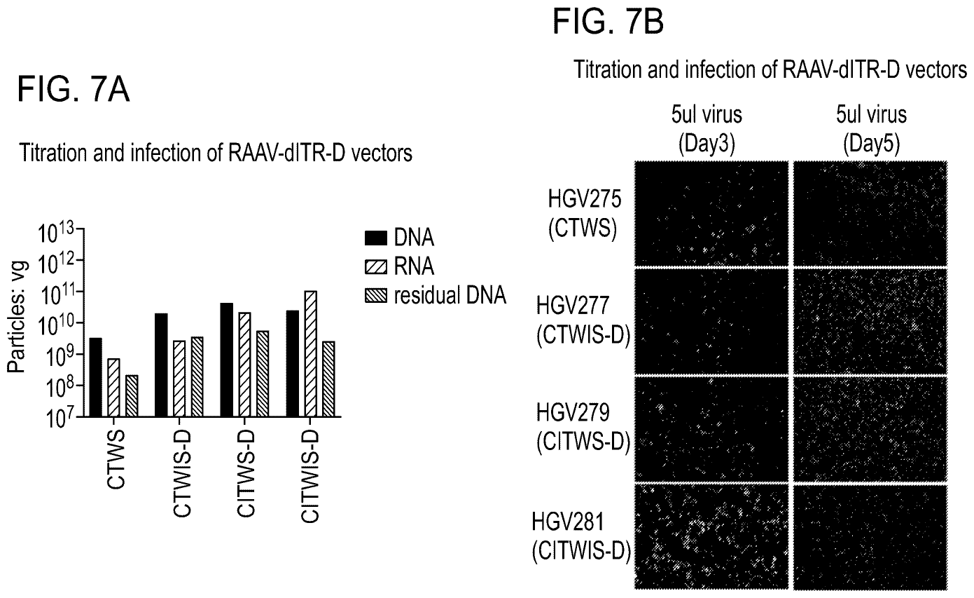

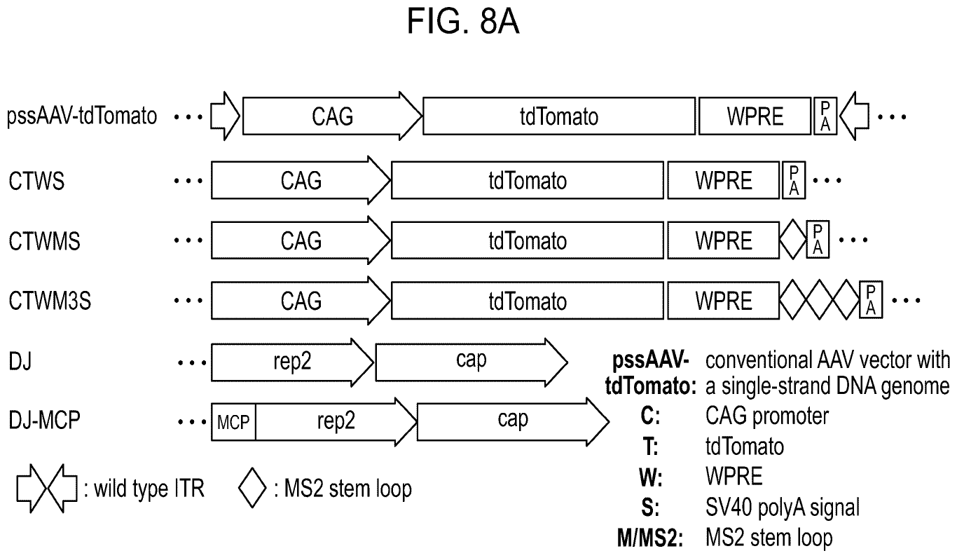

The results showed that, tdTomato expression by the CITWIS-D vector was quicker than that of the other vectors, but the expression was down-regulated rapidly too (see FIG. 7B). This quick expression and degradation phenomenon may be due to the short lifetime of its mRNA genome (FIG. 7B).

It is interesting to note that the CTWS construct without any ITR sequences were apparently packaged to some degree, though the precise mechanism underlying this packaging remains unclear. At least two possibilities can explain the packaging of mRNA vector genome when the CTWS vectors were used: overexpressed cellular mRNAs could be packaged into the RAAV vectors non-specifically, or CTWS mRNA might have some RNA structures that interact with Rep2 or Cap-DJ. Meanwhile, CTWS DNA packaging may be due to the small size of the plasmid CTWS, and DNA packaging may be reduced by increasing the size of the CTWS plasmid.

### Example 3 Efficient Packaging of RNAs into RAAV Particles

This Example demonstrated that RNA genomes can be efficiently packaged into AAV capsids, especially with the modified/recombinant RNA constructs designed herein for direct packaging into AAV capsids to produce RAAV particles. 1. Design

The inventors have designed a strategy to utilize the strong interaction between bacteriophage-derived MS2 coat protein (MCP) and its recognizing stem loop MS2 as a novel packaging signal for packaging heterologous RNA into DNA virus viral particles.

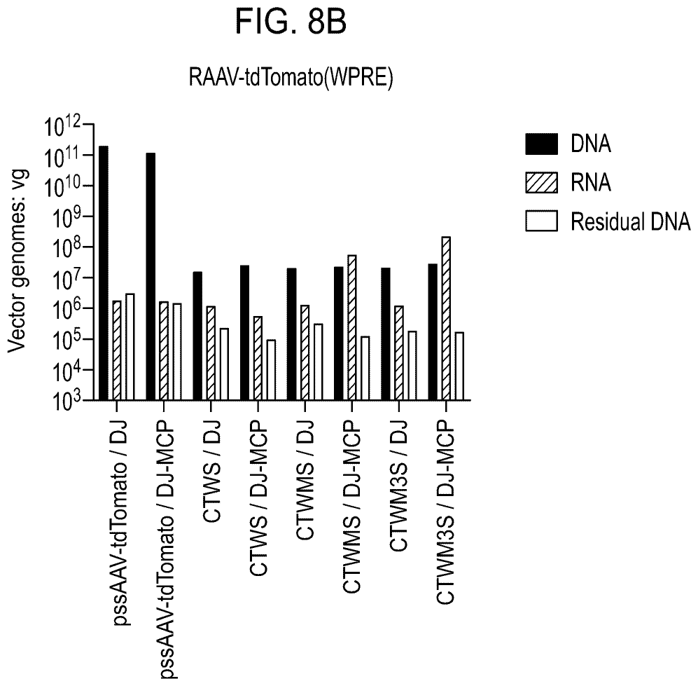

First, in order to inhibit/reduce the production of conventional AAV particles with packaged ssDNA genomes during RAAV production, the conventional AAV packaging signals-ITRs-were removed. Instead, one copy or three copies of RNA packaging signals (RPS), MS2 stem loop (or “MS2” for short, its RNA sequence is set forth in the sequence tables below), were inserted into a tdTomato expression cassette, in-between the Woodchuck Hepatitis Virus (WHP) Posttranscriptional Regulatory Element (WPRE) and SV40 polyA signal, in order to ensure that all the transcribed mRNAs would have the RPS, so as to be recognized by the binding protein, bacteriophage-derived MS2 coat protein (or “MCP” for short, its amino acid sequence is set forth in the sequence tables below), corresponding to the MS2 (FIG. 8A).

Since AAV Rep proteins are non-structural proteins, and they conventionally serve as bridges between the ITRs of the ssDNA genomes and the AAV capsids during AAV packaging, MCP was fused to the N-terminus of Rep78 protein and Rep68 protein from AAV2 (Rep 68 is a C-terminal truncation of Rep 78, the amino acid sequences of the two fusions are set forth in the sequence tables below). The ability of these MCP-Rep78/68 fusions to interact with the MS2 sequence harbored inside the RNA genomes, and to facilitate the packaging of the RNA genomes into the AAV capsids was verified.

The conventional AAV vector pssAAV-tdTomato (with two wild type functional ITRs) and CTWS without functional wild-type ITRs (“CTWS,” which stands for the sequence elements CAG promoter, tdTomato transgene, WPRE sequence, and SV40 polyA signal sequence) were used as controls (FIG. 8A). In this Example, AAV serotype DJ

(“AAV-DJ” or “DJ”) was selected for use because of its excellent transduction efficiency in the cultured cells, HEK293T cells, used in this Example. AAV-DJ is a synthetic serotype with a chimeric capsid of AAV-2, 8, and 9.

### 2. RAAV Packaging and Production

Both the RAAV and control AAV particles herein were produced by using conventional triple-plasmid transfection system mutatis mutandis, by co-transfecting the respective transgene plasmids, packaging plasmids, and helper plasmids in a weight ratio of 1:1:2 into HEK293T cells.

Specifically, the HEK293T cells were cultured in competent DMEM medium, and the cells were plated 24 hrs before transfection. Shortly before transfection, the culture medium was replaced with fresh DMEM containing 2% FBS. PEI-MAX was used as the transfection reagent. Transcription of the RPS-harboring transgene plasmids to generate the RNA genomes to be packaged occurred after the transfection into the infected cells. The supernatant was then collected at Day 2 and Day 5 post transfection, and the transfected cells were harvested on Day 5. The RAAV and control AAV particles were purified by using iodixanol density gradient ultracentrifugation.

### 3. Detection of Packaged Genomes

The purified RAAV and control AAV particles were first subjected to nuclease treatment, including DNase I and RNase I treatment, at 37° C. for 2 hours, in order to remove possibly existed nucleic acids outside the viral particles. Next, the nucleases and the RAAV or control AAV particles were denatured by proteinase K/SDS digestion at 65° C. for about 3 hrs to rupture the viral particles in order to release the genomes packaged therein. The nuclease-resistant polynucleotides containing the released viral genomes were then extracted and purified by phenol/chloroform extraction.

To detect the DNA genome titer of the control AAV and RAAV particles, Q-PCR was used to analyze the nuclease-resistant polynucleotides directly. A pair of WPRE primers (as set forth in the sequence tables below) specific for the WPRE sequence on the viral genomes was used in the Q-PCR to detect and quantitate any DNA genomes encapsidated in the control AAV or RAAV particles.

To detect the RNA genome titer of the control AAV and RAAV particles, any control AAV—or RAAV-encapsidated DNA genomes was removed by DNA removal through Dnase I digestion, before the encapsidated RNA genomes were subjected to reverse-transcription, and the resulting cDNA was used as Q-PCR templates for the detection and quantitation of

WPRE sequences with the same pair of WPRE primers aforementioned. To detect and quantitate any potentially residual DNA that might be present due to the incomplete DNA removal, a sample after the DNA removal step was directly amplified (without reverse-transcription) using Q-PCR to detect the WPRE (DNA) sequence with the same pair of WPRE primers aforementioned, which was also used for all the other PCR reactions specific for WPRE sequence.

### 4. Comparison of Packaging Efficiency

When the conventional transgene plasmid containing the pssAAV-tdTomato construct (with wild-type ITRs on both ends) and the conventional packaging plasmid for AAV-DJ were used to produce the control AAV particles, the vast majority of the particles contained DNA genomes with the WPRE sequences. Very occasionally, RNA genomes were also packaged into the particles (see the bar labelled as “RNA,” which was about 5 orders of magnitude lower than the bar labelled as “DNA”). The presence of residual DNA is comparable to that of RNA, which may be due to the inefficient digestion of packaged DNA genomes with DNase I before reverse-transcription.

Interestingly, when the recombinant packaging plasmid DJ-MCP (MCP fused to the N-terminus of Rep78 and 68 proteins) was used instead of DJ, the packaging of DNA genomes was slightly reduced (about 0.5 order of magnitude lower), but the pattern of viral genome distribution was almost the same, which DNA packaging was about 5 orders of magnitude higher than RNA packaging. This result indicated that fusing MCP to the N-terminus of Rep78/68 proteins did not significantly impair their natural functions (FIG. 8B).

Meanwhile, removing the ITRs from both ends of the pssAAV-tdTomato construct, leading to the CTWS construct, significantly abolished DNA packaging. The CTWS construct (ITR-free) produced about 4 orders of magnitude less of packaged DNA, and even less packaged RNA, no matter which packaging plasmid (DJ or DJ-MCP) was used.

Further, by adding one or three copies of the RPS (MS2) between the 3′ of the WPRE and 5′ of the SV40 polyA signal on the viral genomes without ITRs, the RAAV transgene plasmids, CTWMS and CTWM3S respectively, were obtained. In the absence of MCP, the CTWMS and CTWM3S constructs could barely be encapsidated as DNA or RNA genomes, just like the genome distribution pattern of CTWS. Surprisingly, the use of DJ-MCP as the packaging plasmid instead of DJ significantly reversed the packaging efficiency difference between DNA and RNA genomes, and the vast majority of the packaged genomes were RNA.

Compared to CTWS/DJ-MCP, the numbers of the packaged RNA genomes of CTWMS/DJ-MCP and CTWM3S/DJ-MCP were about 100—and 400-fold higher, respectively, whereas no significant difference was observed in the DNA-packaged number of the three. This result suggested that the MCP-Rep78/68 fusions could recognize the RPS, MS2, embedded in the RNA transcripts of the CTWMS and CTWM3S plasmids specifically and facilitate their RNA packaging into RAAV particles, and three copies of RPS in the CTWM3S construct provided an even better RNA packaging efficiency than one copy (FIG. 8B).

In conclusion, the introduction of the MS2/MCP pair into conventional AAV packaging system enabled the packaging of MS2-harboring RNA genomes into AAV particles in the presence of the MCP-Rep78/68 fusions, leading to the generation of RAAV particles. The undesired DNA packaging only constituted about 10% of the whole viral particle population produced by using CTWM3S/DJ-MCP.

In other words, the artificial/heterologous RNA packaging signal (RPS)—the MS2 sequence—can be used with its cognate binding protein MCP to replace the native DNA virus packaging signal pair (i.e., ITR and Rep), in order to dramatically boost the packaging efficiency of RNA into an otherwise DNA virus, while suppressing its inherent packaging of DNA into the same DNA virus.

### Example 4 Enlarged Plasmid Backbone Reduced Undesired DNA Packaging of RAAV

This example demonstrates that increasing the backbone size of the AAV transgene plasmid by inserting a stuffer sequence into the backbone of the plasmid could reduce undesired DNA packaging into RAAV particles.

Although the CTWMS and CTWM3S constructs for RAAV particles in Example 3 did not have ITRs and no reverse packaging existed in the RAAV production, it was speculated that the relative small size (5˜6 kb) of the RAAV transgene plasmids might still lead to undesired DNA packaging.

Therefore, a 3266 bp non-coding sequence (stuffer sequence; see the sequence tables below) was inserted upstream of the tdTomato expression cassette of CTWM3S in order to increase the backbone length of the CTWM3S transgene plasmid, and the resulting construct was named L-CTWM3S. The schematic diagram of the plasmid is shown in FIG. 9A.

The conventional AAV genome construct, pssAAV-tdTomato, and the RAAV genome construct, CTWM3S, used in Example 3 were used as controls herein. In the same way as in Example 3, RAAV particles were produced by co-transfecting CTWM3S or L-CTWM3S transgene plasmid together with the packaging plasmid DJ-MCP and the helper plasmid into HEK293T cells, and the resulting RAAV particles were purified and the viral genomes were quantified. The same pair of WPRE primers were used to detect and quantitate any DNA and RNA genomes encapsidated in the AAV and RAAV particles, and an additional pair of CAG primers specific for the CAG promoter sequence in the viral genomes were used in Q-PCR to detect and quantitate any functional DNA (meaning DNA containing the CAG promoter sequence and able to express functional transgene proteins).

It was noted that the packaged RNA genomes cannot be detected with the CAG primers since they did not contain the CAG promoter, and the RNA columns on the drawings with CAG primers represented background RNA signals (see FIG. 9B).

Surprisingly, the DNA genome titer of the L-CTWM3S group was about 2 times lower than that of the CTWM3S group, no matter which pair of primers was used in Q-PCR (see FIGS. 9B and 9C). The RNA genome titers of the CTWM3S and L-CTWM3S groups were substantially equivalent (see FIG. 9C). Since there is no CAG promoter sequence in the transcribed RNA from the CTWM3S and L-CTWM3S transgene plasmids, the packaged RNA genomes could only be detected with the pair of WPRE primers (FIG. 9C).

In conclusion, increasing the backbone length of the transgene plasmid could reduce undesired DNA packaging of the RAAV particles without interfering with their RNA packaging, showing that the deconstruction of the DNA packaging system and the establishment of the RNA packaging system in AAV particles are two separate lines, and this long-stuffer sequence was used to the RAAV transgene plasmids in the subsequent Examples.

### Example 5 Using RAAV-MS2/MCP System for Additional Transgenes

In order to verify the general applicability of the RAAV-MS2/MCP system to additional transgenes, and to ensure that the observed RNA packaging is not a mere artifact associated with the reporter gene used, a series of AAV and RAAV transgene plasmids containing a Cre recombinase expression cassette were generated.

Conventional pssAAV-Cre (with the tdTomate coding sequence in the pssAAV-tdTomato construct in FIG. 8A replaced with a Cre coding sequence) and a corresponding L-CCWS construct (with the tdTomate coding sequence in CTWS in FIG. 8A replaced with the Cre coding sequence and inserted with the stuffer sequence in Example 4) were used as controls, with the second C standing for the Cre recombinase transgene. The L-CTWM3S construct in Example 4 was also redesigned as L-CCWM3S construct, after replacing the tdTomate coding sequence with the Cre coding sequence.

The Cre transgene plasmids were co-transfected with the packing plasmid DJ or DJ-MCP, and together with the helper plasmid in HEK293T cells, respectively, to produce AAV and RAAV particles. The resulting viral particles were purified, and the viral genomes were quantified as described in Example 3.

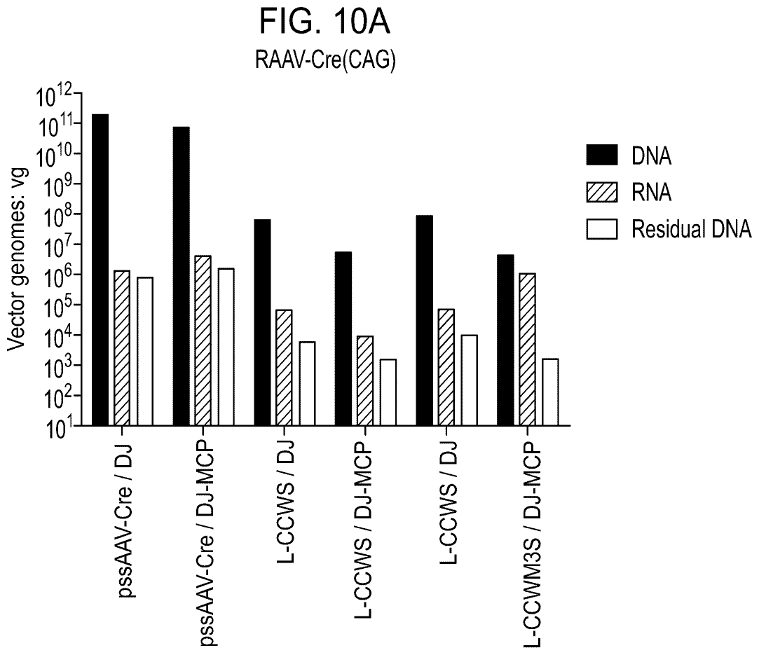

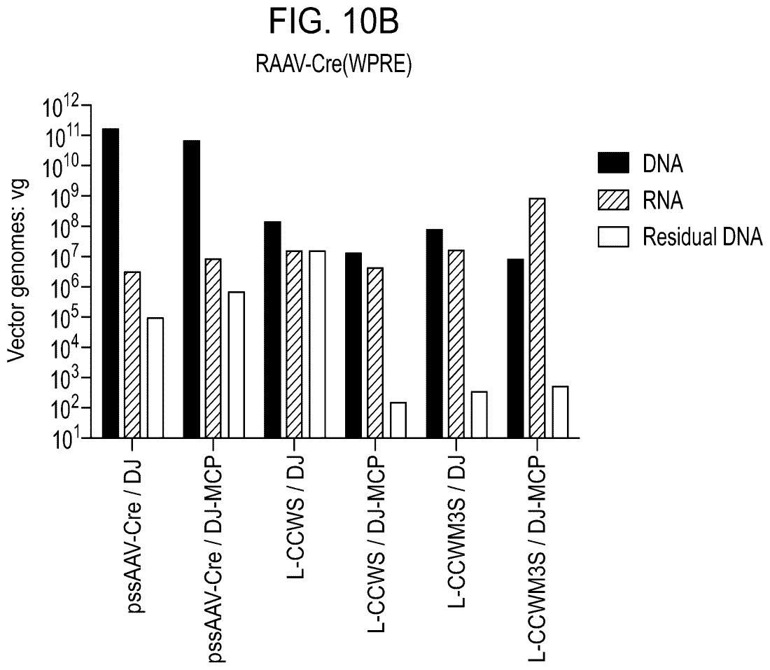

The same viral genome distribution results as AAV-tdTomato and RAAV-tdTomato were achieved for AAV-Cre and RAAV-Cre. For pssAAV-Cre, most viral particles contained DNA genomes, and the DNA genome titer was about 4˜5 orders of magnitude higher than that of RNA genomes. For L-CCWS, DNA and RNA genomes were barely encapsidated, due to the lack of both DNA and RNA packaging signals. For L-CCWM3S, RNA packaging was significantly improved with DJ-MCP by about 200-fold compared to that of L-CCWS/DJ-MCP, and the undesired DNA-harboring viral particles only constituted about 1% of the whole viral particle population (FIGS. 10A and 10B).

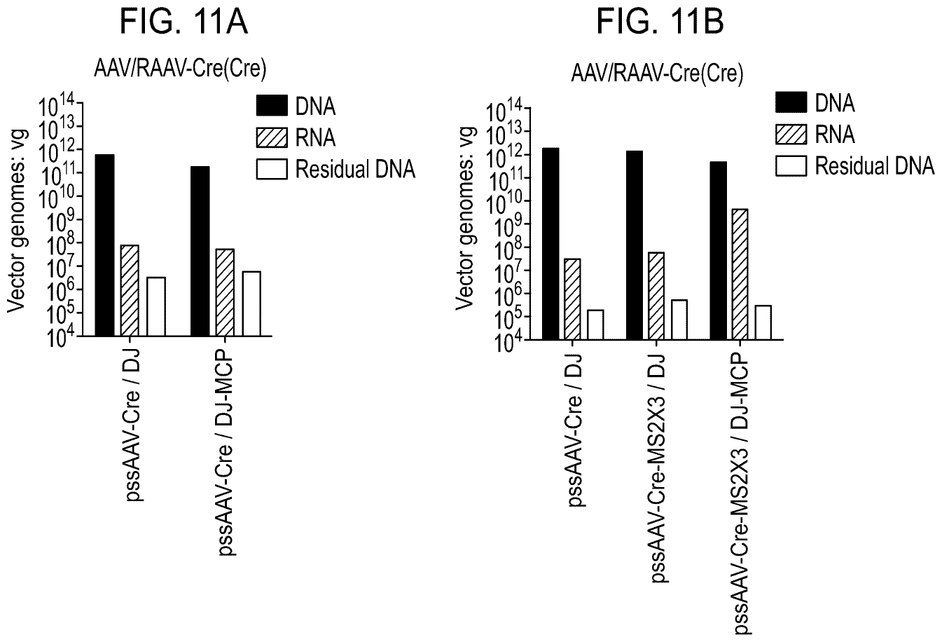

Since the DJ-MCP fusion not only assisted the RNA packaging but also retained the DNA packaging ability, its performance was also assessed in a construct containing both DNA packaging signals (ITRs) and RNA packaging signals (3 copies of MS2) designated as pssAAV-Cre-MS2X3, which was constructed by inserting 3 copies of MS2 in-between WPRE and SV40 polyA of the pssAAV-Cre construct. The results showed that in the absence of MCP, most viral particles contained packaged DNA genomes, and only a negligible amount of RNA genomes was packaged with or without the RNA packaging signals. The RNA packaging was remarkably improved when DJ-MCP was used instead of DJ as the packaging plasmid in combination with the RNA binding signals, and surprisingly, the increased RNA packaging did not significantly interfere with the DNA packaging of the pssAAV-Cre-MS2X3 construct (FIG. 11B). In another view, it was also demonstrated that the introduction of the MS2/MCP pair could significantly increase RNA packaging even without removing the DNA packaging signal-ITRs, indicating that the deconstruction of the DNA packaging system and the establishment of the RNA packaging system in AAV particles are two separate lines and the removal of ITRs is not the essential basis for the increased RNA packaging by the introduction of RPS/RBP pair.

In conclusion, the subject RAAV-MS2/MCP system can be applied to any transgenes in general, such as the Cre recombinase as demonstrated above. Interestingly, the RAAV-Cre construct produced a better yield than that of the RAAV-tdTomato construct. While not wishing to be bound by any particular theory, this may be due to the simpler secondary structure of the Cre mRNA comparing to the tdTomato mRNA, based on online RNA secondary structure prediction such as that found at rna.tbi.univie.ac.at/cgi-bin/RNA WebSuite/RNAfold.cgi.

### Example 6 Optimization of RAAV Production System and Identification of the Properties of Optimized RAAV Particles

The endonuclease activity of the Rep68 and Rep78 proteins (Rep68/78) is essential for the DNA genome replication during the conventional DNA packaging of AAV particles. Without the functional trs-endonuclease, the newly-synthesized viral ssDNA cannot be released for packaging. It was investigated in this Example whether the undesired DNA packaging of RAAV particles could be further reduced by disrupting the activity of the trs-endonuclease.

To investigate this, three trs-endonuclease negative mutants were constructed, namely DJ-MCP (Y156F, wherein the Y156F mutation was in the common sequence of Rep68 and Rep78 proteins, i.e., Rep68-Y156F and Rep78-Y156F), DJ-MCP (KDE-mu) and DJ-MCP (EKE-mu) (see the sequence tables below).

The DNA and RNA packaging efficiencies for DJ-MCP (Y156F) were firstly assessed with the transgene plasmid, pssAAV-Cre-MS2X3 containing both the DNA and RNA packaging signals, as described in Example 5. DJ and DJ-MCP were set as packaging plasmid controls. Viral particles were produced, purified, and titrated as described in Example 3.

The results demonstrated that the Y156F mutation in DJ-MCP significantly reduced the ITR-mediated DNA packaging for pssAAV-Cre-MS2X3 without interfering with the RNA packaging.

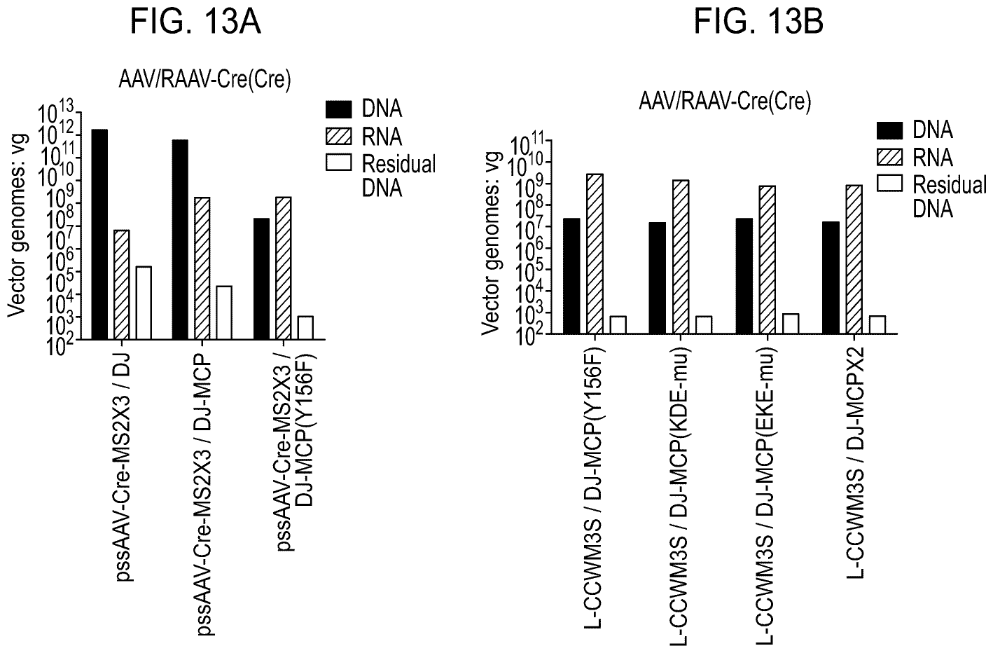

Therefore, in addition to the removal of DNA packaging signals ITR as shown in the previous Examples, modifying, for example, mutating the functional proteins like Rep78/68 proteins participating in the DNA packaging process to weaken or eliminate their DNA-packaging-associated functions could serve as another strategy to reduce or inhibit the conventional DNA packaging of AAV particles (FIG. 13A).

Then, L-CCWM3S in Example 5 was used as a RAAV transgene plasmid to provide viral genomes in place of pssAAV-Cre-MS2X3. The DJ-MCP, which was trs-endonuclease positive, was used as a control against DJ-MCP (Y156F). Viral particles were produced, purified, and titrated as described in Example 3. Two pairs of primers were used here to titrate viral genomes, one pair for targeting WPRE sequence as above and one pair (see sequence tables below) for targeting the 5′ terminus of the Cre coding sequence.

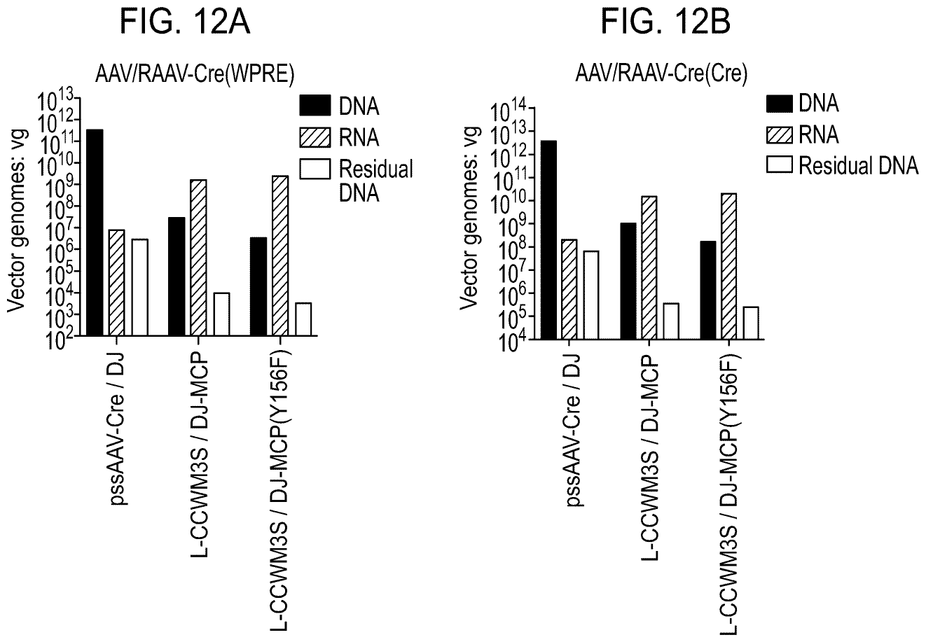

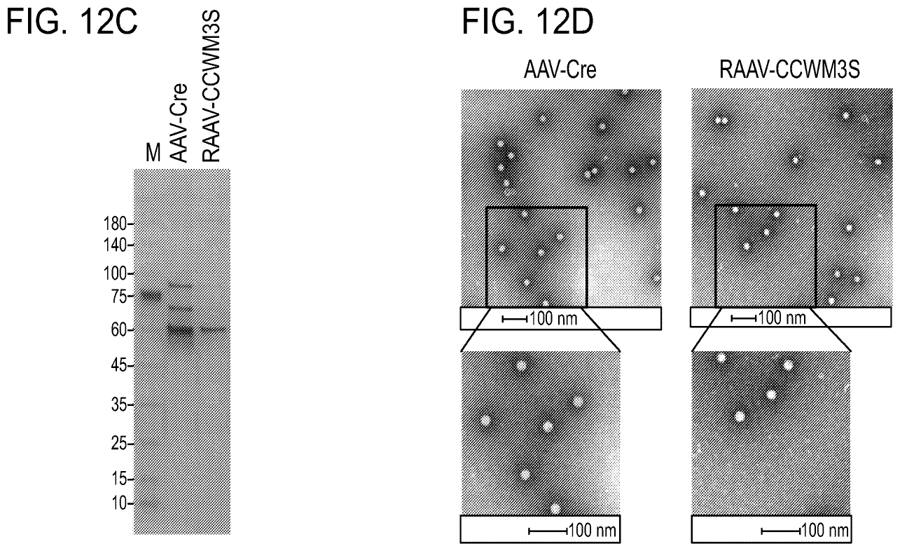

The results showed that the Y156F mutation in Rep78/68 protein not only reduced the undesired DNA packaging by about 10-fold, but also increased the desired RNA packaging by about 50%. The patterns of the packaging efficiency difference between the packaged DNA and RNA genomes were substantially the same for both pairs of primers (i.e., WPRE primer pairs and Cre primer pairs) used in Q-PCR (FIGS. 12A-12B).

Two other trs-endonuclease mutants, DJ-MCP (KDE-mu) and DJ-MCP (EKE-mu), were also tested, and were demonstrated to have the same ability to reduce undesired DNA packaging as DJ-MCP (Y156F), but only DJ-MCP (Y156F) showed improved RNA packaging (FIG. 13B).

It was further demonstrated that fusing two copies of MCP to the N-terminus of Rep 78/68 proteins (MCPx2-Rep78 and MCPx2-Rep68) could also achieve the result of reducing undesired DNA packaging (FIG. 13B).

The compositions of the AAV and RAAV particles were analyzed by silver-stained SDS-PAGE, and the RAAV capsids were also composed of three VP proteins (VP1, VP2 and VP3) with a similar VP1/2/3 ratio to conventional AAV particles (FIG. 12C).

In order to analyze the morphology of the RAAV particles, 10 μL of the purified AAV and RAAV particles were placed on a 300 μm carbon-over-Pioloform-coated copper grid and incubated for 2 min. at room temperature. The excess of the sample was blotted with filter paper and immediately replaced by 10 μL of staining agent (3% phosphotungstic acid), which was allowed to settle for 2 min. and then blotted again. Visualization of the samples was performed by using a Talos L120C transmission electron microscope. The RAAV particles were morphologically similar to the conventional AAV vectors, where full viral particles encapsidating genomes were viewed as 25-nm solid spheres, and empty viral particles without genomes encapsidated were 25-nm donut-like structures (FIG. 12D).

In conclusion, the mutation of functional proteins including Rep proteins participating in the DNA packaging process of AAV production to weaken or eliminate their DNA-packaging-associated functions in combination with the removal of DNA packaging signals, ITRs, is an optimized strategy to reduce or inhibit undesired DNA packaging of RAAV particles. The produced RAAV particles have similar compositions and morphology to the conventional AAV particles.

### Example 7 RAAV Vectors Expressing Functional Proteins

This Example demonstrated that the subject RAAV vectors were infectious and can be used as gene delivery vectors.

Cre-loxP system, a highly sensitive system, was used for investigating the infectivity of the inventive RAAV vectors. Mouse embryonic fibroblast (MEF) cells isolated from homo-Ai9 (bearing loxP-tdTomato-reporter system) mice were incubated with the purified AAV (pssAAV-Cre/DJ) or RAAV (L-CCWM3S/DJ-MCP (Y156F)) vectors in Example 5 overnight, and Multiplicity of Infections (MOIs) (the number of virions added per cell during infection) were set, including 7 MOIs for conventional AAV vectors and 3 MOIs for RAAV vectors. The dominant genome titer quantified by detecting Cre coding sequence with the 5′-terminus Cre primers aforementioned was used as the infection titer. In other words, the DNA genome titer was used for the conventional AAV vectors, and the RNA genome titer was used for the RAAV vectors.

Specifically, Ai9-MEF cells were plated into 48-well plates in about 5×10⁴ cells per well about 24 hrs before infection. AAV vectors or RAAV vectors were mixed completely with 0.5 mL of DMEM containing 2% FBS. The culture medium of the plated cells was removed, and then the cells were incubated with mixed AAV or RAAV vectors overnight at 37° C. The infected cells were collected at different time points and subjected to RNA and DNA analysis. A pair of primers targeting the 5′-terminus of Cre-coding sequence as aforementioned was used for detecting the specific Cre-coding DNA and mRNA derived from the vectors. Fluorescence photos were taken daily post infection (p.i), and the fluorescence-positive cells were quantified by flow cytometry 5 days p.i.

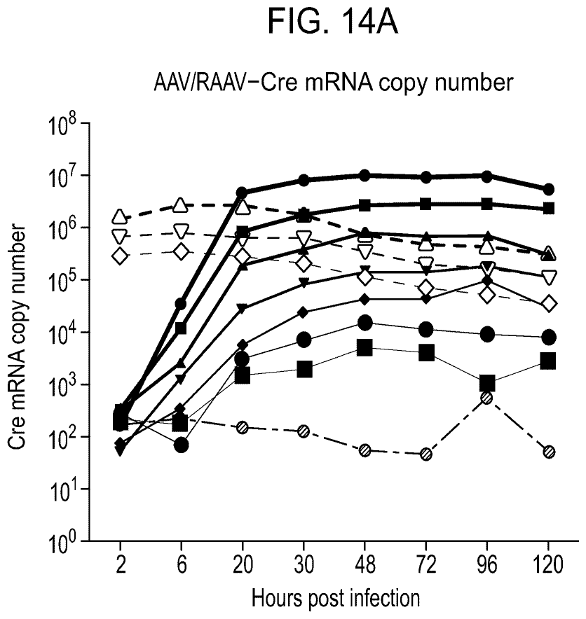

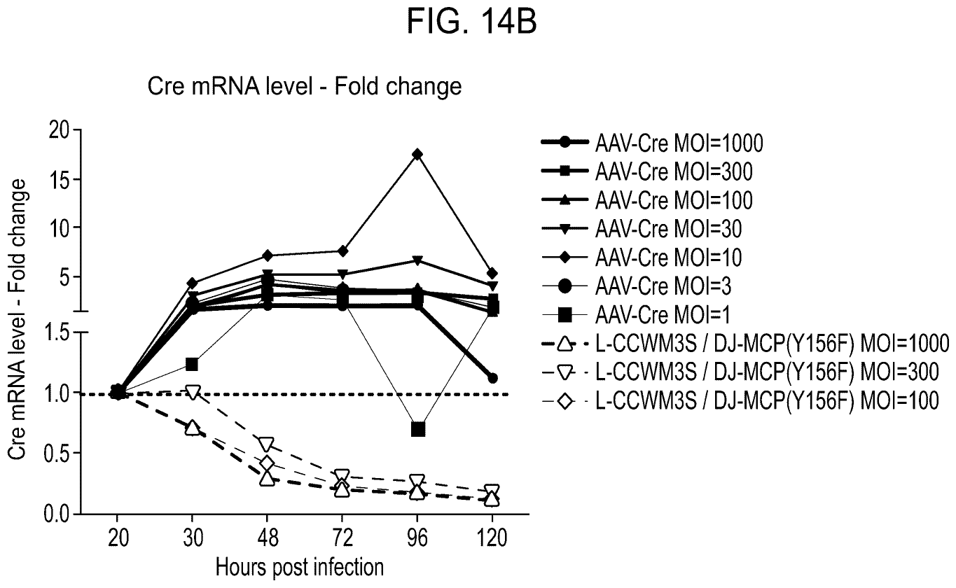

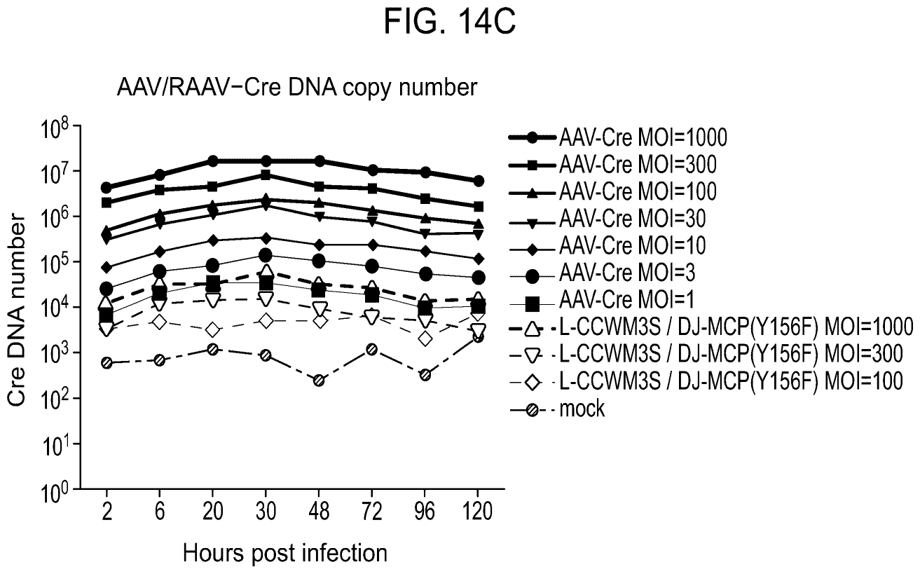

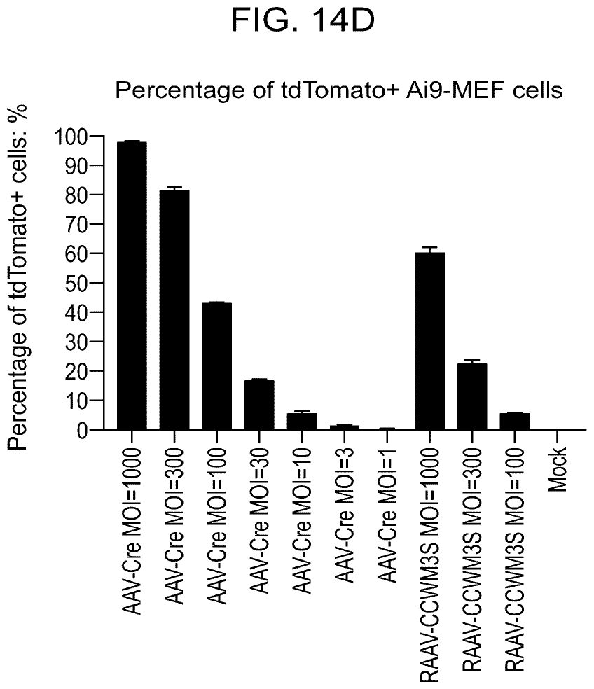

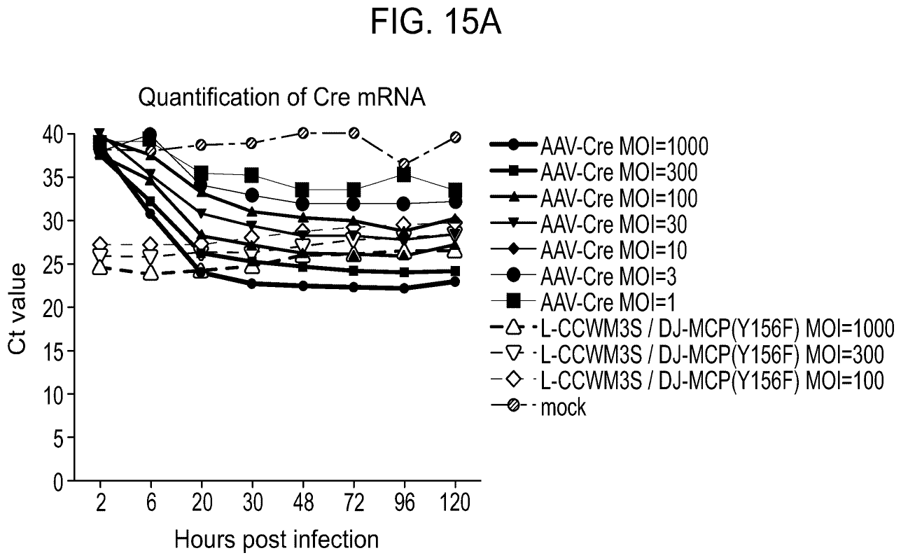

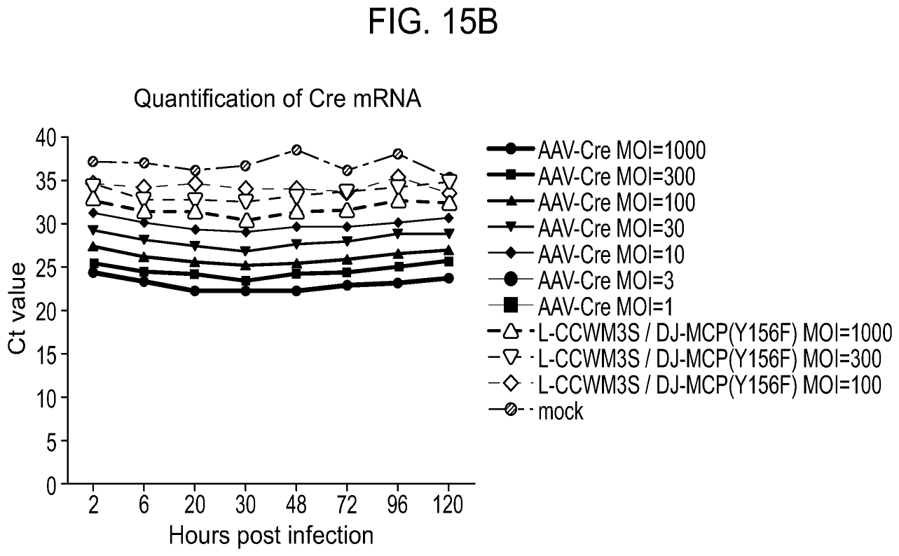

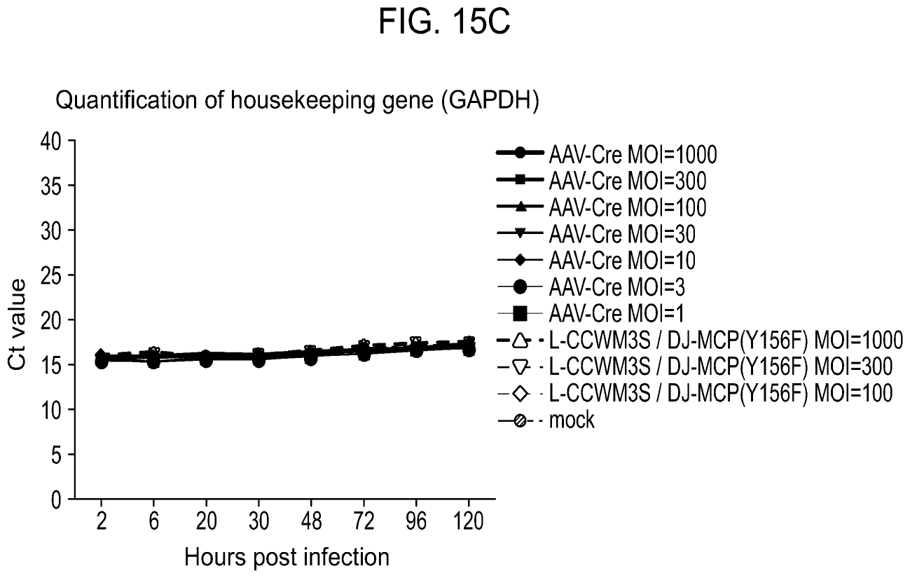

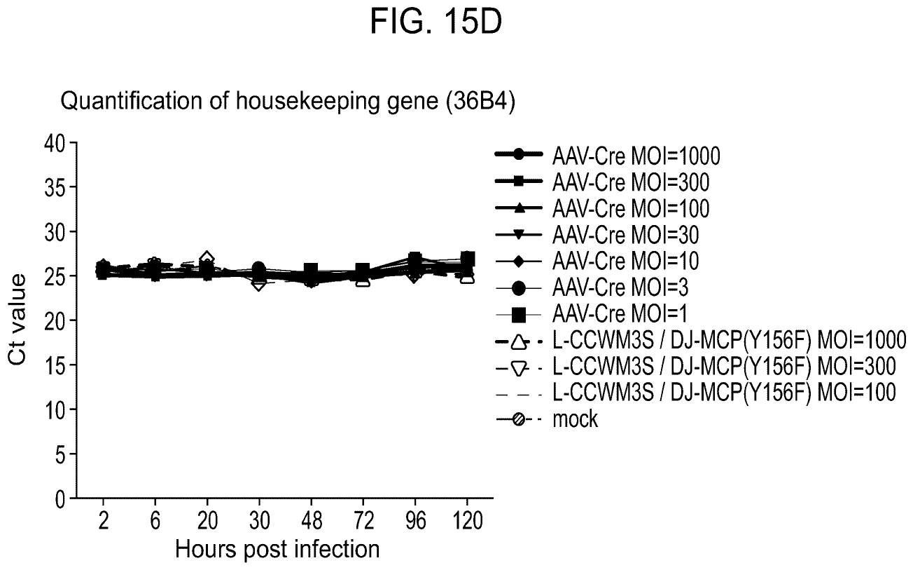

The mRNA analysis results showed that the specific mRNA was detected in the RAAV-infected cells as early as 2 hrs p.i, peaked at 6 hrs p.i, and then decreased. In the cells infected with the conventional AAV vectors, no apparent transcription was detected at 2 hrs p.i, but a rapid increase of transcribed mRNA was observed from 6 hrs to 20 hrs p.i, reaching a plateau at 30 hrs. In contrast to the results for the RAAV vectors, the mRNA level in the cells infected with the conventional AAV vectors did not decrease after reaching the plateau. The copy numbers of the Cre mRNAs were positively correlated with MOIs in all the samples (FIGS. 14A-14B and FIG. 15A). Meanwhile, mGAPDH mRNA was tested as a reference transcript (housekeeping gene), and as expected, there was no difference in mGAPDH mRNA levels among all the samples (FIG. 15C).

The DNA results were quite different from the mRNA results. Conventional AAV and RAAV vectors had substantially the same DNA copy number pattern, the majority of DNA genomes was detected in the infected cells as early as 2 hrs post infection, and then a slight increase followed from 2 hrs to 20 hrs p.i, which was very similar to the trend of the mRNA levels in the RAAV-infected cells. After that, the DNA level reached a plateau or descended slowly. The copy numbers of the Cre DNA were also positively correlated with MOIs in all the samples, but much lower numbers of the Cre DNA were detected in the RAAV-infected cells (the DNA copy number of RAAV-CCWM3S MOI=100 or 300 group was less than that of AAV-Cre MOI=1 group, and the DNA copy number of RAAV-CCWM3S MOI=1000 group was less than that of AAV-Cre MOI=3 group) (FIG. 14C and FIG. 15B). Similarly, the DNA level of another housekeeping gene 36B4 was quantified as a reference gene, and as expected, no obvious difference in the DNA levels was observed among all the samples (FIG. 15D).

Successful infection of AAV-Cre or RAAV-CCWM3S vectors would lead to the expression of functional Cre recombinase and rescue the tdTomato expression in Ai9-MEF cells, and thus the fluorescence photos of the infected cells were taken and analyzed to assess the infectivity of the viral vectors by counting cells emitting tdTomato red fluorescence. The results showed that the number of fluorescence-positive cells generated by RAAV-CCWM3S was comparable to that generated by AAV-Cre with a 10-fold lower MOI (FIG. 14D). The lower fluorescent intensity of tdTomato in the RAAV-infected cells was possibly due to the short lifetime of the Cre mRNAs delivered thereinto and the inability of the limited amount of the translated Cre recombinase to rescue both of the two copies of tdTomato expression cassettes in the homo-Ai9-MEF cells (FIG. 16).

By comparing the results of the DNA titer and cytometric data, it was indicated that the majority of the tdTomato red fluorescence signals in the RAAV-CCWM3S infected cells were generated by the Cre mRNA-harboring RAAV particles.

In conclusion, the inventive RAAV vector could deliver functional Cre mRNAs into cells and express functional Cre recombinase.

### Example 8 In vitro Transient Transfer of Functional Gene by RAAV Particles into Cells

To determine the exact lifespan of the Cre protein produced via AAV-Cre or RAAV-CCWM3S delivery as in Example 7, Ai9-MEF cells were seeded 24 hrs before infection at a cell confluence of 5×10⁴ cells per well, and then incubated with AAV-Cre (MOI=300) or RAAV-CCWM3S (MOI=10,000) vectors overnight as described above. After infection, cells were collected at several time points, and fluorescence photos were taken prior to the cell collection.

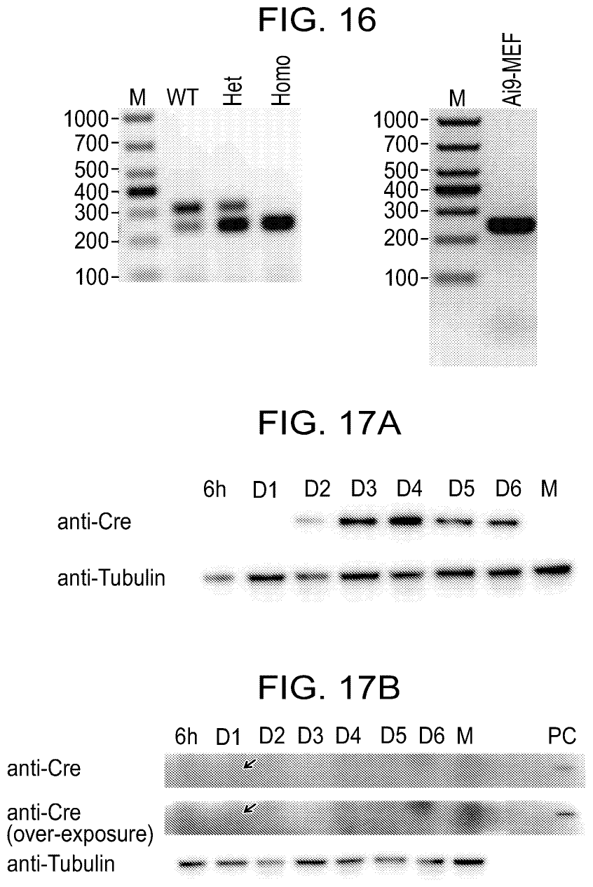

For AAV-Cre, Cre expression increased during the first 4 days, but then decreased. By contrast, a small amount of Cre was detected at as early as about 24 hrs after RAAV-CCWM3S transfer and disappeared after Day 2. This quick expression and degradation phenomenon may be due to the instant appearance and short lifetime of the delivered functional Cre mRNA (FIGS. 17A and 17B).

### Example 9 In vivo Transient Transfer of Functional Gene by RAAV Particles into Ai9-mouse

This example demonstrates that the RAAV particles can be used as a tool for in vivo gene delivery and to express the functional Cre recombinase transiently.

To investigate the infectivity of RAAV particles in vivo, Ai9-Mice (2.5-4 months old) were anesthetized and injected with 1 μL AAV-Cre (pssAAV-Cre/DJ) (high dose: 1E9 vg/mouse; low dose: 3E6 vg/mouse) or 1 μL RAAV-Cre (L-CCWM3S/DJ-MCP (Y156F)) (1E9 vg/mouse) into the right hippocampus according to the following coordinates: anteroposterior (A/P)=−1.7 mm, mediolateral (M/L)=−1.0 mm, dorsoventral (D/V)=−2.1 mm. Also, AAV capsid-DJ was used in this assay as a control.

Six weeks after AAV or RAAV injection, mice were anesthetized and transcardially perfused with PBS at room temperature at pH 7.4 and then with freshly prepared, ice-cold 4% paraformaldehyde (PFA) in phosphate buffers (PB). The brains were post-fixed in 4% PFA overnight. The fixed brains were embedded with OCT for frozen section after dehydration. Brains were sectioned in 20 μm thickness using a freezing microtome (Leica CM1950), and the sections were mounted to slides directly. The slides were baked at 60° C. for 1-2 hours followed by blocking with 5% BSA serum in PBS for 1 h. Subsequently, the slides were incubated with the primary antibody against Cre (10536; Cell Signaling Technology; 1:800 dilution) in 5% BSA in PBS (0.1% Triton-X) overnight at room temperature. After five washes with PBS, the slides were incubated in 1% BSA in PBS containing secondary antibody against the primary antibody and DAPI (D3571, Invitrogen). The secondary antibody used was Alexa Fluor 488 donkey anti-rabbit IgG (711-545-152, Jackson ImmunoResearch) (at 1:1000 dilution). Images were acquired with Nikon C2si+ Confocal Microscope.

The acquired images showing fluorescence from tdTomato expression system demonstrated that RAAV-Cre infected the cells in Ai9-mice hippocampus and rescued the expression of tdTomato. The number of the infected cells in the RAAV-Cre group was less than that of the AAV-Cre group at the same dose. However, the RAAV-Cre infection generated much more tdTomato positive cells relative to the low-dose group (30-fold lower dose) of AAV-Cre infection.

Very interestingly, the Cre expression was easily detected in both the high-dose and low-dose groups of AAV-Cre infection, but no significant Cre expression was detected in the RAAV-Cre infected cells despite of the detected tdTomato fluorescence proving the once existence of Cre.

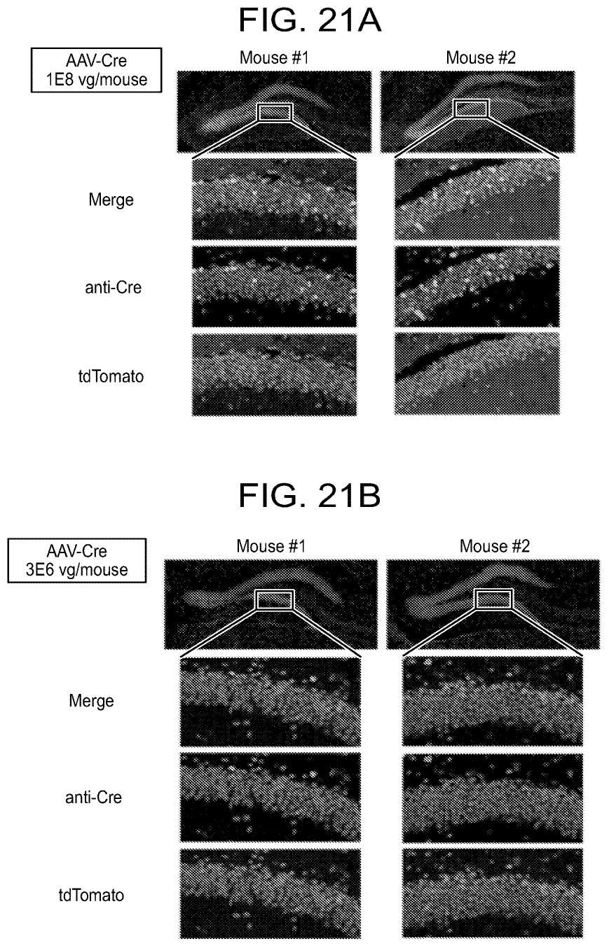

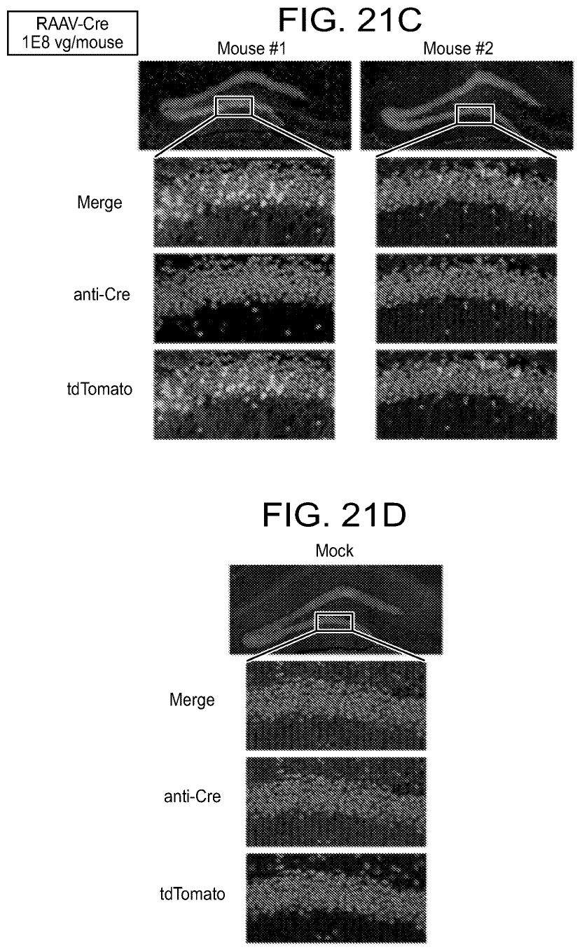

Overall, the RAAV-Cre had an inferior transduction efficiency compared to the conventional AAV-Cre as shown by the fluorescent photos (positive cell counts) for the two at the same high dose of 1E9 vg/mouse (FIG. 21A vs. FIG. 21C), since multiple mRNAs for protein translation can be transcribed from one successfully transduced AAV DNA genome. To further evaluate the Cre expression levels, the transduction efficiency of the AAV-Cre was normalized to that of the RAAV-Cre by reducing the high dose of AAV-Cre of 1E9 vg/mouse to a low dose of 3E6 vg/mouse (FIG. 21B), and the results showed that although the RAAV-Cre had a superior transduction efficiency to the low dose group of AAV-Cre as shown by the fluorescent photos (positive cell counts), no Cre expression was detected in the RAAV-Cre infected cells as compared with the AAV-Cre group, which indicated that the Cre expression in RAAV-Cre group was transient but functional.

### Example 10 Additional RPS/RBP Pairs for RAAV System

In addition to the MS2/MCP pair used in Examples 3-8, two additional pairs of RNA aptamer/aptamer-binding proteins (or RNA packaging signal/RNA binding protein, “RPS/RBP” herein) were tested for RAAV packaging: (1) PP7 binding site/PP7 bacteriophage coat protein (“PP7/PCP,” or “PCP” or “P” for short, or “P” in L-CCWP3S) and (2) Com binding site/phage COM protein (“com/COM,” or “COM” for short, or “C” in L-CCWC3S). Unlike MS2/MCP and PP7/PCP that are natural viral packaging systems, com/COM is not a natural viral packaging system but known to be transcription regulators that play roles in the transcription initiation of the bacteriophage Mu mom gene.

Transgene plasmids harboring three copies of RPS (L-CCWP3S and L-CCWC3S) and their corresponding packaging plasmids [DJ-PCP (Y156F) with PP7 bacteriophage coat protein (PCP) fused to the N-terminus of Rep78-Y156F and Rep68-Y156F and DJ-COM (Y156F) with phage COM protein (COM) fused to the N-terminus of Rep78-Y156F and Rep68-Y156F] were constructed. Viral particles were produced, purified, and titrated as described in Example 3.

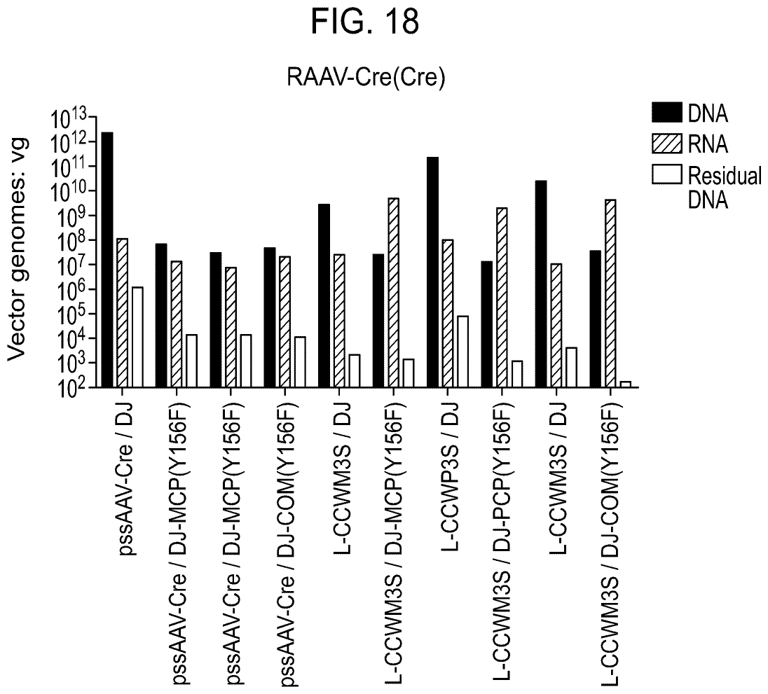

The results showed that similar to the MS2/MCP pair well demonstrated in various aspects in Examples 3-8, the two pairs of PP7/PCP and com/COM also led to the remarkable RNA packaging of RAAV particles (FIG. 18), thereby expanding the scope of various RAAV packaging system.

### Example 11 Application of RAAV System to Various AAV Serotypes

To investigate the application of the inventive RAAV packaging system to various AAV serotypes in addition to AAV-DJ tested in Examples 3-9, two pairs of RPS/RBP (MS2/MCP and com/COM) were examined in AAV-DJ and another three different AAV serotypes (AAV5, AAV8 and AAV9). Viral particles were produced, purified, and titrated as described in Example 3.

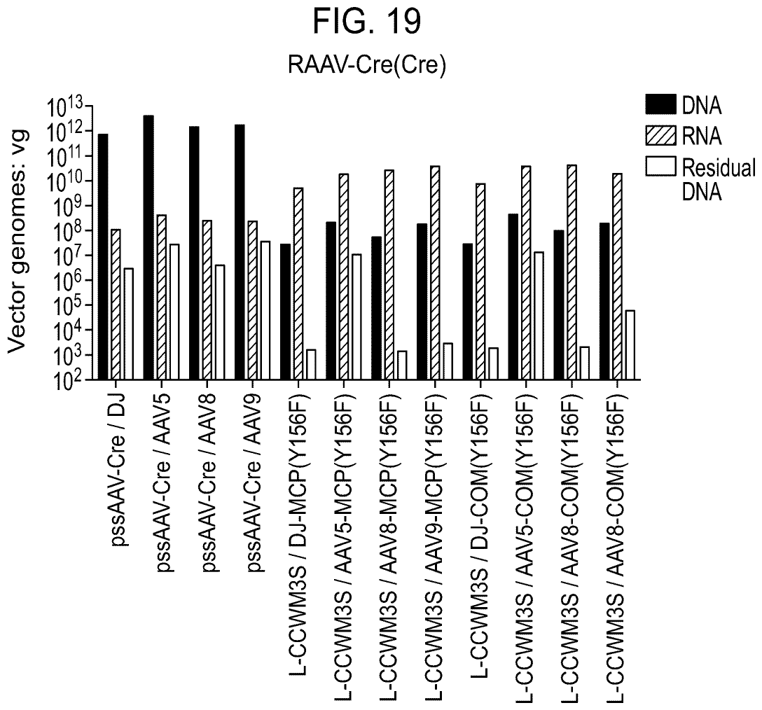

Both RAAV-MS2/MCP and RAAV-com/COM system worked well in all the four serotypes, suggesting the general applicability of the RAAV packaging systems to different AAV serotypes (i.e., not limited to AAV-DJ). In the presence of the RBP, Cre RNA genomes containing the corresponding RPS were efficiently encapsidated into the respective RAAV particles. Though the yields of the RNA-packaged RAAV particles varied from serotype to serotype, all of the RAAV5, RAAV8 and RAAV9 particles had a higher productivity than RAAV-DJ (FIG. 19).

### Example 12 AAP and MCP Fusion Protein Increased RAAV Yield

Generally, AAV encodes a unique assembly-activating protein (AAP) within their natural viral genomes that is essential for capsid assembly. Specifically, AAP was found to be essential for capsid protein stabilization and generation of functional AAV particles.

An AAP-MCP (with MCP fused to the C-terminus of AAP) or MCP-AAP (with MCP fused to the N-terminus of AAP) fusion protein expression cassette was inserted inversely into the backbone of the packaging plasmid DJ-MCP (Y156F) used in Examples 6-10, and the resulting constructs were named DJ-MCP (Y156F)-AM and DJ-MCP (Y156F)-MA, respectively. Such constructs then expressed both MCP-Rep78/68 (Y156F) fusion and AAP-MCP or MCP-AAP fusion, increasing the amount of RNA binding proteins (RBPs) assisting in RNA packaging compared with MCP-Rep78/68 fusion alone. Viral particles were produced, purified, and titrated as described in Example 3.

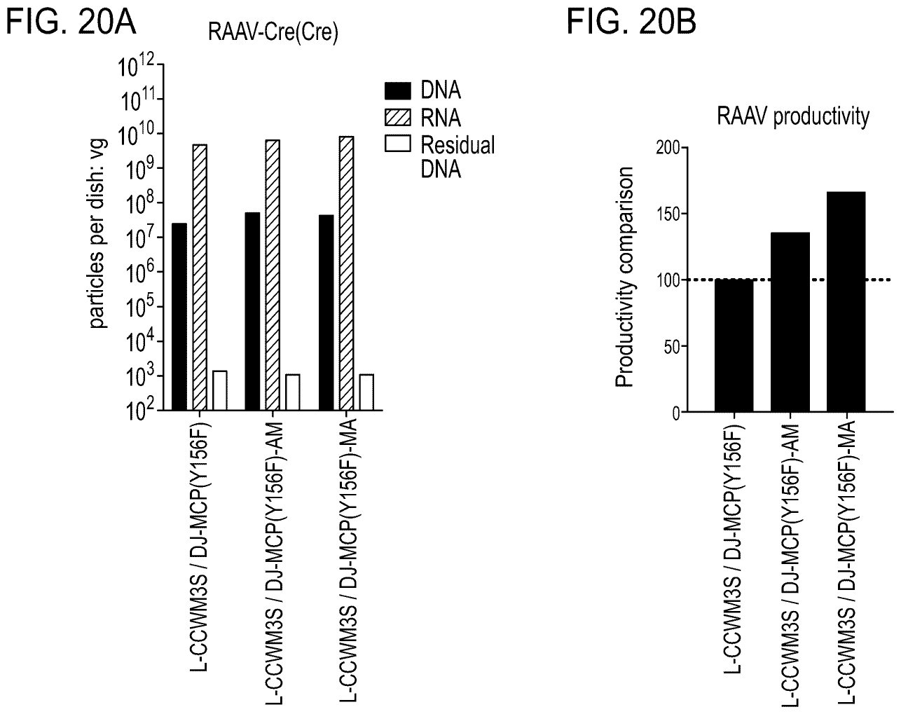

The results showed that the yields of RNA-packaged RAAV particles were increased by about 65% in DJ-MCP (Y156F)-MA and about 35% in DJ-MCP (Y156F)-AM compared with MCP-Rep78/68 fusion alone (FIGS. 20A and 20B), suggesting that RBPs could be additionally fused to or associated with any other proteins which play roles in the packaging or assembly of AAV particles in order to enhance the RNA packaging of RAAV particles.

Using AAP-MCP or MCP-AAP alone, without MCP-Rep78/68, are also within the scope of the invention.

### Sequences

Certain sequences, including those referenced in the examples above, are provided herein below.

TABLE A
Nucleic Acid Sequence and Amino Acid Sequence of RBP

| RNA binding | |
|---|---|
| protein | Sequences |
| MCP nucleic acid | GCTTCTAACTTTACTCAGTTCGTTCTCGTCGACAATGGCGGAACTGGCG |
| sequence | ACGTGACTGTCGCCCCAAGCAACTTCGCTAACGGGGTCGCTGAATGGAT |
| SEQ ID NO: 109 | CAGCTCTAACTCGCGTTCACAGGCTTACAAAGTAACCTGTAGCGTTCGT |
| | CAGAGCTCTGCGCAGAATCGCAAATACACCATCAAAGTCGAGGTGCCTA |
| | AAGTGGCAACCCAGACTGTTGGTGGAGTAGAGCTTCCTGTAGCCGCATG |
| | GCGTTCGTACTTAAATATGGAACTAACCATTCCAATTTTCGCTACGAAT |
| | TCCGACTGCGAGCTTATTGTTAAGGCAATGCAAGGTCTCCTAAAAGATG |
| | GAAACCCGATTCCCTCAGCAATCGCAGCAAACTCCGGCATCTAC |
| MCP amino acid | ASNFTQFVLVDNGGTGDVTVAPSNFANGVAEWISSNSRSQAYKVTCSVR |
| sequence | QSSAQNRKYTIKVEVPKVATQTVGGVELPVAAWRSYLNMELTIPIFATN |
| SEQ ID NO: 110 | SDCELIVKAMQGLLKDGNPIPSAIAANSGIY |
| PCP nucleic acid | TCCAAAACAATAGTCCTCTCCGTAGGGGAGGCAACACGGACTTTGACCG |
| sequence | AAATCCAGTCAACCGCTGACCGACAAATCTTTGAAGAGAAAGTAGGGCC |
| SEQ ID NO: 111 | TCTTGTGGGCCGACTGCGCTTGACTGCAAGCTTGCGACAAAACGGCGCA |
| | AAGACTGCCTATAGGGTCAACCTTAAACTCGACCAAGCCGACGTGGTCG |
| | ATAGCGGTCTCCCTAAGGTTCGGTATACGCAGGTCTGGAGTCATGACGT |
| | AACAATCGTAGCAAACAGCACAGAAGCCTCCCGAAAAAGCCTCTACGAT |
| | CTGACGAAATCCTTGGTGGCTACGTCACAGGTGGAAGACCTCGTTGTCA |
| | ACCTTGTACCTCTGGGTCGA |
| PCP amino acid | SKTIVLSVGEATRTLTEIQSTADRQIFEEKVGPLVGRLRLTASLRQNGA |
| sequence | KTAYRVNLKLDQADVVDSGLPKVRYTQVWSHDVTIVANSTEASRKSLYD |
| SEQ ID NO: 112 | LTKSLVATSQVEDLVVNLVPLGR |
| COM nucleic acid | ATGAAATCAATTCGCTGTAAAAACTGCAACAAACTGTTATTTAAGGCGG |
| sequence | ATTCCTTTGATCACATTGAAATCAGGTGTCCGCGTTGCAAACGTCACAT |
| SEQ ID NO: 113 | CATAATGCTGAATGCCTGCGAGCATCCCACGGAGAAACATTGTGGGAAA |
| | AGAGAAAAAATCACGCATTCTGACGAAACCGTGCGTTAT |
| COM amino acid | MKSIRCKNCNKLLFKADSFDHIEIRCPRCKRHIIMLNACEHPTEKHCGK |
| sequence | REKITHSDETVRY |
| SEQ ID NO: 114 | |

TABLE B
Nucleic Acid Sequences of RPS

| Packaging signal | Nucleic acid sequences |
|---|---|
| MS2 | ACATGAGGATCACCCATGT |
| SEQ ID NO: 115 | |
| MS2 X3 (MS2-linker- | ACATGAGGATCACCCATGTCTGCAGGTCGACTCTAGAAAA |
| MS2-linker-MS2) | CATGAGGATCACCCATGTCTGCAGTATTCCCGGGTTCATTA |
| SEQ ID NO: 116 | GATCCTAAGGTACCTAATTGCCTAGAAAACATGAGGATCA |
| | CCCATGT |
| PP7 | GGAGCAGACGATATGGCGTCGCTCC |
| SEQ ID NO: 117 | |
| PP7 X3 (PP7-linker- | GGAGCAGACGATATGGCGTCGCTCCCTGCAGGTCGACTCT |
| PP7-linker-PP7) | AGAAAGGAGCAGACGATATGGCGTCGCTCCCTGCAGTATT |
| SEQ ID NO: 118 | CCCGGGTTCATTAGATCCTAAGGTACCTAATTGCCTAGAA |
| | AGGAGCAGACGATATGGCGTCGCTCC |
| Com | GAATGCCTGCGAGCATCC |
| SEQ ID NO: 119 | |
| com X3 (com-linker- | GAATGCCTGCGAGCATCCCTGCAGGTCGACTCTAGAAAGA |
| com-linker-com) | ATGCCTGCGAGCATCCCTGCAGTATTCCCGGGTTCATTAG |
| SEQ ID NO: 120 | ATCCTAAGGTACCTAATTGCCTAGAAAGAATGCCTGCGAG |
| | CATCC |

* Sequence elements are matched based on formatting styles (e.g., bold and/or italic fonts, etc.)

TABLE C
Nucleic Acid Sequence and Amino Acid Sequence of Rep proteins

| Rep proteins | Sequences |
|---|---|
| Rep2 nucleic | ATGCCGGGGTTTTACGAGATTGTGATTAAGGTCCCCAGCGACCTTGACG |
| acid sequence | AGCATCTGCCCGGCATTTCTGACAGCTTTGTGAACTGGGTGGCCGAGAA |
| SEQ ID NO: 121 | GGAATGGGAGTTGCCGCCAGATTCTGACATGGATCTGAATCTGATTGAG |
| | CAGGCACCCCTGACCGTGGCCGAGAAGCTGCAGCGCGACTTTCTGACGG |
| | AATGGCGCCGTGTGAGTAAGGCCCCGGAGGCCCTTTTCTTTGTGCAATT |
| | TGAGAAGGGAGAGAGCTACTTCCACATGCACGTGCTCGTGGAAACCACC |
| | GGGGTGAAATCCATGGTTTTGGGACGTTTCCTGAGTCAGATTCGCGAAA |
| | AACTGATTCAGAGAATTTACCGCGGGATCGAGCCGACTTTGCCAAACTG |
| | GTTCGCGGTCACAAAGACCAGAAATGGCGCCGGAGGCGGGAACAAGGTG |
| | GTGGATGAGTGCTACATCCCCAATTACTTGCTCCCCAAAACCCAGCCTG |
| | AGCTCCAGTGGGCGTGGACTAATATGGAACAGTATTTAAGCGCCTGTTT |
| | GAATCTCACGGAGCGTAAACGGTTGGTGGCGCAGCATCTGACGCACGTG |
| | TCGCAGACGCAGGAGCAGAACAAAGAGAATCAGAATCCCAATTCTGATG |
| | CGCCGGTGATCAGATCAAAAACTTCAGCCAGGTACATGGAGCTGGTCGG |
| | GTGGCTCGTGGACAAGGGGATTACCTCGGAGAAGCAGTGGATCCAGGAG |
| | GACCAGGCCTCATACATCTCCTTCAATGCGGCCTCCAACTCGCGGTCCC |
| | AAATCAAGGCTGCCTTGGACAATGCGGGAAAGATTATGAGCCTGACTAA |
| | AACCGCCCCCGACTACCTGGTGGGCCAGCAGCCCGTGGAGGACATTTCC |
| | AGCAATCGGATTTATAAAATTTTGGAACTAAACGGGTACGATCCCCAAT |
| | ATGCGGCTTCCGTCTTTCTGGGATGGGCCACGAAAAAGTTCGGCAAGAG |
| | GAACACCATCTGGCTGTTTGGGCCTGCAACTACCGGGAAGACCAACATC |
| | GCGGAGGCCATAGCCCACACTGTGCCCTTCTACGGGTGCGTAAACTGGA |
| | CCAATGAGAACTTTCCCTTCAACGACTGTGTCGACAAGATGGTGATCTG |
| | GTGGGAGGAGGGGAAGATGACCGCCAAGGTCGTGGAGTCGGCCAAAGCC |
| | ATTCTCGGAGGAAGCAAGGTGCGCGTGGACCAGAAATGCAAGTCCTCGG |
| | CCCAGATAGACCCGACTCCCGTGATCGTCACCTCCAACACCAACATGTG |
| | CGCCGTGATTGACGGGAACTCAACGACCTTCGAACACCAGCAGCCGTTG |
| | CAAGACCGGATGTTCAAATTTGAACTCACCCGCCGTCTGGATCATGACT |
| | TTGGGAAGGTCACCAAGCAGGAAGTCAAAGACTTTTTCCGGTGGGCAAA |
| | GGATCACGTGGTTGAGGTGGAGCATGAATTCTACGTCAAAAAGGGTGGA |
| | GCCAAGAAAAGACCCGCCCCCAGTGACGCAGATATAAGTGAGCCCAAAC |
| | GGGTGCGCGAGTCAGTTGCGCAGCCATCGACGTCAGACGCGGAAGCTTC |
| | GATCAACTACGCAGACAGGTACCAAAACAAATGTTCTCGTCACGTGGGC |
| | ATGAATCTGATGCTGTTTCCCTGCAGACAATGCGAGAGAATGAATCAGA |
| | ATTCAAATATCTGCTTCACTCACGGACAGAAAGACTGTTTAGAGTGCTT |
| | TCCCGTGTCAGAATCTCAACCCGTTTCTGTCGTCAAAAAGGCGTATCAG |
| | AAACTGTGCTACATTCATCATATCATGGGAAAGGTGCCAGACGCTTGCA |
| | CTGCCTGCGATCTGGTCAATGTGGATTTGGATGACTGCATCTTTGAACA |
| | ATAA |
| Rep2 amino acid | MPGFYEIVIKVPSDLDEHLPGISDSFVNWVAEKEWELPPDSDMDLNLIE |
| sequence | QAPLTVAEKLQRDFLTEWRRVSKAPEALFFVQFEKGESYFHMHVLVETT |
| SEQ ID NO: 122 | GVKSMVLGRFLSQIREKLIQRIYRGIEPTLPNWFAVTKTRNGAGGGNKV |
| | VDECYIPNYLLPKTQPELQWAWINMEQYLSACLNLTERKRLVAQHLTHV |
| | SQTQEQNKENQNPNSDAPVIRSKTSARYMELVGWLVDKGITSEKQWIQE |
| | DQASYISFNAASNSRSQIKAALDNAGKIMSLIKTAPDYLVGQQPVEDIS |
| | SNRIYKILELNGYDPQYAASVELGWATKKFGKRNTIWLFGPATTGKTNI |
| | AEAIAHTVPFYGCVNWTNENFPFNDCVDKMVIWWEEGKMTAKVVESAKA |
| | ILGGSKVRVDQKCKSSAQIDPTPVIVTSNTNMCAVIDGNSTTFEHQQPL |
| | QDRMFKFELTRRLDHDFGKVTKQEVKDFFRWAKDHVVEVEHEFYVKKGG |
| | AKKRPAPSDADISEPKRVRESVAQPSTSDAEASINYADRYQNKCSRHVG |
| | MNLMLFPCRQCERMNQNSNICFTHGQKDCLECFPVSESQPVSVVKKAYQ |
| | KLCYIHHIMGKVPDACTACDLVNVDLDDCIFEQ |
| MCP- | ATGCCCGGCAGCTCCGGCAGTAGCGCTTCTAACTTTACTCAGTTCGTTC |
| Rep nucleic acid | TCGTCGACAATGGCGGAACTGGCGACGTGACTGTCGCCCCAAGCAACTT |
| sequence (linker- | CGCTAACGGGGTCGCTGAATGGATCAGCTCTAACTCGCGTTCACAGGCT |
| MCP-linker-Rep) | TACAAAGTAACCTGTAGCGTTCGTCAGAGCTCTGCGCAGAATCGCAAAT |
| SEQ ID NO: 123 | ACACCATCAAAGTCGAGGTGCCTAAAGTGGCAACCCAGACTGTTGGTGG |
| | AGTAGAGCTTCCTGTAGCCGCATGGCGTTCGTACTTAAATATGGAACTA |
| | ACCATTCCAATTTTCGCTACGAATTCCGACTGCGAGCTTATTGTTAAGG |
| | CAATGCAAGGTCTCCTAAAAGATGGAAACCCGATTCCCTCAGCAATCGC |
| | AGCAAACTCCGGCATCTACGGCAGTAGTGGGTCCTCTGGGTTTTACGAG |
| | ATTGTGATTAAGGTCCCCAGCGACCTTGACGAGCATCTGCCCGGCATTT |
| | CTGACAGCTTTGTGAACTGGGTGGCCGAGAAGGAATGGGAGTTGCCGCC |
| | AGATTCTGACATGGATCTGAATCTGATTGAGCAGGCACCCCTGACCGTG |
| | GCCGAGAAGCTGCAGCGCGACTTTCTGACGGAATGGCGCCGTGTGAGTA |
| | AGGCCCCGGAGGCCCTTTTCTTTGTGCAATTTGAGAAGGGAGAGAGCTA |
| | CTTCCACATGCACGTGCTCGTGGAAACCACCGGGGTGAAATCCATGGTT |
| | TTGGGACGTTTCCTGAGTCAGATTCGCGAAAAACTGATTCAGAGAATTT |
| | ACCGCGGGATCGAGCCGACTTTGCCAAACTGGTTCGCGGTCACAAAGAC |
| | CAGAAATGGCGCCGGAGGCGGGAACAAGGTGGTGGATGAGTGCTACATC |
| | CCCAATTACTTGCTCCCCAAAACCCAGCCTGAGCTCCAGTGGGCGTGGA |
| | CTAATATGGAACAGTATTTAAGCGCCTGTTTGAATCTCACGGAGCGTAA |
| | ACGGTTGGTGGCGCAGCATCTGACGCACGTGTCGCAGACGCAGGAGCAG |
| | AACAAAGAGAATCAGAATCCCAATTCTGATGCGCCGGTGATCAGATCAA |
| | AAACTTCAGCCAGGTACATGGAGCTGGTCGGGTGGCTCGTGGACAAGGG |
| | GATTACCTCGGAGAAGCAGTGGATCCAGGAGGACCAGGCCTCATACATC |
| | TCCTTCAATGCGGCCTCCAACTCGCGGTCCCAAATCAAGGCTGCCTTGG |
| | ACAATGCGGGAAAGATTATGAGCCTGACTAAAACCGCCCCCGACTACCT |
| | GGTGGGCCAGCAGCCCGTGGAGGACATTTCCAGCAATCGGATTTATAAA |
| | ATTTTGGAACTAAACGGGTACGATCCCCAATATGCGGCTTCCGTCTTTC |
| | TGGGATGGGCCACGAAAAAGTTCGGCAAGAGGAACACCATCTGGCTGTT |
| | TGGGCCTGCAACTACCGGGAAGACCAACATCGCGGAGGCCATAGCCCAC |
| | ACTGTGCCCTTCTACGGGTGCGTAAACTGGACCAATGAGAACTTTCCCT |
| | TCAACGACTGTGTCGACAAGATGGTGATCTGGTGGGAGGAGGGGAAGAT |
| | GACCGCCAAGGTCGTGGAGTCGGCCAAAGCCATTCTCGGAGGAAGCAAG |
| | GTGCGCGTGGACCAGAAATGCAAGTCCTCGGCCCAGATAGACCCGACTC |
| | CCGTGATCGTCACCTCCAACACCAACATGTGCGCCGTGATTGACGGGAA |
| | CTCAACGACCTTCGAACACCAGCAGCCGTTGCAAGACCGGATGTTCAAA |
| | TTTGAACTCACCCGCCGTCTGGATCATGACTTTGGGAAGGTCACCAAGC |
| | AGGAAGTCAAAGACTTTTTCCGGTGGGCAAAGGATCACGTGGTTGAGGT |
| | GGAGCATGAATTCTACGTCAAAAAGGGTGGAGCCAAGAAAAGACCCGCC |
| | CCCAGTGACGCAGATATAAGTGAGCCCAAACGGGTGCGCGAGTCAGTTG |
| | CGCAGCCATCGACGTCAGACGCGGAAGCTTCGATCAACTACGCAGACAG |
| | GTACCAAAACAAATGTTCTCGTCACGTGGGCATGAATCTGATGCTGTTT |
| | CCCTGCAGACAATGCGAGAGAATGAATCAGAATTCAAATATCTGCTTCA |
| | CTCACGGACAGAAAGACTGTTTAGAGTGCTTTCCCGTGTCAGAATCTCA |
| | ACCCGTTTCTGTCGTCAAAAAGGCGTATCAGAAACTGTGCTACATTCAT |
| | CATATCATGGGAAAGGTGCCAGACGCTTGCACTGCCTGCGATCTGGTCA |
| | ATGTGGATTTGGATGACTGCATCTTTGAACAATAA |
| MCP- | MPGSSGSSASNFTQFVLVDNGGTGDVTVAPSNFANGVAEWISSNSRSQA |
| Rep amino acid | YKVTCSVRQSSAQNRKYTIKVEVPKVATQTVGGVELPVAAWRSYLNMEL |
| sequence(linker- | TIPIFATNSDCELIVKAMQGLLKDGNPIPSAIAANSGIYGSSGSSGFYE |
| MCP-linker-Rep) | IVIKVPSDLDEHLPGISDSFVNWVAEKEWELPPDSDMDLNLIEQAPLTV |
| SEQ ID NO: 124 | AEKLQRDFLTEWRRVSKAPEALFFVQFEKGESYFHMHVLVETTGVKSMV |
| | LGRFLSQIREKLIQRIYRGIEPTLPNWFAVTKTRNGAGGGNKVVDECYI |
| | PNYLLPKTQPELQWAWTNMEQYLSACLNLTERKRLVAQHLTHVSQTQEQ |
| | NKENQNPNSDAPVIRSKTSARYMELVGWLVDKGITSEKQWIQEDQASYI |
| | SFNAASNSRSQIKAALDNAGKIMSLIKTAPDYLVGQQPVEDISSNRIYK |
| | ILELNGYDPQYAASVELGWATKKFGKRNTIWLFGPATTGKINIAEAIAH |
| | TVPFYGCVNWTNENFPFNDCVDKMVIWWEEGKMTAKVVESAKAILGGSK |
| | VRVDQKCKSSAQIDPTPVIVTSNTNMCAVIDGNSTTFEHQQPLQDRMFK |
| | FELTRRLDHDFGKVTKQEVKDFFRWAKDHVVEVEHEFYVKKGGAKKRPA |
| | PSDADISEPKRVRESVAQPSTSDAEASINYADRYQNKCSRHVGMNLMLF |
| | PCRQCERMNQNSNICFTHGQKDCLECFPVSESQPVSVVKKAYQKLCYIH |
| | HIMGKVPDACTACDLVNVDLDDCIFEQ |
| MCP-Rep-Y156F | ATGCCCGGCAGCTCCGGCAGTAGCGCTTCTAACTTTACTCAGTTCGTTC |
| nucleic acid | TCGTCGACAATGGCGGAACTGGCGACGTGACTGTCGCCCCAAGCAACTT |
| sequence (linker- | CGCTAACGGGGTCGCTGAATGGATCAGCTCTAACTCGCGTTCACAGGCT |
| MCP-linker-Rep) | TACAAAGTAACCTGTAGCGTTCGTCAGAGCTCTGCGCAGAATCGCAAAT |
| SEQ ID NO: 125 | ACACCATCAAAGTCGAGGTGCCTAAAGTGGCAACCCAGACTGTTGGTGG |
| | AGTAGAGCTTCCTGTAGCCGCATGGCGTTCGTACTTAAATATGGAACTA |
| | ACCATTCCAATTTTCGCTACGAATTCCGACTGCGAGCTTATTGTTAAGG |
| | CAATGCAAGGTCTCCTAAAAGATGGAAACCCGATTCCCTCAGCAATCGC |
| | AGCAAACTCCGGCATCTACGGCAGTAGTGGGTCCTCTGGGTTTTACGAG |
| | ATTGTGATTAAGGTCCCCAGCGACCTTGACGAGCATCTGCCCGGCATTT |
| | CTGACAGCTTTGTGAACTGGGTGGCCGAGAAGGAATGGGAGTTGCCGCC |
| | AGATTCTGACATGGATCTGAATCTGATTGAGCAGGCACCCCTGACCGTG |
| | GCCGAGAAGCTGCAGCGCGACTTTCTGACGGAATGGCGCCGTGTGAGTA |
| | AGGCCCCGGAGGCCCTTTTCTTTGTGCAATTTGAGAAGGGAGAGAGCTA |
| | CTTCCACATGCACGTGCTCGTGGAAACCACCGGGGTGAAATCCATGGTT |
| | TTGGGACGTTTCCTGAGTCAGATTCGCGAAAAACTGATTCAGAGAATTT |
| | ACCGCGGGATCGAGCCGACTTTGCCAAACTGGTTCGCGGTCACAAAGAC |
| | CAGAAATGGCGCCGGAGGCGGGAACAAGGTGGTGGATGAGTGCTACATC |
| | CCCAATTTCTTGCTCCCCAAAACCCAGCCTGAGCTCCAGTGGGCGTGGA |
| | CTAATATGGAACAGTATTTAAGCGCCTGTTTGAATCTCACGGAGCGTAA |
| | ACGGTTGGTGGCGCAGCATCTGACGCACGTGTCGCAGACGCAGGAGCAG |
| | AACAAAGAGAATCAGAATCCCAATTCTGATGCGCCGGTGATCAGATCAA |
| | AAACTTCAGCCAGGTACATGGAGCTGGTCGGGTGGCTCGTGGACAAGGG |
| | GATTACCTCGGAGAAGCAGTGGATCCAGGAGGACCAGGCCTCATACATC |
| | TCCTTCAATGCGGCCTCCAACTCGCGGTCCCAAATCAAGGCTGCCTTGG |
| | ACAATGCGGGAAAGATTATGAGCCTGACTAAAACCGCCCCCGACTACCT |
| | GGTGGGCCAGCAGCCCGTGGAGGACATTTCCAGCAATCGGATTTATAAA |
| | ATTTTGGAACTAAACGGGTACGATCCCCAATATGCGGCTTCCGTCTTTC |
| | TGGGATGGGCCACGAAAAAGTTCGGCAAGAGGAACACCATCTGGCTGTT |
| | TGGGCCTGCAACTACCGGGAAGACCAACATCGCGGAGGCCATAGCCCAC |
| | ACTGTGCCCTTCTACGGGTGCGTAAACTGGACCAATGAGAACTTTCCCT |
| | TCAACGACTGTGTCGACAAGATGGTGATCTGGTGGGAGGAGGGGAAGAT |
| | GACCGCCAAGGTCGTGGAGTCGGCCAAAGCCATTCTCGGAGGAAGCAAG |
| | GTGCGCGTGGACCAGAAATGCAAGTCCTCGGCCCAGATAGACCCGACTC |
| | CCGTGATCGTCACCTCCAACACCAACATGTGCGCCGTGATTGACGGGAA |
| | CTCAACGACCTTCGAACACCAGCAGCCGTTGCAAGACCGGATGTTCAAA |
| | TTTGAACTCACCCGCCGTCTGGATCATGACTTTGGGAAGGTCACCAAGC |
| | AGGAAGTCAAAGACTTTTTCCGGTGGGCAAAGGATCACGTGGTTGAGGT |
| | GGAGCATGAATTCTACGTCAAAAAGGGTGGAGCCAAGAAAAGACCCGCC |
| | CCCAGTGACGCAGATATAAGTGAGCCCAAACGGGTGCGCGAGTCAGTTG |
| | CGCAGCCATCGACGTCAGACGCGGAAGCTTCGATCAACTACGCAGACAG |
| | GTACCAAAACAAATGTTCTCGTCACGTGGGCATGAATCTGATGCTGTTT |
| | CCCTGCAGACAATGCGAGAGAATGAATCAGAATTCAAATATCTGCTTCA |
| | CTCACGGACAGAAAGACTGTTTAGAGTGCTTTCCCGTGTCAGAATCTCA |
| | ACCCGTTTCTGTCGTCAAAAAGGCGTATCAGAAACTGTGCTACATTCAT |
| | CATATCATGGGAAAGGTGCCAGACGCTTGCACTGCCTGCGATCTGGTCA |
| | ATGTGGATTTGGATGACTGCATCTTTGAACAATAA |
| MCP-Rep-Y156F | MPGSSGSSASNFTQFVLVDNGGTGDVTVAPSNFANGVAEWISSNSRSQA |
| amino acid | YKVTCSVRQSSAQNRKYTIKVEVPKVATQTVGGVELPVAAWRSYLNMEL |
| sequence (linker- | TIPIFATNSDCELIVKAMQGLLKDGNPIPSAIAANSGIYGSSGSSGFYE |
| MCP-linker-Rep) | IVIKVPSDLDEHLPGISDSFVNWVAEKEWELPPDSDMDLNLIEQAPLTV |
| SEQ ID NO: 126 | AEKLQRDFLTEWRRVSKAPEALFFVQFEKGESYFHMHVLVETTGVKSMV |
| | LGRFLSQIREKLIQRIYRGIEPTLPNWFAVTKTRNGAGGGNKVVDECYI |
| | PNFLLPKTQPELQWAWTNMEQYLSACLNLTERKRLVAQHLTHVSQTQEQ |
| | NKENQNPNSDAPVIRSKTSARYMELVGWLVDKGITSEKQWIQEDQASYI |
| | SFNAASNSRSQIKAALDNAGKIMSLIKTAPDYLVGQQPVEDISSNRIYK |
| | ILELNGYDPQYAASVELGWATKKFGKRNTIWLFGPATTGKTNIAEAIAH |
| | TVPFYGCVNWTNENFPFNDCVDKMVIWWEEGKMTAKVVESAKAILGGSK |
| | VRVDQKCKSSAQIDPTPVIVTSNTNMCAVIDGNSTTFEHQQPLQDRMFK |
| | FELTRRLDHDFGKVTKQEVKDFFRWAKDHVVEVEHEFYVKKGGAKKRPA |
| | PSDADISEPKRVRESVAQPSTSDAEASINYADRYQNKCSRHVGMNLMLF |
| | PCRQCERMNONSNICFTHGQKDCLECFPVSESQPVSVVKKAYQKLCYIH |
| | HIMGKVPDACTACDLVNVDLDDCIFEQ |
| MCP-Rep-KDE- | ATGCCCGGCAGCTCCGGCAGTAGCGCTTCTAACTTTACTCAGTTCGTTC |
| mu nucleic acid | TCGTCGACAATGGCGGAACTGGCGACGTGACTGTCGCCCCAAGCAACTT |
| sequence (linker- | CGCTAACGGGGTCGCTGAATGGATCAGCTCTAACTCGCGTTCACAGGCT |
| MCP-linker-Rep) | TACAAAGTAACCTGTAGCGTTCGTCAGAGCTCTGCGCAGAATCGCAAAT |
| SEQ ID NO: 127 | ACACCATCAAAGTCGAGGTGCCTAAAGTGGCAACCCAGACTGTTGGTGG |
| | AGTAGAGCTTCCTGTAGCCGCATGGCGTTCGTACTTAAATATGGAACTA |
| | ACCATTCCAATTTTCGCTACGAATTCCGACTGCGAGCTTATTGTTAAGG |
| | CAATGCAAGGTCTCCTAAAAGATGGAAACCCGATTCCCTCAGCAATCGC |
| | AGCAAACTCCGGCATCTACGGCAGTAGTGGGTCCTCTGGGTTTTACGAG |
| | ATTGTGATTAAGGTCCCCAGCGACCTTGACGAGCATCTGCCCGGCATTT |
| | CTGACAGCTTTGTGAACTGGGTGGCCGAGAAGGAATGGGAGTTGCCGCC |
| | AGATTCTGACATGGATCTGAATCTGATTGAGCAGGCACCCCTGACCGTG |
| | GCCGAGAAGCTGCAGCGCGACTTTCTGACGGAATGGCGCCGTGTGAGTA |
| | AGGCCCCGGAGGCCCTTTTCTTTGTGCAATTTGAGAAGGGAGAGAGCTA |
| | CTTCCACATGCACGTGCTCGTGGAAACCACCGGGGTGAAATCCATGGTT |
| | TTGGGACGTTTCCTGAGTCAGATTCGCGAAAAACTGATTCAGAGAATTT |
| | ACCGCGGGATCGAGCCGACTTTGCCAAACTGGTTCGCGGTCACAAAGAC |
| | CAGAAATGGCGCCGGAGGCGGGAACGCGGTGGTGGCTGCGTGCTACATC |
| | CCCAATTACTTGCTCCCCAAAACCCAGCCTGAGCTCCAGTGGGCGTGGA |
| | CTAATATGGAACAGTATTTAAGCGCCTGTTTGAATCTCACGGAGCGTAA |
| | ACGGTTGGTGGCGCAGCATCTGACGCACGTGTCGCAGACGCAGGAGCAG |
| | AACAAAGAGAATCAGAATCCCAATTCTGATGCGCCGGTGATCAGATCAA |
| | AAACTTCAGCCAGGTACATGGAGCTGGTCGGGTGGCTCGTGGACAAGGG |
| | GATTACCTCGGAGAAGCAGTGGATCCAGGAGGACCAGGCCTCATACATC |
| | TCCTTCAATGCGGCCTCCAACTCGCGGTCCCAAATCAAGGCTGCCTTGG |
| | ACAATGCGGGAAAGATTATGAGCCTGACTAAAACCGCCCCCGACTACCT |
| | GGTGGGCCAGCAGCCCGTGGAGGACATTTCCAGCAATCGGATTTATAAA |
| | ATTTTGGAACTAAACGGGTACGATCCCCAATATGCGGCTTCCGTCTTTC |
| | TGGGATGGGCCACGAAAAAGTTCGGCAAGAGGAACACCATCTGGCTGTT |
| | TGGGCCTGCAACTACCGGGAAGACCAACATCGCGGAGGCCATAGCCCAC |
| | ACTGTGCCCTTCTACGGGTGCGTAAACTGGACCAATGAGAACTTTCCCT |
| | TCAACGACTGTGTCGACAAGATGGTGATCTGGTGGGAGGAGGGGAAGAT |
| | GACCGCCAAGGTCGTGGAGTCGGCCAAAGCCATTCTCGGAGGAAGCAAG |
| | GTGCGCGTGGACCAGAAATGCAAGTCCTCGGCCCAGATAGACCCGACTC |
| | CCGTGATCGTCACCTCCAACACCAACATGTGCGCCGTGATTGACGGGAA |
| | CTCAACGACCTTCGAACACCAGCAGCCGTTGCAAGACCGGATGTTCAAA |
| | TTTGAACTCACCCGCCGTCTGGATCATGACTTTGGGAAGGTCACCAAGC |
| | AGGAAGTCAAAGACTTTTTCCGGTGGGCAAAGGATCACGTGGTTGAGGT |
| | GGAGCATGAATTCTACGTCAAAAAGGGTGGAGCCAAGAAAAGACCCGCC |
| | CCCAGTGACGCAGATATAAGTGAGCCCAAACGGGTGCGCGAGTCAGTTG |
| | CGCAGCCATCGACGTCAGACGCGGAAGCTTCGATCAACTACGCAGACAG |
| | GTACCAAAACAAATGTTCTCGTCACGTGGGCATGAATCTGATGCTGTTT |
| | CCCTGCAGACAATGCGAGAGAATGAATCAGAATTCAAATATCTGCTTCA |
| | CTCACGGACAGAAAGACTGTTTAGAGTGCTTTCCCGTGTCAGAATCTCA |
| | ACCCGTTTCTGTCGTCAAAAAGGCGTATCAGAAACTGTGCTACATTCAT |
| | CATATCATGGGAAAGGTGCCAGACGCTTGCACTGCCTGCGATCTGGTCA |
| | ATGTGGATTTGGATGACTGCATCTTTGAACAATAA |
| MCP-Rep-KDE- | MPGSSGSSASNFTQFVLVDNGGTGDVTVAPSNFANGVAEWISSNSRSQA |
| mu amino acid | YKVTCSVRQSSAQNRKYTIKVEVPKVATQTVGGVELPVAAWRSYLNMEL |
| sequence (linker- | TIPIFATNSDCELIVKAMQGLLKDGNPIPSAIAANSGIYGSSGSSGFYE |
| MCP-linker-Rep) | IVIKVPSDLDEHLPGISDSFVNWVAEKEWELPPDSDMDLNLIEQAPLTV |
| SEQ ID NO: 128 | AEKLQRDFLTEWRRVSKAPEALFFVQFEKGESYFHMHVLVETTGVKSMV |
| | LGRFLSQIREKLIQRIYRGIEPTLPNWFAVTKIRNGAGGGNAVVAACYI |
| | PNYLLPKTQPELQWAWTNMEQYLSACLNLTERKRLVAQHLTHVSQTQEQ |
| | NKENQNPNSDAPVIRSKTSARYMELVGWLVDKGITSEKQWIQEDQASYI |
| | SFNAASNSRSQIKAALDNAGKIMSLIKTAPDYLVGQQPVEDISSNRIYK |
| | ILELNGYDPQYAASVFLGWATKKFGKRNTIWLFGPATTGKINIAEAIAH |
| | TVPFYGCVNWTNENFPFNDCVDKMVIWWEEGKMTAKVVESAKAILGGSK |
| | VRVDQKCKSSAQIDPTPVIVTSNTNMCAVIDGNSTTFEHQQPLQDRMFK |
| | FELTRRLDHDFGKVTKQEVKDFFRWAKDHVVEVEHEFYVKKGGAKKRPA |
| | PSDADISEPKRVRESVAQPSTSDAEASINYADRYQNKCSRHVGMNLMLF |
| | PCRQCERMNQNSNICFTHGQKDCLECFPVSESQPVSVVKKAYQKLCYIH |
| | HIMGKVPDACTACDLVNVDLDDCIFEQ |
| MCP-Rep-EKE- | ATGCCCGGCAGCTCCGGCAGTAGCGCTTCTAACTTTACTCAGTTCGTTC |
| mu nucleic acid | TCGTCGACAATGGCGGAACTGGCGACGTGACTGTCGCCCCAAGCAACTT |
| sequence (linker- | CGCTAACGGGGTCGCTGAATGGATCAGCTCTAACTCGCGTTCACAGGCT |
| MCP-linker-Rep) | TACAAAGTAACCTGTAGCGTTCGTCAGAGCTCTGCGCAGAATCGCAAAT |
| SEQ ID NO: 129 | ACACCATCAAAGTCGAGGTGCCTAAAGTGGCAACCCAGACTGTTGGTGG |
| | AGTAGAGCTTCCTGTAGCCGCATGGCGTTCGTACTTAAATATGGAACTA |
| | ACCATTCCAATTTTCGCTACGAATTCCGACTGCGAGCTTATTGTTAAGG |
| | CAATGCAAGGTCTCCTAAAAGATGGAAACCCGATTCCCTCAGCAATCGC |
| | AGCAAACTCCGGCATCTACGGCAGTAGTGGGTCCTCTGGGTTTTACGAG |
| | ATTGTGATTAAGGTCCCCAGCGACCTTGACGAGCATCTGCCCGGCATTT |
| | CTGACAGCTTTGTGAACTGGGTGGCCGAGAAGGAATGGGAGTTGCCGCC |
| | AGATTCTGACATGGATCTGAATCTGATTGAGCAGGCACCCCTGACCGTG |
| | GCCGAGAAGCTGCAGCGCGACTTTCTGACGGAATGGCGCCGTGTGAGTA |
| | AGGCCCCGGAGGCCCTTTTCTTTGTGCAATTTGCGGCGGGAGCGAGCTA |
| | CTTCCACATGCACGTGCTCGTGGAAACCACCGGGGTGAAATCCATGGTT |
| | TTGGGACGTTTCCTGAGTCAGATTCGCGAAAAACTGATTCAGAGAATTT |
| | ACCGCGGGATCGAGCCGACTTTGCCAAACTGGTTCGCGGTCACAAAGAC |
| | CAGAAATGGCGCCGGAGGCGGGAACAAGGTGGTGGATGAGTGCTACATC |
| | CCCAATTACTTGCTCCCCAAAACCCAGCCTGAGCTCCAGTGGGCGTGGA |
| | CTAATATGGAACAGTATTTAAGCGCCTGTTTGAATCTCACGGAGCGTAA |
| | ACGGTTGGTGGCGCAGCATCTGACGCACGTGTCGCAGACGCAGGAGCAG |
| | AACAAAGAGAATCAGAATCCCAATTCTGATGCGCCGGTGATCAGATCAA |
| | AAACTTCAGCCAGGTACATGGAGCTGGTCGGGTGGCTCGTGGACAAGGG |
| | GATTACCTCGGAGAAGCAGTGGATCCAGGAGGACCAGGCCTCATACATC |
| | TCCTTCAATGCGGCCTCCAACTCGCGGTCCCAAATCAAGGCTGCCTTGG |
| | ACAATGCGGGAAAGATTATGAGCCTGACTAAAACCGCCCCCGACTACCT |
| | GGTGGGCCAGCAGCCCGTGGAGGACATTTCCAGCAATCGGATTTATAAA |
| | ATTTTGGAACTAAACGGGTACGATCCCCAATATGCGGCTTCCGTCTTTC |
| | TGGGATGGGCCACGAAAAAGTTCGGCAAGAGGAACACCATCTGGCTGTT |
| | TGGGCCTGCAACTACCGGGAAGACCAACATCGCGGAGGCCATAGCCCAC |
| | ACTGTGCCCTTCTACGGGTGCGTAAACTGGACCAATGAGAACTTTCCCT |
| | TCAACGACTGTGTCGACAAGATGGTGATCTGGTGGGAGGAGGGGAAGAT |
| | GACCGCCAAGGTCGTGGAGTCGGCCAAAGCCATTCTCGGAGGAAGCAAG |
| | GTGCGCGTGGACCAGAAATGCAAGTCCTCGGCCCAGATAGACCCGACTC |
| | CCGTGATCGTCACCTCCAACACCAACATGTGCGCCGTGATTGACGGGAA |
| | CTCAACGACCTTCGAACACCAGCAGCCGTTGCAAGACCGGATGTTCAAA |
| | TTTGAACTCACCCGCCGTCTGGATCATGACTTTGGGAAGGTCACCAAGC |
| | AGGAAGTCAAAGACTTTTTCCGGTGGGCAAAGGATCACGTGGTTGAGGT |
| | GGAGCATGAATTCTACGTCAAAAAGGGTGGAGCCAAGAAAAGACCCGCC |
| | CCCAGTGACGCAGATATAAGTGAGCCCAAACGGGTGCGCGAGTCAGTTG |
| | CGCAGCCATCGACGTCAGACGCGGAAGCTTCGATCAACTACGCAGACAG |
| | GTACCAAAACAAATGTTCTCGTCACGTGGGCATGAATCTGATGCTGTTT |
| | CCCTGCAGACAATGCGAGAGAATGAATCAGAATTCAAATATCTGCTTCA |
| | CTCACGGACAGAAAGACTGTTTAGAGTGCTTTCCCGTGTCAGAATCTCA |
| | ACCCGTTTCTGTCGTCAAAAAGGCGTATCAGAAACTGTGCTACATTCAT |
| | CATATCATGGGAAAGGTGCCAGACGCTTGCACTGCCTGCGATCTGGTCA |
| | ATGTGGATTTGGATGACTGCATCTTTGAACAATAA |
| MCP-Rep-EKE- | MPGSSGSSASNFTQFVLVDNGGTGDVTVAPSNFANGVAEWISSNSRSQA |
| mu amino acid | YKVTCSVRQSSAQNRKYTIKVEVPKVATQTVGGVELPVAAWRSYLNMEL |
| sequence (linker- | TIPIFATNSDCELIVKAMQGLLKDGNPIPSAIAANSGIYGSSGSSGFYE |
| MCP-linker-Rep) | IVIKVPSDLDEHLPGISDSFVNWVAEKEWELPPDSDMDLNLIEQAPLTV |
| SEQ ID NO: 130 | AEKLQRDFLTEWRRVSKAPEALFFVQFAAGASYFHMHVLVETTGVKSMV |
| | LGRFLSQIREKLIQRIYRGIEPTLPNWFAVTKTRNGAGGGNKVVDECYI |
| | PNYLLPKTQPELQWAWINMEQYLSACLNLTERKRLVAQHLTHVSQTQEQ |
| | NKENQNPNSDAPVIRSKTSARYMELVGWLVDKGITSEKQWIQEDQASYI |
| | SFNAASNSRSQIKAALDNAGKIMSLIKTAPDYLVGQQPVEDISSNRIYK |
| | ILELNGYDPQYAASVELGWATKKFGKRNTIWLFGPATTGKTNIAEAIAH |
| | TVPFYGCVNWTNENFPFNDCVDKMVIWWEEGKMTAKVVESAKAILGGSK |
| | VRVDQKCKSSAQIDPTPVIVTSNTNMCAVIDGNSTTFEHQQPLQDRMFK |
| | FELTRRLDHDFGKVTKQEVKDFFRWAKDHVVEVEHEFYVKKGGAKKRPA |
| | PSDADISEPKRVRESVAQPSTSDAEASINYADRYQNKCSRHVGMNLMLF |
| | PCRQCERMNQNSNICFTHGQKDCLECFPVSESQPVSVVKKAYQKLCYIH |
| | HIMGKVPDACTACDLVNVDLDDCIFEQ |
| 2XMCP- | ATGCCCGGCAGCTCCGGCAGTAGCGCTTCTAACTTTACTCAGTTCGTTC |
| Rep nucleic acid | TCGTCGACAATGGCGGAACTGGCGACGTGACTGTCGCCCCAAGCAACTT |
| sequence (linker- | CGCTAACGGGATCGCTGAATGGATCAGCTCTAACTCGCGTTCACAGGCT |
| 2XMCP-linker- | TACAAAGTAACCTGTAGCGTTCGTCAGAGCTCTGCGCAGAATCGCAAAT |
| Rep) | ACACCATCAAAGTCGAGGTGCCTAAAGGCGCCTGGCGTTCGTACTTAAA |
| SEQ ID NO: 131 | TATGGAACTAACCATTCCAATTTTCGCCACGAATTCCGACTGCGAGCTT |
| | ATTGTTAAGGCAATGCAAGGTCTCCTAAAAGATGGAAACCCGATTCCCT |
| | CAGCAATCGCAGCAAACTCCGGCATCTACGGTGGTGGAGGAGGAATGGC |
| | GTCCAATTTCACGCAGTTCGTCCTGGTTGACAACGGGGGGACTGGGGAC |
| | GTTACGGTCGCTCCGAGCAACTTTGCCAATGGTATTGCGGAGTGGATTT |
| | CTTCTAATTCACGGTCCCAAGCTTACAAAGTGACCTGTTCCGTGCGGCA |
| | AAGTTCTGCTCAGAATAGAAAGTACACTATAAAGGTCGAAGTCCCTAAG |
| | GGGGCCTGGCGATCATATCTCAATATGGAGCTTACCATCCCAATATTTG |
| | CCACTAATTCTGATTGTGAATTGATTGTCAAAGCAATGCAAGGACTCTT |
| | GAAAGACGGAAACCCAATCCCCAGCGCAATCGCAGCCAACTCCGGTATA |
| | TACGGCAGTAGTGGGTCCTCTGGGTTTTACGAGATTGTGATTAAGGTCC |
| | CCAGCGACCTTGACGAGCATCTGCCCGGCATTTCTGACAGCTTTGTGAA |
| | CTGGGTGGCCGAGAAGGAATGGGAGTTGCCGCCAGATTCTGACATGGAT |
| | CTGAATCTGATTGAGCAGGCACCCCTGACCGTGGCCGAGAAGCTGCAGC |
| | GCGACTTTCTGACGGAATGGCGCCGTGTGAGTAAGGCCCCGGAGGCCCT |
| | TTTCTTTGTGCAATTTGAGAAGGGAGAGAGCTACTTCCACATGCACGTG |
| | CTCGTGGAAACCACCGGGGTGAAATCCATGGTTTTGGGACGTTTCCTGA |
| | GTCAGATTCGCGAAAAACTGATTCAGAGAATTTACCGCGGGATCGAGCC |
| | GACTTTGCCAAACTGGTTCGCGGTCACAAAGACCAGAAATGGCGCCGGA |
| | GGCGGGAACAAGGTGGTGGATGAGTGCTACATCCCCAATTACTTGCTCC |
| | CCAAAACCCAGCCTGAGCTCCAGTGGGCGTGGACTAATATGGAACAGTA |
| | TTTAAGCGCCTGTTTGAATCTCACGGAGCGTAAACGGTTGGTGGCGCAG |
| | CATCTGACGCACGTGTCGCAGACGCAGGAGCAGAACAAAGAGAATCAGA |
| | ATCCCAATTCTGATGCGCCGGTGATCAGATCAAAAACTTCAGCCAGGTA |
| | CATGGAGCTGGTCGGGTGGCTCGTGGACAAGGGGATTACCTCGGAGAAG |
| | CAGTGGATCCAGGAGGACCAGGCCTCATACATCTCCTTCAATGCGGCCT |
| | CCAACTCGCGGTCCCAAATCAAGGCTGCCTTGGACAATGCGGGAAAGAT |
| | TATGAGCCTGACTAAAACCGCCCCCGACTACCTGGTGGGCCAGCAGCCC |
| | GTGGAGGACATTTCCAGCAATCGGATTTATAAAATTTTGGAACTAAACG |
| | GGTACGATCCCCAATATGCGGCTTCCGTCTTTCTGGGATGGGCCACGAA |
| | AAAGTTCGGCAAGAGGAACACCATCTGGCTGTTTGGGCCTGCAACTACC |
| | GGGAAGACCAACATCGCGGAGGCCATAGCCCACACTGTGCCCTTCTACG |
| | GGTGCGTAAACTGGACCAATGAGAACTTTCCCTTCAACGACTGTGTCGA |
| | CAAGATGGTGATCTGGTGGGAGGAGGGGAAGATGACCGCCAAGGTCGTG |
| | GAGTCGGCCAAAGCCATTCTCGGAGGAAGCAAGGTGCGCGTGGACCAGA |
| | AATGCAAGTCCTCGGCCCAGATAGACCCGACTCCCGTGATCGTCACCTC |
| | CAACACCAACATGTGCGCCGTGATTGACGGGAACTCAACGACCTTCGAA |
| | CACCAGCAGCCGTTGCAAGACCGGATGTTCAAATTTGAACTCACCCGCC |
| | GTCTGGATCATGACTTTGGGAAGGTCACCAAGCAGGAAGTCAAAGACTT |
| | TTTCCGGTGGGCAAAGGATCACGTGGTTGAGGTGGAGCATGAATTCTAC |
| | GTCAAAAAGGGTGGAGCCAAGAAAAGACCCGCCCCCAGTGACGCAGATA |
| | TAAGTGAGCCCAAACGGGTGCGCGAGTCAGTTGCGCAGCCATCGACGTC |
| | AGACGCGGAAGCTTCGATCAACTACGCAGACAGGTACCAAAACAAATGT |
| | TCTCGTCACGTGGGCATGAATCTGATGCTGTTTCCCTGCAGACAATGCG |
| | AGAGAATGAATCAGAATTCAAATATCTGCTTCACTCACGGACAGAAAGA |
| | CTGTTTAGAGTGCTTTCCCGTGTCAGAATCTCAACCCGTTTCTGTCGTC |
| | AAAAAGGCGTATCAGAAACTGTGCTACATTCATCATATCATGGGAAAGG |
| | TGCCAGACGCTTGCACTGCCTGCGATCTGGTCAATGTGGATTTGGATGA |
| | CTGCATCTTTGAACAATAA |
| 2XMCP-Rep | MPGSSGSSASNFTQFVLVDNGGTGDVTVAPSNFANGIAEWISSNSRSQA |
| amino acid | YKVTCSVRQSSAONRKYTIKVEVPKGAWRSYLNMELTIPIFATNSDCEL |
| sequence (linker- | IVKAMQGLLKDGNPIPSAIAANSGIYGGGGGMASNFTQFVLVDNGGTGD |
| 2XMCP-linker- | VTVAPSNFANGIAEWISSNSRSQAYKVTCSVRQSSAONRKYTIKVEVPK |
| Rep) | GAWRSYLNMELTIPIFATNSDCELIVKAMQGLLKDGNPIPSAIAANSGI |
| SEQ ID NO: 132 | YGSSGSSGFYEIVIKVPSDLDEHLPGISDSFVNWVAEKEWELPPDSDMD |
| | LNLIEQAPLTVAEKLQRDFLTEWRRVSKAPEALFFVQFEKGESYFHMHV |
| | LVETTGVKSMVLGRFLSQIREKLIQRIYRGIEPTLPNWFAVTKTRNGAG |
| | GGNKVVDECYIPNYLLPKTQPELQWAWTNMEQYLSACLNLTERKRLVAQ |
| | HLTHVSQTQEQNKENQNPNSDAPVIRSKTSARYMELVGWLVDKGITSEK |
| | QWIQEDQASYISFNAASNSRSQIKAALDNAGKIMSLTKTAPDYLVGQQP |
| | VEDISSNRIYKILELNGYDPQYAASVFLGWATKKFGKRNTIWLFGPATT |
| | GKTNIAEAIAHTVPFYGCVNWTNENFPFNDCVDKMVIWWEEGKMTAKVV |
| | ESAKAILGGSKVRVDQKCKSSAQIDPTPVIVTSNTNMCAVIDGNSTTFE |
| | HQQPLQDRMFKFELTRRLDHDFGKVTKQEVKDFFRWAKDHVVEVEHEFY |
| | VKKGGAKKRPAPSDADISEPKRVRESVAQPSTSDAEASINYADRYQNKC |
| | SRHVGMNLMLFPCRQCERMNQNSNICFTHGQKDCLECFPVSESQPVSVV |
| | KKAYQKLCYIHHIMGKVPDACTACDLVNVDLDDCIFEQ |
| PCP-Rep-Y156F | ATGCCCGGCAGCTCCGGCAGTAGCTCCAAAACAATAGTCCTCTCCGTAG |
| nucleic acid | GGGAGGCAACACGGACTTTGACCGAAATCCAGTCAACCGCTGACCGACA |
| sequence (linker- | AATCTTTGAAGAGAAAGTAGGGCCTCTTGTGGGCCGACTGCGCTTGACT |
| PCP-linker-Rep) | GCAAGCTTGCGACAAAACGGCGCAAAGACTGCCTATAGGGTCAACCTTA |
| SEQ ID NO: 133 | AACTCGACCAAGCCGACGTGGTCGATAGCGGTCTCCCTAAGGTTCGGTA |
| | TACGCAGGTCTGGAGTCATGACGTAACAATCGTAGCAAACAGCACAGAA |
| | GCCTCCCGAAAAAGCCTCTACGATCTGACGAAATCCTTGGTGGCTACGT |
| | CACAGGTGGAAGACCTCGTTGTCAACCTTGTACCTCTGGGTCGAGGCAG |
| | TAGTGGGTCCTCTGGGTTTTACGAGATTGTGATTAAGGTCCCCAGCGAC |
| | CTTGACGAGCATCTGCCCGGCATTTCTGACAGCTTTGTGAACTGGGTGG |
| | CCGAGAAGGAATGGGAGTTGCCGCCAGATTCTGACATGGATCTGAATCT |
| | GATTGAGCAGGCACCCCTGACCGTGGCCGAGAAGCTGCAGCGCGACTTT |
| | CTGACGGAATGGCGCCGTGTGAGTAAGGCCCCGGAGGCCCTTTTCTTTG |
| | TGCAATTTGAGAAGGGAGAGAGCTACTTCCACATGCACGTGCTCGTGGA |
| | AACCACCGGGGTGAAATCCATGGTTTTGGGACGTTTCCTGAGTCAGATT |
| | CGCGAAAAACTGATTCAGAGAATTTACCGCGGGATCGAGCCGACTTTGC |
| | CAAACTGGTTCGCGGTCACAAAGACCAGAAATGGCGCCGGAGGCGGGAA |
| | CAAGGTGGTGGATGAGTGCTACATCCCCAATTTCTTGCTCCCCAAAACC |
| | CAGCCTGAGCTCCAGTGGGCGTGGACTAATATGGAACAGTATTTAAGCG |
| | CCTGTTTGAATCTCACGGAGCGTAAACGGTTGGTGGCGCAGCATCTGAC |
| | GCACGTGTCGCAGACGCAGGAGCAGAACAAAGAGAATCAGAATCCCAAT |
| | TCTGATGCGCCGGTGATCAGATCAAAAACTTCAGCCAGGTACATGGAGC |
| | TGGTCGGGTGGCTCGTGGACAAGGGGATTACCTCGGAGAAGCAGTGGAT |
| | CCAGGAGGACCAGGCCTCATACATCTCCTTCAATGCGGCCTCCAACTCG |
| | CGGTCCCAAATCAAGGCTGCCTTGGACAATGCGGGAAAGATTATGAGCC |
| | TGACTAAAACCGCCCCCGACTACCTGGTGGGCCAGCAGCCCGTGGAGGA |
| | CATTTCCAGCAATCGGATTTATAAAATTTTGGAACTAAACGGGTACGAT |
| | CCCCAATATGCGGCTTCCGTCTTTCTGGGATGGGCCACGAAAAAGTTCG |
| | GCAAGAGGAACACCATCTGGCTGTTTGGGCCTGCAACTACCGGGAAGAC |
| | CAACATCGCGGAGGCCATAGCCCACACTGTGCCCTTCTACGGGTGCGTA |
| | AACTGGACCAATGAGAACTTTCCCTTCAACGACTGTGTCGACAAGATGG |
| | TGATCTGGTGGGAGGAGGGGAAGATGACCGCCAAGGTCGTGGAGTCGGC |
| | CAAAGCCATTCTCGGAGGAAGCAAGGTGCGCGTGGACCAGAAATGCAAG |
| | TCCTCGGCCCAGATAGACCCGACTCCCGTGATCGTCACCTCCAACACCA |
| | ACATGTGCGCCGTGATTGACGGGAACTCAACGACCTTCGAACACCAGCA |
| | GCCGTTGCAAGACCGGATGTTCAAATTTGAACTCACCCGCCGTCTGGAT |
| | CATGACTTTGGGAAGGTCACCAAGCAGGAAGTCAAAGACTTTTTCCGGT |
| | GGGCAAAGGATCACGTGGTTGAGGTGGAGCATGAATTCTACGTCAAAAA |
| | GGGTGGAGCCAAGAAAAGACCCGCCCCCAGTGACGCAGATATAAGTGAG |
| | CCCAAACGGGTGCGCGAGTCAGTTGCGCAGCCATCGACGTCAGACGCGG |
| | AAGCTTCGATCAACTACGCAGACAGGTACCAAAACAAATGTTCTCGTCA |
| | CGTGGGCATGAATCTGATGCTGTTTCCCTGCAGACAATGCGAGAGAATG |
| | AATCAGAATTCAAATATCTGCTTCACTCACGGACAGAAAGACTGTTTAG |
| | AGTGCTTTCCCGTGTCAGAATCTCAACCCGTTTCTGTCGTCAAAAAGGC |
| | GTATCAGAAACTGTGCTACATTCATCATATCATGGGAAAGGTGCCAGAC |
| | GCTTGCACTGCCTGCGATCTGGTCAATGTGGATTTGGATGACTGCATCT |
| | TTGAACAATAA |
| PCP-Rep-Y156F | MPGSSGSSSKTIVLSVGEATRTLTEIQSTADRQIFEEKVGPLVGRLRLT |
| amino acid | ASLRQNGAKTAYRVNLKLDQADVVDSGLPKVRYTQVWSHDVTIVANSTE |
| sequence (linker- | ASRKSLYDLTKSLVATSQVEDLVVNLVPLGRGSSGSSGFYEIVIKVPSD |
| PCP-linker-Rep) | LDEHLPGISDSFVNWVAEKEWELPPDSDMDLNLIEQAPLTVAEKLQRDE |
| SEQ ID NO: 134 | LTEWRRVSKAPEALFFVQFEKGESYFHMHVLVETTGVKSMVLGRELSQI |
| | REKLIQRIYRGIEPTLPNWFAVTKTRNGAGGGNKVVDECYIPNFLLPKT |
| | QPELQWAWINMEQYLSACLNLTERKRLVAQHLTHVSQTQEQNKENQNPN |
| | SDAPVIRSKTSARYMELVGWLVDKGITSEKQWIQEDQASYISENAASNS |
| | RSQIKAALDNAGKIMSLIKTAPDYLVGQQPVEDISSNRIYKILELNGYD |
| | PQYAASVFLGWATKKFGKRNTIWLFGPATTGKINIAEAIAHTVPFYGCV |
| | NWTNENFPFNDCVDKMVIWWEEGKMTAKVVESAKAILGGSKVRVDQKCK |
| | SSAQIDPTPVIVTSNTNMCAVIDGNSTTFEHQQPLQDRMEKFELTRRLD |
| | HDFGKVTKQEVKDFFRWAKDHVVEVEHEFYVKKGGAKKRPAPSDADISE |
| | PKRVRESVAQPSTSDAEASINYADRYQNKCSRHVGMNLMLFPCRQCERM |
| | NQNSNICFTHGQKDCLECFPVSESQPVSVVKKAYQKLCYIHHIMGKVPD |
| | ACTACDLVNVDLDDCIFEQ |
| COM-Rep-Y156F | ATGCCCGGCAGCTCCGGCAGTAGCATGAAATCAATTCGCTGTAAAAACT |
| nucleic acid | GCAACAAACTGTTATTTAAGGCGGATTCCTTTGATCACATTGAAATCAG |
| sequence (linker- | GTGTCCGCGTTGCAAACGTCACATCATAATGCTGAATGCCTGCGAGCAT |
| COM-linker-Rep) | CCCACGGAGAAACATTGTGGGAAAAGAGAAAAAATCACGCATTCTGACG |
| SEQ ID NO: 135 | AAACCGTGCGTTATGGCAGTAGTGGGTCCTCTGGGTTTTACGAGATTGT |
| | GATTAAGGTCCCCAGCGACCTTGACGAGCATCTGCCCGGCATTTCTGAC |
| | AGCTTTGTGAACTGGGTGGCCGAGAAGGAATGGGAGTTGCCGCCAGATT |
| | CTGACATGGATCTGAATCTGATTGAGCAGGCACCCCTGACCGTGGCCGA |
| | GAAGCTGCAGCGCGACTTTCTGACGGAATGGCGCCGTGTGAGTAAGGCC |
| | CCGGAGGCCCTTTTCTTTGTGCAATTTGAGAAGGGAGAGAGCTACTTCC |
| | ACATGCACGTGCTCGTGGAAACCACCGGGGTGAAATCCATGGTTTTGGG |
| | ACGTTTCCTGAGTCAGATTCGCGAAAAACTGATTCAGAGAATTTACCGC |
| | GGGATCGAGCCGACTTTGCCAAACTGGTTCGCGGTCACAAAGACCAGAA |
| | ATGGCGCCGGAGGCGGGAACAAGGTGGTGGATGAGTGCTACATCCCCAA |
| | TTTCTTGCTCCCCAAAACCCAGCCTGAGCTCCAGTGGGCGTGGACTAAT |
| | ATGGAACAGTATTTAAGCGCCTGTTTGAATCTCACGGAGCGTAAACGGT |
| | TGGTGGCGCAGCATCTGACGCACGTGTCGCAGACGCAGGAGCAGAACAA |
| | AGAGAATCAGAATCCCAATTCTGATGCGCCGGTGATCAGATCAAAAACT |
| | TCAGCCAGGTACATGGAGCTGGTCGGGTGGCTCGTGGACAAGGGGATTA |
| | CCTCGGAGAAGCAGTGGATCCAGGAGGACCAGGCCTCATACATCTCCTT |
| | CAATGCGGCCTCCAACTCGCGGTCCCAAATCAAGGCTGCCTTGGACAAT |
| | GCGGGAAAGATTATGAGCCTGACTAAAACCGCCCCCGACTACCTGGTGG |
| | GCCAGCAGCCCGTGGAGGACATTTCCAGCAATCGGATTTATAAAATTTT |
| | GGAACTAAACGGGTACGATCCCCAATATGCGGCTTCCGTCTTTCTGGGA |
| | TGGGCCACGAAAAAGTTCGGCAAGAGGAACACCATCTGGCTGTTTGGGC |
| | CTGCAACTACCGGGAAGACCAACATCGCGGAGGCCATAGCCCACACTGT |
| | GCCCTTCTACGGGTGCGTAAACTGGACCAATGAGAACTTTCCCTTCAAC |
| | GACTGTGTCGACAAGATGGTGATCTGGTGGGAGGAGGGGAAGATGACCG |
| | CCAAGGTCGTGGAGTCGGCCAAAGCCATTCTCGGAGGAAGCAAGGTGCG |
| | CGTGGACCAGAAATGCAAGTCCTCGGCCCAGATAGACCCGACTCCCGTG |
| | ATCGTCACCTCCAACACCAACATGTGCGCCGTGATTGACGGGAACTCAA |
| | CGACCTTCGAACACCAGCAGCCGTTGCAAGACCGGATGTTCAAATTTGA |
| | ACTCACCCGCCGTCTGGATCATGACTTTGGGAAGGTCACCAAGCAGGAA |
| | GTCAAAGACTTTTTCCGGTGGGCAAAGGATCACGTGGTTGAGGTGGAGC |
| | ATGAATTCTACGTCAAAAAGGGTGGAGCCAAGAAAAGACCCGCCCCCAG |
| | TGACGCAGATATAAGTGAGCCCAAACGGGTGCGCGAGTCAGTTGCGCAG |
| | CCATCGACGTCAGACGCGGAAGCTTCGATCAACTACGCAGACAGGTACC |
| | AAAACAAATGTTCTCGTCACGTGGGCATGAATCTGATGCTGTTTCCCTG |
| | CAGACAATGCGAGAGAATGAATCAGAATTCAAATATCTGCTTCACTCAC |
| | GGACAGAAAGACTGTTTAGAGTGCTTTCCCGTGTCAGAATCTCAACCCG |
| | TTTCTGTCGTCAAAAAGGCGTATCAGAAACTGTGCTACATTCATCATAT |
| | CATGGGAAAGGTGCCAGACGCTTGCACTGCCTGCGATCTGGTCAATGTG |
| | GATTTGGATGACTGCATCTTTGAACAATAA |
| COM-Rep-Y156F | MPGSSGSSMKSIRCKNCNKLLFKADSFDHIEIRCPRCKRHIIMLNACEH |
| amino acid | PTEKHCGKREKITHSDETVRYGSSGSSGFYEIVIKVPSDLDEHLPGISD |
| sequence (linker- | SFVNWVAEKEWELPPDSDMDLNLIEQAPLTVAEKLQRDFLTEWRRVSKA |
| COM-linker-Rep) | PEALFFVQFEKGESYFHMHVLVETTGVKSMVLGRELSQIREKLIQRIYR |
| SEQ ID NO: 136 | GIEPTLPNWFAVTKTRNGAGGGNKVVDECYIPNFLLPKTQPELQWAWTN |
| | MEQYLSACLNLTERKRLVAQHLTHVSQTQEQNKENQNPNSDAPVIRSKT |
| | SARYMELVGWLVDKGITSEKQWIQEDQASYISFNAASNSRSQIKAALDN |
| | AGKIMSLIKTAPDYLVGQQPVEDISSNRIYKILELNGYDPQYAASVFLG |
| | WATKKFGKRNTIWLFGPATTGKTNIAEAIAHTVPFYGCVNWTNENFPFN |
| | DCVDKMVIWWEEGKMTAKVVESAKAILGGSKVRVDQKCKSSAQIDPTPV |
| | IVTSNTNMCAVIDGNSTTFEHQQPLQDRMFKFELTRRLDHDFGKVTKQE |
| | VKDFFRWAKDHVVEVEHEFYVKKGGAKKRPAPSDADISEPKRVRESVAQ |
| | PSTSDAEASINYADRYQNKCSRHVGMNLMLFPCRQCERMNQNSNICFTH |
| | GQKDCLECFPVSESQPVSVVKKAYQKLCYIHHIMGKVPDACTACDLVNV |
| | DLDDCIFEQ |

* Sequence elements are matched based on formatting styles (e.g., double underline, bold, and/or italic fonts, etc.)

TABLE D
Nucleic Acid Sequence and Amino Acid Sequence of AAP and MCP
fusion proteins

| AAP and MCP | |
|---|---|
| fusion proteins | Sequences |
| MCP-AAP(DJ) | ATGGGCAGCTCCGGCAGTAGCGCTTCTAACTTTACTCAGT |
| nucleic acid | TCGTTCTCGTCGACAATGGCGGAACTGGCGACGTGACTGT |
| sequence (linker- | CGCCCCAAGCAACTTCGCTAACGGGGTCGCTGAATGGATC |
| MCP-linker- | AGCTCTAACTCGCGTTCACAGGCTTACAAAGTAACCTGTA |
| AAP) SEQ ID NO: 137 | GCGTTCGTCAGAGCTCTGCGCAGAATCGCAAATACACCAT |
| | CAAAGTCGAGGTGCCTAAAGTGGCAACCCAGACTGTTGGT |
| | GGAGTAGAGCTTCCTGTAGCCGCATGGCGTTCGTACTTAA |
| | ATATGGAACTAACCATTCCAATTTTCGCTACGAATTCCGAC |
| | TGCGAGCTTATTGTTAAGGCAATGCAAGGTCTCCTAAAAG |
| | ATGGAAACCCGATTCCCTCAGCAATCGCAGCAAACTCCGG |
| | CATCTACGGCAGTAGTGGGTCCTCTCTGGAGACGCAGACT |
| | CAGTCCCAGACCCTCAACCAATCGGAGAACCTCCCGCAG |
| | CCCCCTCAGGTGTGGGATCTCTTACAATGGCTGCAGGCG |
| | GTGGCGCACCAATGGCAGACAATAACGAGGGCGCCGAC |
| | GGAGTGGGTAATTCCTCGGGAAATTGGCATTGCGATTCC |
| | ACATGGATGGGCGACAGAGTCATCACCACCAGCACCCGA |
| | ACCTGGGCCCTGCCCACCTACAACAACCACCTCTACAAG |
| | CAAATCTCCAACAGCACATCTGGAGGATCTTCAAATGAC |
| | AACGCCTACTTCGGCTACAGCACCCCCTGGGGGTATTTTG |
| | ACTTTAACAGATTCCACTGCCACTTTTCACCACGTGACTG |
| | GCAGCGACTCATCAACAACAACTGGGGATTCCGGCCCAA |
| | GAGACTCAGCTTCAAGCTCTTCAACATCCAGGTCAAGGA |
| | GGTCACGCAGAATGAAGGCACCAAGACCATCGCCAATAA |
| | CCTCACCAGCACCATCCAGGTGTTTACGGACTCGGAGTA |
| | CCAGCTGCCGTACGTTCTCGGCTCTGCCCACCAGGGCTGC |
| | CTGCCTCCGTTCCCGGCGGACGTGTTCATGA |
| MCP-AAP(DJ) | MMGSSGSSASNFTQFVLVDNGGTGDVTVAPSSNFANGVAEWIS |
| amino acid | SNSRSQAYKVTCSVRQSSAQNRKYTIKVEVPKVATQTVGGVE |
| sequence (linker- | LPVAAWRSYLNMELTIPIFATNSDCELIVKAMQGLLKDGNPI |
| MCP-linker- | PSAIAANSGIYGSSGSSLETQTQSQTLNQSENLPQPPQVWDL |
| AAP) SEQ ID NO: 138 | LQWLQAVAHQWQTITRAPTEWVIPREIGIAIPHGWATESSPP |
| | APEPGPCPPTTTTSTSKSPTAHLEDLQMTTPTSATAPPGGILT |
| | LTDSTATFHHVTGSDSSTTTGDSGPRDSASSSSTSRSRRSRR |
| | MKAPRPSPITSPAPSRCLRTRSTSCRTFSALPTRAACLRSRRTCS |
| AAP-MCP(DJ) | ATGCTGGAGACGCAGACTCAGTCCCAGACCCTCAACCAATCGGAGAACC |
| nucleic acid | TCCCGCAGCCCCCTCAGGTGTGGGATCTCTTACAATGGCTGCAGGCGGT |
| sequence (AAP- | GGCGCACCAATGGCAGACAATAACGAGGGCGCCGACGGAGTGGGTAATT |
| linker-MCP- | CCTCGGGAAATTGGCATTGCGATTCCACATGGATGGGCGACAGAGTCAT |
| linker) | CACCACCAGCACCCGAACCTGGGCCCTGCCCACCTACAACAACCACCTC |
| SEQ ID NO: 139 | TACAAGCAAATCTCCAACAGCACATCTGGAGGATCTTCAAATGACAACG |
| | CCTACTTCGGCTACAGCACCCCCTGGGGGTATTTTGACTTTAACAGATT |
| | CCACTGCCACTTTTCACCACGTGACTGGCAGCGACTCATCAACAACAAC |
| | TGGGGATTCCGGCCCAAGAGACTCAGCTTCAAGCTCTTCAACATCCAGG |
| | TCAAGGAGGTCACGCAGAATGAAGGCACCAAGACCATCGCCAATAACCT |
| | CACCAGCACCATCCAGGTGTTTACGGACTCGGAGTACCAGCTGCCGTAC |
| | GTTCTCGGCTCTGCCCACCAGGGCTGCCTGCCTCCGTTCCCGGCGGACG |
| | TGTTCAGGCAGCTCCGGCAGTAGCGCTTCTAACTTTACTCAG |
| | TTCGTTCTCGTCGACAATGGCGGAACTGGCGACGTGACTG |
| | TCGCCCCAAGCAACTTCGCTAACGGGGTCGCTGAATGGAT |
| | CAGCTCTAACTCGCGTTCACAGGCTTACAAAGTAACCTGTA |
| | GCGTTCGTCAGAGCTCTGCGCAGAATCGCAAATACACCAT |
| | CAAAGTCGAGGTGCCTAAAGTGGCAACCCAGACTGTTGGT |
| | GGAGTAGAGCTTCCTGTAGCCGCATGGCGTTCGTACTTAA |
| | ATATGGAACTAACCATTCCAATTTTCGCTACGAATTCCGAC |
| | TGCGAGCTTATTGTTAAGGCAATGCAAGGTCTCCTAAAAG |
| | ATGGAAACCCGATTCCCTCAGCAATCGCAGCAAACTCCGG |
| | CATCTACGGCAGTAGTGGGTCCTCTTGA |
| AAP-MCP(DJ) | MLETQTQSQTLNQSENLPQPPQVWDLLQWLQAVAHQWQTITRAPTEWVI |
| amino acid | PREIGIAIPHGWATESSPPAPEPGPCPPTTTTSTSKSPTAHLEDLQMTT |
| sequence (AAP- | PTSATAPPGGILTLTDSTATFHHVTGSDSSTTTGDSGPRDSASSSSTSR |
| linker-MCP- | SRRSRRMKAPRPSPITSPAPSRCLRTRSTSCRTFSALPTRAACLRSRRT |
| linker) | CSGSSGSSASNFTQFVLVDNGGTGDVTVAPSNFANGVAEWISSNSRSQA |
| SEQ ID NO: 140 | YKVTCSVRQSSAQNRKYTIKVEVPKVATQTVGGVELPVAAWRSYLNMEL |
| | TIPIFATNSDCELIVKAMQGLLKDRNPIPSAIAANSGIYGSSGSS |

* Sequence elements are matched based on formatting styles (e.g., double underline, bold and/or italic fonts, etc.)

TABLE E
Primer sequences

| Primer | Sequences |
|---|---|
| WPRE-F | CCCGTATGGCTTTCATTTTCTCC |
| SEQ ID NO: 141 | |
| WPRE-R | GGCAATGCCCCAACCAGTG |
| SEQ ID NO: 142 | |
| Cre-F | CCAGTAGATGCCACTAGCGA |
| SEQ ID NO: 143 | |
| Cre-R | GCCTGGAGATACAGCAGGTA |
| SEQ ID NO: 144 | |
| CAG-F | CTTCTCCTCCGGGCTGTAAT |
| SEQ ID NO: 145 | |
| CAG-R | CTTTCACGCAGCCACAGAAA |
| SEQ ID NO: 146 | |

* F and R stand for forward and reverse primers, respectively.

TABLE F
Stuffer sequence

| Stuffer | Sequences |
|---|---|
| Stuffer | ATATTTGGAGGGCAGCTTGATTTCGACTTCGGGAGGGAAGCTGCGCCAT |
| nucleic acid | GCGATGTTATCGGTGCGGTGAATGCAAAGAAGATAACCGCTTCCGACCA |
| sequence | AATCAACCTTACTGGAATCGATGGTGTCTCCGGTGTGAAAGAACACCAA |
| SEQ ID NO: 147 | CAGGGGTGTTACCACTACCGCAGGAAAAGGAGGACGTGCCGCGAGACAG |
| | CGACGAAGTATCACCGACATAATCTGCGAAAACTGCAAATACCTTCCAA |
| | CGAAACGCACCAGAAATAAACCCAAGCCAATCCCAAAAGAATCTGACGT |
| | AAAAACCTTCAACTACACGGCTCACCTGTGGGATATCCGGTGGCTAAGA |
| | CGTCGTGCGAGGAAAACAAGGCCATTGACCAAAATCGAAGTTACGAACA |
| | AGAAAGCGTCGAGCGAGCTTTAACGTGCGCTAACTGCGGTCAGAAGCTG |
| | CATGTGCTGGAAGTTCACGTGTGTGAGCACTGCTGCGCAGAACTGATGA |
| | GCGATCCGAATAGCTCGATGCACGAGGAAGAAGGCCGCCGCTAAACCAG |
| | CGCGAAGACGATGTAAAAACGATGAATGCCGGGAATGGTTTCACCCTGC |
| | ATTCGCTAATCAGTGGTGGTGCTCTCCAGAGTGTGGAACCAAGATAGCA |
| | CTCGAACGACGAAGTAAAGAACGCGAAAAAGCGGAAAAAGCAGCAGAGA |
| | AGAAACGACGACGAGAGGAGCAGAAACAGAAAGATAAACTTAAGATTCG |
| | AAAACTCGCCTTAAAGCCCCGCAGTTACTGGATTAAACAAGCCCAACAA |
| | GCCGTAAACGCCTTCATCAGAGAAAGAGACCGCGACTTACCATGTATCT |
| | CGTGCGGAACGCTCACGTCTGCTCAGTGGGATGCCGGACATTACCGGAC |
| | AACTGCTGCGGCACCTCAACTCCGATTTAATGAACGCAATATTCACAAG |
| | CAATGCGTGGTGTGCAACCAGCACAAAAGCGGAAATCTCGTTCCGTATC |
| | GCGTCGAACTGATTAGCCGCATCGGGCAGGAAGCAGTAGACGAAATCGA |
| | ATCAAACCATAACCGCCATCGCTGGACTATCGAAGAGTGCAAGGCGATC |
| | AAGGCAGAGTACCAACAGAAACTCAAAGACCTGCGAAATAGCAGAAGTG |
| | AGGCCGCGCCACGTTCTCAGTAAAAACCATTCCAGACATGCTCGTTGAA |
| | GCATACGGAAATCAGACAGAAGTAGCACGCAGACTGAAATGTAGTCGCG |
| | GTACGGTCAGAAAATACGTTGATGATAAAGACGGGAAAATGCACGCCAT |
| | CGTCAACGACGTTCTCATGGTTCATCGCGGATGGAGTGAAAGAGGCCCG |
| | CTATTACGAAAAAATTGATGGCAGCAAATACCGAAATATTTGGGTAGTT |
| | GGCGATCTGCACGGATGCTACACGAACCTGATGAACAAACTGGATACGA |
| | TTGGATTCGACAACAAAAAAGACCTGCTTATCTCGGTGGGCGATTTGGT |
| | TGATCGTGGTGCAGAGAACGTTGAATGCCTGGAATTAATCACATTCCCC |
| | TGGTTCAGAGCTGTACGTGGAAACCATGAGCAAATGATGATTGATGGCT |
| | TATCAGAGCGTGGAAACGTTAATCACTGGCTGCTTAATGGCGGTGGCTG |
| | GTTCTTTAATCTCGATTACGACAAAGAAATTCTGGCTAAAGCTCTTGCC |
| | CATAAAGCAGATGAACTTCCGTTAATCATCGAACTGGTGAGCAAAGATA |
| | AAAAATATGTTATCTGCCACGCCGATTATCCCTTTGACGAATACGAGTT |
| | TGGAAAGCCAGTTGATCATCAGCAGGTAATCTGGAACCGCGAACGAATC |
| | AGCAACTCACAAAACGGGATCGTGAAAGAAATCAAAGGCGCGGACACGT |
| | TCATCTTTGGTCATACGCCAGCAGTGAAACCACTCAAGTTTGCCAACCA |
| | AATGTATATCGATACCGGCGCAGTGTTCTGCGGAAACCTAACATTGATT |
| | CAGGTACAGGGAGAAGGCGCGCCAGACTCGAAAGCGTAGCTAAATTTCA |
| | TTCGCCAAAAAGCCCGATGATGAGCGACTCACCACGGGCCACGGCTTCT |
| | GACTCTCTTTCCGGTACTGATGTGATGGCTGCTATGGGGATGGCGCAAT |
| | CACAAGCCGGATTCGGTATGGCTGCATTCTGCGGTAAGCACGAACTCAG |
| | CCAGAACGACAAACAAAAGGCTATCAACTATCTGATGCAATTTGCACAC |
| | AAGGTATCGGGGAAATACCGTGGTGTGGCAAAGCTTGAAGGAAATACTA |
| | AGGCAAAGGTACTGCAAGTGCTCGCAACATTCGCTTATGCGGATTATTG |
| | CCGTAGTGCCGCGACGCCGGGGGCAAGATGCAGAGATTGCCATGGTACA |
| | GGCCGTGCGGTTGATATTGCCAAAACAGAGCTGTGGGGGAGAGTTGTCG |
| | AGAAAGAGTGCGGAAGATGCAAAGGCGTCGGCTATTCAAGGATGCCAGC |
| | AAGCGCAGCATATCGCGCTGTGACGATGCTAATCCCAAACCTTACCCAA |
| | CCCACCTGGTCACGCACTGTTAAGCCGCTGTATGACGCTCTGGTGGTGC |
| | AATGCCACAAAGAAGAGTCAATCGCAGACAACATTTTGAATGCGGTCAC |
| | ACGTTAGCAGCATGATTGCCACGGATGGCAACATATTAACGGCATGATA |
| | TTGACTTATTGAATAAAATTGGGTAAATTTGACTCAACGATGGGTTAAT |
| | TCGCTCGTTGTGGTAGTGAGGCCAAAAGAGGCGGCGCTTACTACCGATT |
| | CCGCCTAGTTGGTCACTTCGACGTATCGTCTGGAACTCCAACCATCGCA |
| | GGCAGAGAGGTCTGCAAAATGCAATCCCGAAACAGTTCGCAGGTAATAG |
| | TTAGAGCCTGCATAACGGTTTCGGGATTTTTTATATCTGCACAACAGGT |
| | AAGAGCATTGAGTCGATAATCGTGAAGAGTCGGCGAGCCTGGTTAGCCA |
| | GTGCTCTTTCCGTTGTGCTGAATTAAGCGAATACCGGAAGCAGAACCGG |
| | ATCACCAAATGCGTACAGGCGTCATCGCCGCCCAGCAACAGCACAACCC |
| | AAACTGAGCCGTAGCCACTGTCTGTCCTAAATTCATTAGTAATAGTTAC |
| | GCTGCGGCCTTTTACACATGACCTTCGTGAAAGCGGGTGGCAGGAGGTC |
| | GCGCTAACAACCTCCTGCCGTTTTGCCCGTGCATATCGGTCACGAACAA |
| | ATCTGATTACTAAACACAGTAGCCTGGATTTGTTCTATCAGTAATCGAC |
| | CTTATTCCTAATTAAATAGAGCAAATCCCCTT |

### INCORPORATION BY REFERENCE

The entire disclosure of each of the patent documents, including patent application documents, scientific articles, governmental reports, websites, and other references referred to herein is incorporated by reference herein in its entirety for all purposes. In case of a conflict in terminology, the present specification controls. All sequence listings, or SEQ ID NOs. disclosed herein are incorporated herein in their entirety.

The following references, to the extent that they provide exemplary procedural or other details supplementary to those set forth herein, are specifically incorporated herein by reference.

Although illustrative embodiments of the present invention have been described herein, it should be understood that the invention is not limited to those described, and that various other changes or modifications may be made by one skilled in the art without departing from the scope or spirit of the invention.

1-30. (canceled)

31. A recombinant DNA virus viral particle comprising a ribonucleotide (RNA) sequence packaged within the protein shell of a DNA virus viral particle, wherein the RNA sequence is capable of being packaged into the DNA virus viral particle, wherein said RNA sequence comprises:
(1) an RNA sequence of interest (RSI); and,
(2) an RNA-packaging signal (RPS) capable of binding directly or indirectly to an RPS-interacting molecule that facilitates packaging of the RNA sequence into the DNA virus viral particle.

32. The recombinant DNA virus viral particle of claim 31, wherein the DNA virus viral particle is an AAV, and the recombinant DNA virus viral particle is a recombinant RNA adeno-associated virus (rRAAV) viral particle, comprising:
(1) an AAV capsid; and,
(2) the RNA sequence packaged within said AAV capsid.

33. The recombinant DNA virus viral particle of claim 32, wherein the AAV capsid comprises a capsid from an AAV of the serotype AAV1, AAV2, AAV3A, AAV3B, AAV4, AAV5, AAV6, AAV7, AAV8, AAV9, AAV10, AAV11, AAV12, AAV13, AAV-DJ, AAV PHP.cB, Anc80L65, Anc80L65AAP, AAVrh74, or 7m8.

34. A population of recombinant DNA virus viral particles comprising a plurality of recombinant DNA virus viral particle of claim 31, wherein at least about 10%, 20%, 30%, 40%, 50%, 60%, 70%, 80%, 90%, 95%, 96%, 97%, 98%, 99% or more of the recombinant DNA virus viral particles within said population have the RNA sequence packaged therein.

35. A host cell comprising the recombinant DNA virus viral particle of claim 31.

36-47. (canceled)

48. The recombinant DNA virus viral particle of claim 31, wherein the DNA virus viral particle is an AAV viral particle or an oncolytic viral particle.

49. The recombinant DNA virus viral particle of claim 31, wherein the RPS is located at or near the 5′ end of the RSI, at or near the 3′ end of the RSI, or internal to the RSI.

50. The recombinant DNA virus viral particle of claim 31, wherein the RNA sequence comprises more than one RPS that are identical or different, and wherein two or more of said more than one RPS are
(1) adjacent to each other, or are in tandem, via the same or different linkers; or
(2) not adjacent to each other.

51. The recombinant DNA virus viral particle of claim 31, wherein the recombinant DNA virus viral particle is an AAV viral particle, and wherein the RPS comprises a transcribed modified AAV inverted terminal repeat (ITR), wherein said transcribed modified AAV ITR:
(a) comprises a transcribed functional Rep-Binding Element (RBE);
(b) lacks either a transcribed terminal resolution site (TRS), or a transcribed reverse complement TRS (rcTRS), or both; and
(c) comprises a transcribed D region sequence.

52. The recombinant DNA virus viral particle of claim 51, wherein the transcribed modified AAV ITR is modified from a transcribed wild-type flip or flop ITR.

53. The recombinant DNA virus viral particle of claim 52, wherein said wild-type flop ITR has the nucleotide sequence of SEQ ID NO: 1.

54. The recombinant DNA virus viral particle of claim 52, wherein the wild-type flip or flop ITR is from AAV1, AAV2, AAV3A, AAV3B, AAV4, AAV5, AAV6, AAV7, AAVrh74, AAV8, AAV9, AAV10, AAV11, AAV12, or AAV13.

55. The recombinant DNA virus viral particle of claim 51, wherein said transcribed modified AAV ITR has the nucleotide sequence of SEQ ID NO: 3.

56. The recombinant DNA virus viral particle of claim 31, wherein the RPS comprises an MS2 sequence, an PP7 binding site, or a com binding site, and the RPS-interacting molecule comprises an RPS-interacting protein (RPSIP) capable of binding directly or indirectly to the RPS.

57. The recombinant DNA virus viral particle of claim 56, wherein:
(a) the RPS comprises the MS2 sequence, and the RPSIP comprises a bacteriophage MS2 coat protein (MCP);
(b) the RPS comprises the PP7 binding site, and the RPSIP comprises a PP7 bacteriophage coat protein (PCP); or,
(c) The RPS comprises the com binding site, and the RPSIP comprises a phage COM protein (COM).

58. The recombinant DNA virus viral particle of claim 56, wherein the RPSIP is associated directly or indirectly with a protein component of the viral packaging system for the DNA virus viral particle.

59. The RNA sequence of claim 58, wherein the protein component of the viral packaging system for the DNA virus viral particle comprises Rep78 and/or Rep68 of adeno-associated virus 2 (AAV2), or an assembly-activating protein (AAP).

60. The recombinant DNA virus viral particle of claim 31, wherein the RPS-interacting molecule is or comprises Rep78, Rep68, Rep52, and/or Rep40.

61. The recombinant DNA virus viral particle of claim 31, wherein the Rep78 and/or the Rep68 comprises a Y156F mutation.

62. The recombinant DNA virus viral particle of claim 31, wherein said RNA sequence is a single-stranded RNA less than about 8,900 nucleotides in length, less than about 8,000 nucleotides in length, less than about 7,000 nucleotides in length, less than about 6,000 nucleotides in length, less than about 5,200 nucleotides in length, less than about 4,000 nucleotides in length, less than about 3,000 nucleotides in length, less than about 2,000 nucleotides in length, about 4,700-5,200 nucleotides in length, about 4,700-5,000 nucleotide in length, about 4,700-4,800 nucleotides in length, or about 4,700 nucleotides in length.

63. The recombinant DNA virus viral particle of claim 31, wherein said RNA sequence of interest is an RNA coding sequence for a gene of interest (GOI), a protein-encoding RNA, a non-coding functional RNA, or a precursor thereof.

64. The recombinant DNA virus viral particle of claim 63, wherein the gene of interest (GOI) encodes a protein, an enzyme, a structural protein, an mRNA, a non-coding RNA (ncRNA), an siRNA, a piRNA, a short hairpin RNA or shRNA, a microRNA (miRNA) or a precursor thereof, a ribosomal RNA (rRNA), an antisense sequence or oligonucleotide (ASO), an RNA component of a CRISPR-Cas system, an rRNA, a tRNA, a snoRNA, a snRNA, an exRNA, a scaRNA, a lncRNA, an X inactive specific transcript (Xist), or a HOX transcript antisense RNA (HOTAIR).

65. The recombinant DNA virus viral particle of claim 64, wherein said protein is a fluorescent protein, a therapeutic protein, an antigen protein, or a gene-editing protein.

66. The recombinant DNA virus viral particle of claim 64, wherein said enzyme is a Cre protein, or a CRISPR/Cas effector enzyme.

67. The recombinant DNA virus viral particle of claim 64, wherein said RNA component of the CRISPR-Cas system includes a guide RNA (gRNA), a CRISPR RNA (crRNA), and/or a tracr RNA.

68. The recombinant DNA virus viral particle of claim 63, wherein the protein-encoding RNA encodes a therapeutic protein, an antigen protein, or a gene-editing protein.

69. The recombinant DNA virus viral particle of claim 68, wherein the gene-editing protein is a CRISPR/Cas effector enzyme, a ZFN protein, or a TALEN protein.

70. The recombinant DNA virus viral particle of claim 63, wherein the non-coding functional RNA is a transfer RNA (tRNA), a ribosomal RNA (rRNA), a small interfering RNA (siRNA), a short hairpin RNA (shRNA), an antisense RNA, an antisense oligonucleotide, a micro RNA (miRNA), or an RNA component of a CRISPR-Cas system.

71. The recombinant DNA virus viral particle of claim 70, wherein the CRISPR-Cas system comprises Cas9, Cas12, or Cas13.

72. The recombinant DNA virus viral particle of claim 70, wherein the RNA component of the CRISPR-Cas system comprises a guide RNA (gRNA), a CRISPR RNA (crRNA), and/or a tracr RNA.

73. The recombinant DNA virus viral particle of claim 31, wherein said RNA sequence further comprises:
(1) a transcribed transcription enhancer;
(2) a transcribed intron sequence or exon sequence;
(3) a 5′ UTR sequence;
(4) a 3′ UTR sequence;
(5) a polyA sequence, or a polyadenylation (polyA) signal sequence, and optionally a GU-rich region downstream of the polyA signal sequence;
(6) a posttranscriptional regulatory element or sequence; and/or,
(7) a transcription termination sequence.

74. The recombinant DNA virus viral particle of claim 73, wherein said RNA sequence comprises, in 5′ to 3′ orientation, the RSI, an optional transcribed woodchuck hepatitis virus posttranscriptional regulatory element (WPRE) sequence, the RPS, and a polyA sequence or a polyA signal sequence.

75. The recombinant DNA virus viral particle of claim 73, wherein the RNA sequence comprises an RPS located 3′ to the posttranscriptional regulatory element or sequence, and 5′ to the polyA sequence or the polyA signal sequence.

76. The recombinant DNA virus viral particle of claim 31, wherein a DNA sequence encoding said RNA sequence, or a reverse complement of said DNA sequence, has reduced, diminished, or no capacity of being packaged into the DNA virus viral particle.

77. The recombinant DNA virus viral particle of claim 76, wherein the recombinant DNA virus viral particle is an AAV viral particle, and wherein said DNA sequence or the reverse complement thereof lacks a DNA packaging signal for AAV packaging.

78. The recombinant DNA virus viral particle of claim 77, wherein said DNA packaging signal comprises a functional AAV ITR.

79. A method of producing a ribonucleotide (RNA) sequence capable of being packaged into a DNA virus viral particle, the method comprising operably linking:
(1) an RNA sequence of interest (RSI); and,
(2) an RNA-packaging signal (RPS) capable of binding directly or indirectly to an RPS-interacting molecule that facilitates packaging of the RNA sequence into the DNA virus viral particle,
thereby producing the RNA sequence.

80. A method of producing a recombinant DNA virus viral particle comprising a ribonucleotide (RNA) sequence packaged within the protein shell of a DNA virus viral particle, wherein the RNA sequence is capable of being packaged into the DNA virus viral particle, wherein the RNA sequence comprises:
(1) an RNA sequence of interest (RSI); and,
(2) an RNA-packaging signal (RPS) capable of binding directly or indirectly to an RPS-interacting molecule that facilitates packaging of the RNA sequence into the DNA virus viral particle,
the method comprising contacting the RPS-interacting molecule with the RNA sequence to facilitate the packaging of the RNA sequence into the protein shell of the DNA virus viral particle, thereby producing the recombinant DNA virus viral particle.

81. A host cell comprising the population of recombinant DNA virus viral particles of claim 34.
